(19) United States
(12) Patent Application Publication
(10) Pub. No.: US 2010/0249071 A1
(43) Pub. Date: Sep. 30, 2010

(54) Modulators of S1P and Methods of Making And Using

(75) Inventors: Lynne Canne Bannen, Lucerne, CA (US); Diva Sze-Ming Chan, Emeryville, CA (US); Morrison B. Mac, San Francisco, CA (US); Stephanie Ng, Palo Alto, CA (US); John M. Nuss, Danville, CA (US); Yong Wang, Foster City, CA (US); Wei Xu, Danville, CA (US)

Correspondence Address: MCDONNELL BOEHNEN HULBERT @ BERGHOFF LLP, 300 SOUTH WACKER DRIVE, SUITE 3100, CHICAGO, IL, 60606, US

(73) Assignee: Exelixis, Inc., South San Francisco, CA (US)

(21) Appl. No.: 12/731,997

(22) Filed: Mar. 25, 2010

Related U.S. Application Data
(60) Provisional application No. 61/211,400, filed on Mar. 30, 2009.

(51) Int. Cl. A61K 31/4439; C07D 417/04; C07D 513/04; A61K 31/433; C07D 285/12; C07D 417/12; C07F 9/09; A61K 31/675; C07D 417/14; A61K 31/506; A61P 37/06; A61P 17/06; A61P 19/02; A61P 29/00; A61P 19/10

(52) U.S. Cl. 514/92; 546/268.7; 514/342; 548/137; 514/363; 548/136; 548/119; 544/331; 514/275

(57) ABSTRACT

The invention is directed to Compounds of Formula I:

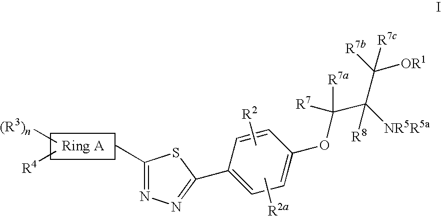

as well as methods of making and using the compounds.

### BACKGROUND OF THE INVENTION

1. Field of the Invention

This invention relates to the field of agonists of Sphingosine 1-Phosphate Type 1 Receptor (S1P1R or S1P1) and/or Type 5 Receptor (S1P5R or S1P5), and methods of their use.

2. Summary of the Related Art

Sphingosine 1-phosphate (S1P) is a biologically active lysophospholipid that serves as a key regulator of cellular differentiation and survival. Circulation of mature lymphocytes between blood and secondary lymphoid tissues plays an important role in the immune system. Agonism of S1P1R has been shown to lead to the sequestration of peripheral lymphocytes into secondary lymphoid tissue. Such sequestration of lymphocytes has been shown to result in immunosuppressive activity in animal models. Known S1P1 receptor agonists, such as FTY720, have been shown to markedly decrease peripheral blood lymphocytes through the sequestration of lymphocytes into secondary lymphoid tissues. Potent agonists of the S1P1 receptor are thought to induce long-term down-regulation of S1P1 on lymphocytes, thereby inhibiting the migration of lymphocytes toward S1P. The consequential decrease in trafficking and infiltration of antigen-specific T cells provides a means of immunomodulating activity that can be useful in the treatment of various immune-related conditions such as graft versus host disease and autoimmune diseases such as multiple sclerosis, rheumatoid arthritis, and systemic lupus erythematosis. Therefore, agonists of S1P1R are potentially useful immunosuppressants for the treatment of a variety of autoimmune conditions.

The myelin sheath that surrounds neural cell axons is required to insulate neural axons and allow rapid movement of electrical impulses through the myelinated nerve fiber. Demyelination, or loss of the integrity of the myelin sheath is the hallmark of autoimmune neurodegenerative diseases, including multiple sclerosis. The myelin sheath in the central nervous system is produced by oligodendrocytes. Mature, myelin-producing oligodendrocytes express S1P receptor transcripts in relative abundance of S1P5>S1P3>S1P1, with undetectable levels of S1P4. Fingolimod (FTY720), a sphingosine-1-phosphate (S1P) analogue that has been used successfully in clinical trials as a systemic immunomodulatory therapy for multiple sclerosis, readily accesses the central nervous system. In vitro studies using either mature adult human oligodendrocytes or oligodendrocyte precursor cells demonstrate that fingolomid or S1P has significant effects on oligodendrocyte process elongation and retraction as well as on cell survival and apoptosis. These studies further demonstrated that the effects seen were, at least in part, due to S1P5. These results indicate that S1P5 may play an important role in the beneficial effect observed with fingolomid treatment of multiple sclerosis.

Sphingosine-1-phosphate (S1P) has been shown to regulate the migration of osteoclast precursors, demonstrating a role in bone mineral homeostasis and suggesting a role in treating bone-destroying disorders such as rheumatoid arthritis and osteoporosis (Nature 2009, 458(7237), 524-528).

S1P3 has been associated with acute toxicty and bradycardia in rodents (Hale, et. al. Bioorganic & Med Chem Lett., 2004, 14(13), 3501-3505; J. Pharmacol. Exp. Ther. 2004, 309(2), 758-768; J. Med Chem 2005, 48(20), 6168-6173; J. Biol. Chem. 2004, 279(14), 13839-13848). Therefore agonists which are selective for S1P1 and/or S1P5, without being active for S1P3, are desirable.

### SUMMARY OF THE INVENTION

The following only summarizes certain aspects of the invention and is not intended to be limiting in nature. These aspects and other aspects and embodiments are described more fully below. All references cited in this specification are hereby incorporated by reference in their entirety. In the event of a discrepancy between the express disclosure of this specification and the references incorporated by reference, the express disclosure of this specification shall control.

The invention provides compounds that are agonists of S1P1 and/or S1P5 and that are useful in the treatment of graft versus host disease and autoimmune diseases, such as multiple sclerosis, rheumatoid arthritis, psoriasis, inflammatory bowel disease, and systemic lupus erythematosis, in mammals. This invention also provides methods of making the compound, methods of using such compounds in the treatment of graft versus host disease and autoimmune diseases, especially in humans, and to pharmaceutical compositions containing such compounds.

A first aspect of the invention provides a compound of Formula I:

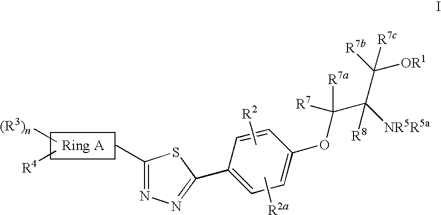

or a stereoisomer or mixture of isomers thereof and optionally as a pharmaceutically acceptable salt thereof, where
R¹ is hydrogen or —P(O)(OR⁶)₂;
R² and R^{2a} are independently hydrogen, cyano, halo, alkyl, haloalkyl, alkoxy, or haloalkoxy;
Ring A is phenyl, 5-membered heteroaryl, 6-membered heteroaryl, or 8-10-membered heteroaryl;
each R³ is independently cyano, halo, alkyl, haloalkyl, hydroxy, alkoxy, haloalkoxy, alkoxyalkyloxy, cycloalkyloxy, heterocycloalkyloxy, nitro, phenyl, amino, alkylamino, dialkylamino, aminocarbonyl, alkylcarbonylamino, or alkoxycarbonylamino;
n is 0, 1, or 2;
R⁴ is hydrogen, alkyl, cyano, halo, or haloalkyl;
R⁵ is hydrogen or alkyl;
R^{5a} is hydrogen, alkyl, alkylcarbonyl, or alkoxycarbonyl;
each R⁶ is independently hydrogen or alkyl;
R⁷, R^{7a}, R^{7b}, and R^{7c} are independently hydrogen or alkyl; and
R⁸ is hydrogen, alkyl, or hydroxyalkyl.

In a second aspect, the invention is directed to a pharmaceutical composition which comprises 1) a compound of Formula I or a single stereoisomer or mixture of isomers thereof, optionally as a pharmaceutically acceptable salt thereof and 2) a pharmaceutically acceptable carrier, excipient, or diluent.

In a third aspect, the invention provides a method for treating a disease, disorder, or syndrome which method comprises administering to a patient a therapeutically effective amount of a compound of Formula I or a single stereoisomer or mixture of isomers thereof, optionally as a pharmaceutically acceptable salt or solvate thereof, or a pharmaceutical composition comprising a therapeutically effective amount of a compound of Formula I or a single stereoisomer or mixture of isomers thereof, optionally as a pharmaceutically acceptable salt or solvate thereof, and a pharmaceutically acceptable carrier, excipient, or diluent.

In a fourth aspect, the Invention is directed to a method of making a Compound of the Invention which method comprises:

(a) reacting an intermediate of formula 116, or a salt thereof:

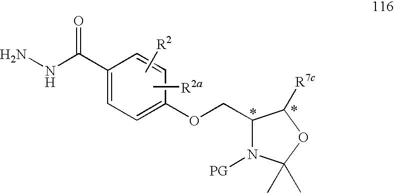

where PG is a nitrogen-protecting group, and R², R^{2a}, and R^{7c} are as defined in the Summary of the Invention for a Compound of Formula I; with an intermediate of formula 117:

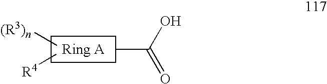

where n, Ring A, R³, and R⁴ are as defined in the Summary of the Invention for a Compound of Formula I, followed by treatment with Lawesson's reagent, to yield an intermediate of formula 118:

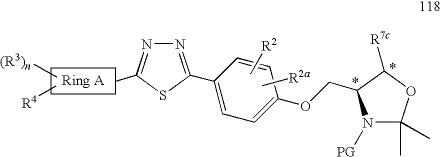

and followed by deprotection and ring opening to yield a Compound of the Invention of Formula I(g):

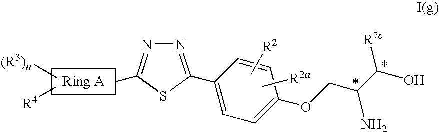

and optionally separating individual isomers; and optionally modifying any of the R², R^{2a}, R³, R⁴, and R^{7c} groups; and optionally forming a pharmaceutically acceptable salt, hydrate, solvate or combination thereof; or

(b) reacting an intermediate of formula 119, or a salt thereof:

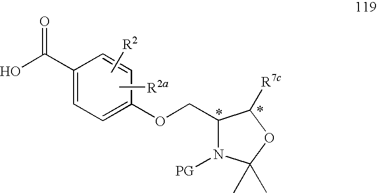

where PG is a nitrogen-protecting group, and R², R^{2a}, and R^{7c} are as defined in the Summary of the Invention for a Compound of Formula I; with an intermediate of formula 10:

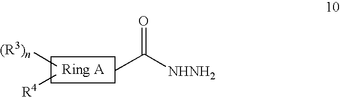

where n, Ring A, R³, and R⁴ are as defined in the Summary of the Invention for a Compound of Formula I, to yield an intermediate of formula 120:

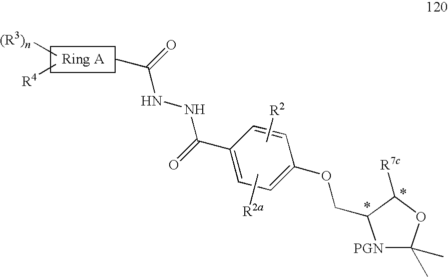

followed by treatment with Lawesson's reagent, deprotection, and ring opening, to yield a Compound of formula I(g)

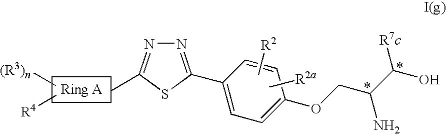

and optionally separating individual isomers; and optionally modifying any of the R², R^{2a}, R³, R⁴, and R^{7c} groups; and optionally forming a pharmaceutically acceptable salt, hydrate, solvate or combination thereof.

### DETAILED DESCRIPTION OF THE INVENTION

### Abbreviations and Definitions

The following abbreviations and terms have the indicated meanings throughout:

| Abbreviation | Meaning |
|---|---|
| br | broad |
| ° C. | degrees Celsius |
| conc. | concentrated |
| d | doublet |
| dd | doublet of doublet |
| dt | doublet of triplet |
| DCM | dichloromethane |
| DMA | N,N-dimethylacetamide |
| DMF | N,N-dimethylformamide |
| DMSO | dimethyl sulfoxide |
| EDCI | 1-(3-dimethylaminopropyl)-3-ethylcarbodiimide |
| EI | Electron Impact ionization |
| EtOAc | ethyl acetate |
| EtOH | ethanol |
| g | gram(s) |
| h or hr | hour(s) |
| HOBt | N-hydroxybenzotriazole |
| HPLC | high pressure liquid chromatography |
| L | liter(s) |
| M | molar or molarity |
| m | Multiplet |
| MCPBA | meta-chloroperoxybenzoic acid |
| MeOH | methanol |
| mg | milligram(s) |
| MHz | megahertz (frequency) |
| Min | minute(s) |
| mL | milliliter(s) |
| μL | microliter(s) |
| μM | Micromole(s) or micromolar |
| mM | Millimolar |
| mmol | millimole(s) |
| mol | mole(s) |
| MS | mass spectral analysis |
| N | normal or normality |
| nM | Nanomolar |
| NMR | nuclear magnetic resonance spectroscopy |
| q | Quartet |
| rt, RT | Room temperature |
| s | Singlet |
| t or tr | Triplet |
| THF | tetrahydrofuran |

The symbol “—” means a single bond, “═” means a double bond, “≡” means a triple bond, [symbol] means a single or double bond. The symbol [symbol] refers to a group on a double-bond as occupying either position on the terminus of a double bond to which the symbol is attached; that is, the geometry, E- or Z—, of the double bond is ambiguous. When a group is depicted removed from its parent formula, the [symbol] symbol will be used at the end of the bond which was theoretically cleaved in order to separate the group from its parent structural formula.

When chemical structures are depicted or described, unless explicitly stated otherwise, all carbons are assumed to have hydrogen substitution to conform to a valence of four. For example, in the structure on the left-hand side of the schematic below there are nine hydrogens implied. The nine hydrogens are depicted in the right-hand structure. Sometimes a particular atom in a structure is described in textual formula as having a hydrogen or hydrogens as substitution (expressly defined hydrogen), for example, —CH₂CH₂—. It is understood by one of ordinary skill in the art that the aforementioned descriptive techniques are common in the chemical arts to provide brevity and simplicity to description of otherwise complex structures.

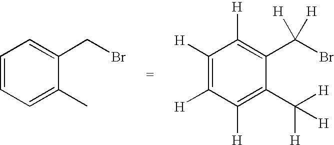

If a group “R” is depicted as “floating” on a ring system, as for example in the formula:

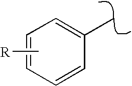

then, unless otherwise defined, a substituent “R” may reside on any atom of the ring system, assuming replacement of a depicted, implied, or expressly defined hydrogen from one of the ring atoms, so long as a stable structure is formed.

If a group “R” is depicted as floating on a fused ring system, as for example in the formulae:

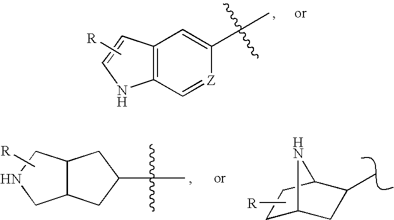

then, unless otherwise defined, a substituent “R” may reside on any atom of the fused ring system, assuming replacement of a depicted hydrogen (for example the —NH— in the formula above), implied hydrogen (for example as in the formula above, where the hydrogens are not shown but understood to be present), or expressly defined hydrogen (for example where in the formula above, “Z” equals ═CH—) from one of the ring atoms, so long as a stable structure is formed. In the example depicted, the “R” group may reside on either the 5-membered or the 6-membered ring of the fused ring system.

When a group “R” is depicted as existing on a ring system containing saturated carbons, as for example in the formula:

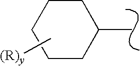

where, in this example, “y” can be more than one, assuming each replaces a currently depicted, implied, or expressly defined hydrogen on the ring; then, unless otherwise defined, where the resulting structure is stable, two “R's” may reside on the same carbon. A simple example is when R is a methyl group; there can exist a geminal dimethyl on a carbon of the depicted ring (an “annular” carbon). In another example, two R's on the same carbon, including that carbon, may form a ring, thus creating a spirocyclic ring (a “spirocyclyl” group) structure with the depicted ring as for example in the formula:

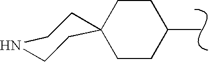

Although all moieties are generally referred to as their monovalent form (e.g., alkyl, aryl), those skilled in the art will understand from the context and standard valence rules when di-, tri-, etc., valent radicals are intended. So, for example, alkyl can refer to a monovalent alkyl radical or a divalent radical (i.e., alkylene).

“Administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., surgery, radiation, and chemotherapy, etc.), “administration” and its variants are each understood to include concurrent and sequential introduction of the compound or prodrug thereof and other agents.

“Alkenyl” means a linear hydrocarbon radical of two to six carbon atoms or a branched hydrocarbon radical of three to 6 carbon atoms which radical contains at least one double bond, e.g., ethenyl, propenyl, 1-but-3-enyl, and 1-pent-3-enyl, and the like.

“Alkoxy” means an —OR group where R is alkyl group as defined herein. Examples include methoxy, ethoxy, propoxy, isopropoxy, and the like.

“Alkoxyalkyl” means an alkyl group, as defined herein, substituted with at least one, specifically one, two, or three, alkoxy groups as defined herein. Representative examples include methoxymethyl and the like.

“Alkoxyalkyloxy” means an —OR group where R is alkoxyalkyl as defined herein.

“Alkoxycarbonyl” means a —C(O)R group where R is alkoxy, as defined herein.

“Alkoxycarbonylamino” means an —NHR group where R is alkoxycarbonyl as defined herien.

“Alkyl” means a linear saturated hydrocarbon radical of one to six carbon atoms or a branched saturated hydrocarbon radical of three to 6 carbon atoms, e.g., methyl, ethyl, propyl, 2-propyl, butyl (including all isomeric forms), or pentyl (including all isomeric forms), and the like.

“Alkylamino” means an —NHR group where R is alkyl, as defined herein.

“Alkylaminoalkyl” means an alkyl group substituted with one or two alkylamino groups, as defined herein.

“Alkylcarbonyl” means a —C(O)R group where R is alkyl, as defined herein.

“Alkylcarbonylamino” means an —NHR group where R is alkylcarbonyl, as defined herein.

“Alkylsulfonyl” means an —S(O)₂R group where R is alkyl, as defined herein, e.g. methylsulfonyl, isopropylsulfonyl.

“Alkynyl” means a linear hydrocarbon radical of two to six carbon atoms or a branched hydrocarbon radical of three to 6 carbon atoms which radical contains at least one triple bond, e.g., ethynyl, propynyl, butynyl, pentyn-2-yl and the like.

“Amino” means —NH₂.

“Aminocarbonyl” means a —C(O)NH₂ group.

“Aryl” means a six- to fourteen-membered, mono- or bi-carbocyclic ring, wherein the monocyclic ring is aromatic and at least one of the rings in the bicyclic ring is aromatic. Unless stated otherwise, the valency of the group may be located on any atom of any ring within the radical, valency rules permitting. Representative examples include phenyl, naphthyl, and indanyl, and the like.

“Arylalkyl” means an alkyl radical, as defined herein, substituted with one or two aryl groups, as defined herein, e.g., benzyl and phenethyl, and the like.

“Carboxy” means a —C(O)OH group.

“Cycloalkyl” means a monocyclic or fused bicyclic, saturated or partially unsaturated (but not aromatic), hydrocarbon radical of three to ten carbon ring atoms. Fused bicyclic hydrocarbon radical includes bridged ring systems. Unless stated otherwise, the valency of the group may be located on any atom of any ring within the radical, valency rules permitting. One or two ring carbon atoms may be replaced by a —C(O)—, —C(S)—, or —C(═NH)— group. More specifically, the term cycloalkyl includes, but is not limited to, cyclopropyl, cyclobutyl, cyclopentyl, cyclohexyl, cyclohexyl, or cyclohex-3-enyl, and the like.

“Cycloalkyloxy” means an —OR group where R is cycloalkyl as defined herein.

“Dialkylamino” means a —NRR′ radical where R and R′ are alkyl as defined herein, or an N-oxide derivative, or a protected derivative thereof, e.g., dimethylamino, diethylamino, N,N-methylpropylamino or N,N-methylethylamino, and the like.

“Fused ring” means a polycyclic ring that contains bridged or fused rings; that is, where two rings have more than one shared atom in their ring structures. In this application, fused ring systems are not necessarily all aromatic ring systems. Typically, but not necessarily, fused rings share a vicinal set of atoms, for example naphthalene or 1,2,3,4-tetrahydro-naphthalene. A spiro ring system is not a fused ring system by this definition, but fused ring systems of the invention may themselves have spiro rings attached thereto via a single ring atom of the fused ring system. In some examples, as appreciated by one of ordinary skill in the art, two adjacent groups on an aromatic system may be fused together to form a ring structure. The fused ring structure may contain heteroatoms and may be optionally substituted with one or more groups. It should additionally be noted that saturated carbons of such fused groups (i.e. saturated ring structures) can contain two substitution groups.

“Halogen” or “halo” refers to fluorine, chlorine, bromine and iodine.

“Haloalkoxy” means an —OR′ group where R′ is haloalkyl as defined herein, e.g., trifluoromethoxy or 2,2,2-trifluoroethoxy, and the like.

“Haloalkyl” mean an alkyl group substituted with one or more halogens, specifically one to five halo atoms, e.g., trifluoromethyl, 2-chloroethyl, and 2,2-difluoroethyl, and the like.

“Heteroaryl” means a monocyclic, fused bicyclic, or fused tricyclic, radical of 5 to 14 ring atoms containing one or more, specifically one, two, three, or four ring heteroatoms independently selected from —O—, —S(O)ₙ— (n is 0, 1, or 2), —N—, —N(R^{x})—, and the remaining ring atoms being carbon, wherein the ring comprising a monocyclic radical is aromatic and wherein at least one of the fused rings comprising a bicyclic or tricyclic radical is aromatic. One or two ring carbon atoms of any nonaromatic rings comprising a bicyclic or tricyclic radical may be replaced by a —C(O)—, —C(S)—, or —C(═NH)— group. R^{x} is hydrogen, alkyl, hydroxy, alkoxy, acyl, or alkylsulfonyl. Fused bicyclic radical includes bridged ring systems. Unless stated otherwise, the valency may be located on any atom of any ring of the heteroaryl group, valency rules permitting. When the point of valency is located on the nitrogen, R^{x} is absent. More specifically, the term heteroaryl includes, but is not limited to, 1,2,4-triazolyl, 1,3,5-triazolyl, phthalimidyl, pyridinyl, pyrrolyl, imidazolyl, thienyl, furanyl, indolyl, 2,3-dihydro-1H-indolyl (including, for example, 2,3-dihydro-1H-indol-2-yl or 2,3-dihydro-1H-indol-5-yl, and the like), isoindo indolinyl, isoindolinyl, benzimidazolyl, benzodioxol-4-yl, benzofuranyl, cinnolinyl, indolizinyl, naphthyridin-3-yl, phthalazin-3-yl, phthalazin-4-yl, pteridinyl, purinyl, quinazolinyl, quinoxalinyl, tetrazoyl, pyrazolyl, pyrazinyl, pyrimidinyl, pyridazinyl, oxazolyl, isooxazolyl, oxadiazolyl, benzoxazolyl, quinolinyl, isoquinolinyl, tetrahydroisoquinolinyl (including, for example, tetrahydroisoquinolin-4-yl or tetrahydroisoquinolin-6-yl, and the like), pyrrolo[3,2-c]pyridinyl (including, for example, pyrrolo[3,2-c]pyridin-2-yl or pyrrolo[3,2-c]pyridin-7-yl, and the like), benzopyranyl, thiazolyl, isothiazolyl, thiadiazolyl, benzothiazolyl, benzothienyl, and the derivatives thereof, or N-oxide or a protected derivative thereof

“Heteroatom” refers to O, S, N, and P.

“Heterocycloalkyl” means a saturated or partially unsaturated (but not aromatic) monocyclic group of 3 to 8 ring atoms or a saturated or partially unsaturated (but not aromatic) fused bicyclic group of 5 to 12 ring atoms in which one or more, specifically one, two, three, or four ring heteroatoms independently selected from O, S(O)ₙ (n is 0, 1, or 2), N, N(R^{y}) (where R^{y} is hydrogen, alkyl, hydroxy, alkoxy, acyl, or alkylsulfonyl), the remaining ring atoms being carbon. One or two ring carbon atoms may be replaced by a —C(O)—, —C(S)—, or —C(═NH)— group. Fused bicyclic radical includes bridged ring systems. Unless otherwise stated, the valency of the group may be located on any atom of any ring within the radical, valency rules permitting. When the point of valency is located on a nitrogen atom, R^{y} is absent. More specifically the term heterocycloalkyl includes, but is not limited to, azetidinyl, pyrrolidinyl, 2-oxopyrrolidinyl, 2,5-dihydro-1H-pyrrolyl, piperidinyl, 4-piperidonyl, morpholinyl, piperazinyl, 2-oxopiperazinyl, tetrahydropyranyl, 2-oxopiperidinyl, thiomorpholinyl, thiamorpholinyl, perhydroazepinyl, pyrazolidinyl, imidazolinyl, imidazolidinyl, dihydropyridinyl, tetrahydropyridinyl, oxazolinyl, oxazolidinyl, isoxazolidinyl, thiazolinyl, thiazolidinyl, quinuclidinyl, isothiazolidinyl, octahydroindolyl, octahydroisoindolyl, decahydroisoquinolyl, tetrahydrofuryl, and tetrahydropyranyl, and the derivatives thereof and N-oxide or a protected derivative thereof

“Heterocycloalkyloxy” means an —OR group where R is hetero cycloalkyl as defined herein.

“Hydroxyalkyl” means an alkyl group substituted with at least one, in another example with one, two, or three, hydroxy groups.

“Spirocyclyl” or “spirocyclic ring” refers to a ring originating from a particular annular carbon of another ring. For example, as depicted below, a ring atom of a saturated bridged ring system (rings B and B′), but not a bridgehead atom, can be a shared atom between the saturated bridged ring system and a spirocyclyl (ring A) attached thereto. A spirocyclyl can be carbocyclic or heteroalicyclic.

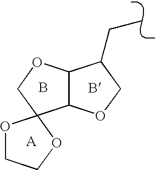

“Optional” or “optionally” means that the subsequently described event or circumstance may or may not occur, and that the description includes instances where said event or circumstance occurs and instances in which it does not. One of ordinary skill in the art would understand that with respect to any molecule described as containing one or more optional substituents, only sterically practical and/or synthetically feasible compounds are meant to be included. “Optionally substituted” refers to all subsequent modifiers in a term. So, for example, in the term “optionally substituted arylC₁₋₈ alkyl,” optional substitution may occur on both the “C₁₋₈ alkyl” portion and the “aryl” portion of the molecule may or may not be substituted.

“Metabolite” refers to the break-down or end product of a compound or its salt produced by metabolism or biotransformation in the animal or human body; for example, biotransformation to a more polar molecule such as by oxidation, reduction, or hydrolysis, or to a conjugate (see Goodman and Gilman, “The Pharmacological Basis of Therapeutics” 8.sup.th Ed., Pergamon Press, Gilman et al. (eds), 1990 for a discussion of biotransformation). As used herein, the metabolite of a compound of the invention or its salt may be the biologically active form of the compound in the body. In one example, a prodrug may be used such that the biologically active form, a metabolite, is released in vivo. In another example, a biologically active metabolite is discovered serendipitously, that is, no prodrug design per se was undertaken. An assay for activity of a metabolite of a compound of the present invention is known to one of skill in the art in light of the present disclosure.

“Patient” for the purposes of the present invention includes humans and other animals, particularly mammals, and other organisms. Thus the methods are applicable to both human therapy and veterinary applications. In a specific embodiment the patient is a mammal, and in a more specific embodiment the patient is human.

A “pharmaceutically acceptable salt” of a compound means a salt that is pharmaceutically acceptable and that possesses the desired pharmacological activity of the parent compound. It is understood that the pharmaceutically acceptable salts are non-toxic. Additional information on suitable pharmaceutically acceptable salts can be found in Remington's Pharmaceutical Sciences, 17^{th} ed., Mack Publishing Company, Easton, Pa., 1985, which is incorporated herein by reference or S. M. Berge, et al., “Pharmaceutical Salts,” J. Pharm. Sci., 1977; 66:1-19 both of which are incorporated herein by reference.

Examples of pharmaceutically acceptable acid addition salts include those formed with inorganic acids such as hydrochloric acid, hydrobromic acid, sulfuric acid, nitric acid, phosphoric acid, and the like; as well as organic acids such as acetic acid, trifluoroacetic acid, propionic acid, hexanoic acid, cyclopentanepropionic acid, glycolic acid, pyruvic acid, lactic acid, oxalic acid, maleic acid, malonic acid, succinic acid, fumaric acid, tartaric acid, citric acid, benzoic acid, cinnamic acid, 3-(4-hydroxybenzoyl)benzoic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, p-toluenesulfonic acid, and salicylic acid and the like.

Examples of a pharmaceutically acceptable base addition salts include those formed when an acidic proton present in the parent compound is replaced by a metal ion, such as sodium, potassium, lithium, ammonium, calcium, magnesium, iron, zinc, copper, manganese, aluminum salts and the like. Specific salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins. Examples of organic bases include isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, tromethamine, N-methylglucamine, polyamine resins, and the like. Exemplary organic bases are isopropylamine, diethylamine, ethanolamine, trimethylamine, dicyclohexylamine, choline, and caffeine.“Platin(s),” and “platin-containing agent(s)” include, for example, cisplatin, carboplatin, and oxaliplatin.

“Prodrug” refers to compounds that are transformed (typically rapidly) in vivo to yield the parent compound of the above formulae, for example, by hydrolysis in blood. Common examples include, but are not limited to, ester and amide forms of a compound having an active form bearing a carboxylic acid moiety. Examples of pharmaceutically acceptable esters of the compounds of this invention include, but are not limited to, alkyl esters (for example with between about one and about six carbons) the alkyl group is a straight or branched chain. Acceptable esters also include cycloalkyl esters and arylalkyl esters such as, but not limited to benzyl. Examples of pharmaceutically acceptable amides of the compounds of this invention include, but are not limited to, primary amides, and secondary and tertiary alkyl amides (for example with between about one and about six carbons). Amides and esters of the compounds of the present invention may be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

“Stereoisomer” means any of two or more isomers containing the same atoms bonded to each other in an identical manner but differing from each other in the spatial arrangement of the atoms or groups of atoms. “Stereoisomer” includes, for example, an enantiomer, a geometric isomer, a diastereomer, a rotamer, cis-isomer, trans-isomer, and conformational isomer. The names and illustration used in this application to describe compounds of the invention, unless indicated otherwise, are meant to encompass all possible stereoisomers and any mixture, racemic or otherwise, thereof.

“Therapeutically effective amount” is an amount of a compound of the invention, that when administered to a patient, ameliorates a symptom of the disease. The amount of a compound of the invention which constitutes a “therapeutically effective amount” will vary depending on the compound, the disease state and its severity, the age of the patient to be treated, and the like. The therapeutically effective amount can be determined routinely by one of ordinary skill in the art having regard to their knowledge and to this disclosure.

“Treating” or “treatment” of a disease, disorder, or syndrome, as used herein, includes (i) preventing the disease, disorder, or syndrome from occurring in a human, i.e. causing the clinical symptoms of the disease, disorder, or syndrome not to develop in an animal that may be exposed to or predisposed to the disease, disorder, or syndrome but does not yet experience or display symptoms of the disease, disorder, or syndrome; (ii) inhibiting the disease, disorder, or syndrome, i.e., arresting its development; and (iii) relieving the disease, disorder, or syndrome, i.e., causing regression of the disease, disorder, or syndrome. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art.

“Yield” for each of the reactions described herein is expressed as a percentage of the theoretical yield.

### Embodiments of the Invention

The following paragraphs present a number of embodiments of compounds of the invention. In each instance the embodiment includes the recited compounds, as well as a single stereoisomer or mixture of stereoisomers thereof, as well as a pharmaceutically acceptable salt thereof.

One embodiment of the Invention (A) is directed to a Compound of Formula I where Ring A is phenyl, thienyl, pyridyl, pyrimidinyl, or imidazo[2,1-b]thiazolyl, each of which is substituted with (R³)ₙ and R⁴; and n, R³, and R⁴, all other groups are as defined in the Summary of the Invention for a Compound of Formula I. Another embodiment of the Invention (A1) is directed to a Compound of Formula I where Ring A is phenyl, pyridyl, or pyrimidinyl, each of which is substituted with (R³)ₙ and R⁴; and n, R³, R⁴, and all other groups are as defined in the Summary of the Invention for a Compound of Formula I. Another embodiment of the Invention (A2) is directed to a Compound of Formula I where Ring A is phenyl or pyridyl, each of which is substituted with (R³)ₙ and R⁴; and n, R³, R⁴, and all other groups are as defined in the Summary of the Invention for a Compound of Formula I.

Another embodiment of the Invention (A3) is directed to a Compound of Formula I where Ring A is phenyl substituted with (R³)ₙ and R⁴; and n, R³, R⁴, and all other groups are as defined in the Summary of the Invention for a Compound of Formula I.

Another embodiment of the Invention (A4) is directed to a Compound of Formula I where Ring A is a 6-membered heteroaryl substituted with (R³)ₙ and R⁴; and n, R³, R⁴, and all other groups are as defined in the Summary of the Invention for a Compound of Formula I. Another embodiment of the Invention (A5) is directed to a Compound of Formula I where Ring A is pyrimidinyl or pyridinyl, each of which is substituted with (R³)ₙ and R⁴; and n, R³, R⁴, and all other groups are as defined in the Summary of the Invention for a Compound of Formula I. Another embodiment of the Invention (A6) is directed to a Compound of Formula I where Ring A is pyridinyl substituted with (R³)ₙ and R⁴; and n, R³, R⁴, and all other groups are as defined in the Summary of the Invention for a Compound of Formula I.

Another embodiment of the Invention (A7) is directed to a Compound of Formula I where Ring A is 8-10-membered heteroaryl substituted with (R³)ₙ and R⁴; and n, R³, R⁴, and all other groups are as defined in the Summary of the Invention for a Compound of Formula I. Another embodiment of the Invention (A8) is directed to a Compound of Formula I where Ring A is imidazo[2,1-b]thiazolyl, n is 0, R⁴ is hydrogen, and all other groups are as defined in the Summary of the Invention for a Compound of Formula I.

In another embodiment of the Invention (B) is directed to a Compound of Formula I where n is 1 or 2; and all other groups are as defined in the Summary of the Invention for a Compound of Formula I or as defined in any of embodiments A-A8. In another embodiment of the Invention (B1) is directed to a Compound of Formula I where n is 1; and all other groups are as defined in the Summary of the Invention for a Compound of Formula I or as defined in any of embodiments A-A8. In another embodiment of the Invention (B2) is directed to a Compound of Formula I where n is 2; and all other groups are as defined in the Summary of the Invention for a Compound of Formula I or as defined in any of embodiments A-A8.

Another embodiment of the Invention (C) is directed to a Compound of Formula I where n is 1 or 2 and each R³ is independently cyano, halo, alkyl, haloalkyl, hydroxy, alkoxy, haloalkoxy, alkoxyalkyloxy, cycloalkyloxy, heterocycloalkyloxy, nitro, phenyl, amino, alkylamino, dialkylamino, aminocarbonyl, alkylcarbonylamino, or alkoxycarbonylamino; and all other groups are as defined in the Summary of the Invention for a Compound of Formula I or as defined in any of embodiments A-A8, and B-B2. Another embodiment of the Invention (C1) is directed to a Compound of Formula I where n is 1 or 2 and each R³ is independently cyano, halo, alkyl, haloalkyl, alkoxy, haloalkoxy, alkoxyalkyloxy, heterocycloalkyloxy, phenyl, alkylamino, dialkylamino, or aminocarbonyl; and all other groups are as defined in the Summary of the Invention for a Compound of Formula I or as defined in any of embodiments A-A8, and B-B2. Another embodiment of the Invention (C2) is directed to a Compound of Formula I where n is 1 or 2 and each R³ is independently cyano, bromo, chloro, fluoro, methyl, isobutyl, sec-butyl, trifluoromethyl, ethoxy, isopropoxy, n-propoxy, trifluoromethoxy, 2-methoxy-ethyloxy, oxetanyloxy, phenyl, isopropylamino, N-methyl-N-isopropylamino, or aminocarbonyl; and all other groups are as defined in the Summary of the Invention for a Compound of Formula I or as defined in any of embodiments A-A8, and B-B2.

Another embodiment of the Invention (C3) is directed to a Compound of Formula I where R⁴ is hydrogen, n is 1 or 2, and each R³ is independently cyano, halo, alkyl, haloalkyl, alkoxy, haloalkoxy, heterocycloalkyl, or alkylamino; and all other groups are as defined in the Summary of the Invention for a Compound of Formula I or as defined in any of embodiments A-A8, and B-B2. Another embodiment of the Invention (C3a) is directed to a Compound of Formula I where R⁴ is hydrogen, n is 1 or 2, and each R³ is independently cyano, bromo, chloro, methyl, trifluoromethyl, isopropoxy, oxetanyloxy, or isopropylamino; and all other groups are as defined in the Summary of the Invention for a Compound of Formula I or as defined in any of embodiments A-A8, and B-B2.

In another embodiment of the Invention (C4) is directed to a Compound of Formula I where R⁴ is hydrogen, n is 1, and R³ is alkylamino; and all other groups are as defined in the Summary of the Invention for a Compound of Formula I or as defined in any of embodiments A-A8, and B-B2. In another embodiment of the Invention (C5) is directed to a Compound of Formula I where R⁴ is hydrogen, n is 1, and R³ is isopropylamino; and all other groups are as defined in the Summary of the Invention for a Compound of Formula I or as defined in any of embodiments A-A8, and B-B2.

In another embodiment of the Invention (C6) is directed to a Compound of Formula I where R⁴ is hydrogen, n is 2, and one R³ is halo, cyano, alkyl, or haloalkyl, and the other R³ is alkoxy or alkylamino; and all other groups are as defined in the Summary of the Invention for a Compound of Formula I or as defined in any of embodiments A-A8, and B-B2. In another embodiment of the Invention (C7) is directed to a Compound of Formula I where R⁴ is hydrogen, n is 2, and one R³ is bromo, chloro, cyano, methyl, or trifluoromethyl, and the other R³ is ethoxy, isopropoxy, oxetanyloxy, or isopropylamino; and all other groups are as defined in the Summary of the Invention for a Compound of Formula I or as defined in any of embodiments A-A8, and B-B2. In another embodiment of the Invention (C8) is directed to a Compound of Formula I where R⁴ is hydrogen, n is 2, and one R³ is halo, cyano, alkyl, or haloalkyl and the other R³ is alkoxy; and all other groups are as defined in the Summary of the Invention for a Compound of Formula I or as defined in any of embodiments A-A8, and B-B2. In another embodiment of the Invention (C9) is directed to a Compound of Formula I where R⁴ is hydrogen, n is 2, and one R³ is bromo, chloro, cyano, methyl, or trifluoromethyl and the other R³ is isopropoxy; and all other groups are as defined in the Summary of the Invention for a Compound of Formula I or as defined in any of embodiments A-A8, and B-B2. In another embodiment of the Invention (C10) is directed to a Compound of Formula I where R⁴ is hydrogen, n is 2, and one R³ is halo or alkyl, and the other R³ is alkylamino; and all other groups are as defined in the Summary of the Invention for a Compound of Formula I or as defined in any of embodiments A-A8, and B-B2. In another embodiment of the Invention (C11) is directed to a Compound of Formula I where R⁴ is hydrogen, n is 2, and one R³ is chloro or methyl and the other R³ is isopropylamino; and all other groups are as defined in the Summary of the Invention for a Compound of Formula I or as defined in any of embodiments A-A8, and B-B2.

Another embodiment of the Invention (C12) is directed to a Compound of Formula I where R⁴ is hydrogen, n is 1 or 2, and each R³ is independently halo, cyano, alkyl, haloalkyl, alkoxy, heterocycloalkyloxy, alkoxyalkyloxy, alkylamino, or dialkylamino; and all other groups are as defined in the Summary of the Invention for a Compound of Formula I or as defined in any of embodiments A-A8, and B-B2. Another embodiment of the Invention (C13) is directed to a Compound of Formula I where R⁴ is hydrogen, n is 1 or 2, and each R³ is independently bromo, chloro, cyano, methyl, trifluoromethyl, ethoxy, isopropoxy, oxetanyloxy, 2-methoxy-ethyloxy, N-isopropyl-amino, or N-methyl-N-isopropyl-amino; and all other groups are as defined in the Summary of the Invention for a Compound of Formula I or as defined in any of embodiments A-A8, and B-B2.

In another embodiment of the Invention (C14) is directed to a Compound of Formula I where R⁴ is hydrogen, n is 1, and R³ is halo, alkoxy, alkylamino, or dialkylamino; and all other groups are as defined in the Summary of the Invention for a Compound of Formula I or as defined in any of embodiments A-A8, and B-B2. In another embodiment of the Invention (C15) is directed to a Compound of Formula I where R⁴ is hydrogen, n is 1, and R³ is chloro, isopropoxy, isopropylamino, or N-methyl-N-isopropyl-amino; and all other groups are as defined in the Summary of the Invention for a Compound of Formula I or as defined in any of embodiments A-A8, and B-B2.

In another embodiment of the Invention (C16) is directed to a Compound of Formula I where R⁴ is hydrogen, n is 2, and one R³ is halo, cyano, alkyl, or haloalkyl, and the other R³ is alkoxy, alkylamino, dialkylamino, alkoxyalkyloxy, or heterocycloalkyloxy; and all other groups are as defined in the Summary of the Invention for a Compound of Formula I or as defined in any of embodiments A-A8, and B-B2. In another embodiment of the Invention (C17) is directed to a Compound of Formula I where R⁴ is hydrogen, n is 2, and one R³ is bromo, chloro, cyano, methyl, or trifluoromethyl and the other R³ is ethoxy, isopropoxy, 2-methoxy-ethyloxy, oxetanyloxy, isopropylamino, or N-methyl-N-isopropylamino; and all other groups are as defined in the Summary of the Invention for a Compound of Formula I or as defined in any of embodiments A-A8, and B-B2.

In another embodiment of the Invention (C18) is directed to a Compound of Formula I where R⁴ is hydrogen, n is 2, and one R³ is halo, cyano, or alkyl and the other R³ is alkoxy; and all other groups are as defined in the Summary of the Invention for a Compound of Formula I or as defined in any of embodiments A-A8, and B-B2. In another embodiment of the Invention (C19) is directed to a Compound of Formula I where R⁴ is hydrogen, n is 2, and one R³ is bromo, chloro, cyano, or methyl and the other R³ is ethoxy or isopropoxy; and all other groups are as defined in the Summary of the Invention for a Compound of Formula I or as defined in any of embodiments A-A8, and B-B2. In another embodiment of the Invention (C20) is directed to a Compound of Formula I where R⁴ is hydrogen, n is 2, and one R³ is halo or alkyl, and the other R³ is alkylamino or dialkylamino; and all other groups are as defined in the Summary of the Invention for a Compound of Formula I or as defined in any of embodiments A-A8, and B-B2. In another embodiment of the Invention (C21) is directed to a Compound of Formula I where R⁴ is hydrogen, n is 2, and one R³ is chloro or methyl and the other R³ is isopropylamino or N-methyl-N-isopropylamino; and all other groups are as defined in the Summary of the Invention for a Compound of Formula I or as defined in any of embodiments A-A8, and B-B2. In another embodiment of the Invention (C22) is directed to a Compound of Formula I where R⁴ is hydrogen, n is 2, and one R³ is halo or cyano, and the other R³ is alkoxyalkyl or heterocycloalkyloxy; and all other groups are as defined in the Summary of the Invention for a Compound of Formula I or as defined in any of embodiments A-A8, and B-B2. In another embodiment of the Invention (C23) is directed to a Compound of Formula I where R⁴ is hydrogen, n is 2, and one R³ is chloro or cyano and the other R³ is 2-methoxy-ethyloxy or oxetanyloxy; and all other groups are as defined in the Summary of the Invention for a Compound of Formula I or as defined in any of embodiments A-A8, and B-B2.

Another embodiment of the Invention (D) is directed to a Compound of Formula I where R⁴ is hydrogen or halo; and all other groups are as defined in the Summary of the Invention for a Compound of Formula I or as defined in any of embodiments A-A8, B-B2, and C-C23. Another embodiment of the Invention (D1) is directed to a Compound of Formula I where R⁴ is hydrogen or chloro; and all other groups are as defined in the Summary of the Invention for a Compound of Formula I or as defined in any of embodiments A-A8, B-B2, and C-C23. Another embodiment of the Invention (D2) is directed to a Compound of Formula I where R⁴ is hydrogen; and all other groups are as defined in the Summary of the Invention for a Compound of Formula I or as defined in any of embodiments A-A8, B-B2, and C-C23.

Another embodiment of the Invention (E) is directed to a Compound of Formula I where R² is halo; and all other groups are as defined in the Summary of the Invention for a Compound of Formula I or as defined in any of embodiments A-A8, B-B2, C-C23, and D-D2. Another embodiment of the Invention (E1) is directed to a Compound of Formula I where R² is chloro; and all other groups are as defined in the Summary of the Invention for a Compound of Formula I or as defined in any of embodiments A-A8, B-B2, C-C23, and D-D2.

Another embodiment of the Invention (F) is directed to a Compound of Formula I where R^{2a} is hydrogen or halo; and all other groups are as defined in the Summary of the Invention for a Compound of Formula I or as defined in any of embodiments A-A8, B-B2, C-C23, D-D2, E, and E1. Another embodiment of the Invention (F1) is directed to a Compound of Formula I where R^{2a} is hydrogen, chloro, or fluoro; and all other groups are as defined in the Summary of the Invention for a Compound of Formula I or as defined in any of embodiments A-A8, B-B2, C-C23, D-D2, E, and E1.

Another embodiment of the Invention (G) is directed to a Compound of Formula I where R² and R^{2a} are halo; and all other groups are as defined in the Summary of the Invention for a Compound of Formula I or as defined in any of embodiments A-A8, B-B2, C-C23, and D-D2. Another embodiment of the Invention (G1) is directed to a Compound of Formula I where R² is chloro and R^{2a} is fluoro; and all other groups are as defined in the Summary of the Invention for a Compound of Formula I or as defined in any of embodiments A-A8, B-B2, C-C23, and D-D2. Another embodiment of the Invention (G2) is directed to a Compound of Formula I where R² is chloro and is located at the ortho-position with respect to the thiadiazolyl ring and R^{2a} is fluoro and is located para to the R² position; and all other groups are as defined in the Summary of the Invention for a Compound of Formula I or as defined in any of embodiments A-A8, B-B2, C-C23, and D-D2. Another embodiment of the Invention (G3) is directed to a Compound of Formula I where R² is chloro and is located at the meta-position with respect to the thiadiazolyl ring and R^{2a} is chloro or fluoro and is located meta to the R² position; and all other groups are as defined in the Summary of the Invention for a Compound of Formula I or as defined in any of embodiments A-A8, B-B2, C-C23, and D-D2.

Another embodiment of the Invention (H) is directed to a Compound of Formula I where zero, one, or two of R⁷, R^{7a}, R^{7b}, and R^{7c} is alkyl and the remaining are hydrogen; and all other groups are as defined in the Summary of the Invention for a Compound of Formula I or as defined in any of embodiments A-A8, B-B2, C-C23, D-D2, E, E1, F, F1, and G-G3. Another embodiment of the Invention (H1) is directed to a Compound of Formula I where R⁷, R^{7a}, R^{7b}, and R^{7c} are hydrogen; and all other groups are as defined in the Summary of the Invention for a Compound of Formula I or as defined in any of embodiments A-A8, B-B2, C-C23, D-D2, E, E1, F, F1, and G-G3. Another embodiment of the Invention (H2) is directed to a Compound of Formula I where R⁷, R^{7a}, and R^{7b} are hydrogen and R^{7c} is alkyl; and all other groups are as defined in the Summary of the Invention for a Compound of Formula I or as defined in any of embodiments A-A8, B-B2, C-C23, D-D2, E, E1, F, F1, and G-G3. Another embodiment of the Invention (H3) is directed to a Compound of Formula I where R⁷, R^{7a}, and R^{7b} are hydrogen and R^{7c} is methyl; and all other groups are as defined in the Summary of the Invention for a Compound of Formula I or as defined in any of embodiments A-A8, B-B2, C-C23, D-D2, E, E1, F, F1, and G-G3.

Another embodiment of the Invention (J) is directed to a Compound of Formula I where R⁸ is hydrogen; and all other groups are as defined in the Summary of the Invention for a Compound of Formula I or as defined in any of embodiments A-A8, B-B2, C-C23, D-D2, E, E1, F, F1, G-G3, and H-H3.

Another embodiment of the Invention (K) is directed to a Compound of Formula I where R⁵ is hydrogen and R^{5a} is hydrogen or alkoxycarbonyl; and all other groups are as defined in the Summary of the Invention for a Compound of Formula I or as defined in any of embodiments A-A8, B-B2, C-C23, D-D2, E, E1, F, F1, G-G3, H-H3, and J. Another embodiment of the Invention (K1) is directed to a Compound of Formula I where R⁵ is hydrogen and R^{5a} is hydrogen or tert-butoxycarbonyl; and all other groups are as defined in the Summary of the Invention for a Compound of Formula I or as defined in any of embodiments A-A8, B-B2, C-C23, D-D2, E, E1, F, F1, G-G3, H-H3, and J. Another embodiment of the Invention (K2) is directed to a Compound of Formula I where R⁵ and R^{5a} are hydrogen; and all other groups are as defined in the Summary of the Invention for a Compound of Formula I or as defined in any of embodiments A-A8, B-B2, C-C23, D-D2, E, E1, F, F1, G-G3, H-H3, and J.

Another embodiment of the Invention (L) is directed to a Compound of Formula I where R¹ is hydrogen; and all other groups are as defined in the Summary of the Invention for a Compound of Formula I or as defined in any of embodiments A-A8, B-B2, C-C23, D-D2, E, E1, F, F1, G-G3, H-H3, J, and K-K2.

Another embodiment of the Invention (M) is directed to a Compound of Formula I where R¹ is —P(O)(OR⁶)₂; and R⁶ and all other groups are as defined in the Summary of the Invention for a Compound of Formula I or as defined in any of embodiments A-A8, B-B2, C-C23, D-D2, E, E1, F, F1, G-G3, H-H3, J, and K-K2. Another embodiment of the Invention (M1) is directed to a Compound of Formula I where R¹ is —P(O)(OR⁶)₂; both R⁶ are hydrogen or both R⁶ are tert-butyl; and all other groups are as defined in the Summary of the Invention for a Compound of Formula I or as defined in any of embodiments A-A8, B-B2, C-C23, D-D2, E, E1, F, F1, G-G3, H-H3, J, and K-K2. Another embodiment of the Invention (M2) is directed to a Compound of Formula I where R¹ is —P(O)(OR⁶)₂; both R⁶ are hydrogen; and all other groups are as defined in the Summary of the Invention for a Compound of Formula I or as defined in any of embodiments A-A8, B-B2, C-C23, D-D2, E, E1, F, F1, G-G3, H-H3, J, and K-K2.

Another embodiment (N) of the Invention is directed to a Compound of Formula I according to Formula II

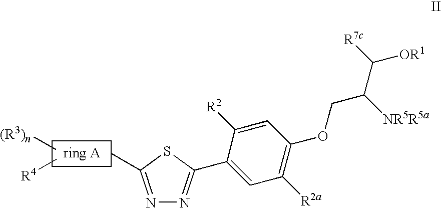

or a pharmaceutically acceptable salt thereof where n, R¹, R², R^{2a}, R³, R⁴, R⁵, R^{5a}, R^{7c}, and Ring A are as defined in the Summary of the Invention for a Compound of Formula I.

Another embodiment (N1) of the Invention is directed to a Compound of Formula II where n, R¹, R², R^{2a}, each R³ (independently of each other), R⁴, R⁵, R^{5a}, R^{7c}, and Ring A are as defined in any of embodiments A-A8, B-B2, C-C23, D-D2, E, E1, F, F1, G-G3, K-K2, L, and M-M2.

Another embodiment (N2) of the Invention is directed to a Compound of Formula II where Ring A is phenyl or 6-membered heteroaryl; and n, R¹, R², R^{2a}, each R³ (independently of each other), R⁴, R⁵, R^{5a}, and R^{7c} are as defined in the Summary of the Invention for a Compound of Formula I or as defined in any of embodiments B-B2, C-C23, D-D2, E, E1, F, F1, G-G3, K-K2, L, and M-M2. Another embodiment (N3) of the Invention is directed to a Compound of Formula II where Ring A is phenyl or pyridinyl; and n, R¹, R², R^{2a}, each R³ (independently of each other), R⁴, R⁵, R^{5a}, and R^{7c} are as defined in the Summary of the Invention for a Compound of Formula I or as defined in any of embodiments B-B2, C-C23, D-D2, E, E1, F, F1, G-G3, K-K2, L, and M-M2.

Another embodiment (N4) of the Invention is directed to a Compound of Formula II where R² and R^{2a} are halo; Ring A is as defined in embodiment N2 or N3; and n, R¹, each R³ (independently of each other), R⁴, R⁵, R^{5a}, and R^{7c} are as defined in the Summary of the Invention for a Compound of Formula I or as defined in any of embodiments B-B2, C-C23, D-D2, K-K2, L, and M-M2.

Another embodiment (N5) of the Invention is directed to a Compound of Formula II where n is 2; R² and R^{2a} are halo; Ring A is as defined in embodiment N2 or N3; and R¹, each R³ (independently of each other), R⁴, R⁵, R^{5a}, and R^{7c} are as defined in the Summary of the Invention for a Compound of Formula I or as defined in any of embodiments C6-C11, C16-C23, D-D2, K-K2, L, and M-M2.

Another embodiment (N6) of the Invention is directed to a Compound of Formula II where R¹ is hydrogen or —P(O)(OR⁶)₂; n is 2; R² and R^{2a} are halo; Ring A is as defined in embodiment N2 or N3; and each R³ (independently of each other), R⁴, R⁵, R^{5a}, each R⁶ (independently of each other), and R^{7c} are as defined in the Summary of the Invention for a Compound of Formula I or as defined in any of embodiments C6-C11, C16-C23, D-D2, and K-K2. Another embodiment (N7) of the Invention is directed to a Compound of Formula II where R¹ is hydrogen; n is 2; R² and R^{2a} are halo; Ring A is as defined in embodiment N2 or N3; and each R³ (independently of each other), R⁴, R⁵, R^{5a}, and R^{7c} are as defined in the Summary of the Invention for a Compound of Formula I or as defined in any of embodiments C6-C11, C16-C23, D-D2, and K-K2. Another embodiment (N8) of the Invention is directed to a Compound of Formula II where R¹ is —P(O)(OR⁶)₂; n is 2; R² and R^{2a} are halo; Ring A is as defined in embodiment N2 or N3; and each R³ (independently of each other), R⁴, R⁵, R^{5a}, each R⁶ (independently of each other), and R^{7c} are as defined in the Summary of the Invention for a Compound of Formula I or as defined in any of embodiments C6-C11, C16-C23, D-D2, and K-K2. Another embodiment (N9) of the Invention is directed to a Compound of Formula II where R¹ is —P(O)(OR⁶)₂; both R⁶ are hydrogen or both R⁶ are tert-butyl; n is 2; R² and R^{2a} are halo; Ring A is as defined in embodiment N2 or N3; and each R³ (independently of each other), R⁴, R⁵, R^{5a}, and R^{7c} are as defined in the Summary of the Invention for a Compound of Formula I or as defined in any of embodiments C6-C11, C16-C23, D-D2, and K-K2. Another embodiment (N10) of the Invention is directed to a Compound of Formula II where R¹ is —P(O)(OR⁶)₂; both R⁶ are hydrogen; n is 2; R² and R^{2a} are halo; Ring A is as defined in embodiment N2 or N3; and each R³ (independently of each other), R⁴, R⁵, R^{5a}, and R^{7c} are as defined in the Summary of the Invention for a Compound of Formula I or as defined in any of embodiments C6-C11, C16-C23, D-D2, and K-K2.

Another embodiment (N11) of the Invention is directed to a Compound of Formula II where R¹ is as defined in any of N6-N10; n is 2; R² and R^{2a} are halo; R⁵ is hydrogen; R^{5a} is hydrogen or alkoxycarbonyl; Ring A is as defined in embodiment N2 or N3; and each R³ (independently of each other) and R⁴ are as defined in the Summary of the Invention for a Compound of Formula I or as defined in any of embodiments C6-C11, C16-C23, and D-D2. Another embodiment (N12) of the Invention is directed to a Compound of Formula II where R¹ is as defined in any of N6-N10; n is 2; R² and R^{2a} are halo; R⁵ is hydrogen; R^{5a} is hydrogen or tert-butoxycarbonyl; Ring A is as defined in embodiments N2 or N3; and each R³ (independently of each other) and R⁴ are as defined in the Summary of the Invention for a Compound of Formula I or as defined in any of embodiments C6-C11, C16-C23, and D-D2. Another embodiment (N13) of the Invention is directed to a Compound of Formula II where R¹ is as defined in any of N6-N10; n is 2; R² and R^{2a} are halo; R⁵ and R^{5a} are hydrogen; Ring A is as defined in embodiment N2 or N3; and each R³ (independently of each other) and R⁴ are as defined in the Summary of the Invention for a Compound of Formula I or as defined in any of embodiments C6-C11, C16-C23, and D-D2.

Another embodiment (N14) of the Invention is directed to a Compound of Formula II where where R¹ is as defined in any of N6-N10; n is 2; R² and R^{2a} are halo; R⁵ and R^{5a} are as defined in any of N11-N13; Ring A is as defined in embodiment N2 or N3; R⁴ is hydrogen; and each R³ is independently as defined in the Summary of the Invention for a Compound of Formula I or as defined in any of embodiments C6-C11, C16-C23.

Another embodiment (N15) of the Invention is directed to a Compound of Formula II where where R¹ is as defined in any of N6-N10; n is 2; R² and R^{2a} are halo; R⁵ and R^{5a} are as defined in any of N11-N13; Ring A is as defined in embodiment N2 or N3; R⁴ is hydrogen; and each R³ is independently halo, cyano, alkyl, haloalkyl, alkoxy, or alkylamino. Another embodiment (N16) of the Invention is directed to a Compound of Formula II where where R¹ is as defined in any of N6-N10; n is 2; R² and R^{2a} are halo; R⁵ and R^{5a} are as defined in any of N11-N13; Ring A is as defined in embodiments N2 or N3; R⁴ is hydrogen; and one R³ is alkoxy and the other R³ is halo, cyano, alkyl, or haloalkyl. Another embodiment (N17) of the Invention is directed to a Compound of Formula II where where R¹ is as defined in any of N6-N10; n is 2; R² and R^{2a} are halo; R⁵ and R^{5a} are as defined in any of N11-N13; Ring A is as defined in embodiment N2 or N3; R⁴ is hydrogen; and one R³ is alkylamino and the other R³ is halo or alkyl.

Another embodiment (N18) of the Invention is directed to a Compound of Formula II where R^{7c} is hydrogen and all other groups are as defined in the Summary of the Invention for a Compound of Formula I or as defined in any of embodiments N1-N17.

Another embodiment (N19) of the Invention is directed to a Compound of Formula II where R^{7c} is alkyl and all other groups are as defined in the Summary of the Invention for a Compound of Formula I or as defined in any of embodiments N1-N17. Another embodiment (N20) of the Invention is directed to a Compound of Formula II where R^{7c} is methyl and all other groups are as defined in the Summary of the Invention for a Compound of Formula I or as defined in any of embodiments N1-N17.

In one embodiment of the invention the Compound of Formula I is according to Formula II(a)

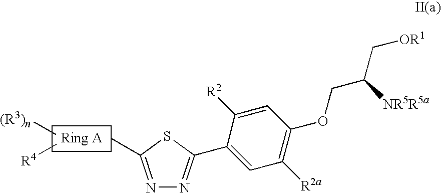

or a pharmaceutically acceptable salt thereof where n, R¹, R², R^{2a}, each R³ (independently of each other), R⁴, R⁵, and R^{5a} are as defined in the Summary of the Invention for a Compound of Formula I or as defined in any of embodiments N1-N17.

In one embodiment of the invention the Compound of Formula I is according to Formula II(b)

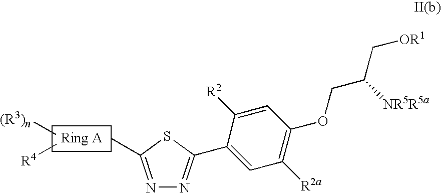

or a pharmaceutically acceptable salt thereof where n, R¹, R², R^{2a}, each R³ (independently of each other), R⁴, R⁵, and R^{5a} are as defined in the Summary of the Invention for a Compound of Formula I or as defined in any of embodiments N1-N17.

In one embodiment of the invention the Compound of Formula I is according to Formula II(c)

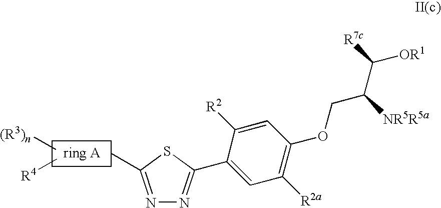

or a pharmaceutically acceptable salt thereof where R^{7c} is alkyl, n, R¹, R², R^{2a}, each R³ (independently of each other), R⁴, R⁵, and R^{5a} are as defined in the Summary of the Invention for a Compound of Formula I or as defined in any of embodiments N1-N17. In another embodiment of the invention the Compound of Formula I is according to Formula II(c) where R^{7c} is methyl, n, R¹, R², R^{2a}, R³, R⁴, R⁵, and R^{5a} are as defined in the Summary of the Invention for a Compound of Formula I or as defined in any of embodiments N1-N17.

In one embodiment of the invention the Compound of Formula I is according to Formula II(d)

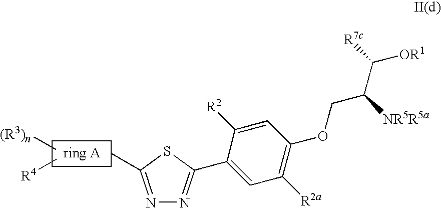

or a pharmaceutically acceptable salt thereof where R^{7c} is alkyl, n, R¹, R², R^{2a}, each R³ (independently of each other), R⁴, R⁵, and R^{5a} are as defined in the Summary of the Invention for a Compound of Formula I or as defined in any of embodiments N1-N17. In another embodiment of the invention the Compound of Formula I is according to Formula II(d) where R^{7c} is methyl, n, R¹, R², R^{2a}, R³ (independently of each other), R⁴, R⁵, and R^{5a} are as defined in the Summary of the Invention for a Compound of Formula I or as defined in any of embodiments N1-N17.

In one embodiment of the invention the Compound of Formula I is according to Formula II(e)

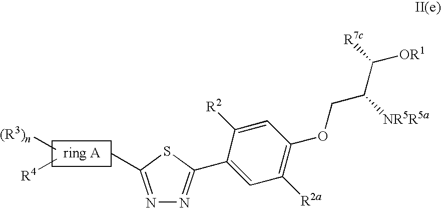

or a pharmaceutically acceptable salt thereof where R^{7c} is alkyl, n, R¹, R², R^{2a}, each R³ (independently of each other), R⁴, R⁵, and R^{5a} are as defined in the Summary of the Invention for a Compound of Formula I or as defined in any of embodiments N1-N17. In another embodiment of the invention the Compound of Formula I is according to Formula II(e) where R^{7c} is methyl, n, R¹, R², R^{2a}, R³ (independently of each other), R⁴, R⁵, and R^{5a} are as defined in the Summary of the Invention for a Compound of Formula I or as defined in any of embodiments N1-N17.

In one embodiment of the invention the Compound of Formula I is according to Formula II(f)

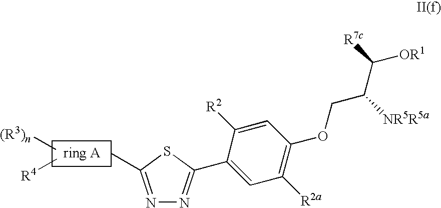

or a pharmaceutically acceptable salt thereof where R^{7c} is alkyl, n, R¹, R², R^{2a}, each R³ (independently of each other), R⁴, R⁵, and R^{5a} are as defined in the Summary of the Invention for a Compound of Formula I or as defined in any of embodiments N1-N17. In another embodiment of the invention the Compound of Formula I is according to Formula II(f) where R^{7c} is methyl, n, R¹, R², R^{2a}, R³ (independently of each other), R⁴, R⁵, and R^{5a} are as defined in the Summary of the Invention for a Compound of Formula I or as defined in any of embodiments N1-N17.

In another embodiment (M), the invention provides a Compound of Formula I, or a pharmaceutically acceptable salt thereof, where
R¹ is hydrogen or —P(O)(OR⁶)₂;
R² and R^{2a} are independently hydrogen or halo;
Ring A is phenyl, thienyl, pyridinyl, pyrimidinyl, or imidazo[2,1-b]thiazolyl;
each R³ is independently halo, alkyl, alkoxy, haloalkoxy, alkoxyalkyloxy, heterocycloalkyloxy, phenyl, amino, alkylamino, dialkylamino, or aminocarbonyl;
n is 0, 1, or 2;
R⁴ is hydrogen, alkyl, cyano, halo, or haloalkyl;
R⁵ is hydrogen or alkyl;
R^{5a} is hydrogen, alkyl, or alkoxycarbonyl;
each R⁶ is independently hydrogen or alkyl;
R⁷, R^{7a}, and R^{7b} are hydrogen; and
R^{7c} is hydrogen or alkyl.

Another embodiment (N) of the Invention provides a pharmaceutical composition which comprises a compound of any one of Formulae I, I(a), I(b), I(c), I(d), I(e), I(f), I(g), or a compound selected from Table 1 or a pharmaceutically acceptable salt or solvate thereof and a pharmaceutically acceptable carrier, excipient, or diluent.

Another embodiment (P) of the Invention is a method of treating disease, disorder, or syndrome where the disease is associated with uncontrolled, abnormal, and/or unwanted cellular activities effected directly or indirectly by S1P1 and/or S1P5 which method comprises administering to a human in need thereof a therapeutically effective amount of a compound of Formula I, I(a), I(b), I(c), I(d), I(e), I(f), I(g), or a compound selected from Table 1 or selected from any of the above embodiments, or a pharmaceutically acceptable salt or pharmaceutical composition thereof.

Another embodiment (Q) of the invention is directed to a method of treating a disease, disorder, or syndrome which method comprises administering to a patient a therapeutically effective amount of a compound of Formula I, I(a), I(b), I(c), I(d), I(e), I(f), I(g), or a compound selected from Table 1 or selected from any of the above embodiments, optionally as a pharmaceutically acceptable salt or pharmaceutical composition thereof. In another embodiment, the disease is an autoimmune disease. In another embodiment the autoimmune disease is multiple sclerosis. In another embodiment, the disease is psoriasis. In another embodiment, the disease is inflammatory bowel disease. In another embodiment the autoimmune disease is graft-versus-host disease. In another embodiment, the disease is inflammation caused by an autoimmune disease.

### Representative Compounds

Representative compounds of Formula I are depicted below. The examples are merely illustrative and do not limit the scope of the invention in any way. Compounds of the invention are named according to systematic application of the nomenclature rules agreed upon by the International Union of Pure and Applied Chemistry (IUPAC), International Union of Biochemistry and Molecular Biology (IUBMB), and the Chemical Abstracts Service (CAS). Names were generated using ACD/Labs naming software.

TABLE 1

| Entry | | |
|---|---|---|
| No. | Structure | ACD-generated Name |
| 1 | 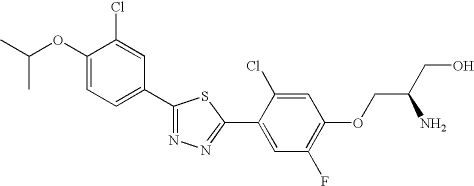 | (2R)-2-amino-3-{[5-chloro-4-(5-{3- chloro-4-[(1-methylethyl)oxy]phenyl}- 1,3,4-thiadiazol-2-yl)-2- fluorophenyl]oxy}propan-1-ol |
| 2 | 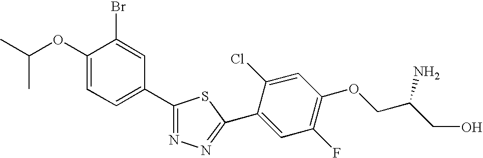 | (2R)-2-amino-3-{[4-(5-{3-bromo-4-[(1- methylethyl)oxy]phenyl}-1,3,4- thiadiazol-2-yl)-5-chloro-2- fluorophenyl]oxy}propan-1-ol |
| 3 | 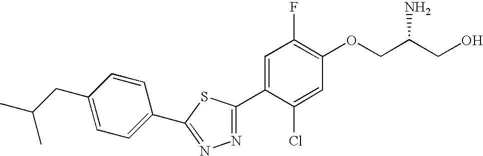 | (2R)-2-amino-3-[(5-chloro-2-fluoro-4- {5-[4-(2-methylpropyl)phenyl]-1,3,4- thiadiazol-2-yl}phenyl)oxy]propan-1-ol |
| 4 | 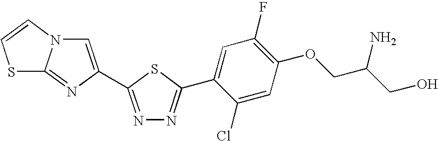 | 2-amino-3-{[5-chloro-2-fluoro-4-(5- imidazo[2,1-b][1,3]thiazol-6-yl-1,3,4- thiadiazol-2-yl)phenyl]oxy}propan-1-ol |
| 5 | 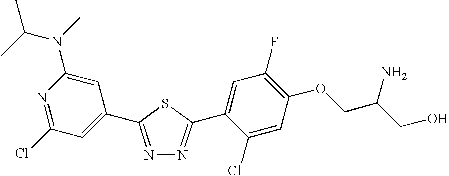 | 2-amino-3-{[5-chloro-4-(5-{2-chloro-6- [methyl(1-methylethyl)amino]pyridin-4- yl}-1,3,4-thiadiazol-2-yl)-2- fluorophenyl]oxy}propan-1-ol |
| 6 | 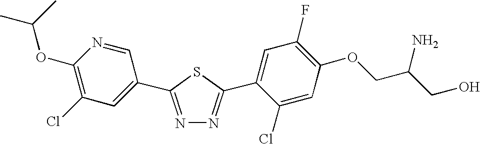 | 2-amino-3-{[5-chloro-4-(5-{5-chloro-6- [(1-methylethyl)oxy]pyridin-3-yl}-1,3,4- thiadiazol-2-yl)-2- fluorophenyl]oxy}propan-1-ol |
| 7 | 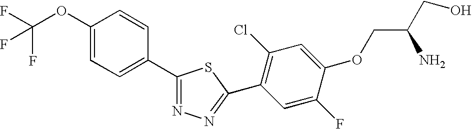 | (2R)-2-amino-3-{[5-chloro-2-fluoro-4- (5-{4-[(trifluoromethyl)oxy]phenyl}- 1,3,4-thiadiazol-2- yl)phenyl]oxy}propan-1-ol |
| 8 | 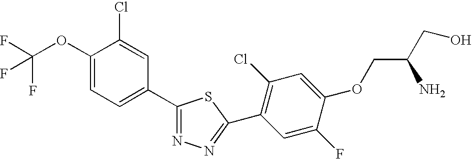 | (2R)-2-amino-3-{[5-chloro-4-(5-{3- chloro-4-[(trifluoromethyl)oxy]phenyl}- 1,3,4-thiadiazol-2-yl)-2- fluorophenyl]oxy}propan-1-ol |
| 9 | 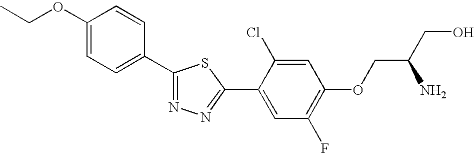 | (2R)-2-amino-3-{[5-chloro-2-fluoro-4- (5-{4-[(1-methylethyl)oxy]phenyl}- 1,3,4-thiadiazol-2- yl)phenyl]oxy}propan-1-ol |
| 10 | 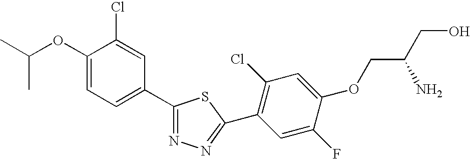 | (2S)-2-amino-3-{[5-chloro-4-(5-{3- chloro-4-[(1-methylethyl)oxy]phenyl}- 1,3,4-thiadiazol-2-yl)-2- fluorophenyl]oxy}propan-1-ol |
| 11 | 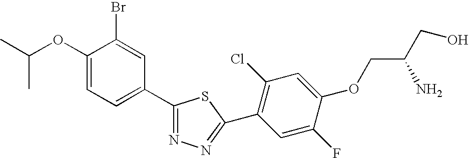 | (2S)-2-amino-3-{[4-(5-{3-bromo-4-[(1- methylethyl)oxy]phenyl}-1,3,4- thiadiazol-2-yl)-5-chloro-2- fluorophenyl]oxy}propan-1-ol |
| 12 | 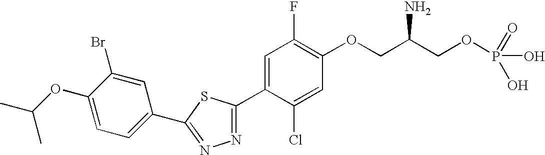 | (2R)-2-amino-3-{[4-(5-{3-bromo-4-[(1- methylethyl)oxy]phenyl}-1,3,4- thiadiazol-2-yl)-5-chloro-2- fluorophenyl]oxy}propyl dihydrogen phosphate |
| 13 | 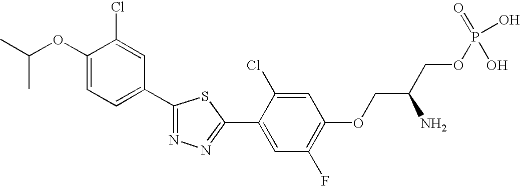 | (2S)-2-amino-3-{[5-chloro-4-(5-{3- chloro-4-[(1-methylethyl)oxy]phenyl}- 1,3,4-thiadiazol-2-yl)-2- fluorophenyl]oxy}propyl dihydrogen phosphate |
| 14 | 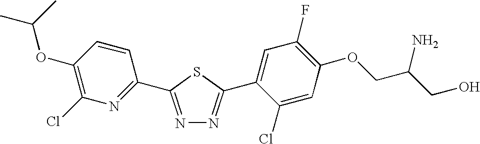 | 2-amino-3-{[5-chloro-4-(5-{6-chloro-5- [(1-methylethyl)oxy]pyridin-2-yl}-1,3,4- thiadiazol-2-yl)-2- fluorophenyl]oxy}propan-1-ol |
| 15 | 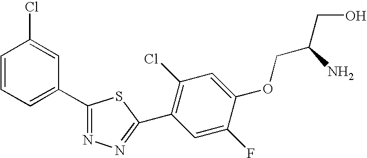 | (2R)-2-amino-3-({5-chloro-4-[5-(3- chlorophenyl)-1,3,4-thiadiazol-2-yl]-2- fluorophenyl}oxy)propan-1-ol |
| 16 | 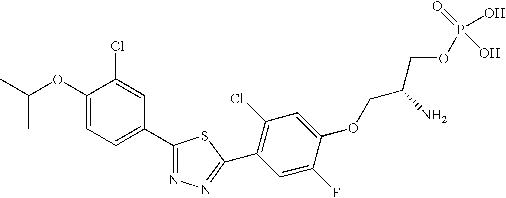 | (2R)-2-amino-3-{[5-chloro-4-(5-{3- chloro-4-[(1-methylethyl)oxy]phenyl}- 1,3,4-thiadiazol-2-yl)-2- fluorophenyl]oxy}propyl dihydrogen phosphate |
| 17 | 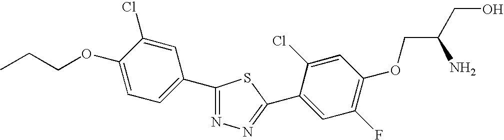 | (2R)-2-amino-3-[(5-chloro-4-{5-[3- chloro-4-(propyloxy)phenyl]-1,3,4- thiadiazol-2-yl}-2- fluorophenyl)oxy]propan-1-ol |
| 18 | 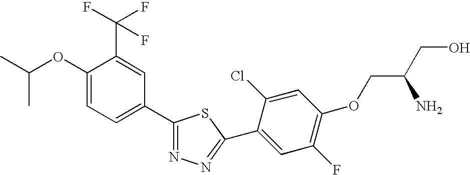 | (2R)-2-amino-3-{[5-chloro-2-fluoro-4- (5-{4-[(1-methylethyl)oxy]-3- (trifluoromethyl)phenyl}-1,3,4- thiadiazol-2-yl)phenyl]oxy}propan-1-ol |
| 19 | 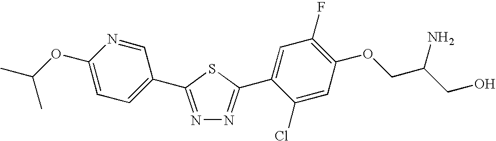 | 2-amino-3-{[5-chloro-2-fluoro-4-(5-{6- [(1-methylethyl)oxy]pyridin-3-yl}-1,3,4- thiadiazol-2-yl)phenyl]oxy}propan-1-ol |
| 20 | 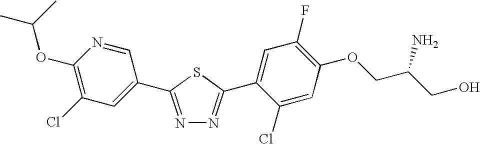 | (2R)-2-amino-3-{[5-chloro-4-(5-{5- chloro-6-[(1-methylethyl)oxy]pyridin-3- yl}-1,3,4-thiadiazol-2-yl)-2- fluorophenyl]oxy}propan-1-ol |
| 21 | 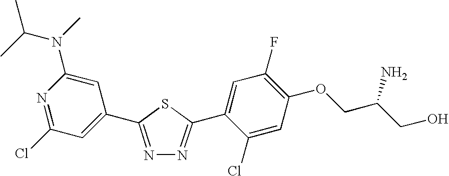 | (2R)-2-amino-3-{[5-chloro-4-(5-{2- chloro-6-[methyl(1- methylethyl)amino]pyridin-4-yl}-1,3,4- thiadiazol-2-yl)-2- fluorophenyl)oxy}propan-1-ol |
| 22 | 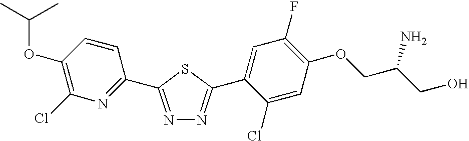 | (2R)-2-amino-3-{[5-chloro-4-(5-{6- chloro-5-[(1-methylethyl)oxy]pyridin-2- yl}-1,3,4-thiadiazol-2-yl)-2- fluorophenyl]oxy}propan-1-ol |
| 23 | 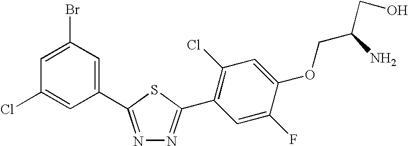 | (2R)-2-amino-3-({4-[5-(3-bromo-5- chlorophenyl)-1,3,4-thiadiazol-2-yl]-5- chloro-2-fluorophenyl}oxy)propan-1-ol |
| 24 | 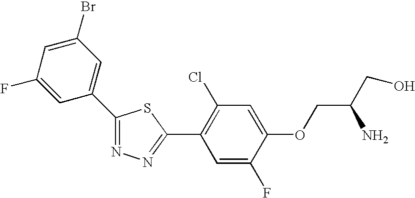 | (2R)-2-amino-3-({4-[5-(3-bromo-5- fluorophenyl)-1,3,4-thiadiazol-2-yl]-5- chloro-2-fluorophenyl}oxy)propan-1-ol |
| 25 | 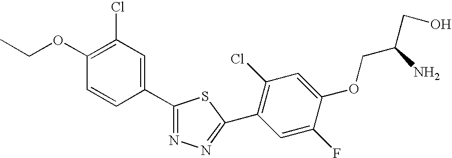 | (2R)-2-amino-3-[(5-chloro-4-{5-[3- chloro-4-(ethyloxy)phenyl]-1,3,4- thiadiazol-2-yl}-2- fluorophenyl)oxy]propan-1-ol |
| 26 | 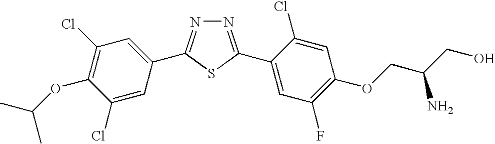 | (2R)-2-amino-3-{[5-chloro-4-(5-{3,5- dichloro-4-[(1- methylethyl)oxy]phenyl}-1,3,4- thiadiazol-2-yl)-2- fluorophenyl]oxy}propan-1-ol |
| 27 | 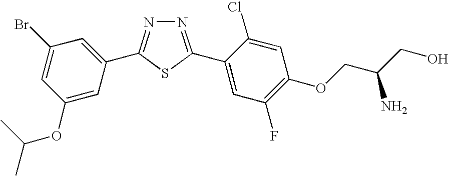 | (2R)-2-amino-3-{[4-(5-{3-bromo-5-[(1- methylethyl)oxy]phenyl}-1,3,4- thiadiazol-2-yl)-5-chloro-2- fluorophenyl]oxy}propan-1-ol |
| 28 | 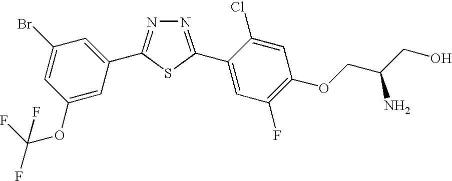 | (2R)-2-amino-3-{[4-(5-{3-bromo-5- [(trifluoromethyl)oxy]phenyl}-1,3,4- thiadiazol-2-yl)-5-chloro-2- fluorophenyl]oxy}propan-1-ol |
| 29 | 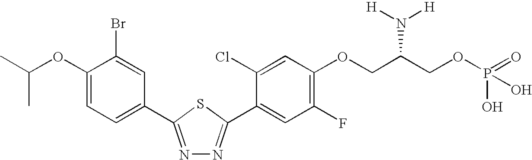 | (2S)-2-amino-3-{[4-(5-{3-bromo-4-[(1- methylethyl)oxy]phenyl}-1,3,4- thiadiazol-2-yl)-5-chloro-2- fluorophenyl]oxy}propyl dihydrogen phosphate |
| 30 | 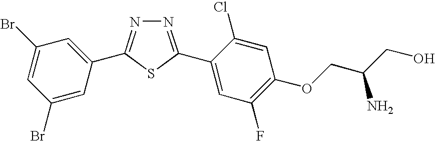 | (2R)-2-amino-3-({5-chloro-4-[5-(3,5- dibromophenyl)-1,3,4-thiadiazol-2-yl]- 2-fluorophenyl}oxy)propan-1-ol |
| 31 | 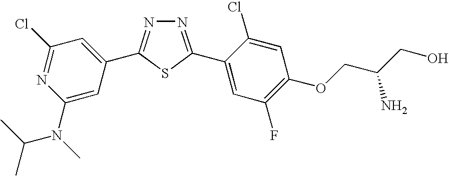 | (2S)-2-amino-3-{[5-chloro-4-(5-{2- chloro-6-[methyl(1- methylethyl)amino]pyridin-4-yl}-1,3,4- thiadiazol-2-yl)-2- fluorophenyl]oxy}propan-1-ol |
| 32 | 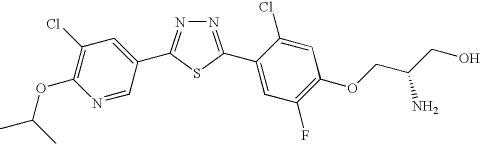 | (2S)-2-amino-3-{[5-chloro-4-(5-{5- chloro-6-[(1-methylethyl)oxy]pyridin-3- yl}-1,3,4-thiadiazol-2-yl)-2- fluorophenyl]oxy}propan-1-ol |
| 33 | 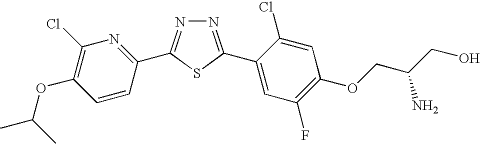 | (2S)-2-amino-3-{[5-chloro-4-(5-{6- chloro-5-[(1-methylethyl)oxy]pyridin-2- yl}-1,3,4-thiadiazol-2-yl)-2- fluorophenyl]oxy}propan-1-ol |
| 34 | 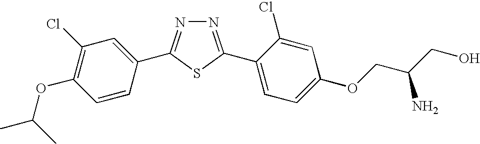 | (2R)-2-amino-3-{[3-chloro-4-(5-{3- chloro-4-[(1-methylethyl)oxy]phenyl}- 1,3,4-thiadiazol-2- yl)phenyl]oxy}propan-1-ol |
| 35 | 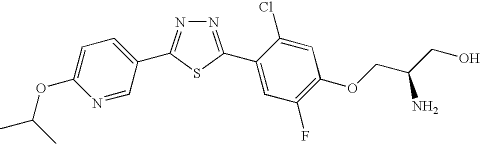 | (2R)-2-amino-3-{[5-chloro-2-fluoro-4- (5-{6-[(1-methylethyl)oxy]pyridin-3- yl}-1,3,4-thiadiazol-2- yl)phenyl]oxy}propan-1-ol |
| 36 | 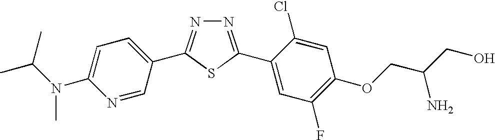 | 2-amino-3-{[5-chloro-2-fluoro-4-(5-{6- [methyl(1-methylethyl)amino]pyridin-3- yl}-1,3,4-thiadiazol-2- yl)phenyl]oxy}propan-1-ol |
| 37 | 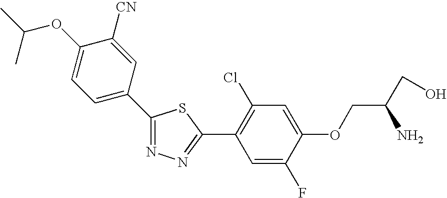 | 5-[5-(4-{[(2R)-2-amino-3- hydroxypropyl]oxy}-2-chloro-5- fluorophenyl)-1,3,4-thiadiazol-2-yl]-2- [(1-methylethyl)oxy]benzonitrile |
| 38 | 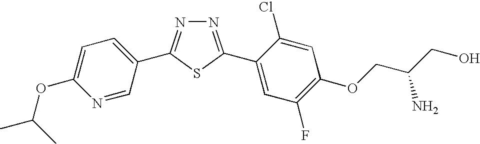 | (2S)-2-amino-3-{[5-chloro-2-fluoro-4- (5-{6-[(1-methylethyl)oxy]pyridin-3- yl}-1,3,4-thiadiazol-2- yl)phenyl]oxy}propan-1-ol |
| 39 | 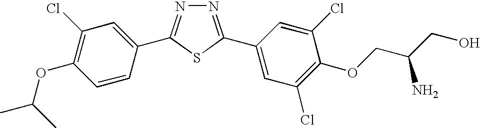 | (2R)-2-amino-3-{[2,6-dichloro-4-(5-{3- chloro-4-[(1-methylethyl)oxy]phenyl}- 1,3,4-thiadiazol-2- yl)phenyl]oxy}propan-1-ol |
| 40 | 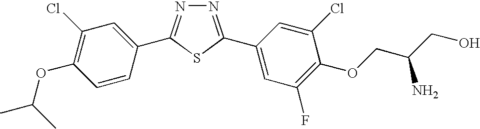 | (2R)-2-amino-3-{[2-chloro-4-(5-{3- chloro-4-[(1-methylethyl)oxy]phenyl}- 1,3,4-thiadiazol-2-yl)-6- fluorophenyl]oxy}propan-1-ol |
| 41 | 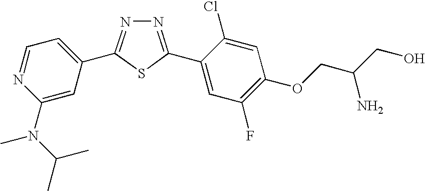 | 2-amino-3-{[5-chloro-2-fluoro-4-(5-{2- [methyl(1-methylethyl)amino]pyridin-4- yl}-1,3,4-thiadiazol-2- yl)phenyl]oxy}propan-1-ol |
| 42 | 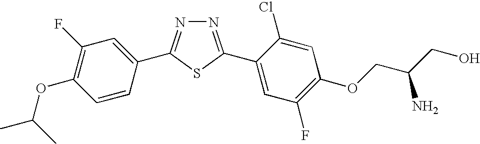 | (2R)-2-amino-3-{[5-chloro-2-fluoro-4- (5-{3-fluoro-4-[(1- methylethyl)oxy]phenyl}-1,3,4- thiadiazol-2-yl)phenyl]oxy}propan-1-ol |
| 43 | 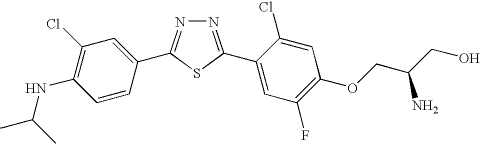 | (2R)-2-amino-3-{[5-chloro-4-(5-{3- chloro-4-[(1- methylethyl)amino]phenyl}-1,3,4- thiadiazol-2-yl)-2- fluorophenyl]oxy}propan-1-ol |
| 44 | 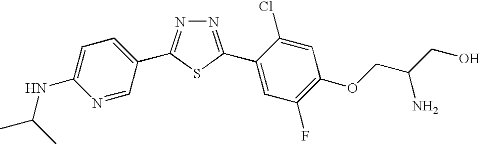 | 2-amino-3-{[5-chloro-2-fluoro-4-(5-{6- [(1-methylethyl)amino]pyridin-3-yl}- 1,3,4-thiadiazol-2- yl)phenyl]oxy}propan-1-ol |
| 45 | 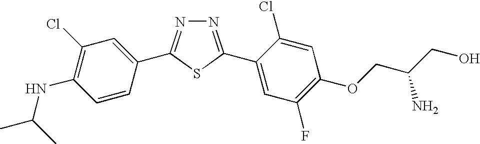 | (2S)-2-amino-3-{[5-chloro-4-(5-{3- chloro-4-[(1- methylethyl)amino]phenyl}-1,3,4- thiadiazol-2-yl)-2- fluorophenyl]oxy}propan-1-ol |
| 46 | 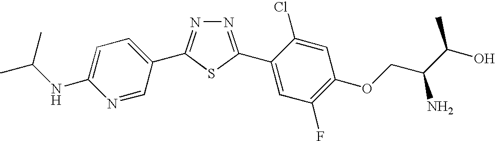 | (2R,3S)-3-amino-4-{[5-chloro-2-fluoro- 4-(5-{6-[(1-methylethyl)amino]pyridin- 3-yl}-1,3,4-thiadiazol-2- yl)phenyl]oxy}butan-2-ol |
| 47 | 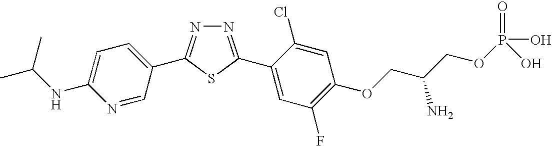 | (2R)-2-amino-3-{[5-chloro-2-fluoro-4- (5-{6-[(1-methylethyl)amino]pyridin-3- yl}-1,3,4-thiadiazol-2- yl)phenyl]oxy}propyl dihydrogen phosphate |
| 48 | 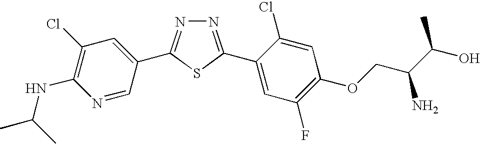 | (2R,3S)-3-amino-4-{[5-chloro-4-(5-{5- chloro-6-[(1-methylethyl)amino]pyridin- 3-yl}-1,3,4-thiadiazol-2-yl)-2- fluorophenyl]oxy}butan-2-ol |
| 49 | 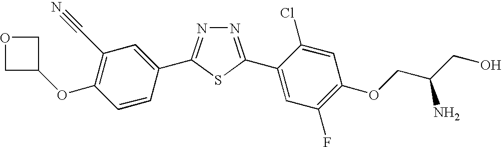 | 5-[5-(4-{[(2R)-2-amino-3- hydroxypropyl]oxy}-2-chloro-5- fluorophenyl)-1,3,4-thiadiazol-2-yl]-2- (oxetan-3-yloxy)benzonitrile |
| 50 | 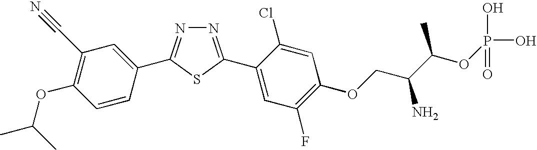 | (1R,2S)-2-amino-3-{[5-chloro-4-(5-{3- cyano-4-[(1-methylethyl)oxy]phenyl}- 1,3,4-thiadiazol-2-yl)-2- fluorophenyl]oxy}-1-methylpropyl dihydrogen phosphate |
| 51 | 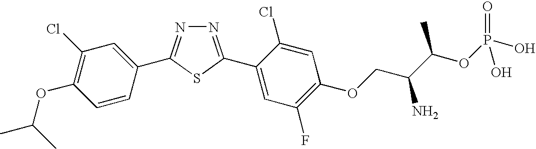 | (1R,2S)-2-amino-3-{[5-chloro-4-(5-{3- chloro-4-[(1-methylethyl)oxy]phenyl}- 1,3,4-thiadiazol-2-yl)-2- fluorophenyl]oxy}-1-methylpropyl dihydrogen phosphate |
| 52 | 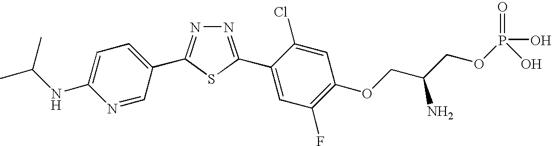 | (2S)-2-amino-3-{[5-chloro-2-fluoro-4- (5-{6-[(1-methylethyl)amino]pyridin-3- yl}-1,3,4-thiadiazol-2- yl)phenyl]oxy}propyl dihydrogen phosphate |
| 53 | 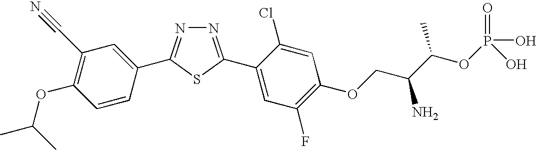 | (1S,2S)-2-amino-3-{[5-chloro-4-(5-{3- cyano-4-[(1-methylethyl)oxy]phenyl}- 1,3,4-thiadiazol-2-yl)-2- fluorophenyl]oxy}-1-methylpropyl dihydrogen phosphate |
| 54 | 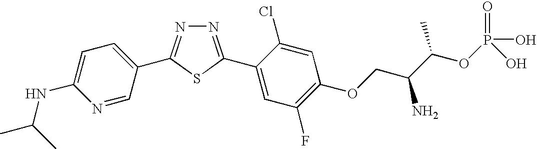 | (1S,2S)-2-amino-3-{[5-chloro-2-fluoro- 4-(5-{6-[(1-methylethyl)amino]pyridin- 3-yl}-1,3,4-thiadiazol-2-yl)phenyl]oxy}- 1-methylpropyl dihydrogen phosphate |
| 55 | 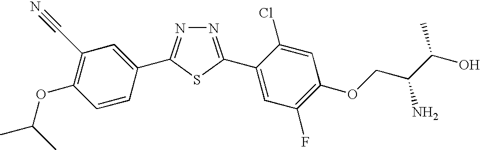 | 5-[5-(4-{[(2R,3S)-2-amino-3- hydroxybutyl]oxy}-2-chloro-5- fluorophenyl)-1,3,4-thiadiazol-2-yl]-2- [(1-methylethyl)oxy]benzonitrile |
| 56 | 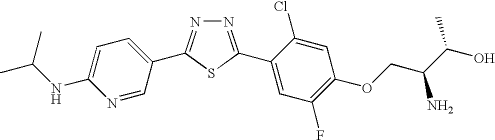 | (2S,3S)-3-amino-4-{[5-chloro-2-fluoro- 4-(5-{6-[(1-methylethyl)amino]pyridin- 3-yl}-1,3,4-thiadiazol-2- yl)phenyl]oxy}butan-2-ol |
| 57 | 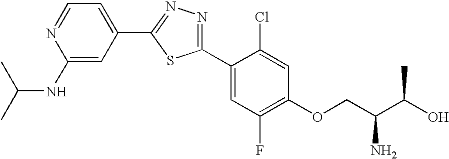 | (2R,3S)-3-amino-4-{5-chloro-2-fluoro- 4-(5-{2-[(1-methylethyl)amino]pyridin- 4-yl}-1,3,4-thiadiazol-2- yl)phenyl]oxy}butan-2-ol |
| 58 | 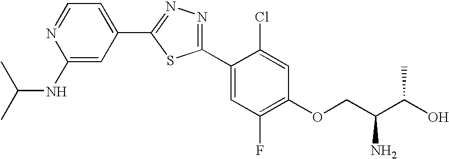 | (2S,3S)-3-amino-4-{[5-chloro-2-fluoro- 4-(5-{2-[(1-methylethyl)amino]pyridin- 4-yl}-1,3,4-thiadiazol-2- yl)phenyl]oxy}butan-2-ol |
| 59 | 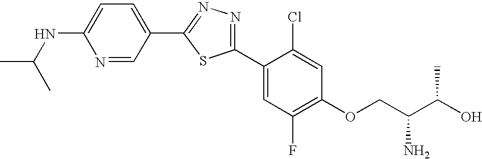 | (2S,3R)-3-amino-4-{[5-chloro-2-fluoro- 4-(5-{6-[(1-methylethyl)amino]pyridin- 3-yl}-1,3,4-thiadiazol-2- yl)phenyl]oxy}butan-2-ol |
| 60 | 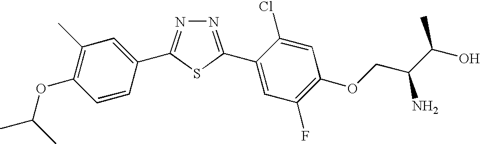 | (2R,3S)-3-amino-4-{[5-chloro-2-fluoro- 4-(5-{3-methyl-4-[(1- methylethyl)oxy]phenyl}-1,3,4- thiadiazol-2-yl)phenyl]oxy}butan-2-ol |
| 61 | 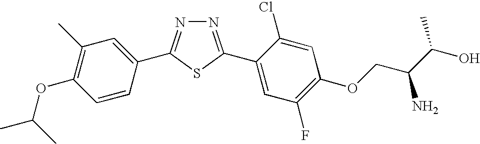 | (2S,3S)-3-amino-4-{[5-chloro-2-fluoro- 4-(5-{3-methyl-4-[(1- methylethyl)oxy]phenyl}-1,3,4- thiadiazol-2-yl)phenyl]oxy}butan-2-ol |
| 62 | 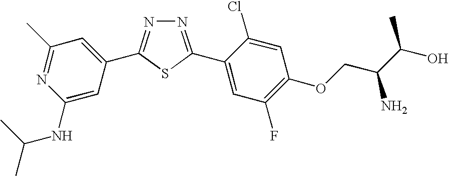 | (2R,3S)-3-amino-4-{[5-chloro-2-fluoro- 4-(5-{2-methyl-6-[(1- methylethyl)amino]pyridin-4-yl}-1,3,4- thiadiazol-2-yl)phenyl]oxy}butan-2-ol |
| 63 | 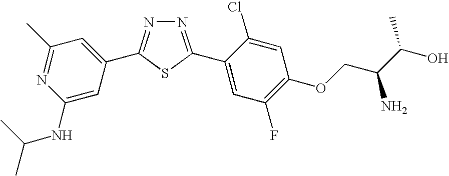 | (2S,3S)-3-amino-4-{[5-chloro-2-fluoro- 4-(5-{2-methyl-6-[(1- methylethyl)amino]pyridin-4-yl}-1,3,4- thiadiazol-2-yl)phenyl]oxy}butan-2-ol |
| 64 | 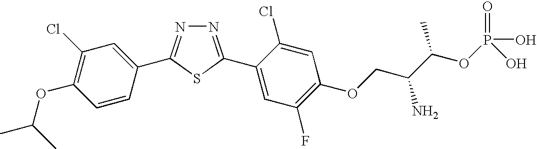 | (1S,2R)-2-amino-3-{[5-chloro-4-(5-{3- chloro-4-[(1-methylethyl)oxy]phenyl}- 1,3,4-thiadiazol-2-yl)-2- fluorophenyl]oxy}-1-methylpropyl dihydrogen phosphate |
| 65 | 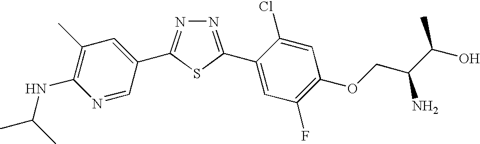 | (2R,3S)-3-amino-4-{[5-chloro-2-fluoro- 4-(5-{5-methyl-6-[(1- methylethyl)amino]pyridin-3-yl}-1,3,4- thiadiazol-2-yl)phenyl]oxy}butan-2-ol |
| 66 | 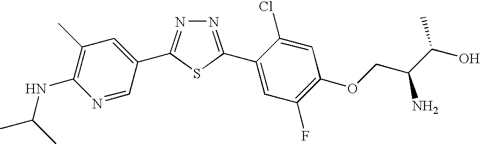 | (2S,3S)-3-amino-4-{[5-chloro-2-fluoro- 4-(5-{5-methyl-6-[(1- methylethyl)amino]pyridin-3-yl}-1,3,4- thiadiazol-2-yl)phenyl]oxy}butan-2-ol |
| 67 | 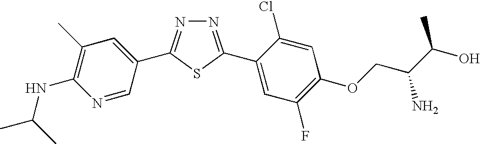 | (2R,3R)-3-amino-4-{[5-chloro-2-fluoro- 4-(5-{5-methyl-6-[(1- methylethyl)amino]pyridin-3-yl}-1,3,4- thiadiazol-2-yl)phenyl]oxy}butan-2-ol |
| 68 | 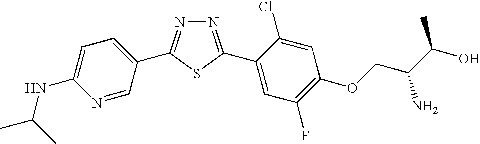 | (2R,3R)-3-amino-4-{[5-chloro-2-fluoro- 4-(5-{6-[(1-methylethyl)amino]pyridin- 3-yl}-1,3,4-thiadiazol-2- yl)phenyl]oxy}butan-2-ol |
| 69 | 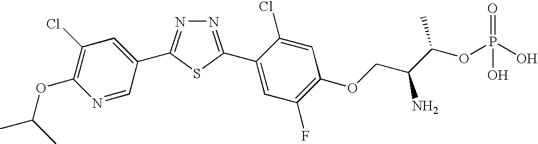 | (1S,2S)-2-amino-3-{[5-chloro-4-(5-{3- chloro-4-[(1-methylethyl)oxy]phenyl}- 1,3,4-thiadiazol-2-yl)-2- fluorophenyl]oxy}-1-methylpropyl dihydrogen phosphate |
| 70 | 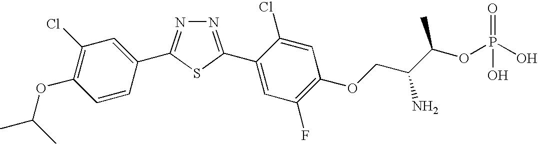 | (1R,2R)-2-amino-3-{[5-chloro-4-(5-{3- chloro-4-[(1-methylethyl)oxy]phenyl}- 1,3,4-thiadiazol-2-yl)-2- fluorophenyl]oxy}-1-methylpropyl dihydrogen phosphate |
| 71 | 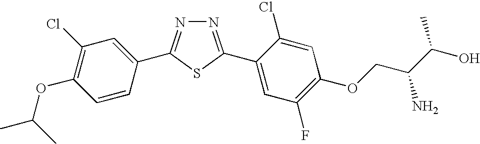 | (2S,3R)-3-amino-4-{[5-chloro-4-(5-{3- chloro-4-[(1-methylethyl)oxy]phenyl}- 1,3,4-thiadiazol-2-yl)-2- fluorophenyl]oxy}butan-2-ol |
| 72 | 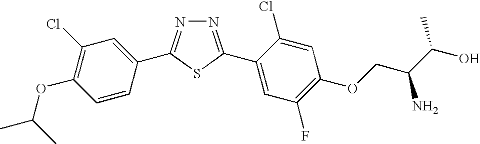 | (2S,3S)-3-amino-4-{[5-chloro-4-(5-{3- chloro-4-[(1-methylethyl)oxy]phenyl}- 1,3,4-thiadiazol-2-yl)-2- fluorophenyl]oxy}butan-2-ol |
| 73 | 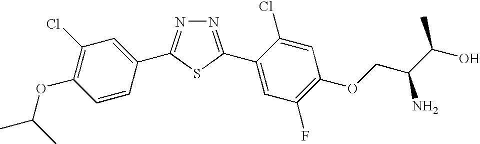 | (2R,3S)-3-amino-4-{[5-chloro-4-(5-{3- chloro-4-[(1-methylethyl)oxy]phenyl}- 1,3,4-thiadiazol-2-yl)-2- fluorophenyl]oxy}butan-2-ol |
| 74 | 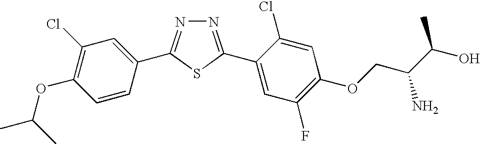 | (2R,3R)-3-amino-4-{[5-chloro-4-(5-{3- chloro-4-[(1-methylethyl)oxy]phenyl}- 1,3,4-thiadiazol-2-yl)-2- fluorophenyl]oxy}butan-2-ol |
| 75 | 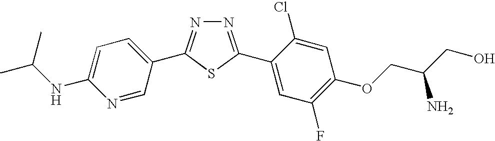 | (2R)-2-amino-3-{[5-chloro-2-fluoro-4- (5-{6-[(1-methylethyl)amino]pyridin-3- yl}-1,3,4-thiadiazol-2- yl)phenyl]oxy}propan-1-ol |
| 76 | 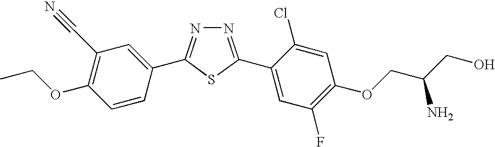 | 5-[5-(4-{[(2R)-2-amino-3- hydroxyporpyl]oxy}-2-chloro-5- fluorophenyl)-1,3,4-thiadiazol-2-yl]-2- (ethyloxy)benzonitrile |
| 77 | 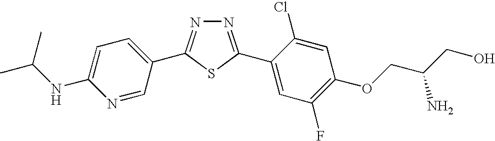 | (2S)-2-amino-3-{[5-chloro-2-fluoro-4- (5-{6-[(1-methylethyl)amino]pyridin-3- yl}-1,3,4-thiadiazol-2- yl)phenyl]oxy}propan-1-ol |
| 78 | 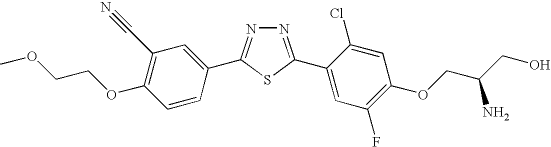 | 5-[5-(4-{[(2R)-2-amino-3- hydroxyporpyl]oxy}-2-chloro-5- fluorophenyl)-1,3,4-thiadiazol-2-yl]-2- {[2-(methyloxy)ethyl]oxy}benzonitrile |
| 79 | 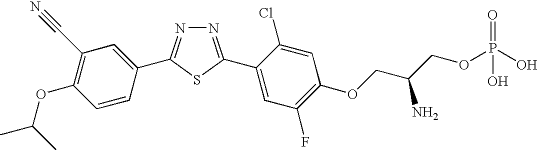 | (2S)-2-amino-3-{[5-chloro-4-(5-{3- cyano-4-[(1-methylethyl)oxy]phenyl}- 1,3,4-thiadiazol-2-yl)-2- fluorophenyl]oxy}propyl dihydrogen phosphate |
| 80 | 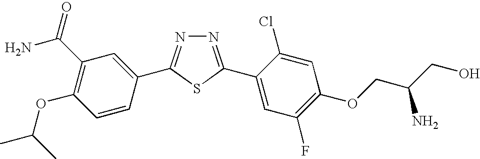 | 5-[5-(4-{[(2R)-2-amino-3- hydroxypropyl]oxy}-2-chloro-5- fluorophenyl)-1,3,4-thiadiazol-2-yl]-2- [(1-methylethyl)oxy]benzamide |
| 81 | 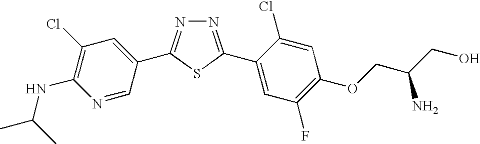 | (2R)-2-amino-3-{[5-chloro-4-(5-{5- chloro-6-[(1-methylethyl)amino]pyridin- 3-yl}-1,3,4-thiadiazol-2-yl)-2- fluorophenyl]oxy}propan-1-ol |
| 82 | 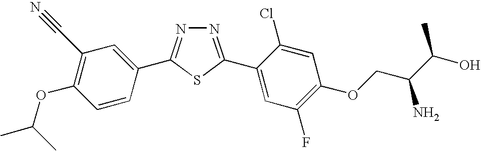 | 5-[5-(4-{[(2S,3R)-2-amino-3- hydroxybutyl]oxy}-2-chloro-5- fluorophenyl)-1,3,4-thiadiazol-2-yl]-2- [(1-methylethyl)oxy]benzonitrile |
| 83 | 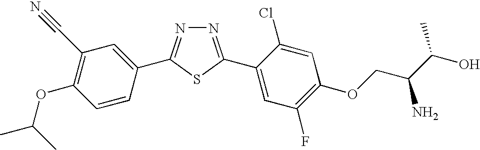 | 5-[5-(4-{[(2S,3S)-2-amino-3- hydroxybutyl]oxy}-2-chloro-5- fluorophenyl)-1,3,4-thiadiazol-2-yl]-2- [(1-methylethyl)oxy]benzonitrile |
| 84 | 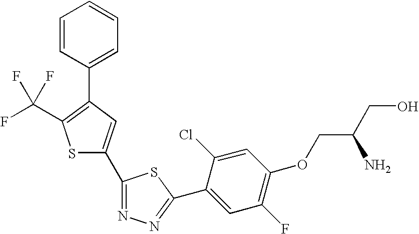 | (2R)-2-amino-3-[(5-chloro-2-fluoro-4- {5-[4-phenyl-5-(trifluoromethyl)-2- thienyl]-1,3,4-thiadiazol-2- yl}phenyl)oxy]propan-1-ol |
| 85 | 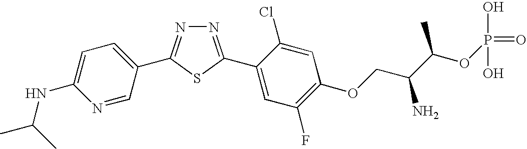 | (1R,2S)-2-amino-3-{[5-chloro-2-fluoro- 4-(5-{6-[(1-methylethyl)amino]pyridin- 3-yl}-1,3,4-thiadiazol-2-yl)phenyl]oxy}- 1-methylpropyl dihydrogen phosphate |
| 86 | 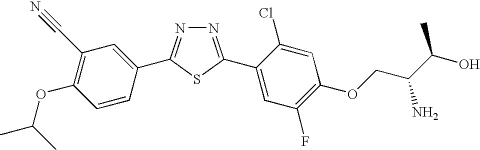 | 5-[5-(4-{[(2R,3R)-2-amino-3- hydroxybutyl]oxy}-2-chloro-5- fluorophenyl)-1,3,4-thiadiazol-2-yl]-2- [(1-methylethyl)oxy]benzonitrile |
| 87 | 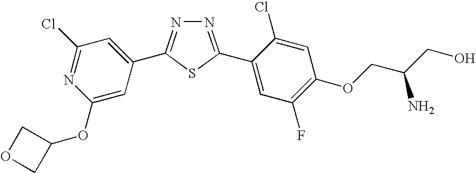 | (2R)-2-amino-3-(5-chloro-4-{5-[2- chloro-6-(oxetan-3-yloxy)pyriidn-4-yl]- 1,3,4-thiadiazol-2-yl}-2- fluorophenoxy)propan-1-ol |
| 88 | 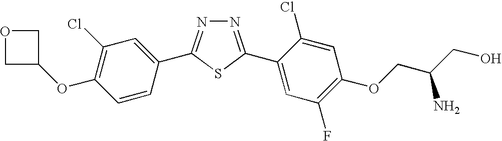 | (2R)-2-amino-3-(5-chloro-4-{5-[3- chloro-4-(oxetan-3-yloxy)phenyl]-1,3,4- thiadiazol-2-yl}-2- fluorophenoxy)propan-1-ol |
| 89 | 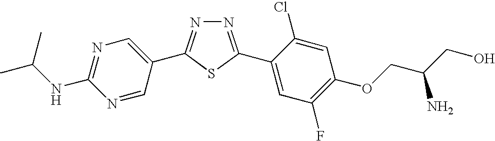 | (2R)-2-amino-3-{[5-chloro-2-fluoro-4- (5-{2-[(1-methylethyl)amino]pyrimidin- 5-yl}-1,3,4-thiadiazol-2- yl)phenyl]oxy}propan-1-ol |
| 90 | 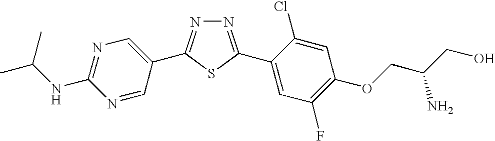 | (2S)-2-amino-3-{[5-chloro-2-fluoro-4- (5-{2-[(1-methylethyl)amino]pyrimidin- 5-yl}-1,3,4-thiadiazol-2- yl)phenyl]oxy}propan-1-ol |
| 91 | 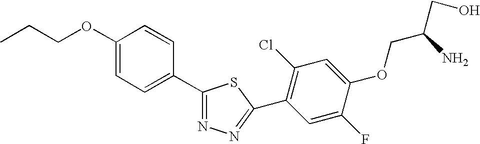 | (2R)-2-amino-3-[(5-chloro-2-fluoro-4- {5-[4-(propyloxy)phenyl]-1,3,4- thiadiaozl-2-yl}phenyl)oxy]propan-1-ol |
| 92 | 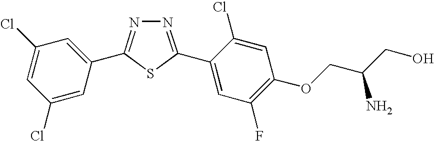 | (2R)-2-amino-3-({5-chloro-4-[5-(3,5- dichlorophenyl)-1,3,4-thiadiazol-2-yl]-2- fluorophenyl}oxy)propan-1-ol |
| 93 | 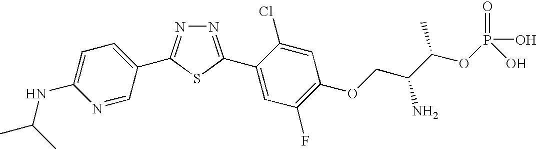 | (1S,2R)-2-amino-3-{[5-chloro-2-fluoro- 4-(5-{6-[(1-methylethyl)amino]pyridin- 3-yl}-1,3,4-thiadiazol-2-yl)phenyl]oxy}- 1-methylpropyl dihydrogen phosphate |
| 94 | 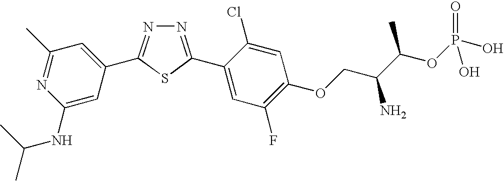 | (1R,2S)-2-amino-3-{[5-chloro-2-fluoro- 4-(5-{2-methyl-6-[(1- methylethyl)amino]pyridin-4-yl}-1,3,4- thiadiazol-2-yl)phenyl]oxy}-1- methylpropyl dihydrogen phosphate |
| 95 | 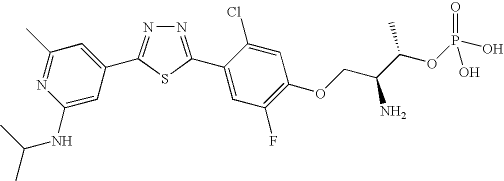 | (1S,2S)-2-amino-3-{[5-chloro-2-fluoro- 4-(5-{2-methyl-6-[(1- methylethyl)amino]pyridin-4-yl}-1,3,4- thiadiazol-2-yl)phenyl]oxy}-1- methylpropyl dihydrogen phosphate |
| 96 | 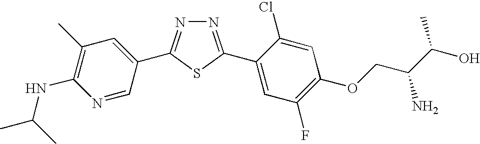 | (2S,3R)-3-amino-4-{[5-chloro-2-fluoro- 4-(5-{5-methyl-6-[(1- methylethyl)amino]pyridin-3-yl}-1,3,4- thiadiazol-2-yl)phenyl]oxy}butan-2-ol |
| 97 | 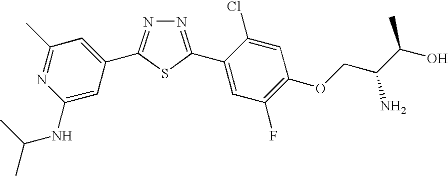 | (2R,3R)-3-amino-4-{[5-chloro-2-fluoro- 4-(5-{2-methyl-6-[(1- methylethyl)amino]pyridin-4-yl}-1,3,4- thiadiazol-2-yl)phenyl]oxy}butan-2-ol |
| 98 | 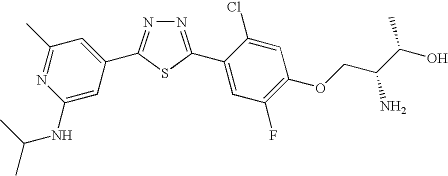 | (2S,3R)-3-amino-4-{[5-chloro-2-fluoro- 4-(5-{2-methyl-6-[(1- methylethyl)amino]pyridin-4-yl}-1,3,4- thiadiazol-2-yl)phenyl]oxy}butan-2-ol |
| 99 | 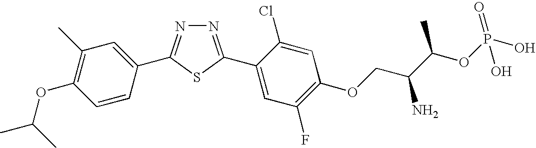 | (1R,2S)-2-amino-3-{[5-chloro-2-fluoro- 4-(5-{3-methyl-4-[(1- methylethyl)oxy]phenyl}-1,3,4- thiadiazol-2-yl)phenyl]oxy}-1- methylpropyl dihydrogen phosphate |
| 100 | 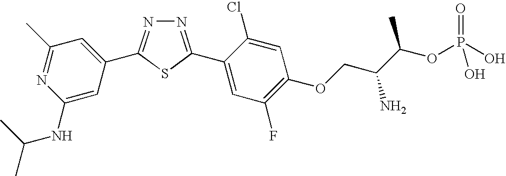 | (1R,2R)-2-amino-3-{[5-chloro-2-fluoro- 4-(5-{2-methyl-6-[(1- methylethyl)amino]pyridin-4-yl}-1,3,4- thiadiazol-2-yl)phenyl]oxy}-1- methylpropyl dihydrogen phosphate |
| 101 | 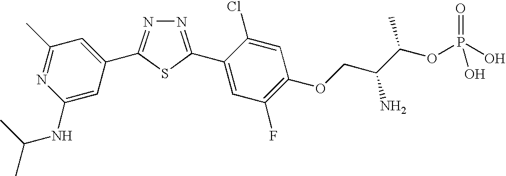 | (1S,2R)-2-amino-3-{[5-chloro-2-fluoro- 4-(5-{2-methyl-6-[(1- methylethyl)amino]pyridin-4-yl}-1,3,4- thiadiazol-2-yl)phenyl]oxy}-1- methylpropyl dihydrogen phosphate |
| 102 | 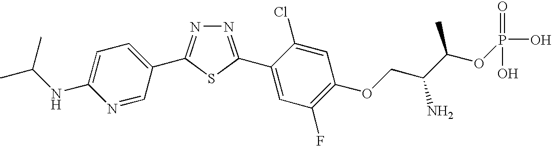 | (1R,2R)-2-amino-3-{[5-chloro-2-fluoro- 4-(5-{6-[(1-methylethyl)amino]pyridin- 3-yl}-1,3,4-thiadiazol-2-yl)phenyl]oxy}- 1-methylpropyl dihydrogen phosphate |
| 103 | 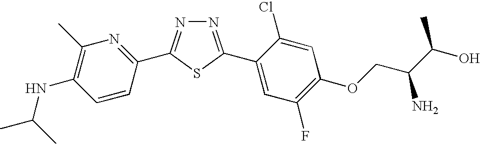 | (2R,3S)-3-amino-4-{[5-chloro-2-fluoro- 4-(5-{6-methyl-5-[(1- methylethyl)amino]pyridin-2-yl}-1,3,4- thiadiazol-2-yl)phenyl]oxy}butan-2-ol |
| 104 | 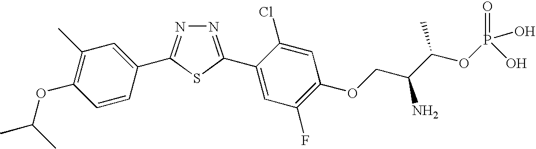 | (1S,2S)-2-amino-3-{[5-chloro-2-fluoro- 4-(5-{3-methyl-4-[(1- methylethyl)oxy]phenyl}-1,3,4- thiadiazol-2-yl)phenyl]oxy}-1- methylpropyl dihydrogen phosphate |
| 105 | 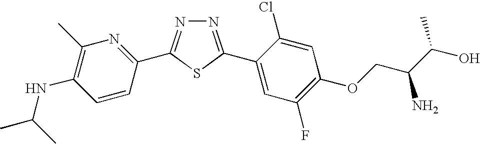 | (2S,3S)-3-amino-4-(5-chloro-2-fluoro-4- {5-[6-methyl-5-(propan-2- ylamino)pyridin-2-yl]-1,3,4-thiadiazol- 2-yl}phenoxy)butan-2-ol |
| 106 | 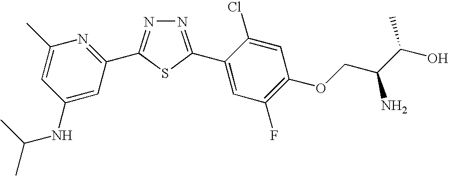 | (2S,3S)-3-amino-4-(5-chloro-2-fluoro-4- {5-[6-methyl-4-(propan-2- ylamino)pyridin-2-yl]-1,3,4-thiadiazol- 2-yl}phenoxy)butan-2-ol |

### Table 1a: Useful Intermediates

The following are useful in the synthesis of compounds of Formula I.

TABLE 1a

| Entry | |
|---|---|
| No. | Name |
| 200 | tert-butyl 4-((5-chloro-4-(5-(6-chloro-5-isopropoxypyridin-2-yl)-1,3,4-thiadiazol-2-yl)-2- |
| | fluorophenoxy)methyl)-2,2-dimethyloxazolidine-3-carboxylate |
| 201 | (S)-tert-butyl 4-((5-chloro-4-(5-(6-chloro-5-isopropoxypyridin-2-yl)-1,3,4-thiadiazol-2-yl)-2- |
| | fluorophenoxy)methyl)-2,2-dimethyloxazolidine-3-carboxylate |
| 202 | (R)-tert-butyl 4-((5-chloro-4-(5-(6-chloro-5-isopropoxypyridin-2-yl)-1,3,4-thiadiazol-2-yl)-2- |
| | fluorophenoxy)methyl)-2,2-dimethyloxazolidine-3-carboxylate |
| 203 | tert-butyl 4-((5-chloro-4-(5-(5-chloro-6-isopropoxypyridin-3-yl)-1,3,4-thiadiazol-2-yl)-2- |
| | fluorophenoxy)methyl)-2,2-dimethyloxazolidine-3-carboxylate |
| 204 | (S)-tert-butyl 4-((5-chloro-4-(5-(5-chloro-6-isopropoxypyridin-3-yl)-1,3,4-thiadiazol-2-yl)-2- |
| | fluorophenoxy)methyl)-2,2-dimethyloxazolidine-3-carboxylate |
| 205 | (R)-tert-butyl 4-((5-chloro-4-(5-(5-chloro-6-isopropoxypyridin-3-yl)-1,3,4-thiadiazol-2-yl)-2- |
| | fluorophenoxy)methyl)-2,2-dimethyloxazolidine-3-carboxylate |
| 206 | tert-butyl 4-((5-chloro-4-(5-(2-chloro-6-(isopropyl(methyl)amino)pyridin-4-yl)-1,3,4-thiadiazol-2- |
| | yl)-2-fluorophenoxy)methyl)-2,2-dimethyloxazolidine-3-carboxylate |
| 207 | (S)-tert-butyl 4-((5-chloro-4-(5-(2-chloro-6-(isopropyl(methyl)amino)pyridin-4-yl)-1,3,4-thiadiazol- |
| | 2-yl)-2-fluorophenoxy)methyl)-2,2-dimethyloxazolidine-3-carboxylate |
| 208 | (R)-tert-butyl 4-((5-chloro-4-(5-(2-chloro-6-(isopropyl(methyl)amino)pyridin-4-yl)-1,3,4-thiadiazol- |
| | 2-yl)-2-fluorophenoxy)methyl)-2,2-dimethyloxazolidine-3-carboxylate |
| 209 | tert-butyl 4-((5-chloro-2-fluoro-4-(5-(imidazo[2,1-b]thiazol-6-yl)-1,3,4-thiadiazol-2- |
| | yl)phenoxy)methyl)-2,2-dimethyloxazolidine-3-carboxylate |
| 210 | (S)-tert-butyl 4-((5-chloro-2-fluoro-4-(5-(4-isobutylphenyl)-1,3,4-thiadiazol-2-yl)phenoxy)methyl)- |
| | 2,2-dimethyloxazolidine-3-carboxylate |
| 211 | (S)-tert-butyl 4-((5-chloro-4-(5-(3-chloro-4-isopropoxyphenyl)-1,3,4-thiadiazol-2-yl)-2- |
| | fluorophenoxy)methyl)-2,2-dimethyloxazolidine-3-carboxylate |
| 212 | (S)-tert-butyl 4-((4-(5-(3-bromo-4-isopropoxyphenyl)-1,3,4-thiadiazol-2-yl)-5-chloro-2- |
| | fluorophenoxy)methyl)-2,2-dimethyloxazolidine-3-carboxylate |
| 213 | (S)-tert-butyl 4-((5-chloro-2-fluoro-4-(5-(4-(trifluoromethoxy)phenyl)-1,3,4-thiadiazol-2- |
| | yl)phenoxy)methyl)-2,2-dimethyloxazolidine-3-carboxylate |
| 214 | (S)-tert-butyl 4-((5-chloro-4-(5-(3-chloro-4-(trifluoromethoxy)phenyl)-1,3,4-thiadiazol-2-yl)-2- |
| | fluorophenoxy)methyl)-2,2-dimethyloxazolidine-3-carboxylate |
| 215 | (S)-tert-butyl 4-((4-(5-(3-bromo-5-chlorophenyl)-1,3,4-thiadiazol-2-yl)-5-chloro-2- |
| | fluorophenoxy)methyl)-2,2-dimethyloxazolidine-3-carboxylate |
| 216 | (R)-tert-butyl 4-((4-(5-(3-bromo-4-isopropoxyphenyl)-1,3,4-thiadiazol-2-yl)-5-chloro-2- |
| | fluorophenoxy)methyl)-2,2-dimethyloxazolidine-3-carboxylate |
| 217 | (S)-tert-butyl 4-((5-chloro-2-fluoro-4-(5-(4-isopropoxy-3-(trifluoromethyl)phenyl)-1,3,4-thiadiazol- |
| | 2-yl)phenoxy)methyl)-2,2-dimethyloxazolidine-3-carboxylate |
| 218 | (S)-tert-butyl 4-((5-chloro-2-fluoro-4-(5-(4-isopropoxyphenyl)-1,3,4-thiadiazol-2- |
| | yl)phenoxy)methyl)-2,2-dimethyloxazolidine-3-carboxylate |
| 219 | (R)-tert-butyl 4-((5-chloro-4-(5-(3-chloro-4-isopropoxyphenyl)-1,3,4-thiadiazol-2-yl)-2- |
| | fluorophenoxy)methyl)-2,2-dimethyloxazolidine-3-carboxylate |
| 220 | (S)-tert-butyl 4-((5-chloro-4-(5-(3-chloro-4-propoxyphenyl)-1,3,4-thiadiazol-2-yl)-2- |
| | fluorophenoxy)methyl)-2,2-dimethyloxazolidine-3-carboxylate |
| 221 | (R)-tert-butyl 4-((4-(5-(3-bromo-4-isopropoxyphenyl)-1,3,4-thiadiazol-2-yl)-5-chloro-2- |
| | fluorophenoxy)methyl)-2,2-dimethyloxazolidine-3-carboxylate |
| 222 | (S)-tert-butyl 4-((5-chloro-2-fluoro-4-(5-(4-isopropoxy-3-(trifluoromethyl)phenyl)-1,3,4-thiadiazol- |
| | 2-yl)phenoxy)methyl)-2,2-dimethyloxazolidine-3-carboxylate |
| 223 | (S)-tert-butyl 4-((5-chloro-2-fluoro-4-(5-(4-isopropoxyphenyl)-1,3,4-thiadiazol-2- |
| | yl)phenoxy)methyl)-2,2-dimethyloxazolidine-3-carboxylate |
| 224 | (R)-tert-butyl 4-((5-chloro-4-(5-(3-chloro-4-isopropoxyphenyl)-1,3,4-thiadiazol-2-yl)-2- |
| | fluorophenoxy)methyl)-2,2-dimethyloxazolidine-3-carboxylate |
| 225 | (S)-tert-butyl 4-((5-chloro-4-(5-(3-chloro-4-propoxyphenyl)-1,3,4-thiadiazol-2-yl)-2- |
| | fluorophenoxy)methyl)-2,2-dimethyloxazolidine-3-carboxylate |
| 226 | (S)-tert-butyl 4-((5-chloro-4-(5-(3-chlorophenyl)-1,3,4-thiadiazol-2-yl)-2-fluorophenoxy)methyl)- |
| | 2,2-dimethyloxazolidine-3-carboxylate |
| 227 | (S)-tert-butyl 4-((4-(5-(3-bromo-5-fluorophenyl)-1,3,4-thiadiazol-2-yl)-5-chloro-2- |
| | fluorophenoxy)methyl)-2,2-dimethyloxazolidine-3-carboxylate |
| 228 | (S)-tert-butyl 4-((5-chloro-4-(5-(3-chloro-4-ethoxyphenyl)-1,3,4-thiadiazol-2-yl)-2- |
| | fluorophenoxy)methyl)-2,2-dimethyloxazolidine-3-carboxylate |
| 229 | (S)-tert-butyl 4-((5-chloro-4-(5-(3,5-dichloro-4-isopropoxyphenyl)-1,3,4-thiadiazol-2-yl)-2- |
| | fluorophenoxy)methyl)-2,2-dimethyloxazolidine-3-carboxylate |
| 230 | (S)-tert-butyl 4-((4-(5-(3-bromo-5-isopropoxyphenyl)-1,3,4-thiadiazol-2-yl)-5-chloro-2- |
| | fluorophenoxy)methyl)-2,2-dimethyloxazolidine-3-carboxylate |
| 231 | (S)-tert-butyl 4-((4-(5-(3-bromo-5-(trifluoromethoxy)phenyl)-1,3,4-thiadiazol-2-yl)-5-chloro-2- |
| | fluorophenoxy)methyl)-2,2-dimethyloxazolidine-3-carboxylate |
| 232 | (S)-tert-butyl 4-((5-chloro-4-(5-(3,5-dibromophenyl)-1,3,4-thiadiazol-2-yl)-2- |
| | fluorophenoxy)methyl)-2,2-dimethyloxazolidine-3-carboxylate |
| 233 | (S)-tert-butyl 4-((3-chloro-4-(5-(3-chloro-4-isopropoxyphenyl)-1,3,4-thiadiazol-2- |
| | yl)phenoxy)methyl)-2,2-dimethyloxazolidine-3-carboxylate |
| 234 | (S)-tert-butyl 4-((5-chloro-4-(5-(3-cyano-4-isopropoxyphenyl)-1,3,4-thiadiazol-2-yl)-2- |
| | fluorophenoxy)methyl)-2,2-dimethyloxazolidine-3-carboxylate |
| 235 | (S)-tert-butyl 4-((2,6-dichloro-4-(5-(3-chloro-4-isopropoxyphenyl)-1,3,4-thiadiazol-2- |
| | yl)phenoxy)methyl)-2,2-dimethyloxazolidine-3-carboxylate |
| 236 | (S)-tert-butyl 4-((2-chloro-4-(5-(3-chloro-4-isopropoxyphenyl)-1,3,4-thiadiazol-2-yl)-6- |
| | fluorophenoxy)methyl)-2,2-dimethyloxazolidine-3-carboxylate |
| 237 | (S)-tert-butyl 4-((5-chloro-2-fluoro-4-(5-(3-fluoro-4-isopropoxyphenyl)-1,3,4-thiadiazol-2- |
| | yl)phenoxy)methyl)-2,2-dimethyloxazolidine-3-carboxylate |
| 238 | (S)-tert-butyl 4-((5-chloro-4-(5-(3-cyano-4-(oxetan-3-yloxy)phenyl)-1,3,4-thiadiazol-2-yl)-2- |
| | fluorophenoxy)methyl)-2,2-dimethyloxazolidine-3-carboxylate |
| 239 | (S)-tert-butyl 4-((5-chloro-4-(5-(3-cyano-4-ethoxyphenyl)-1,3,4-thiadiazol-2-yl)-2- |
| | fluorophenoxy)methyl)-2,2-dimethyloxazolidine-3-carboxylate |
| 240 | (S)-tert-butyl 4-((5-chloro-4-(5-(3-cyano-4-(2-methoxyethoxy)phenyl)-1,3,4-thiadiazol-2-yl)-2- |
| | fluorophenoxy)methyl)-2,2-dimethyloxazolidine-3-carboxylate |
| 241 | (S)-tert-butyl 4-((4-(5-(3-carbamoyl-4-isopropoxyphenyl)-1,3,4-thiadiazol-2-yl)-5-chloro-2- |
| | fluorophenoxy)methyl)-2,2-dimethyloxazolidine-3-carboxylate |
| 242 | (S)-tert-butyl 4-((5-chloro-2-fluoro-4-(5-(4-propoxyphenyl)-1,3,4-thiadiazol-2-yl)phenoxy)methyl)- |
| | 2,2-dimethyloxazolidine-3-carboxylate |
| 243 | (S)-tert-butyl 4-((5-chloro-4-(5-(3,5-dichlorophenyl)-1,3,4-thiadiazol-2-yl)-2- |
| | fluorophenoxy)methyl)-2,2-dimethyloxazolidine-3-carboxylate |
| 244 | (S)-tert-butyl 4-((5-chloro-4-(5-(3-chloro-4-(oxetan-3-yloxy)phenyl)-1,3,4-thiadiazol-2-yl)-2- |
| | fluorophenoxy)methyl)-2,2-dimethyloxazolidine-3-carboxylate |
| 245 | (R)-tert-butyl 1-(4-(5-(3-bromo-4-isopropoxyphenyl)-1,3,4-thiadiazol-2-yl)-5-chloro-2- |
| | fluorophenoxy)-3-(di-tert-butoxyphosphoryloxy)propan-2-ylcarbamate |
| 246 | (S)-tert-butyl 1-(4-(5-(3-bromo-4-isopropoxyphenyl)-1,3,4-thiadiazol-2-yl)-5-chloro-2- |
| | fluorophenoxy)-3-hydroxypropan-2-ylcarbamate |
| 247 | (S)-tert-butyl 1-(5-chloro-4-(5-(3-chloro-4-isopropoxyphenyl)-1,3,4-thiadiazol-2-yl)-2- |
| | fluorophenoxy)-3-(di-tert-butoxyphosphoryloxy)propan-2-ylcarbamate |
| 248 | (R)-tert-butyl 1-(5-chloro-4-(5-(3-chloro-4-isopropoxyphenyl)-1,3,4-thiadiazol-2-yl)-2- |
| | fluorophenoxy)-3-hydroxypropan-2-ylcarbamate |
| 249 | (R)-tert-butyl 1-(5-chloro-4-(5-(3-chloro-4-isopropoxyphenyl)-1,3,4-thiadiazol-2-yl)-2- |
| | fluorophenoxy)-3-(di-tert-butoxyphosphoryloxy)propan-2-ylcarbamate |
| 250 | (R)-tert-butyl 1-(5-chloro-4-(5-(3-chloro-4-isopropoxyphenyl)-1,3,4-thiadiazol-2-yl)-2- |
| | fluorophenoxy)-3-(phosphonooxy)propan-2-ylcarbamate |
| 251 | (S)-tert-butyl 1-(4-(5-(3-bromo-4-isopropoxyphenyl)-1,3,4-thiadiazol-2-yl)-5-chloro-2- |
| | fluorophenoxy)-3-(di-tert-butoxyphosphoryloxy)propan-2-ylcarbamate |
| 252 | (S)-tert-butyl 1-(4-(5-(3-bromo-4-isopropoxyphenyl)-1,3,4-thiadiazol-2-yl)-5-chloro-2- |
| | fluorophenoxy)-3-(phosphonooxy)propan-2-ylcarbamate |
| 253 | (S)-tert-butyl 1-(5-chloro-2-fluoro-4-(5-(6-(isopropylamino)pyridin-3-yl)-1,3,4-thiadiazol-2- |
| | yl)phenoxy)-3-(phosphonooxy)propan-2-ylcarbamate |
| 254 | (S)-tert-butyl 1-(5-chloro-2-fluoro-4-(5-(6-(isopropylamino)pyridin-3-yl)-1,3,4-thiadiazol-2- |
| | yl)phenoxy)-3-(di-tert-butoxyphosphoryloxy)propan-2-ylcarbamate |
| 256 | (S)-tert-butyl 1-(5-chloro-4-(5-(3-cyano-4-isopropoxyphenyl)-1,3,4-thiadiazol-2-yl)-2- |
| | fluorophenoxy)-3-(phosphonooxy)propan-2-ylcarbamate |
| 257 | (S)-tert-butyl 1-(5-chloro-4-(5-(3-cyano-4-isopropoxyphenyl)-1,3,4-thiadiazol-2-yl)-2- |
| | fluorophenoxy)-3-(di-tert-butoxyphosphoryloxy)propan-2-ylcarbamate |
| 258 | tert-butyl (2S,3S)-1-(5-chloro-4-(5-(3-chloro-4-isopropoxyphenyl)-1,3,4-thiadiazol-2-yl)-2- |
| | fluorophenoxy)-3-(phosphonooxy)butan-2-ylcarbamate |
| 259 | tert-butyl (2S,3S)-1-(5-chloro-4-(5-(3-chloro-4-isopropoxyphenyl)-1,3,4-thiadiazol-2-yl)-2- |
| | fluorophenoxy)-3-(di-tert-butoxyphosphoryloxy)butan-2-ylcarbamate |
| 260 | tert-butyl (2R,3R)-1-(5-chloro-4-(5-(3-chloro-4-isopropoxyphenyl)-1,3,4-thiadiazol-2-yl)-2- |
| | fluorophenoxy)-3-(phosphonooxy)butan-2-ylcarbamate |
| 261 | tert-butyl (2R,3R)-1-(5-chloro-4-(5-(3-chloro-4-isopropoxyphenyl)-1,3,4-thiadiazol-2-yl)-2- |
| | fluorophenoxy)-3-(di-tert-butoxyphosphoryloxy)butan-2-ylcarbamate |
| 262 | (R)-tert-butyl 1-(5-chloro-2-fluoro-4-(5-(6-(isopropylamino)pyridin-3-yl)-1,3,4-thiadiazol-2- |
| | yl)phenoxy)-3-(phosphonooxy)propan-2-ylcarbamate |
| 263 | (R)-tert-butyl 1-(5-chloro-2-fluoro-4-(5-(6-(isopropylamino)pyridin-3-yl)-1,3,4-thiadiazol-2- |
| | yl)phenoxy)-3-(di-tert-butoxyphosphoryloxy)propan-2-ylcarbamate |
| 264 | tert-butyl (2S,3R)-1-(5-chloro-4-(5-(3-cyano-4-isopropoxyphenyl)-1,3,4-thiadiazol-2-yl)-2- |
| | fluorophenoxy)-3-(phosphonooxy)butan-2-ylcarbamate |
| 265 | tert-butyl (2S,3R)-1-(5-chloro-4-(5-(3-cyano-4-isopropoxyphenyl)-1,3,4-thiadiazol-2-yl)-2- |
| | fluorophenoxy)-3-(di-tert-butoxyphosphoryloxy)butan-2-ylcarbamate |
| 266 | tert-butyl (2S,3R)-1-(5-chloro-4-(5-(3-chloro-4-isopropoxyphenyl)-1,3,4-thiadiazol-2-yl)-2- |
| | fluorophenoxy)-3-(phosphonooxy)butan-2-ylcarbamate |
| 267 | tert-butyl (2S,3R)-1-(5-chloro-4-(5-(3-chloro-4-isopropoxyphenyl)-1,3,4-thiadiazol-2-yl)-2- |
| | fluorophenoxy)-3-(di-tert-butoxyphosphoryloxy)butan-2-ylcarbamate |
| 268 | tert-butyl (2S,3S)-1-(5-chloro-4-(5-(3-cyano-4-isopropoxyphenyl)-1,3,4-thiadiazol-2-yl)-2- |
| | fluorophenoxy)-3-(phosphonooxy)butan-2-ylcarbamate |
| 269 | tert-butyl (2S,3S)-1-(5-chloro-4-(5-(3-cyano-4-isopropoxyphenyl)-1,3,4-thiadiazol-2-yl)-2- |
| | fluorophenoxy)-3-(di-tert-butoxyphosphoryloxy)butan-2-ylcarbamate |
| 270 | tert-butyl (2S,3R)-1-(5-chloro-2-fluoro-4-(5-(6-(isopropylamino)pyridin-3-yl)-1,3,4-thiadiazol-2- |
| | yl)phenoxy)-3-(phosphonooxy)butan-2-ylcarbamate |
| 271 | tert-butyl (2S,3R)-1-(5-chloro-2-fluoro-4-(5-(6-(isopropylamino)pyridin-3-yl)-1,3,4-thiadiazol-2- |
| | yl)phenoxy)-3-(di-tert-butoxyphosphoryloxy)butan-2-ylcarbamate |
| 272 | tert-butyl (2S,3S)-1-(5-chloro-2-fluoro-4-(5-(6-(isopropylamino)pyridin-3-yl)-1,3,4-thiadiazol-2- |
| | yl)phenoxy)-3-(phosphonooxy)butan-2-ylcarbamate |
| 273 | tert-butyl (2S,3S)-1-(5-chloro-2-fluoro-4-(5-(6-(isopropylamino)pyridin-3-yl)-1,3,4-thiadiazol-2- |
| | yl)phenoxy)-3-(di-tert-butoxyphosphoryloxy)butan-2-ylcarbamate |
| 274 | tert-butyl (2R,3S)-1-(5-chloro-2-fluoro-4-(5-(6-(isopropylamino)pyridin-3-yl)-1,3,4-thiadiazol-2- |
| | yl)phenoxy)-3-(phosphonooxy)butan-2-ylcarbamate |
| 275 | tert-butyl (2R,3S)-1-(5-chloro-2-fluoro-4-(5-(6-(isopropylamino)pyridin-3-yl)-1,3,4-thiadiazol-2- |
| | yl)phenoxy)-3-(di-tert-butoxyphosphoryloxy)butan-2-ylcarbamate |
| 276 | tert-butyl (2R,3S)-1-(5-chloro-4-(5-(3-chloro-4-isopropoxyphenyl)-1,3,4-thiadiazol-2-yl)-2- |
| | fluorophenoxy)-3-(phosphonooxy)butan-2-ylcarbamate |
| 277 | tert-butyl (2R,3S)-1-(5-chloro-4-(5-(3-chloro-4-isopropoxyphenyl)-1,3,4-thiadiazol-2-yl)-2- |
| | fluorophenoxy)-3-(di-tert-butoxyphosphoryloxy)butan-2-ylcarbamate |
| 278 | tert-butyl (2S,3R)-1-(5-chloro-2-fluoro-4-(5-(2-(isopropylamino)-6-methylpyridin-4-yl)-1,3,4- |
| | thiadiazol-2-yl)phenoxy)-3-(phosphonooxy)butan-2-ylcarbamate |
| 279 | tert-butyl (2S,3R)-1-(5-chloro-2-fluoro-4-(5-(2-(isopropylamino)-6-methylpyridin-4-yl)-1,3,4- |
| | thiadiazol-2-yl)phenoxy)-3-(di-tert-butoxyphosphoryloxy)butan-2-ylcarbamate |
| 280 | tert-butyl (2S,3S)-1-(5-chloro-2-fluoro-4-(5-(2-(isopropylamino)-6-methylpyridin-4-yl)-1,3,4- |
| | thiadiazol-2-yl)phenoxy)-3-(phosphonooxy)butan-2-ylcarbamate |
| 281 | tert-butyl (2S,3S)-1-(5-chloro-2-fluoro-4-(5-(2-(isopropylamino)-6-methylpyridin-4-yl)-1,3,4- |
| | thiadiazol-2-yl)phenoxy)-3-(di-tert-butoxyphosphoryloxy)butan-2-ylcarbamate |
| 282 | (S)-tert-butyl 4-((5-chloro-2-fluoro-4-(5-(6-isopropoxypyridin-3-yl)-1,3,4-thiadiazol-2- |
| | yl)phenoxy)methyl)-2,2-dimethyloxazolidine-3-carboxylate |
| 283 | tert-butyl 4-((5-chloro-2-fluoro-4-(5-(6-isopropoxypyridin-3-yl)-1,3,4-thiadiazol-2- |
| | yl)phenoxy)methyl)-2,2-dimethyloxazolidine-3-carboxylate |
| 284 | (R)-tert-butyl 4-((5-chloro-2-fluoro-4-(5-(6-isopropoxypyridin-3-yl)-1,3,4-thiadiazol-2- |
| | yl)phenoxy)methyl)-2,2-dimethyloxazolidine-3-carboxylate |
| 285 | tert-butyl 4-((5-chloro-2-fluoro-4-(5-(6-(isopropyl(methyl)amino)pyridin-3-yl)-1,3,4-thiadiazol-2- |
| | yl)phenoxy)methyl)-2,2-dimethyloxazolidine-3-carboxylate |
| 286 | (S)-tert-butyl 4-((5-chloro-2-fluoro-4-(5-(2-(isopropyl(methyl)amino)pyridin-4-yl)-1,3,4-thiadiazol- |
| | 2-yl)phenoxy)methyl)-2,2-dimethyloxazolidine-3-carboxylate |
| 287 | (S)-tert-butyl 4-((4-(5-(4-(allyl(isopropyl)amino)-3-chlorophenyl)-1,3,4-thiadiazol-2-yl)-5-chloro-2- |
| | fluorophenoxy)methyl)-2,2-dimethyloxazolidine-3-carboxylate |
| 288 | (S)-tert-butyl 4-((5-chloro-4-(5-(3-chloro-4-(isopropylamino)phenyl)-1,3,4-thiadiazol-2-yl)-2- |
| | fluorophenoxy)methyl)-2,2-dimethyloxazolidine-3-carboxylate |
| 289 | (R)-tert-butyl 4-((4-(5-(4-(allyl(isopropyl)amino)-3-chlorophenyl)-1,3,4-thiadiazol-2-yl)-5-chloro-2- |
| | fluorophenoxy)methyl)-2,2-dimethyloxazolidine-3-carboxylate |
| 290 | (R)-tert-butyl 4-((5-chloro-4-(5-(3-chloro-4-(isopropylamino)phenyl)-1,3,4-thiadiazol-2-yl)-2- |
| | fluorophenoxy)methyl)-2,2-dimethyloxazolidine-3-carboxylate |
| 291 | tert-butyl 4-((4-(5-(6-(allyl(isopropyl)amino)pyridin-3-yl)-1,3,4-thiadiazol-2-yl)-5-chloro-2- |
| | fluorophenoxy)methyl)-2,2-dimethyloxazolidine-3-carboxylate |
| 292 | (S)-tert-butyl 4-((5-chloro-2-fluoro-4-(5-(6-(isopropylamino)pyridin-3-yl)-1,3,4-thiadiazol-2- |
| | yl)phenoxy)methyl)-2,2-dimethyloxazolidine-3-carboxylate |
| 293 | (S)-tert-butyl 4-((4-(5-(6-(allyl(isopropyl)amino)pyridin-3-yl)-1,3,4-thiadiazol-2-yl)-5-chloro-2- |
| | fluorophenoxy)methyl)-2,2-dimethyloxazolidine-3-carboxylate |
| 294 | (S)-tert-butyl 4-((5-chloro-2-fluoro-4-(5-(6-(isopropylamino)pyridin-3-yl)-1,3,4-thiadiazol-2- |
| | yl)phenoxy)methyl)-2,2-dimethyloxazolidine-3-carboxylate |
| 295 | (R)-tert-butyl 4-((4-(5-(6-(allyl(isopropyl)amino)pyridin-3-yl)-1,3,4-thiadiazol-2-yl)-5-chloro-2- |
| | fluorophenoxy)methyl)-2,2-dimethyloxazolidine-3-carboxylate |
| 296 | (R)-tert-butyl 4-((5-chloro-2-fluoro-4-(5-(6-(isopropylamino)pyridin-3-yl)-1,3,4-thiadiazol-2- |
| | yl)phenoxy)methyl)-2,2-dimethyloxazolidine-3-carboxylate |
| 297 | (4S,5R)-tert-butyl 4-((4-(5-(6-(allyl(isopropyl)amino)pyridin-3-yl)-1,3,4-thiadiazol-2-yl)-5-chloro- |
| | 2-fluorophenoxy)methyl)-2,2,5-trimethyloxazolidine-3-carboxylate |
| 298 | (4S,5R)-tert-butyl 4-((5-chloro-2-fluoro-4-(5-(6-(isopropylamino)pyridin-3-yl)-1,3,4-thiadiazol-2- |
| | yl)phenoxy)methyl)-2,2,5-trimethyloxazolidine-3-carboxylate |
| 299 | (4S,5S)-tert-butyl 4-((4-(5-(6-(allyl(isopropyl)amino)pyridin-3-yl)-1,3,4-thiadiazol-2-yl)-5-chloro-2- |
| | fluorophenoxy)methyl)-2,2,5-trimethyloxazolidine-3-carboxylate |
| 300 | (4S,5S)-tert-butyl 4-((5-chloro-2-fluoro-4-(5-(6-(isopropylamino)pyridin-3-yl)-1,3,4-thiadiazol-2- |
| | yl)phenoxy)methyl)-2,2,5-trimethyloxazolidine-3-carboxylate |
| 301 | (4R,5S)-tert-butyl 4-((4-(5-(6-(allyl(isopropyl)amino)pyridin-3-yl)-1,3,4-thiadiazol-2-yl)-5-chloro- |
| | 2-fluorophenoxy)methyl)-2,2,5-trimethyloxazolidine-3-carboxylate |
| 302 | (4R,5S)-tert-butyl 4-((5-chloro-2-fluoro-4-(5-(6-(isopropylamino)pyridin-3-yl)-1,3,4-thiadiazol-2- |
| | yl)phenoxy)methyl)-2,2,5-trimethyloxazolidine-3-carboxylate |
| 303 | (4R,5R)-tert-butyl 4-((4-(5-(6-(allyl(isopropyl)amino)pyridin-3-yl)-1,3,4-thiadiazol-2-yl)-5-chloro- |
| | 2-fluorophenoxy)methyl)-2,2,5-trimethyloxazolidine-3-carboxylate |
| 304 | (4R,5R)-tert-butyl 4-((5-chloro-2-fluoro-4-(5-(6-(isopropylamino)pyridin-3-yl)-1,3,4-thiadiazol-2- |
| | yl)phenoxy)methyl)-2,2,5-trimethyloxazolidine-3-carboxylate |
| 305 | (4S,5R)-tert-butyl 4-((4-(5-(6-(allyl(isopropyl)amino)-5-chloropyridin-3-yl)-1,3,4-thiadiazol-2-yl)- |
| | 5-chloro-2-fluorophenoxy)methyl)-2,2,5-trimethyloxazolidine-3-carboxylate |
| 306 | (4S,5R)-tert-butyl 4-((5-chloro-4-(5-(5-chloro-6-(isopropylamino)pyridin-3-yl)-1,3,4-thiadiazol-2- |
| | yl)-2-fluorophenoxy)methyl)-2,2,5-trimethyloxazolidine-3-carboxylate |
| 307 | (S)-tert-butyl 4-((5-chloro-4-(5-(5-chloro-6-(isopropylamino)pyridin-3-yl)-1,3,4-thiadiazol-2-yl)-2- |
| | fluorophenoxy)methyl)-2,2-dimethyloxazolidine-3-carboxylate |
| 308 | (4R,5S)-tert-butyl 4-((5-chloro-4-(5-(3-cyano-4-isopropoxyphenyl)-1,3,4-thiadiazol-2-yl)-2- |
| | fluorophenoxy)methyl)-2,2,5-trimethyloxazolidine-3-carboxylate |
| 309 | (4R)-tert-butyl 4-((5-chloro-4-(5-(3-chloro-4-isopropoxyphenyl)-1,3,4-thiadiazol-2-yl)-2- |
| | fluorophenoxy)methyl)-2,2,5-trimethyloxazolidine-3-carboxylate |
| 310 | (4S)-tert-butyl 4-((5-chloro-4-(5-(3-chloro-4-isopropoxyphenyl)-1,3,4-thiadiazol-2-yl)-2- |
| | fluorophenoxy)methyl)-2,2,5-trimethyloxazolidine-3-carboxylate |
| 311 | (4S,5R)-tert-butyl 4-((5-chloro-4-(5-(3-chloro-4-isopropoxyphenyl)-1,3,4-thiadiazol-2-yl)-2- |
| | fluorophenoxy)methyl)-2,2,5-trimethyloxazolidine-3-carboxylate |
| 312 | (4R,5R)-tert-butyl 4-((5-chloro-4-(5-(3-chloro-4-isopropoxyphenyl)-1,3,4-thiadiazol-2-yl)-2- |
| | fluorophenoxy)methyl)-2,2,5-trimethyloxazolidine-3-carboxylate |
| 313 | (4S,5R)-tert-butyl 4-((5-chloro-4-(5-(3-cyano-4-isopropoxyphenyl)-1,3,4-thiadiazol-2-yl)-2- |
| | fluorophenoxy)methyl)-2,2,5-trimethyloxazolidine-3-carboxylate |
| 314 | (4S,5S)-tert-butyl 4-((5-chloro-4-(5-(3-cyano-4-isopropoxyphenyl)-1,3,4-thiadiazol-2-yl)-2- |
| | fluorophenoxy)methyl)-2,2,5-trimethyloxazolidine-3-carboxylate |
| 315 | (4S,5R)-tert-butyl 4-((5-chloro-2-fluoro-4-(5-(4-isopropoxy-3-methylphenyl)-1,3,4-thiadiazol-2- |
| | yl)phenoxy)methyl)-2,2,5-trimethyloxazolidine-3-carboxylate |
| 316 | (4S,5S)-tert-butyl 4-((5-chloro-2-fluoro-4-(5-(4-isopropoxy-3-methylphenyl)-1,3,4-thiadiazol-2- |
| | yl)phenoxy)methyl)-2,2,5-trimethyloxazolidine-3-carboxylate |
| 317 | (4S,5R)-tert-butyl 4-((4-(5-(2-(allyl(isopropyl)amino)pyridin-4-yl)-1,3,4-thiadiazol-2-yl)-5-chloro- |
| | 2-fluorophenoxy)methyl)-2,2,5-trimethyloxazolidine-3-carboxylate |
| 318 | (4S,5R)-tert-butyl 4-((5-chloro-2-fluoro-4-(5-(2-(isopropylamino)pyridin-4-yl)-1,3,4-thiadiazol-2- |
| | yl)phenoxy)methyl)-2,2,5-trimethyloxazolidine-3-carboxylate |
| 319 | (4S,5S)-tert-butyl 4-((5-chloro-2-fluoro-4-(5-(2-(isopropylamino)pyridin-4-yl)-1,3,4-thiadiazol-2- |
| | yl)phenoxy)methyl)-2,2,5-trimethyloxazolidine-3-carboxylate |
| 320 | (4S,5R)-tert-Butyl 4-((5-chloro-2-fluoro-4-(5-(2-(isopropylamino)-6-methylpyridin-4-yl)-1,3,4- |
| | thiadiazol-2-yl)phenoxy)methyl)-2,2,5-trimethyloxazolidine-3-carboxylate |
| 321 | (4S,5S)-tert-butyl 4-((5-chloro-2-fluoro-4-(5-(2-(isopropylamino)-6-methylpyridin-4-yl)-1,3,4- |
| | thiadiazol-2-yl)phenoxy)methyl)-2,2,5-trimethyloxazolidine-3-carboxylate |
| 322 | (4S,5R)-tert-Butyl 4-((5-chloro-2-fluoro-4-(5-(6-(isopropylamino)-5-methylpyridin-3-yl)-1,3,4- |
| | thiadiazol-2-yl)phenoxy)methyl)-2,2,5-trimethyloxazolidine-3-carboxylate |
| 323 | (4S,5S)-tert-butyl 4-((5-chloro-2-fluoro-4-(5-(6-(isopropylamino)-5-methylpyridin-3-yl)-1,3,4- |
| | thiadiazol-2-yl)phenoxy)methyl)-2,2,5-trimethyloxazolidine-3-carboxylate |
| 324 | (4R,5R)-tert-butyl 4-((5-chloro-2-fluoro-4-(5-(6-(isopropylamino)-5-methylpyridin-3-yl)-1,3,4- |
| | thiadiazol-2-yl)phenoxy)methyl)-2,2,5-trimethyloxazolidine-3-carboxylate |
| 325 | (S)-tert-butyl 4-((4-(5-(2-(allyloxy)-6-chloropyridin-4-yl)-1,3,4-thiadiazol-2-yl)-5-chloro-2- |
| | fluorophenoxy)methyl)-2,2-dimethyloxazolidine-3-carboxylate |
| 326 | (S)-tert-butyl 4-((5-chloro-4-(5-(2-chloro-6-hydroxypyridin-4-yl)-1,3,4-thiadiazol-2-yl)-2- |
| | fluorophenoxy)methyl)-2,2-dimethyloxazolidine-3-carboxylate |
| 327 | (S)-tert-butyl 4-((5-chloro-4-(5-(2-chloro-6-(oxetan-3-yloxy)pyridin-4-yl)-1,3,4-thiadiazol-2-yl)-2- |
| | fluorophenoxy)methyl)-2,2-dimethyloxazolidine-3-carboxylate |
| 328 | (S)-tert-butyl 4-((5-chloro-2-fluoro-4-(5-(2-(isopropylamino)pyrimidin-5-yl)-1,3,4-thiadiazol-2- |
| | yl)phenoxy)methyl)-2,2-dimethyloxazolidine-3-carboxylate |
| 329 | (R)-tert-butyl 4-((5-chloro-2-fluoro-4-(5-(2-(isopropylamino)pyrimidin-5-yl)-1,3,4-thiadiazol-2- |
| | yl)phenoxy)methyl)-2,2-dimethyloxazolidine-3-carboxylate |

### General Administration

In one aspect, the invention provides pharmaceutical compositions comprising an inhibitor of S1P1 and/or S1P5 according to the invention and a pharmaceutically acceptable carrier, excipient, or diluent. In certain other specific embodiments, administration is by the oral route. Administration of the compounds of the invention, or their pharmaceutically acceptable salts, in pure form or in an appropriate pharmaceutical composition, can be carried out via any of the accepted modes of administration or agents for serving similar utilities. Thus, administration can be, for example, orally, nasally, parenterally (intravenous, intramuscular, or subcutaneous), topically, transdermally, intravaginally, intravesically, intracistemally, or rectally, in the form of solid, semi-solid, lyophilized powder, or liquid dosage forms, such as for example, tablets, suppositories, pills, soft elastic and hard gelatin capsules, powders, solutions, suspensions, or aerosols, or the like, specifically in unit dosage forms suitable for simple administration of precise dosages.

The compositions will include a conventional pharmaceutical carrier or excipient and a compound of the invention as the/an active agent, and, in addition, may include carriers and adjuvants, etc.

Adjuvants include preserving, wetting, suspending, sweetening, flavoring, perfuming, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.]

If desired, a pharmaceutical composition of the invention may also contain minor amounts of auxiliary substances such as wetting or emulsifying agents, pH buffering agents, antioxidants, and the like, such as, for example, citric acid, sorbitan monolaurate, triethanolamine oleate, butylalted hydroxytoluene, etc.

The choice of formulation depends on various factors such as the mode of drug administration (e.g., for oral administration, formulations in the form of tablets, pills or capsules) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

One specific route of administration is oral, using a convenient daily dosage regimen that can be adjusted according to the degree of severity of the disease-state to be treated.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, magnesium stearate and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid dosage forms as described above can be prepared with coatings and shells, such as enteric coatings and others well known in the art. They may contain pacifying agents, and can also be of such composition that they release the active compound or compounds in a certain part of the intestinal tract in a delayed manner. Examples of embedded compositions that can be used are polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., a compound(s) of the invention, or a pharmaceutically acceptable salt thereof, and optional pharmaceutical adjuvants in a carrier, such as, for example, water, saline, aqueous dextrose, glycerol, ethanol and the like; solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are, for example, suppositories that can be prepared by mixing the compounds of the present invention with for example suitable non-irritating excipients or carriers such as cocoa butter, polyethyleneglycol or a suppository wax, which are solid at ordinary temperatures but liquid at body temperature and therefore, melt while in a suitable body cavity and release the active component therein.

Dosage forms for topical administration of a compound of this invention include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this invention.

Compressed gases may be used to disperse a compound of this invention in aerosol form. Inert gases suitable for this purpose are nitrogen, carbon dioxide, etc.

Generally, depending on the intended mode of administration, the pharmaceutically acceptable compositions will contain about 1% to about 99% by weight of a compound(s) of the invention, or a pharmaceutically acceptable salt thereof, and 99% to 1% by weight of a suitable pharmaceutical excipient. In one example, the composition will be between about 5% and about 75% by weight of a compound(s) of the invention, or a pharmaceutically acceptable salt thereof, with the rest being suitable pharmaceutical excipients.

Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of the invention, or a pharmaceutically acceptable salt thereof, for treatment of a disease-state in accordance with the teachings of this invention.

The compounds of the invention, or their pharmaceutically acceptable salts or solvates, are administered in a therapeutically effective amount which will vary depending upon a variety of factors including the activity of the specific compound employed, the metabolic stability and length of action of the compound, the age, body weight, general health, sex, diet, mode and time of administration, rate of excretion, drug combination, the severity of the particular disease-states, and the host undergoing therapy. The compounds of the present invention can be administered to a patient at dosage levels in the range of about 0.1 to about 1,000 mg per day. For a normal human adult having a body weight of about 70 kilograms, a dosage in the range of about 0.01 to about 100 mg per kilogram of body weight per day is an example. The specific dosage used, however, can vary. For example, the dosage can depend on a number of factors including the requirements of the patient, the severity of the condition being treated, and the pharmacological activity of the compound being used. The determination of optimum dosages for a particular patient is well known to one of ordinary skill in the art.

If formulated as a fixed dose, such combination products employ the compounds of this invention within the dosage range described above and the other pharmaceutically active agent(s) within its approved dosage range. Compounds of the instant invention may alternatively be used sequentially with known pharmaceutically acceptable agent(s) when a combination formulation is inappropriate.

### General Synthesis

Compounds of this invention can be made by the synthetic procedures described below. The starting materials and reagents used in preparing these compounds are either available from commercial suppliers such as Aldrich Chemical Co. (Milwaukee, Wis.), or Bachem (Torrance, Calif.), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4^{th} Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure. The starting materials and the intermediates of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure and over a temperature range from about −78° C. to about 150° C., more specifically from about 0° C. to about 125° C. and more specifically at about room (or ambient) temperature, e.g., about 20° C. Unless otherwise stated (as in the case of an hydrogenation), all reactions are performed under an atmosphere of nitrogen.

Prodrugs can be prepared by techniques known to one skilled in the art. These techniques generally modify appropriate functional groups in a given compound. These modified functional groups regenerate original functional groups by routine manipulation or in vivo. Amides and esters of the compounds of the present invention may be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

The compounds of the invention, or their pharmaceutically acceptable salts, may have asymmetric carbon atoms or quaternized nitrogen atoms in their structure. Compounds of the Invention that may be prepared through the syntheses described herein may exist as single stereoisomers, racemates, and as mixtures of enantiomers and diastereomers. The compounds may also exist as geometric isomers. All such single stereoisomers, racemates and mixtures thereof, and geometric isomers are intended to be within the scope of this invention. Some of the compounds of the invention may exist as tautomers. For example, where a ketone or aldehyde is present, the molecule may exist in the enol form; where an amide is present, the molecule may exist as the imidic acid; and where an enamine is present, the molecule may exist as an imine. All such tautomers are within the scope of the invention.

The present invention also includes N-oxide derivatives and protected derivatives of compounds of the Invention. For example, when compounds of the Invention contain an oxidizable nitrogen atom, the nitrogen atom can be converted to an N-oxide by methods well known in the art. When compounds of the Invention contain groups such as hydroxy, carboxy, thiol or any group containing a nitrogen atom(s), these groups can be protected with a suitable “protecting group” or “protective group”. A comprehensive list of suitable protective groups can be found in T. W. Greene, Protective Groups in Organic Synthesis, John Wiley & Sons, Inc. 1991, the disclosure of which is incorporated herein by reference in its entirety. For example nitrogen protecting groups include, but are not limited to Boc, Fmoc, benzyl, trityl, and the like. The protected derivatives of compounds of the Invention can be prepared by methods well known in the art.

Methods for the preparation and/or separation and isolation of single stereoisomers from racemic mixtures or non-racemic mixtures of stereoisomers are well known in the art. For example, optically active (R)- and (S)-isomers may be prepared using chiral synthons or chiral reagents, or resolved using conventional techniques. Enantiomers (R- and S-isomers) may be resolved by methods known to one of ordinary skill in the art, for example by: formation of diastereoisomeric salts or complexes which may be separated, for example, by crystallization; via formation of diastereoisomeric derivatives which may be separated, for example, by crystallization, selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where a desired enantiomer is converted into another chemical entity by one of the separation procedures described above, a further step may be required to liberate the desired enantiomeric form. Alternatively, specific enantiomer may be synthesized by asymmetric synthesis using optically active reagents, substrates, catalysts or solvents or by converting on enantiomer to the other by asymmetric transformation. For a mixture of enantiomers, enriched in a particular enantiomer, the major component enantiomer may be further enriched (with concomitant loss in yield) by recrystallization.

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

The chemistry for the preparation of the compounds of this invention is known to those skilled in the art. In fact, there may be more than one process to prepare the compounds of the invention. The following examples illustrate but do not limit the invention. All references cited herein are incorporated by reference in their entirety.

A Compound of the Invention where R^{7c} is hydrogen or methyl, R⁷, R^{7a}, R^{7b}, R⁸, R⁵, R^{5a}, and R¹ are hydrogen, and n, Ring A, R², R^{2a}, R³, and R⁴ are as defined in the Summary of the Invention for a Compound of Formula I can be prepared according to Scheme 1 and 2.

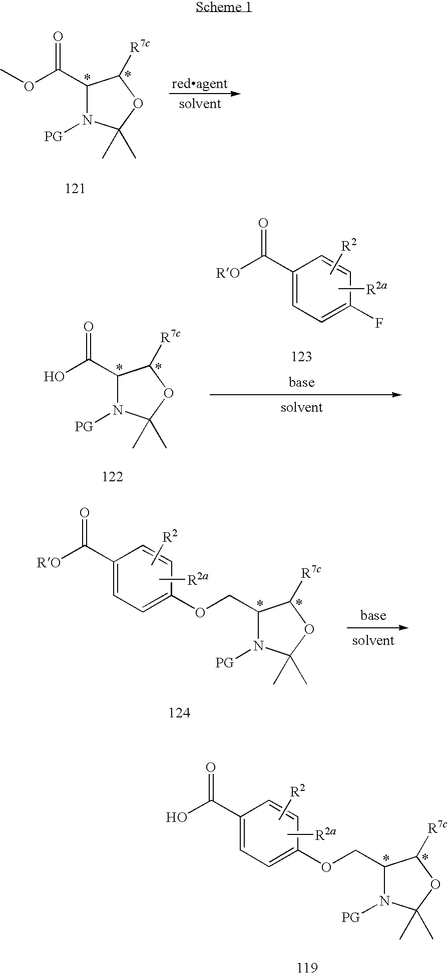

An intermediate of formula 121 (where PG is a nitrogen-protecting group) is commercially available or can be prepared using procedures described in J. Org. Chem. 1987, 52(12), 2361-4. Intermediate 121 is treated with a reducing agent such as LiBH₄, in a solvent(s) such as THF and/or methanol using procedures similar to those described in Synth. Commmun. 1994, 24, 2147 to yield an intermediate of formula 122.

Intermediate 122 is then treated with an intermediate of formula 123 (where R′ is hydrogen, methyl, or ethyl), which is commerically available or can be prepared using procedures known to one of ordinary skill in the art, and a base such as NaH, in the presence of a solvent such as dioxane or DMF and allowed to react at about 0° C. to yield an intermediate of formula 124. The intermediate of formula 124 is then treated with a base such as LiOH, in a solvent(s) such as THF and/or water at about room temperature to yield an intermediate of formula 119.

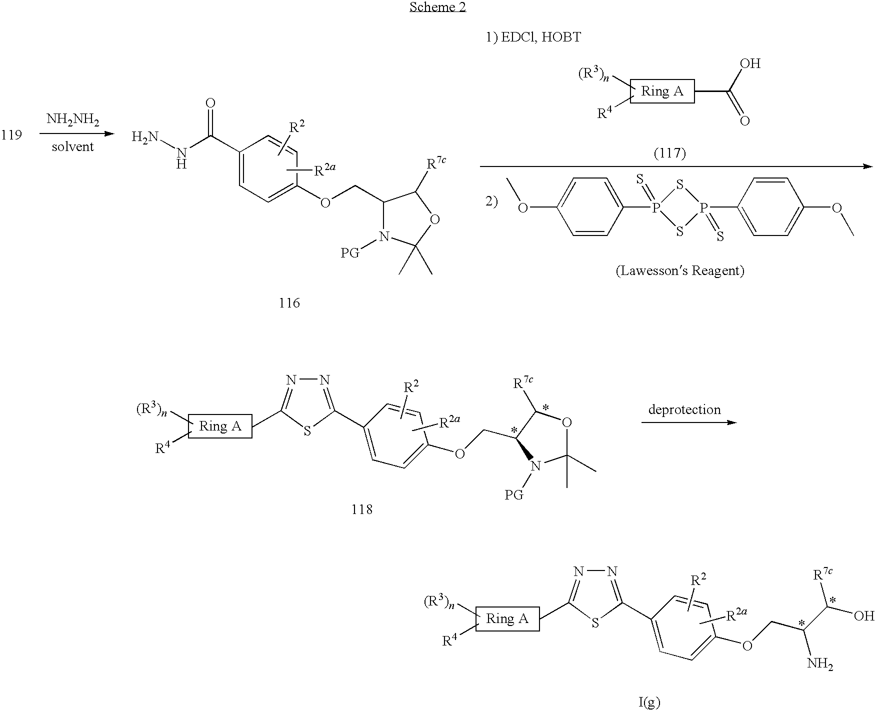

The intermediate of formula 119 is then treated with hydrazine and allowed to react in a solvent such as methanol or ethanol at a temperature of about 65-80° C. to yield an intermediate of formula 116.

The intermediate of formula 116 is then treated with an intermediate of formula 117 in the presence of a coupling agent(s) such as EDCI and/or HOBt, a base such as Hünig's base, and in a solvent such as DMF or DMA to form a hydrazide intermediate. The hydrazide intermediate is then treated with Lawesson's reagent in a solvent such as dioxane at about 90° C. for about 12 hours to yield an intermediate of formula 118.

The intermediate of formula 118 is then deprotected. Where the protecting group is Boc, it can be removed using HCl in a solvent such as dioxane to yield the HCl salt of the Compound of Formula I(g).

A Compound of the Invention where R⁷, R^{7a}, R^{7b}, R^{7c}, R⁸, R⁵, and R^{5a} are hydrogen, R¹ is —P(O)(OR⁶)₂, and n, Ring A, R², R^{2a}, R³, R⁴, and R⁶ are as defined in the Summary of the Invention for a Compound of Formula I can be prepared according to Scheme 3 and 4.

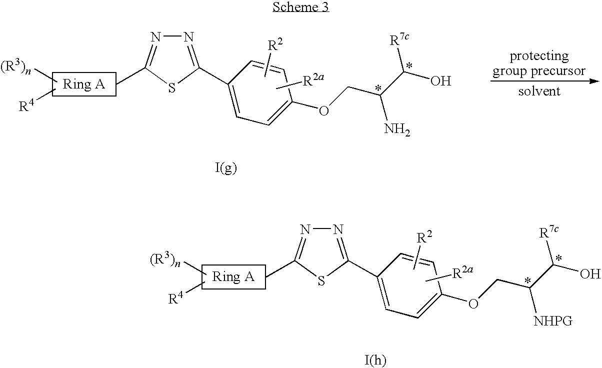

A compound of Formula I(g), prepared as described above, can be used to make other Compounds of the Invention. The free amine of I(g) is protected using a protecting group precursor such as Boc-anhydride in the presence of a base such as triethylamine, and in a solvent such as dioxane at about room temperature to yield a Compound of Formula I(h).

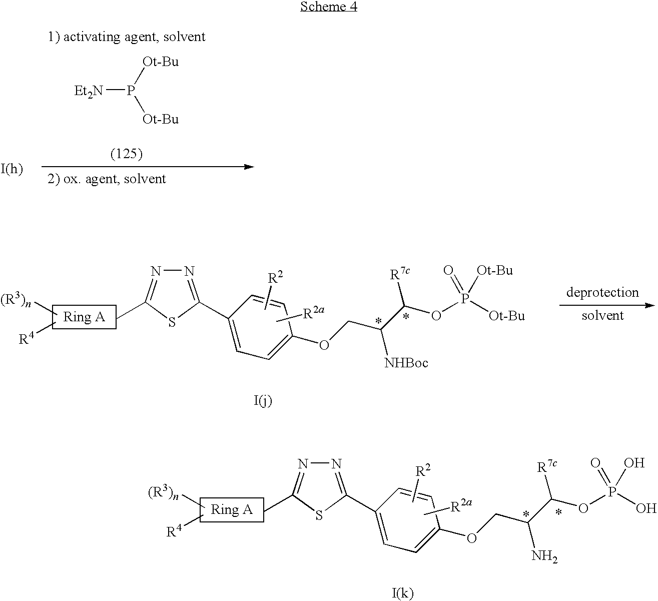

Following procedures in Tetrahedron 2005, 61(3), 609, the Compound of Formula I(h) is then treated with di-tert-butyl-N,N-diethylphosphoramidite (125), in the presence of an activating agent such as tetrazole, and in a solvent such as THF or acetonitrile at room temperature for about an hour. The reaction is then treated with an oxidizing agent such as MCPBA, and carried out in a solvent such as DCM at about 0° C. for about 3 hours to yield a Compound of Formula I(j).

The Compound of Formula I(j) is then deprotected to yield a Compound of Formula I(k). Where PG is Boc, it can be removed in the presence of HCl in a solvent such as dioxane.

An intermediate of formula 10 where n, R³, R⁴, and Ring A are as defined in the Summary of the Invention for a Compound of Formula I can be prepared according to Scheme 5.

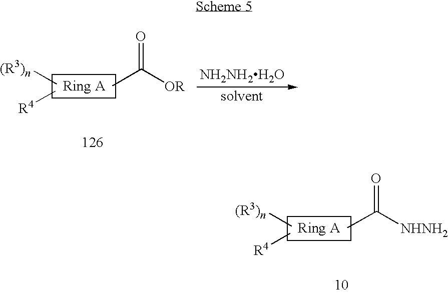

An intermediate of formula 126 (where R is alkyl), which is commercially available or can be prepared using procedures known to one of ordinary skill in the art, is treated with hydrazine monohydrate in a solvent such as ethanol and allowed to reflux for about 2 hours to yield an intermediate of formula 10.

An intermediate of formula 58 is useful in the preparation of Compounds of the Invention and can be prepared according to Scheme 6.

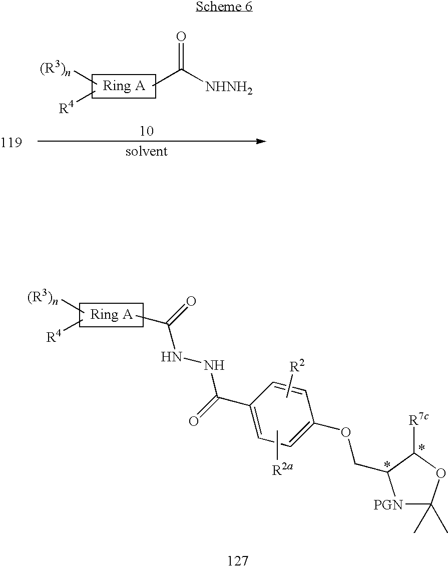

An intermediate of formula 53(a), prepared as described above, is treated with an intermediate of formula 10, prepared as described in Scheme 5, in the presence of a coupling agent(s) such as EDCI and/or HOBt, a base such as Hünig's base, and in a solvent such as DMF or DMA. 58 can then be treated with Lawesson's reagent and further reacted using procedures described in Scheme 2 to yield a Compound of the Invention.

### Synthetic Examples

### Intermediate 6

### 4-((3-(tert-Butoxycarbonyl)-2,2-dimethyloxazolidin-4-yl)methoxy)-2-chloro-5-fluorobenzoic acid

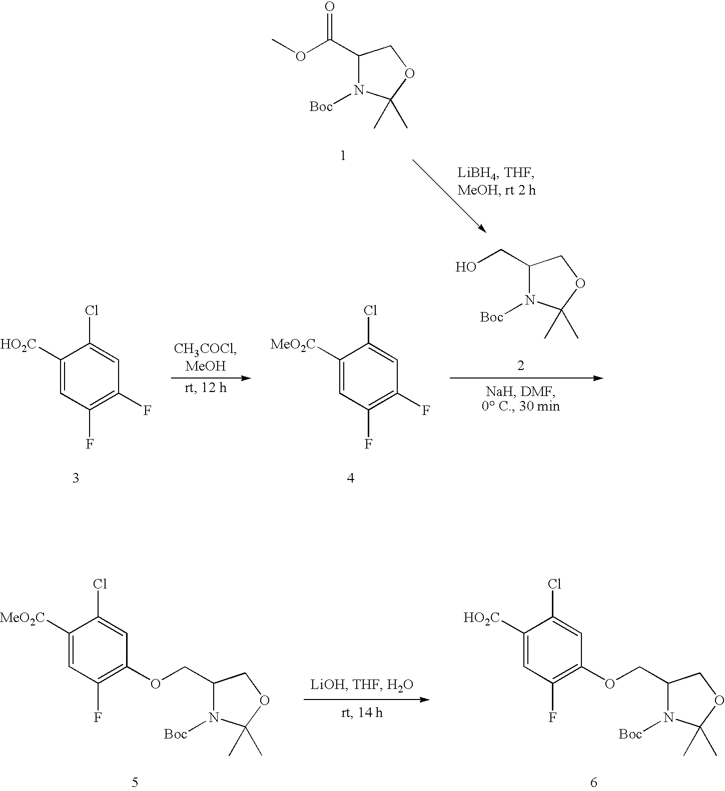

Step 1: tert-Butyl 4-(hydroxymethyl)-2,2-dimethyloxazolidine-3-carboxylate (2). To an ice cold solution of commercially-available 1 (35 g, 135 mmol) in THF/MeOH (500 mL (95:5)), was added LiBH₄ (5.9 g, 271 mmol) portionwise and the suspension was stirred for 2 h at room temperature. The reaction mixture was cooled and quenched with ice. Solvent was removed under reduced pressure, and water was added. The aqueous layer was extracted with EtOAc, concentrated and chromatographed (EtOAc:Hexane, 3:7) to yield 2 (23 g, 74% yield).

Step 2: Methyl 2-chloro-4,5-difluorobenzoate (4). Acetyl chloride (13.92 mL, 196 mmol) was added dropwise to an ice cold solution of commercially-available 3 (10.0 g, 52 mmol) in MeOH (100 mL) and the reaction mixture was stirred for 12 h at room temperature. Solvent was removed in vacuo and the resulting residue was dissolved in EtOAc, washed with aqueous NaHCO₃ and concentrated in vacuo to obtain crude 4 (11 g, ˜100% yield) which was used in subsequent reactions without further purification.

Step 3: tert-Butyl 4-((5-chloro-2-fluoro-4-(methoxycarbonyl)phenoxy)methyl)-2,2-dimethyloxazolidine-3-carboxylate (5). To a stirred solution of 4 (16 g, 77 mmol) in DMF (100 mL) at 0° C. was added 2 (19.5 g, 84 mmol) followed by 60% NaH (5.3 g, 132 mmol). The reaction mixture was stirred for another 30 min at the same temperature and then it was quenched with ice water and extracted with EtOAc. The organic layer was dried over Na₂SO₄ and concentrated to afford crude 5 (11 g, ˜34% yield) which was used in subsequent reactions without further purification.

Step 4: 4-((3-(tert-Butoxycarbonyl)-2,2-dimethyloxazolidin-4-yl)methoxy)-2-chloro-5-fluorobenzoic acid (6). To a stirred solution of intermediate 5 (16 g, 38 mmol) in THF/water (100 mL, 1:1) was added LiOH monohydrate (6.2 g, 148 mmol) at 0° C., followed by stirring at room temperature for 14 h. The reaction was then neutralized with 10% citric acid solution. THF was removed in vacuo and the aqueous layer was extracted with EtOAc. The organic layer was dried over Na₂SO₄ and concentrated to afford crude 6 (14 g, ˜90% yield), which was used in subsequent reactions without further purification.

### Intermediate 9

### tert-Butyl 4-((5-chloro-2-fluoro-4-(hydrazinecarbonyl)phenoxy)methyl)-2,2-dimethyloxazolidine-3-carboxylate

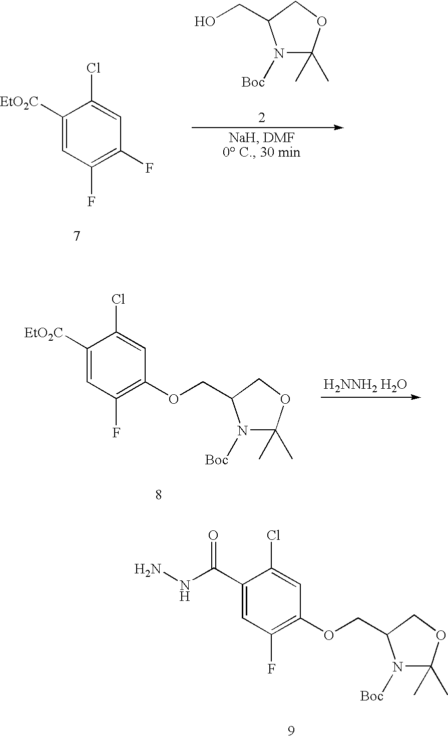

Step 1: tert-Butyl 4-((5-chloro-4-(ethoxycarbonyl)-2-fluorophenoxy)methyl)-2,2-dimethyloxazolidine-3-carboxylate (8). Intermediate 7 was synthesized in an analogous fashion to intermediate 4 using ethanol instead of methanol. Compound 8 was synthesized in an analogous manner to intermediate 5.

Step 2: tert-Butyl 4-((5-chloro-2-fluoro-4-(hydrazinecarbonyl)phenoxy)methyl)-2,2-dimethyloxazolidine-3-carboxylate (9). To a solution of 8 (3.7 g, 8.6 mmol) in EtOH (30 mL) was added hydrazine monohydrate (8.3 mL, 171 mmol) and the reaction mixture was heated at 80° C. for 2 h. The solvent was then removed in vacuo and the resulting residue dissolved in EtOAc, washed with water and concentrated to obtain crude 9 (2 g, ˜56% yield) which was used in subsequent reactions without further purification.

### Example 1

### General Procedure for the Coupling of Hydrazides to Intermediate 6

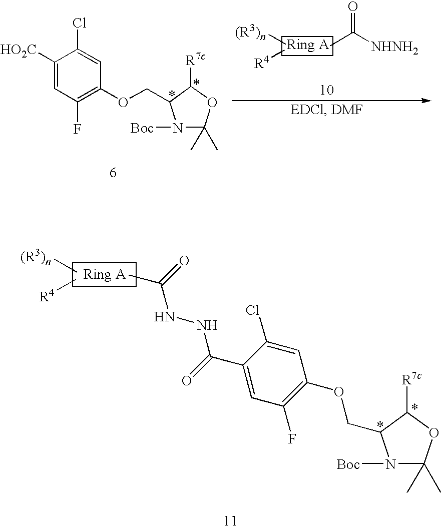

To a stirred solution of acid 6 (1 equiv) in DMF was added EDCI (about 1.5 equiv) and the mixture stirred at room temperature for 15 min. The intermediate of formula 10 (about 1.2 equiv) (which can be prepared as described for Intermediates 9, 21, 31, and 36) was added and the resulting mixture further stirred at room temperature for 1 h, followed by heating to 100° C. for 14 h. After completion, solvent was removed in vacuo and the resulting residue dissolved in EtOAc, washed with water, brine, dried over Na₂SO₄ and concentrated under high vacuum. The crude compound was purified by column chromatography to give 11 in typically 65-85% yield.

### Example 2

### General Procedure for the Coupling of Carboxylic Acids to Intermediate 9

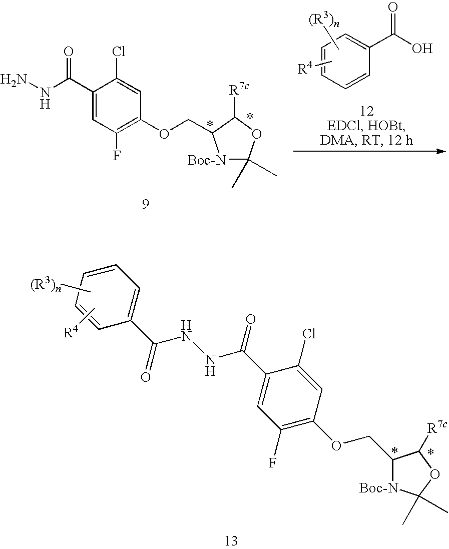

To a solution of 9 (about 1.5 equiv) in DMA, prepared as described in intermediate 9, was added intermediate 12 (1 equiv), which is commercially available or can be prepared using procedures known to one of ordinary skill in the art, EDCI (about 1.4 equiv), and HOBt (about 1.4 equiv). The resulting reaction mixture was allowed to stir at room temperature for 12 h. Upon completion as determined by LCMS analysis, the reaction mixture was poured into a solution of aqueous sat. NaHCO₃. The resulting mixture was extracted with EtOAc (2×), and the organic mixture was washed with 10% LiCl, followed by brine. The organic layer was separated, dried over MgSO₄, filtered, and concentrated. The crude mixture was purified by flash column chromatography to give 13 in typically 70-90% yield.

### Example 3

### General Procedure for Thiadiazole Ring Cyclization Using Lawesson's Reagent

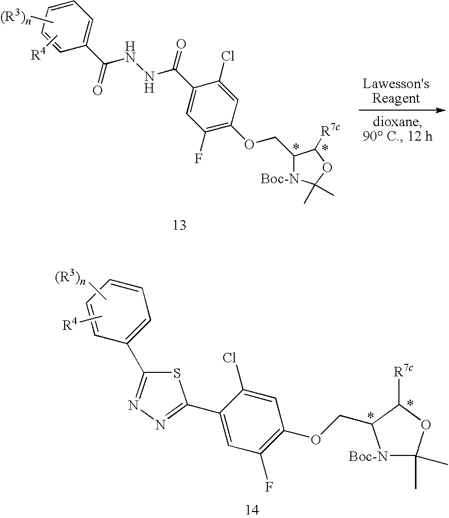

A heavy walled sealed tube was charged with 13 (0.31 mmol) in 1,4-dioxane (4.1 mL) and Lawesson's reagent (75.6 mg, 0.187 mmol) was added. The resulting reaction mixture was sealed, heated to 90° C., and allowed to stir for 12 h. After completion as determined by LCMS analysis, the reaction mixture was cooled to room temperature and water was added to the reaction mixture. The resulting mixture was extracted with EtOAc (2×) and the organic layers were combined, dried over MgSO₄, filtered, and concentrated. The resulting crude product was either washed with methanol and water and dried if a filterable solid or purified by flash chromatography to give 14 in typically 45-65% yield.

### Example 4

### General Procedure for Deprotection of Boc-2,2-dimethyloxazolidine-3-carboxylates

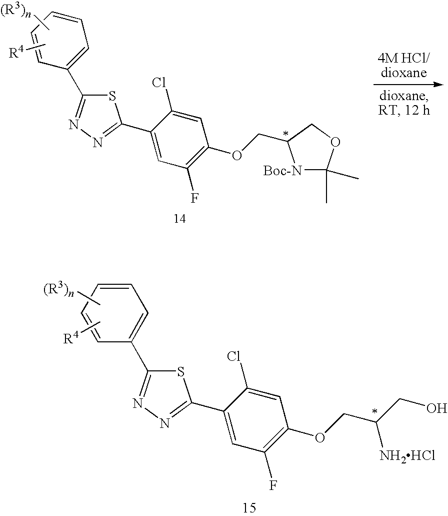

To a solution of 14 (0.14 mmol) in dioxane (2 mL) was added a solution of 4 M HCl in dioxane (2 mL). The resulting reaction mixture was allowed to stir at room temperature. Upon completion as determined by LCMS analysis, the reaction mixture was concentrated and Et₂O was added to the residue. The resulting solid was filtered, washed with water, and dried thoroughly to give the product as the solid HCl salt 15 in typically 70-90% yield. If necessary, the product could be further purified by preparative HPLC to give the product in typically 15-30% yield.

### Example 5

### 2-Amino-3-{[5-chloro-4-(5-{6-chloro-5-[(1-methylethyl)oxy]pyridin-2-yl}-1,3,4-thiadiazol-2-yl)-2-fluorophenyl]oxy}propan-1-ol hydrochloride

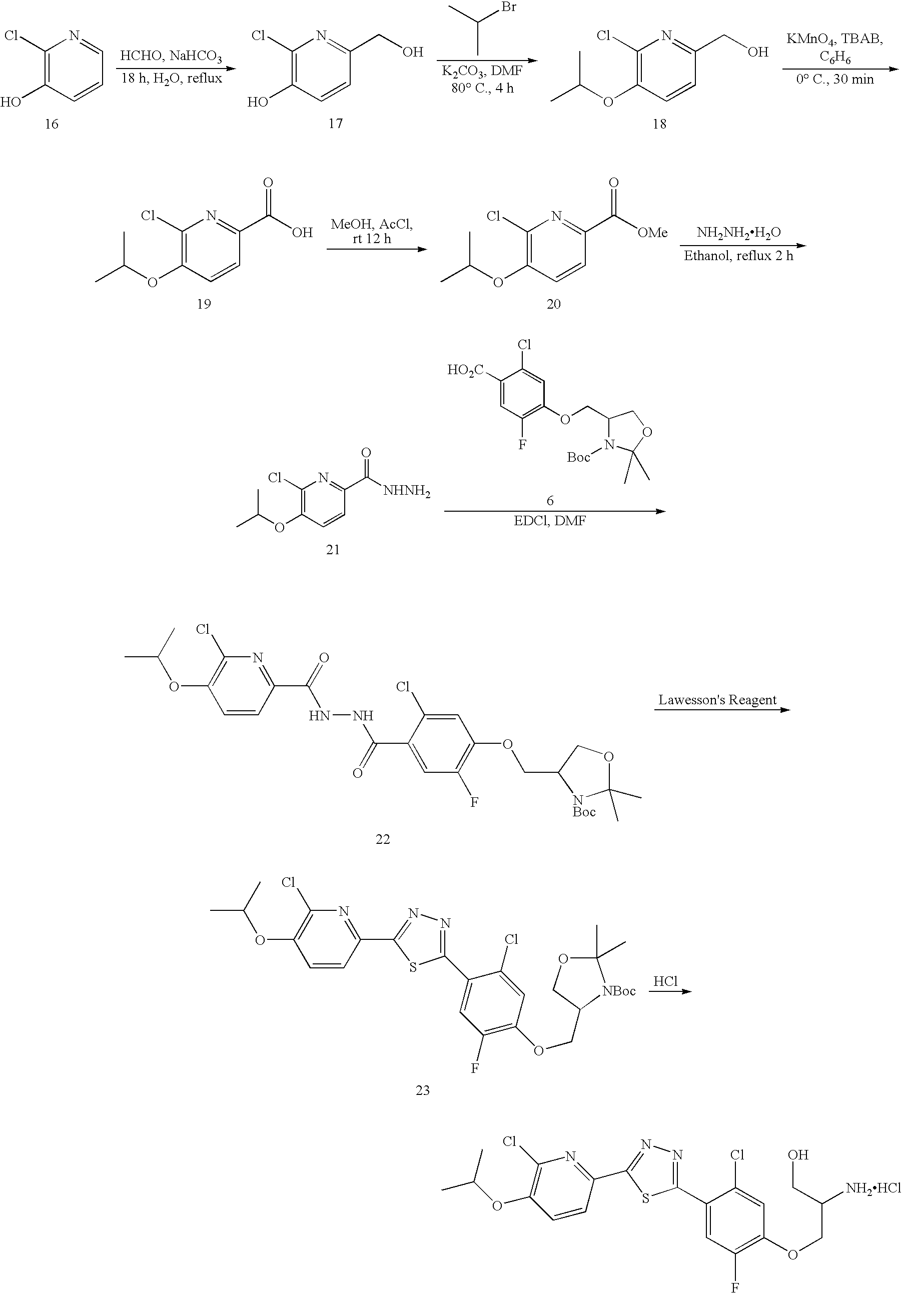

Step 1: 2-Chloro-6-(hydroxymethyl)pyridin-3-ol (17). To a stirring solution of commercially available 16 (10 g, 77 mmol) and NaHCO₃ (9.72 g, 116 mmol) in water (75 mL) at 90° C. was added aqueous formaldehyde (37%) (37.5 mL, 462 mmol) in 6 unequal fractions (12 mL, 3×7 mL, 2 mL) over a period of 90 min and finally 2.5 mL after 18 h. Stirring was continued at the same temperature for 18 h. The reaction mixture was then acidified with 6N HCl to pH 1 at 0° C., and the resulting precipitate was stirred for 90 min and then filtered. The filtrate was extracted with EtOAc and the organic extracts were dried, concentrated and purified by flash chromatography (1:1, EtOAc:hexane) to obtain 17 as a white solid (10 g, 81% yield).

Step 2: (6-Chloro-5-isopropoxypyridin-2-yl)methanol (18). To a stirred suspension of 17 (15 g, 94 mmol) and K₂CO₃ (26 g, 188 mmol) in DMF (50 mL) was added 2-bromopropane (9.8 mL, 104 mmol) and the reaction mixture was heated for 4 h at 80° C. DMF was removed, water was added, and the mixture extracted with EtOAc. The organic layer was dried and concentrated to obtain crude 18 as a thick oil (14.5 g, ˜76% yield) which was used in subsequent reactions without further purification.

Step 3: 6-Chloro-5-isopropoxypicolinic acid (19). To a stirred solution of 18 (14.4 g, 71 mmol) and tetrabutylammonium bromide (1 g, 3 mmol) in benzene (100 mL) at 0° C. was added an aqueous solution of KMnO₄ (15 g, 95 mmol) over a period of 40 min. Stirring was continued for another 20 min. Water was then added and the reaction mixture was quenched with conc. HCl. The aqueous layer was extracted with EtOAc. The organic extracts were dried, concentrated and purified by column chromatography to obtain 19 (7.9 g, 51% yield).

Step 4: Methyl 6-chloro-5-isopropoxypicolinate (20). Acetyl chloride (13.2 mL, 186 mmol) was added dropwise to an ice cold solution of 19 (10.0 g, 46 mmol) in MeOH (100 mL) and the reaction mixture was stirred for another 12 h at room temperature. Solvent was then removed from the reaction mixture and the resulting residue was dissolved in EtOAc, washed with aqueous NaHCO₃ and concentrated to obtain crude 20 (11 g, ˜100%) which was used in subsequent reactions without further purification.

Step 5: 6-Chloro-5-isopropoxypicolinohydrazide (21). To a stirred solution of 20 (9.6 g, 42 mmol) in EtOH (80 mL) was added hydrazine monohydrate (5.1 mL, 105 mmol) and the reaction mixture was refluxed for 2 h. The reaction mixture was then cooled and the resulting solid filtered, washed with cold water and dried to obtain 21 (5.7 g, 59% yield).

Step 6: tert-Butyl 4-((5-chloro-4-(2-(6-chloro-5-isopropoxypicolinoyl)hydrazinecarbonyl)-2-fluorophenoxy)methyl)-2,2-dimethyloxazolidine-3-carboxylate (22). Intermediate 22 was synthesized from compounds 6 and 21 using the same or an analogous synthetic procedure to that of Example 1.

Step 7: tert-Butyl 4-((5-chloro-4-(5-(6-chloro-5-isopropoxypyridin-2-yl)-1,3,4-thiadiazol-2-yl)-2-fluorophenoxy)methyl)-2,2-dimethyloxazolidine-3-carboxylate (23). Intermediate 23 was synthesized from compound 22 using the same or an analogous synthetic procedure to that of Example 3.

Step 8: 2-Amino-3-{[5-chloro-4-(5-{6-chloro-5-[(1-methylethyl)oxy]pyridin-2-yl}-1,3,4-thiadiazol-2-yl)-2-fluorophenyl]oxy}propan-1-ol hydrochloride. The title compound was synthesized from intermediate 23 using the same or an analogous synthetic procedure to that of Example 4. ¹H NMR (400 MHz, DMSO-d₆) δ 8.3 (m, 3H), 8.2 (d, 1H), 7.8 (d, 1H), 7.6 (d, 1H), 5.5 (bs, 1H), 4.9 (m, 1H), 4.4 (m, 2H), 3.7 (m, 2H), 3.6 (s, 1H), 1.4 (s, 6H); MS (EI) for C₁₉H₁₉C₁₂FN₄O₃S, found 472.90 (MH+).

Using the same or analogous synthetic techniques in Example 5 and substituting with appropriate reagents, the following compounds were prepared.

(2R)-2-Amino-3-{[5-chloro-4-(5-{6-chloro-5-[(1-methylethyl)oxy]pyridin-2-yl}-1,3,4-thiadiazol-2-yl)-2-fluorophenyl]oxy}propan-1-ol hydrochloride. ¹H NMR (400 MHz, DMSO-d₆) δ 8.41 (br s, 2H), 8.28 (d, 1H), 8.18 (d, 1H), 7.86 (d, 1H), 7.68 (d, 1H), 5.50 (br s, 1H), 4.90 (m, 1H), 4.42 (m, 2H), 3.72 (m, 2H), 3.57 (m, 1H), 1.36 (d, 6H); MS (EI) for C₁₉H₁₉Cl₂FN₄O₃S, found 472.85 (MH+).

(2S)-2-Amino-3-{[5-chloro-4-(5-{6-chloro-5-[(1-methylethyl)oxy]pyridin-2-yl}-1,3,4-thiadiazol-2-yl)-2-fluorophenyl]oxy}propan-1-ol hydrochloride. ¹H NMR (400 MHz, DMSO-d₆) δ 8.4 (s, 2H), 8.3 (d, 1H), 8.2 (d, 1H), 7.9 (d, 1H), 7.7 (d, 1H), 5.5 (bs, 1H), 4.9 (m, 1H), 4.4 (m, 2H), 3.4-3.8 (m, 3H), 1.4 (d, 6H); MS (EI) for C₁₉H₁₉Cl₂FN₄O₃S, found 473.05 (MH+).

### Example 6

### 2-Amino-3-{[5-chloro-4-(5-{5-chloro-6-[(1-methylethyl)oxy]pyridin-3-yl}-1,3,4-thiadiazol-2-yl)-2-fluorophenyl]oxy}propan-1-ol hydrochloride

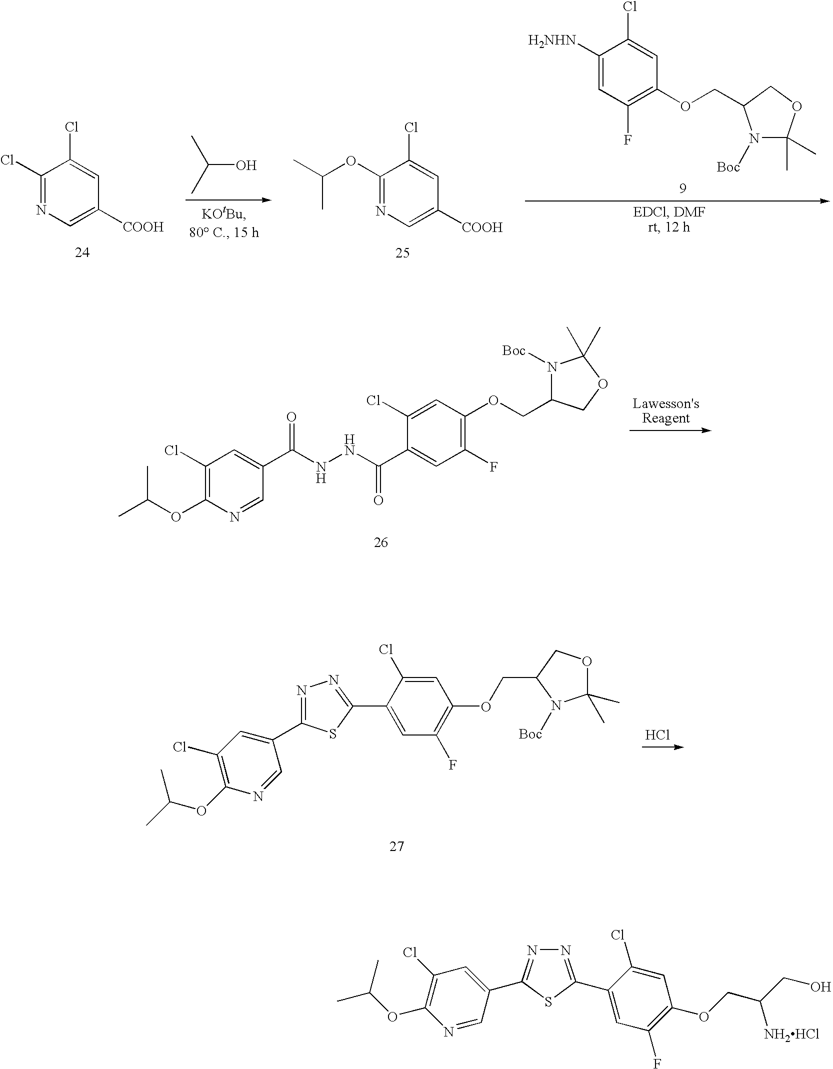

Step 1: 5-Chloro-6-isopropoxynicotinic acid (25). Potassium t-butoxide (7.0 g, 62.5 mmol) was added to isopropanol (60 mL) and the resulting mixture was stirred for 5 min at room temperature. Commercially-available intermediate 24 (6.0 g, 31.25 mmol) was added to the reaction mixture and it was heated for 15 h at 80° C. The reaction mixture was then allowed to cool to room temperature, diluted with water, acidified with 1N HCl and extracted with ether. The organic layer was dried and concentrated in vacuo to afford crude 25 (6.5 g, ˜96% yield) as a white solid which was used in subsequent reactions without further purification.

Step 2: tert-Butyl 4-((5-chloro-4-(2-(5-chloro-6-isopropoxynicotinoyl)hydrazinecarbonyl)-2-fluorophenoxy)methyl)-2,2-dimethyloxazolidine-3-carboxylate (26). Intermediate 26 was synthesized from compounds 9 and 25 using the same or an analogous synthetic procedure to that of Example 2.

Step 3: tert-Butyl 4-((5-chloro-4-(5-(5-chloro-6-isopropoxypyridin-3-yl)-1,3,4-thiadiazol-2-yl)-2-fluorophenoxy)methyl)-2,2-dimethyloxazolidine-3-carboxylate (27). Intermediate 27 was synthesized from compound 26 using the same or an analogous synthetic procedure to that of Example 3.

Step 4: 2-Amino-3-{[5-chloro-4-(5-{5-chloro-6-[(1-methylethyl)oxy]pyridin-3-yl}-1,3,4-thiadiazol-2-yl)-2-fluorophenyl]oxy}propan-1-ol hydrochloride. The title compound was synthesized from intermediate 27 using the same or an analogous synthetic procedure to that of Example 4. ¹H NMR (400 MHz, DMSO-d₆) δ 8.8 (s, 1H), 8.7 (s, 1H), 8.2 (s, 1H), 8.1 (s, 2H), 7.7 (d, 1H), 5.4 (m, 2H), 4.5 (m, 1H), 4.3 (m, 1H), 3.7 (m, 3H), 1.4 (s, 6H); MS (EI) for C₁₉H₁₉C₁₂FN₄O₃S, found 472.95 (MH+).

Using the same or analogous synthetic techniques in Example 6 and substituting with appropriate reagents, the following compounds were prepared.

(2R)-2-Amino-3-{[5-chloro-4-(5-{5-chloro-6-[(1-methylethyl)oxy]pyridin-3yl}-1,3,4-thiadiazol-2-yl)-2-fluorophenyl]oxy}propan-1-ol hydrochloride. ¹H NMR (400 MHz, DMSO-d₆) δ 8.80 (s, 1H), 8.52 (s, 1H), 8.19 (d, 1H), 8.15 (br s, 2H), 7.63 (d, 1H), 5.42 (m, 1H), 4.39 (m, 2H), 3.90-3.60 (complex m, 4H),1.38 (d, 6H); MS (EI) for C₁₉H₁₉C₁₂FN₄O₃S, found 472.85 (MH+).

(2S)-2-Amino-3-{[5-chloro-4-(5-{5-chloro-6-[(1-methylethyl)oxy]pyridin-3-yl}-1,3,4-thiadiazol-2-yl)-2-fluorophenyl]oxy}propan-1-ol hydrochloride. ¹H NMR (400 MHz, DMSO-d₆) δ 8.8 (s, 1H), 8.7 (s, 1H), 8.2 (s, 1H), 8.1 (s, 2H), 7.7 (d, 1H), 5.4 (m, 2H), 4.5 (m, 1H), 4.3 (m, 1H), 3.7 (m, 3H), 1.4 (s, 6H); MS (EI) for C₁₉H₁₉C₁₂FN₄O₃S, found 473.05 (MH+).

### Example 7

### 2-Amino-3-{[5-chloro-4-(5-{2-chloro-6-[methyl(1-methylethyl)amino]pyridin-4-yl}-1,3,4-thiadiazol-2-yl)-2-fluorophenyl]oxy}propan-1-ol hydrochloride

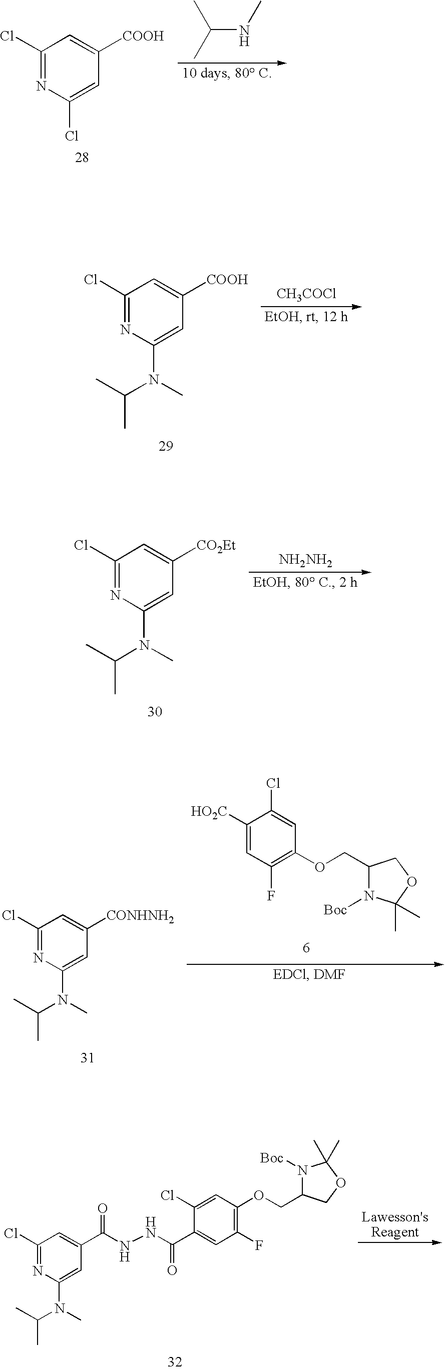

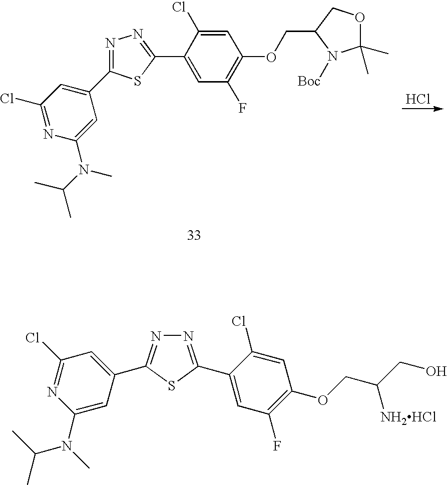

Step 1: 2-Chloro-6-(isopropyl(methyl)amino)isonicotinic acid (29). A stirred mixture of commercially available 28 (7.0 g, 36 mmol) and methyl isopropylamine (19 mL) was heated at 80° C. for 10 days in a sealed tube. Excess amine was removed from the reaction mixture and the resulting residue was diluted with water and extracted with EtOAc. The organic layer was concentrated to obtain crude 29 (4 g, ˜48% yield) which was used in subsequent reactions without further purification.

Step 2: Ethyl 2-chloro-6-(isopropyl(methyl)amino)isonicotinate (30). To an ice cold solution of 29 (4.0 g, 17 mmol) in EtOH (25 mL) was added acetyl chloride (7.4 mL, 104 mmol) and the reaction mixture was stirred 12 h at room temperature. Solvent was then removed, the resulting residue was dissolved in DCM, washed with NaHCO₃ solution and concentrated to obtain crude 30 (3.9 g, ˜87% yield) which was used in subsequent reactions without further purification.

Step 3: 2-Chloro-6-(isopropyl(methyl)amino)isonicotinohydrazide (31). To a solution of 30 (3.7 g, 14 mmol) in EtOH (30 mL) was added hydrazine monohydrate (8.3 mL, 171 mmol) and the reaction mixture was heated at 80° C. for 2 h. Solvent was removed and the resulting residue was dissolved in EtOAc, washed with water and concentrated to obtain crude 31 (2 g, ˜57% yield) which was used in subsequent reactions without further purification.

Step 4: tert-Butyl 4-((5-chloro-4-(2-(2-chloro-6-(isopropyl(methyl)amino)isonicotinoyl)hydrazinecarbonyl)-2-fluorophenoxy)methyl)-2,2-dimethyloxazolidine-3-carboxylate (32). Intermediate 32 was synthesized from compounds 6 and 31 using the same or an analogous synthetic procedure to that of Example 1.

Step 5: tert-Butyl 4-((5-chloro-4-(5-(2-chloro-6-(isopropyl(methyl)amino)pyridin-4-yl)-1,3,4-thiadiazol-2-yl)-2-fluorophenoxy)methyl)-2,2-dimethyloxazolidine-3-carboxylate (33). Intermediate 33 was synthesized from intermediate 32 using the same or an analogous synthetic procedure to that of Example 3.

Step 6: 2-Amino-3-{[5-chloro-4-(5-{2-chloro-6-[methyl(1-methylethyl)amino]pyridin-4-yl}-1,3,4-thiadiazol-2-yl)-2-fluorophenyl]oxy}propan-1-ol hydrochloride. The title compound was synthesized from intermediate 33 using the same or an analogous synthetic procedure to that of Example 4. ¹H NMR (400 MHz, DMSO-d₆) δ 8.2 (d, 1H), 8.1 (s, 2H), 7.8 (d, 1H), 7.1 (s, 1H), 7.0 (s, 1H), 5.4 (bs, 1H), 4.8 (t, 1H), 4.4 (m, 2H), 3.7 (m, 3H), 1.2 (s, 6H); MS (EI) for C₂₀H₂₂C₁₂FN₅O₂S, found 486.00 (MH+).

Using the same or analogous synthetic techniques in Example 7 and substituting with appropriate reagents, the following compounds were prepared.

(2R)-2-Amino-3-{[5-chloro-4-(5-{2-chloro-6-[methyl(1-methylethyl)amino]pyridin-4-yl}-1,3,4-thiadiazol-2-yl)-2-fluorophenyl]oxy}propan-1-ol hydrochloride. ¹H NMR (400 MHz, DMSO-d₆) δ 8.21 (d, 1H), 8.15 (br s, 2H), 7.68 (d, 1H), 7.17 (s, 1H), 7.12 (s, 1H), 5.43 (br s, 1H), 4.81 (m, 1H), 4.38 (m, 2H), 3.80-3.55 (complex m, 3H), 2.90 (s, 3H), 1.15 (d, 6H); MS (EI) for C₂₀H₂₂C₁₂FN₅O₂S, found 486.10 (MH+).

(2S)-2-Amino-3-{[5-chloro-4-(5-{2-chloro-6-[methyl(1-methylethyl)amino]pyridin-4-yl}-1,3,4-thiadiazol-2-yl)-2-fluorophenyl]oxy}propan-1-ol hydrochloride. ¹H NMR (400 MHz, DMSO-d₆) δ 8.2 (d, 1H), 8.1 (s, 2H), 7.8 (d, 1H), 7.1 (s, 1H), 7.0 (s, 1H), 5.4 (bs, 1H), 4.8 (t, 1H), 4.4 (m, 2H), 3.7 (m, 3H), 2.9 (s, 3H), 1.2 (s, 6H); MS (EI) for C₂₀H₂₂Cl₂FN₅O₂S, found 486.05 (MH+).

### Example 8

### 2-Amino-3-{[5-chloro-2-fluoro-4-(5-imidazo[2,1-b][1,3]thiazol-6-yl-1,3,4-thiadiazol-2-yl)phenyl]oxy}propan-1-ol hydrochloride

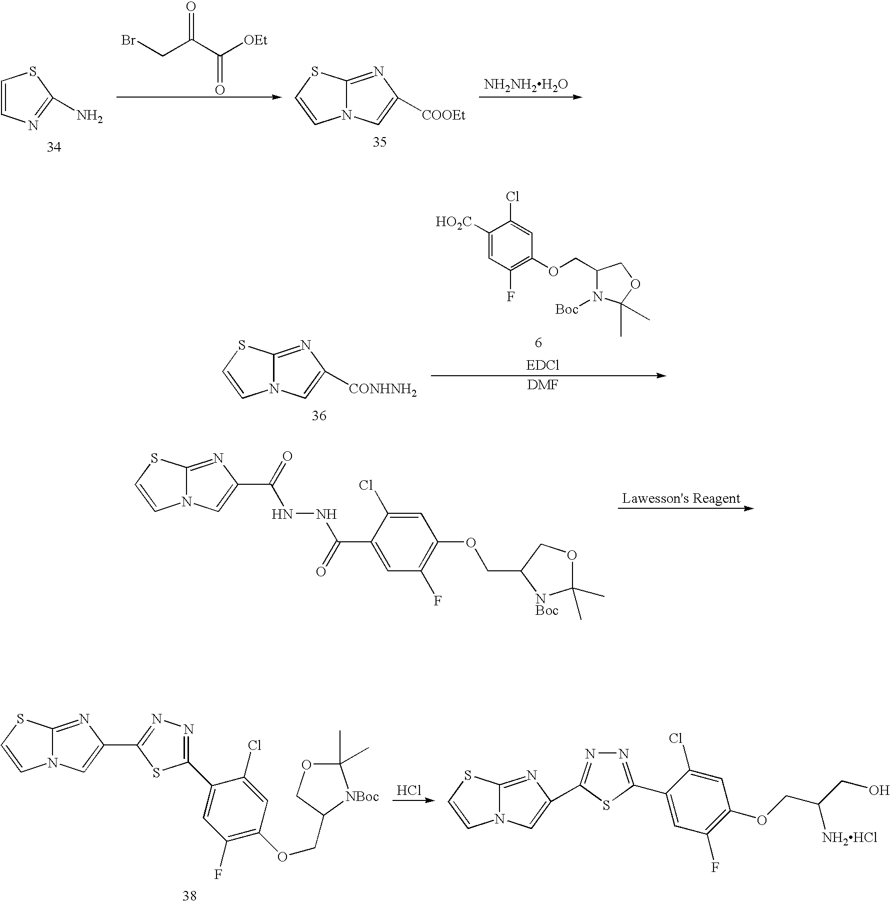

Step 1: Ethyl imidazo[2,1-b]thiazole-6-carboxylate (35). Intermediate 34 (4.0 g, 40 mmol) was dissolved in THF (400 mL) and then ethyl bromopyruvate (5.5 mL, 41 mmol) was added at room temperature and the reaction mixture stirred for 12 h. The resulting precipitate was filtered, suspended in EtOH (200 mL) and refluxed at 80° C. for 4 h. The reaction mixture was concentrated in vacuo and the resulting solid was washed with EtOH and dried to obtain crude 35 (6 g, ˜76%) which was used in subsequent reactions without further purification.

Step 2: Imidazo[2,1-b]thiazole-6-carbohydrazide (36). To a stirred solution of 35 (4 g, 20 mmol) in EtOH (20 mL) was added hydrazine monohydrate (2.4 mL, 49 mmol) and the reaction mixture was refluxed for 6 h. The reaction mixture was then concentrated in vacuo and the resulting residue was partitioned between water and EtOAc. The organic layer was dried and concentrated to obtain crude 36 (2.1 g, ˜56%) which was used in subsequent reactions without further purification.

Step 3: tert-Butyl 4-((5-chloro-2-fluoro-4-(2-(imidazo[2,1-b]thiazole-6-carbonyl)hydrazinecarbonyl)phenoxy)methyl)-2,2-dimethyloxazolidine-3-carboxylate (37). Intermediate 37 was synthesized from intermediates 6 and 36 using the same or an analogous synthetic procedure to that of Example 1.

Step 4: tert-Butyl 4-((5-chloro-2-fluoro-4-(5-(imidazo[2,1-b]thiazol-6-yl)-1,3,4-thiadiazol-2-yl)phenoxy)methyl)-2,2-dimethyloxazolidine-3-carboxylate (38). Intermediate 38 was synthesized from intermediate 37 using the same or an analogous synthetic procedure to that of Example 3.

Step 5: 2-Amino-3-{[5-chloro-2-fluoro-4-(5-imidazo[2,1-b][1,3]thiazol-6-yl-1,3,4-thiadiazol-2-yl)phenyl]oxy}propan-1-ol hydrochloride. The title compound was synthesized from intermediate 38 using the same or an analogous synthetic procedure to that of Example 4. ¹H NMR (400 MHz, DMSO-d₆) δ 8.6 (s, 1H), 8.3 (s, 2H), 8.2 (d, 1H), 8.0 (d, 1H), 7.6 (d, 1H), 7.4 (d, 1H), 5.4 (s, 1H), 4.4 (m, 2H), 3.7 (m, 2H), 3.6 (m, 1H); MS (EI) for C₁₆H₁₃ClFN₅O₂S₂, found 425.85 (MH+).

### Example 9

### (2R)-2-Amino-3-[(5-chloro-2-fluoro-4-{5-[4-(2-methylpropyl)phenyl]-1,3,4-thiadiazol-2-yl}phenyl)oxy]propan-1-ol acetate salt

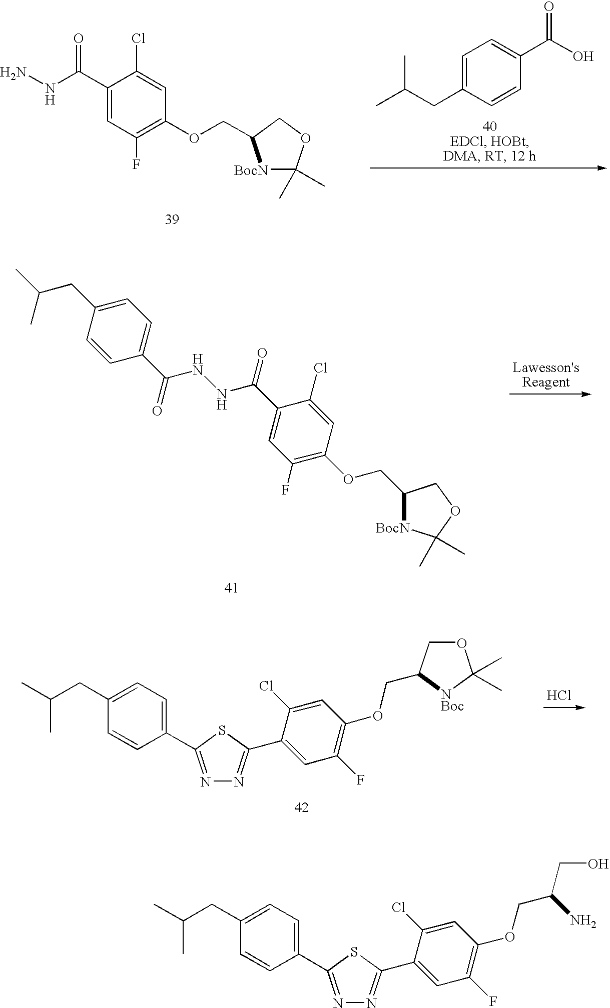

Step 1: (S)-tert-Butyl 4-((5-chloro-2-fluoro-4-(2-(4-isobutylbenzoyl)hydrazinecarbonyl)phenoxy)methyl)-2,2-dimethyloxazolidine-3-carboxylate (41). Intermediate 39 was made in an analogous manner to intermediate 9 using the appropriate enantiomerically pure starting material in place of a racemic mixture. Intermediate 41 was synthesized from intermediates 39 and commercially-available 40 using the same or an analogous synthetic procedure to that of Example 2.

Step 2: (S)-tert-Butyl 4-((5-chloro-2-fluoro-4-(5-(4-isobutylphenyl)-1,3,4-thiadiazol-2-yl)phenoxy)methyl)-2,2-dimethyloxazolidine-3-carboxylate (42). Intermediate 42 was synthesized from intermediate 41 using the same or an analogous synthetic procedure to that of Example 3.

Step 3: (2R)-2-Amino-3-[(5-chloro-2-fluoro-4-{5-[4-(2-methylpropyl)phenyl]-1,3,4-thiadiazol-2-yl}phenyl)oxy]propan-1-ol acetate salt. The title compound was synthesized from intermediate 42 using the same or an analogous synthetic procedure to that of Example 4. ¹H NMR (400 MHz, DMSO-d₆) δ 8.17 (m, 3H), 7.98 (d, 2H), 7.65 (d, 1H), 7.39 (d, 2H), 5.45 (t, 1H), 4.42 (m, 1H), 4.33 (m, 1H), 3.67 (m, 3H), 2.55 (d, 2H), 1.90 (d, 1H), 0.89 (d, 6H). MS (EI) for C₂₁H₂₃ClFN₃O₂S, found 436.1 (MH+).

Using the same or analogous synthetic techniques in Example 9 and substituting with appropriate reagents, the following compounds were prepared.

(2R)-2-Amino-3-{[5-chloro-4-(5-{3-chloro-4-[(1-methylethyl)oxy]phenyl}-1,3,4-thiadiazol-2-yl)-2-fluorophenyl]oxy}propan-1-ol hydrochloride. ¹H NMR (400 MHz, DMSO-d₆) δ 8.25 (br s, 3H), 8.16 (d, 1H), 8.12 (d, 1H), 7.98 (dd, 1H), 7.65 (d, 1H), 7.38 (d, 1H), 5.47 (t, 1H), 4.85 (m, 1H), 4.44 (m, 2H), 3.70 (m, 2H), 3.59 (m, 1H), 1.35 (d, 6H); MS (EI) for C₂₀H₂₀Cl₂FN₃O₃S, found 472.1 (MH+).

(2R)-2-Amino-3-{[4-(5-{3-bromo-4-[(1-methylethyl)oxy]phenyl}-1,3,4-thiadiazol-2-yl)-5-chloro-2-fluorophenyl]oxy}propan-1-ol hydrochloride. ¹H NMR (400 MHz, DMSO-d₆) δ 8.26 (d, 1H), 8.17 (d, 1H), 8.13 (br s, 2H), 8.02 (dd, 1H), 7.65 (d, 1H), 7.35 (d, 1H), 5.45 (t, 1H), 4.84 (m, 1H), 4.38 (m, 2H), 3.70 (m, 2H), 3.60 (m, 1H), 1.35 (d, 6H); MS (EI) for C₂₀H₂₀BrClFN₃O₃S, found 516.0 (MH+).

(2R)-2-Amino-3-{[5-chloro-2-fluoro-4-(5-{4-[(trifluoromethyl)oxy]phenyl}-1,3,4-thiadiazol-2-yl)phenyl]oxy}propan-1-ol hydrochloride. ¹H NMR (400 MHz, DMSO-d₆) δ 8.21 (m, 2H), 8.15 (d, 1H), 7.60 (d, 1H), 4.75 (br s, 1H), 4.10 (m, 2H), 3.43 (m, 2H), 3.08 (m, 1H); MS (EI) for C₁₈H₁₄ClF₄N₃O₃S, found 464.0 (MH+).

(2R)-2-Amino-3-{[5-chloro-4-(5-{3-chloro-4-[(trifluoromethyl)oxy]phenyl}-1,3,4-thiadiazol-2-yl)-2-fluorophenyl]oxy}propan-1-ol hydrochloride. ¹H NMR (400 MHz, DMSO-d₆) δ 8.40 (d, 1H), 8.19 (dd, 1H), 8.16 (d, 1H), 7.81 (m, 1H), 7.61 (d, 1H), 4.80 (br s, 1H), 4.12 (m, 2H), 3.45 (m, 2H), 3.11 (m, 1H); MS (EI) for C₁₈H₁₃Cl₂F₄N₃O₃S, found 498.0 (MH+).

(2R)-2-Amino-3-({4-[5-(3-bromo-5-chlorophenyl)-1,3,4-thiadiazol-2-yl]-5-chloro-2-fluorophenyl}oxy)propan-1-ol hydrochloride. ¹H NMR (400 MHz, DMSO-d₆) δ 8.23 (m, 4H), 8.20 (m, 1H), 8.17 (m, 1H), 7.68 (d, 1H), 5.47 (m, 1H), 4.40 (m, 2H), 3.71 (m, 2H), 3.60 (m, 1H); MS (EI) for C₁₇H₁₃BrCl₂FN₃O₂S, found 491.9 (MH+).

(2S)-2-Amino-3-{[4-(5-{3-bromo-4-[(1-methylethyl)oxy]phenyl}-1,3,4-thiadiazol-2-yl)-5-chloro-2-fluorophenyl]oxy}propan-1-ol hydrochloride. ¹H NMR (400 MHz, DMSO-d₆) δ 8.30 (br s, 3H), 8.24 (d, 1H), 8.16 (d, 1H), 8.02 (dd, 1H), 7.66 (d, 1H), 7.35 (d, 1H), 5.48 (br s, 1H), 4.83 (dq, 1H), 4.40 (m, 2H), 3.70 (m 2H), 3.59 (m, 1H), 1.35 (d, 6H); MS (EI) for C₂₀H₂₀BrClFN₃O₃S, found 518.0 (MH+).

(2R)-2-Amino-3-{[5-chloro-2-fluoro-4-(5-{4-[(1-methylethyl)oxy]-3-(trifluoromethyl)phenyl}-1,3,4-thiadiazol-2-yl)phenyl]oxy}propan-1-ol acetate salt. ¹H NMR (400 MHz, DMSO-d₆) δ 8.27 (dd, 1H), 8.22 (d, 1H), 8.13 (d, 1H), 7.58 (d, 1H), 7.51 (d, 1H), 4.95 (m, 1H), 4.65 (very br s, 1H), 4.10 (m, 2H), 3.50 (very br s, 3H), 3.43 (m, 2H), 3.10 (m, 1H), 1.90 (s, 3H, AcOH), 1.34 (d, 6H); MS (EI) for C₂₁H₂₀ClF₄N₃O₃S, found 506.0 (MH+).

(2R)-2-Amino-3-{[5-chloro-2-fluoro-4-(5-{4-[(1-methylethyl)oxy]phenyl}-1,3,4-thiadiazol-2-yl)phenyl]oxy}propan-1-ol hydrochloride. ¹H NMR (400 MHz, DMSO-d₆) δ 8.22 (br s, 2H), 8.14 (d, 1H), 7.97 (d, 2H), 7.65 (d, 1H), 7.11 (d, 2H), 5.46 (br s, 1H), 4.75 (m, 1H), 4.42 (m, 1H), 4.34 (m, 1H), 3.70 (m, 2H), 3.59 (m, 1H), 1.31 (d, 6H); MS (EI) for C₂₀H₂₁ClFN₃O₃S, found 438.1 (MH+).

(2S)-2-Amino-3-{[5-chloro-4-(5-{3-chloro-4-[(1-methylethyl)oxy]phenyl}-1,3,4-thiadiazol-2-yl)-2-fluorophenyl]oxy}propan-1-ol hydrochloride. ¹H NMR (400 MHz, DMSO-d₆) δ 8.24 (br s, 2H), 8.16 (d, 1H), 8.11 (d, 1H), 7.98 (dd, 1H), 7.65 (d, 1H), 7.38 (d, 1H), 5.47 (t, 1H), 4.84 (m, 1H), 4.42 (m, 1H), 4.34 (m, 1H), 3.70 (m, 2H), 3.59 (m, 1H), 1.35 (d, 6H); MS (EI) for C₂₀H₂₀Cl₂FN₃O₃S, found 472.1 (MH+).

(2R)-2-Amino-3-[(5-chloro-4-{5-[3-chloro-4-(propyloxy)phenyl]-1,3,4-thiadiazol-2-yl}-2-fluorophenyl)oxy]propan-1-ol hydrochloride. ¹H NMR (400 MHz, DMSO-d₆) δ 8.23 (br s, 2H), 8.16 (d, 1H), 8.12 (d, 1H), 8.00 (dd, 1H), 7.65 (d, 1H), 7.35 (d, 1H), 5.45 (br s, 1H), 4.43 (m, 1H), 4.34 (m, 1H), 4.14 (t, 2H), 3.70 (m, 2H), 3.59 (m, 1H), 1.80 (m, 2H), 1.02 (t, 3H). MS (EI) for C₂₀H₂₀Cl₂FN₃O₂S, found 472.0 (MH+).

(2R)-2-Amino-3-({5-chloro-4-[5-(3-chlorophenyl)-1,3,4-thiadiazol-2-yl]-2-fluorophenyl}oxy)propan-1-ol hydrochloride. ¹H NMR (400 MHz, DMSO-d₆) δ 8.19 (m, 4H), 8.13 (m, 1H), 8.04 (m, 1H), 7.66 (m, 3H), 5.46 (t, 1H), 4.42 (m, 1H), 4.34 (m, 1H), 3.68 (m, 3H). MS (EI) for C₁₇H₁₄Cl₂FN₃O₂S, found 414.0 (MH+).

(2R)-2-Amino-3-({4-[5-(3-bromo-5-fluorophenyl)-1,3,4-thiadiazol-2-yl]-5-chloro-2-fluorophenyl}oxy)propan-1-ol hydrochloride. ¹H NMR (400 MHz, DMSO-d₆) δ 8.24 (br s, 3H), 8.20 (d, 1H), 8.13 (m, 1H), 7.99 (m, 1H), 7.87 (m, 1H), 7.67 (d, 1H), 5.47 (t, 1H), 4.40 (m, 2H), 3.71 (m, 2H), 3.60 (br s, 1H); MS (EI) for C₁₇H₁₃BrClF₂N₃O₂S, found 477.9 (MH+).

(2R)-2-Amino-3-[(5-chloro-4-{5-[3-chloro-4-(ethyloxy)phenyl]-1,3,4-thiadiazol-2-yl}-2-fluorophenyl)oxy]propan-1-ol hydrochloride. ¹H NMR (400 MHz, DMSO-d₆) δ 8.27 (br s, 3H), 8.16 (d, 1H), 8.12 (d, 1H), 8.00 (d, 1H), 7.66 (d, 1H), 7.35 (d, 1H), 5.48 (br s, 1H), 4.39 (m, 2H), 4.24 (m, 2H), 3.66 (m, 3H), 1.40 (t, 3H); MS (EI) for C₁₉H₁₈Cl₂FN₃O₃S, found 457.8 (MH+).

(2R)-2-Amino-3-{[5-chloro-4-(5-{3,5-dichloro-4-[(1-methylethyl)oxy]phenyl}-1,3,4-thiadiazol-2-yl)-2-fluorophenyl]oxy}propan-1-ol hydrochloride. ¹H NMR (400 MHz, DMSO-d₆) δ 8.2 (m, 4H), 7.8 (d, 2H), 5.4 (s, 1H), 4.7 (m, 1H), 4.4 (m, 2H) 3.7 (m, 3H), 1.4 (d, 6H); MS (EI) for C₂₀H₁₉Cl₃FN₃O₃S, found 506.12 (MH+).

(2R)-2-Amino-3-{[4-(5-{3-bromo-5-[(1-methylethyl)oxy]phenyl}-1,3,4-thiadiazol-2-yl)-5-chloro-2-fluorophenyl]oxy}propan-1-ol hydrochloride. ¹H NMR (400 MHz, DMSO-d₆) δ 8.2 (m, 3H), 7.8 (s, 1H), 7.6 (d, 1H), 7.5 (s, 1H), 7.4 (s, 1H), 5.4 (m, 1H), 4.8 (m, 1H), 4.4 (m, 2H) 3.7 (m, 3H), 1.3 (d, 6H); MS (EI) for C₂₀H₂₀BrClFN₃O₃S, found 515.8 (MH+).

(2R)-2-Amino-3-{[4-(5-{3-bromo-5-[(trifluoromethyl)oxy]phenyl}-1,3,4-thiadiazol-2-yl)-5-chloro-2-fluorophenyl]oxy}propan-1-ol hydrochloride. ¹H NMR (400 MHz, DMSO-d₆) δ 8.3 (s, 1H), 8.1 (m, 4H), 8.0 (s, 1H), 7.6 (d, 1H), 5.4 (s, 1H), 4.4 (m, 2H), 3.8 (m, 3H); MS (EI) for C₁₈H₁₃BrClF₄N₃O₃S, found 542.00 (MH+).

(2R)-2-Amino-3-({5-chloro-4-[5-(3,5-dibromophenyl)-1,3,4-thiadiazol-2-yl]-2-fluorophenyl}oxy)propan-1-ol hydrochloride. ¹H NMR (400 MHz, DMSO-d₆) δ 8.3 (s, 2H), 8.2 (d, 1H), 8.1 (s, 2H), 7.6 (d, 1H), 7.2 (m, 1H), 5.4 (s, 1H), 4.4 (m, 2H), 3.7 (m, 3H); MS (EI) for C₁₇H₁₃Br₂ClFN₃O₂S, found 535.8 (MH+).

(2R)-2-Amino-3-{[3-chloro-4-(5-{3-chloro-4-[(1-methylethyl)oxy]phenyl}-1,3,4-thiadiazol-2-yl)phenyl]oxy}propan-1-ol hydrochloride. ¹H NMR (400 MHz, DMSO-d₆) δ 8.2 (d, 1H), 8.1 (m, 4H), 7.9 (d, 1H), 7.4 (m, 2H), 7.2 (d, 1H), 5.4 (t, 1H), 4.8 (m, 1H), 4.4 (m, 1H), 4.2 (m, 1H), 3.7 (m, 2H), 3.6 (m, 1H), 1.4 (d, 6H); MS (EI) for C₂₀H₂₁Cl₂N₃O₃S, found 454.00 (MH+).

5-[5-(4-{[(2R)-2-Amino-3-hydroxypropyl]oxy}-2-chloro-5-fluorophenyl)-1,3,4-thiadiazol-2-yl]-2-[(1-methylethyl)oxy]benzonitrile hydrochloride. ¹H NMR (400 MHz, DMSO-d₆) δ 8.43 (d, 1H), 8.33 (dd, 1H), 8.24 (br s, 3H), 8.17 (d, 1H), 7.66 (d, 1H), 7.50 (d, 1H), 5.47 (t, 1H), 4.94 (m, 1H), 4.40 (m, 2H), 3.68 (m, 3H), 1.37 (d, 6H); MS (EI) for C₂₁H₂₀ClFN₄O₃S, found 462.8 (MH+).

(2R)-2-Amino-3-{[2,6-dichloro-4-(5-{3-chloro-4-[(1-methylethyl)oxy]phenyl}-1,3,4-thiadiazol-2-yl)phenyl]oxy}propan-1-ol hydrochloride. ¹H NMR (400 MHz, DMSO-d₆) δ 8.4 (br s, 3H), 8.2 (s, 2H), 8.1 (s, 1H), 7.9 (d, 1H), 7.4 (d, 1H), 5.4 (t, 1H), 4.8 (m, 1H), 4.3 (m, 2H), 3.8 (m, 2H), 3.6 (m, 1H), 1.4 (d, 6H); MS (EI) for C₂₀H₂₀Cl₃N₃O₃S, found 487.85 (MH+).

(2R)-2-Amino-3-{[2-chloro-4-(5-{3-chloro-4-[(1-methylethyl)oxy]phenyl}-1,3,4-thiadiazol-2-yl)-6-fluorophenyl]oxy}propan-1-ol hydrochloride. ¹H NMR (400 MHz, DMSO-d₆) δ 8.4 (br s, 3H), 8.0 (m, 3H), 7.9 (d, 1H), 7.4 (d, 1H), 5.4 (br s, 1H), 4.8 (m, 1H), 4.4 (m, 2H), 3.8 (m, 2H), 3.5 (m, 1H), 1.4 (d, 6H); MS (EI) for C₂₀H₂₀Cl₂FN₃O₃S, found 472.10 (MH+).

(2R)-2-Amino-3-{[5-chloro-2-fluoro-4-(5-{3-fluoro-4-[(1-methylethyl)oxy]phenyl}-1,3,4-thiadiazol-2-yl)phenyl]oxy}propan-1-ol hydrochloride. ¹H NMR (400 MHz, DMSO-d₆) δ 8.2 (m, 3H), 7.9 (d, 1H), 7.8 (d, 1H), 7.6 (d, 1H), 7.4 (t, 1H), 5.4 (t, 1H), 4.8 (m, 1H), 4.2 (m, 2H), 3.7 (m, 3H), 1.4 (d, 6H); MS (EI) for C₂₀H₂₀ClF₂N₃O₃S, found 456.15 (MH+).

5-[5-(4-{[(2R)-2-Amino-3-hydroxypropyl]oxy}-2-chloro-5-fluorophenyl)-1,3,4-thiadiazol-2-yl]-2-(oxetan-3-yloxy)benzonitrile trifluoroacetic acid salt. ¹H NMR (400 MHz, DMSO-d₆) δ 8.5 (s, 1H), 8.3 (d, 1H), 8.2 (m, 3H), 7.6 (d, 1H), 7.1 (d, 1H), 5.6 (m, 1H), 5.4 (m, 1H), 5.0 (m, 2H), 4.6 (m, 2H), 4.4 (m, 2H), 3.8-3.6 (m, 3H); MS (EI) for C₂₁H₁₈ClFN₄O₄S, found 476.95 (MH+).

5-[5-(4-{[(2R)-2-Amino-3-hydroxypropyl]oxy}-2-chloro-5-fluorophenyl)-1,3,4-thiadiazol-2-yl]-2-(ethyloxy)benzonitrile hydrochloride. ¹H NMR (400 MHz, DMSO-d₆) δ 8.45 (s, 1H), 8.35 (d, 1H), 8.15 (d, 1H), 7.8 (bs, —NH₂, 2H), 7.6 (d, 1H), 7.4 (d, 1H), 5.4 (m, 1H), 4.4 (m, 4H), 3.7 (m, 2H), 3.5 (m, 1H), 1.4 (t, 3H); MS (EI) for C₂₀H₁₈ClFN₄O₃S, found 449.00 (MH+).

5-[5-(4-{[(2R)-2-Amino-3-hydroxypropyl]oxy}-2-chloro-5-fluorophenyl)-1,3,4-thiadiazol-2-yl]-2-{[2-(methyloxy)ethyl]oxy}benzonitrile hydrochloride. ¹H NMR (400 MHz, DMSO-d₆) δ 8.45 (s, 1H), 8.4 (d, 1H), 8.2 (m, 3H), 7.65 (d, 1H), 7.5 (d, 1H), 5.5 (m, 1H), 4.4 (m, 4H), 3.7 (m, 5H), 3.3 (s, 3H); MS (EI) for C₂₁H₂₀ClFN₄O₄S, found 478.95 (MH+).

5-[5-(4-{[(2R)-2-Amino-3-hydroxypropyl]oxy}-2-chloro-5-fluorophenyl)-1,3,4-thiadiazol-2-yl]-2-[(1-methylethyl)oxy]benzamide hydrochloride. ¹H NMR (400 MHz, DMSO-d₆) δ 8.43 (s, 1H), 8.20 (m, 2H), 7.8 (m, 4H), 7.65 (m, 2H), 7.40 (d, 1H), 5.42 (br s, 1H), 4.95 (m, 1H), 4.4 (m, 2H), 3.72 (m, 2H), 3.60 (m, 1H), 1.40 (d, 6H); MS (EI) for C₂₁H₂₂ClFN₄O₄S, found 480.95 (MH+).

5-[5-(4-{[(2R,3R)-2-Amino-3-hydroxybutyl]oxy}-2-chloro-5-fluorophenyl)-1,3,4-thiadiazol-2-yl]-2-[(1-methylethyl)oxy]benzonitrile hydrochloride. ¹H NMR (400 MHz, DMSO-d₆) δ 8.40 (s, 1H), 8.35-8.10 (m, 5H), 7.68 d, 1H), 7.50 (d, 1H), 5.60 (br s, 1H), 4.95 (m, 1H), 4.4 (m, 2H), 3.95 (m, 1H), 3.40 (m, 1H), 1.40 (d, 6H), 1.23 (d, 3H); MS (EI) for C₂₂H₂₂ClFN₄O₃S, found 477.10 (MH+).

(2R)-2-Amino-3-[(5-chloro-2-fluoro-4-{5-[4-(propyloxy)phenyl]-1,3,4-thiadiazol-2-yl}phenyl)oxy]propan-1-ol hydrochloride. ¹H NMR (400 MHz, DMSO-d₆) δ 8.18 (s, 2H), 8.13 (d, 1H), 7.97 (d, 2H), 7.63 (d, 1H), 7.12 (d, 2H), 5.44 (m, 1H), 4.36 (m, 2H), 4.02 (t, 2H), 3.69 (m, 2H), 3.59 (m, 1H), 1.75 (m, 2H), 0.98 (t, 3H); MS (EI) for C₂₀H₂₁ClFN₃O₃S, found 438.1 (MH+).

(2R)-2-Amino-3-({5-chloro-4-[5-(3,5-dichlorophenyl)-1,3,4-thiadiazol-2-yl]-2-fluorophenyl}oxy)propan-1-ol hydrochloride. ¹H NMR (400 MHz, DMSO-d₆) δ 8.2 (d, 1H), 8.1 (s, 2H), 7.9 (s, 1H), 7.6 (d, 1H), 7.2 (m, 2H), 5.4 (s, 1H), 4.4 (m, 2H), 3.7 (m, 3H); MS (EI) for C₁₇H₁₃Cl₃FN₃O₂S, found 447.90 (MH+).

(2R)-2-Amino-3-(5-chloro-4-(5-(3-chloro-4-(oxetan-3-yloxy)phenyl)-1,3,4-thiadiazol-2-yl)-2-fluorophenoxy)propan-1-ol trifluoroacetic acid salt. ¹H NMR (400 MHz, DMSO-d₆) δ 8.2 (m, 4H), 8.0 (d, 1H), 7.6 (d, 1H), 7.0 (d, 1H), 5.5 (m, 2H), 5.0 (m, 2H), 4.6 (m, 2H), 4.4 (m, 2H), 3.8-3.6 (m, 3H); MS (EI) for C₂₀H₁₈Cl₂FN₃O₄S, found 485.95 (MH+).

(2R)-2-Amino-3-[(5-chloro-2-fluoro-4-{5-[4-phenyl-5-(trifluoromethyl)-2-thienyl]-1,3,4-thiadiazol-2-yl}phenyl)oxy]propan-1-ol hydrochloride. ¹H NMR (400 MHz, DMSO-d₆) δ 8.30 (br s, 3H), 8.20 (m, 2H), 7.70 (d, 1H), 7.55 (m, 5H), 5.50 (br s, 1H), 4.4 (m, 2H), 3.70 (m, 2H), 3.60 (m, 1H); MS (EI) for C₂₂H₁₆ClF₄N₃O₂S₂, found 529.7 (MH+).

### Example 10

### General Procedure for the Phosphorylation of Amino Alcohols

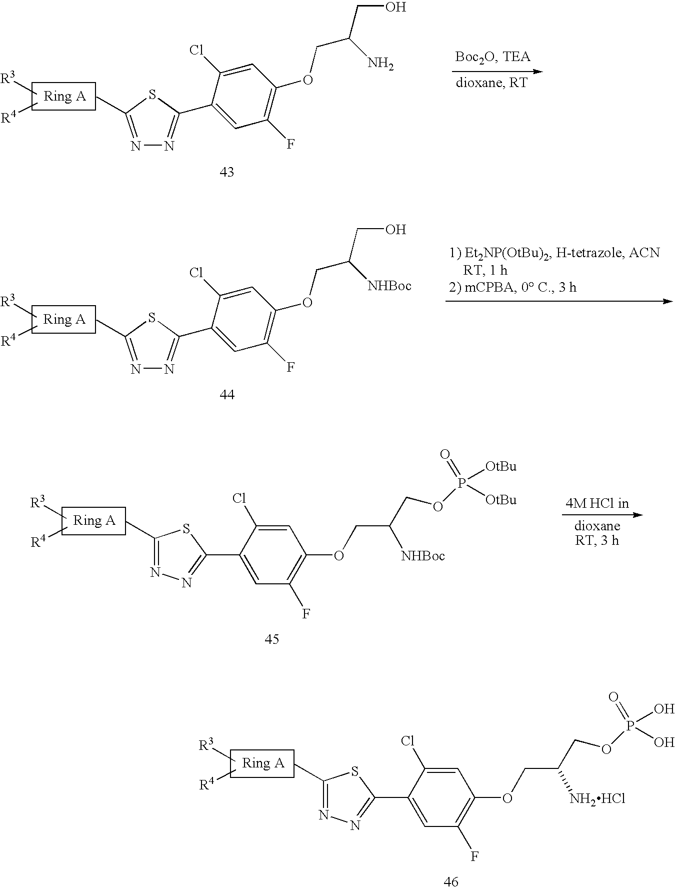

Step 1: Synthesis of Compound 44. To a solution of 43 (0.54 mmol), prepared as described in any of the above examples, in dioxane (5.4 mL) was added Boc₂O (178.5 mg, 0.82 mmol) and TEA (110.3 mg, 1.09 mmol). The resulting reaction mixture was allowed to stir at room temperature for 12 h. Upon completion as determined by LCMS analysis, the reaction mixture was diluted with water and extracted with EtOAc (2×). The combined organic layers were washed with brine, dried over MgSO₄, filtered and concentrated. The crude mixture was purified by recrystallization to yield 44 in typically 75% yield.

Step 2: Synthesis of Compound 45. Alcohol 44 (0.41 mmol) was treated with a solution of H-tetrazole in acetonitrile (3% wt/v, 5.77 mL, 2.47 mmol), and di-tert-butyl diethyl phosphoramidite (410 mg, 1.65 mmol) was added to the reaction mixture. The resulting reaction mixture was allowed to stir at room temperature for 2 h. Upon completion as determined by LCMS analysis, the reaction mixture was cooled to 0° C. and mCPBA (369.8 mg, 1.65 mmol, 77%) was added portionwise. The reaction mixture was allowed to warm to room temperature and stirred for an additional 3 h. Upon completion as determined by LCMS analysis, the reaction was diluted with EtOAc and a solution of aqueous saturated NaHCO₃ was added. The phases were separated and the aqueous phase further extracted with EtOAc (2×). The combined organic layers were dried with MgSO₄, filtered, and concentrated. The resulting crude oil was purified by flash column chromatography and further purified by recrystallization to give 45 in typically 30-35% yield.

Step 3: Synthesis of Compound 46. Phosphate ester 45 (0.08 mmol) was combined with a solution of 4M HCl in dioxane (1 mL). The resulting reaction mixture was allowed to stir at room temperature for 3 h. Upon completion as determined by LCMS analysis, Et₂O was added to the reaction mixture and the resulting solid was allowed to settle. The organic layer was decanted, and the process was repeated again. Acetonitrile and water were added to the resulting solid, which was then lyophilized to give 46 as the HCl salt in typically 90% yield or better.

Using the same or analogous synthetic techniques in Example 10 and substituting with appropriate reagents, the following compounds were prepared.

(2R)-2-Amino-3-{[4-(5-{3-bromo-4-[(1-methylethyl)oxy]phenyl}-1,3,4-thiadiazol-2-yl)-5-chloro-2-fluorophenyl]oxy}propyl dihydrogen phosphate hydrochloride. ¹H NMR (400 MHz, DMSO-d₆) δ 8.25 (d, 1H), 8.17 (d, 1H), 8.02 (dd, 1H), 7.65 (d, 1H), 7.33 (d, 1H), 4.85 (m, 1H), 4.40 (m, 2H), 4.10 (m, 2H), 3.81 (m, 1H), 1.35 (d, 6H).

(2S)-2-Amino-3-{[5-chloro-4-(5-{3-chloro-4-[(1-methylethyl)oxy]phenyl}-1,3,4-thiadiazol-2-yl)-2-fluorophenyl]oxy}propyl dihydrogen phosphate hydrochloride. ¹H NMR (400 MHz, DMSO-d₆) δ 8.15 (dd, 1H), 8.09 (m, 1H), 7.95 (d, 1H), 7.69 (d, 1H), 7.36 (d, 1H), 4.82 (m, 1H), 4.47 (m, 1H), 4.40 (m, 1H), 4.00 (m, 2H), 3.79 (m, 1H), 1.33 (d, 6H).

(2R)-2-Amino-3-{[5-chloro-4-(5-{3-chloro-4-[(1-methylethyl)oxy]phenyl}-1,3,4-thiadiazol-2-yl)-2-fluorophenyl]oxy}propyl dihydrogen phosphate hydrochloride. ¹H NMR (400 MHz, DMSO-d₆) δ 8.17 (d, 1H), 8.11 (m, 1H), 7.98 (d, 1H), 7.66 (d, 1H), 7.38 (d, 1H), 4.84 (m, 1H), 4.45 (m, 1H), 4.37 (m, 1H), 4.11 (m, 2H), 3.82 (m, 1H), 1.35 (d, 6H).

(2S)-2-Amino-3-{[4-(5-{3-bromo-4-[(1-methylethyl)oxy]phenyl}-1,3,4-thiadiazol-2-yl)-5-chloro-2-fluorophenyl]oxy}propyl dihydrogen phosphate. ¹H NMR (400 MHz, DMSO-d₆) δ 8.25 (d, 1H), 8.16 (d, 1H), 8.02 (dd, 1H), 7.65 (d, 1H), 7.34 (d, 1H), 4.84 (m, 1H), 4.40 (m, 2H), 4.16 (m, 2H), 3.86 (m, 1H), 1.35 (d, 6H); MS (EI) for C₂₀H₂₁BrClFN₃O₆PS, found 597.7 (MH+).

(2S)-2-Amino-3-{[5-chloro-2-fluoro-4-(5-{6-[(1-methylethyl)amino]pyridin-3-yl}-1,3,4-thiadiazol-2-yl)phenyl]oxy}propyl dihydrogen phosphate hydrochloride. ¹H NMR (400 MHz, DMSO-d₆) δ 8.60 (m, 3H), 8.17 (m, 2H), 7.68 (d, 1H), 6.85 (m, 1H), 4.40 (m, 2H), 4.15 (m, 2H), 3.82 (m, 1H), 3.40 (M, 1H), 1.22 (d, 6H); MS (EI) for C₁₉H₂₂ClFN₅O₅PS, found 518.00 (MH+).

(2S)-2-Amino-3-{[5-chloro-4-(5-{3-cyano-4-[(1-methylethyl)oxy]phenyl}-1,3,4-thiadiazol-2-yl)-2-fluorophenyl]oxy}propyl dihydrogen phosphate hydrochloride. ¹H NMR (400 MHz, CF₃COOD) δ 8.4 (s, 1H), 8.3 (d, 1H), 8.2 (d, 1H), 7.7 (d, 1H), 7.5 (d, 1H), 4.9 (m, 1H), 4.4 (m, 2H), 4.1 (m, 2H), 3.8 (m, 1H), 1.4 (d, 6H); MS (EI) for C₂₁H₂₁ClFN₄O₆PS, found 542.90 (MH+).

(1S,2S)-2-Amino-3-{[5-chloro-4-(5-{3-chloro-4-[(1-methylethyl)oxy]phenyl}-1,3,4-thiadiazol-2-yl)-2-fluorophenyl]oxy}-1-methylpropyl dihydrogen phosphate. ¹H NMR (400 MHz, DMSO-d₆) δ 8.12 (m, 2H), 7.95 (m, 1H), 7.65 (d, 1H), 7.35 (d, 1H), 6.52 (br s, 2H), 4.82 (m, 2H), 4.25 (m, 3H), 1.40 (d, 6H), 1.22 (d, 3H); MS (EI) for C₂₁H₂₃Cl₂FN₃O₆PS, found 566.15 (MH+).

(1R,2R)-2-Amino-3-{[5-chloro-4-(5-{3-chloro-4-[(1-methylethyl)oxy]phenyl}-1,3,4-thiadiazol-2-yl)-2-fluorophenyl]oxy}-1-methylpropyl dihydrogen phosphate. ¹H NMR (400 MHz, DMSO-d₆) δ 8.12 (m, 2H), 7.97 (m, 1H), 7.65 (d, 1H), 7.38 (d, 1H), 6.52 (br s, 2H), 4.82 (m, 2H), 4.27 (m, 3H), 1.38 (d, 6H), 1.25 (d, 3H); MS (EI) for C₂₁H₂₃Cl₂FN₃O₆PS, found 566.10 (MH+).

(2R)-2-Amino-3-{[5-chloro-2-fluoro-4-(5-{6-[(1-methylethyl)amino]pyridin-3yl}-1,3,4-thiadiazol-2-yl)phenyl]oxy}propyl dihydrogen phosphate. MS (EI) for C₁₉H₂₂ClFN₅O₅PS, found 517.95 (MH+).

(1R,2S)-2-Amino-3-{[5-chloro-4-(5-{3-cyano-4-[(1-methylethyl)oxy]phenyl}-1,3,4-thiadiazol-2-yl)-2-fluorophenyl]oxy}-1-methylpropyl dihydrogen phosphate hydrochloride. ¹H NMR (400 MHz, CF₃COOD) δ 8.35 (m, 2H), 8.12 (d, 1H), 7.35 (m, 2H), 5.20 (m, 1H), 4.91 (m, 1H), 4.65 (m, 2H), 4.22 (m, 1H), 1.60 (d, 3H), 1.45 (d, 6H); MS (EI) for C₂₂H₂₃ClFN₄O₆PS, found 556.95 (MH+).

(1R,2S)-2-Amino-3-{[5-chloro-4-(5-{3-chloro-4-[(1-methylethyl)oxy]phenyl}-1,3,4-thiadiazol-2-yl)-2-fluorophenyl]oxy}-1-methylpropyl dihydrogen phosphate hydrochloride. ¹H NMR (400 MHz, DMSO-d₆) δ 8.1 (m, 2H), 8.0 (d, 1H), 7.7 (m, 1H), 7.4 (s, 1H), 4.8 (m, 1H), 4.6 (m, 2H), 4.4 (m, 1H), 3.7 (m, 1H), 1.4 (m, 9H); MS (EI) for C₂₁H₂₃Cl₂FN₃O₆PS, found 565.85 (MH+).

(1S,2S)-2-Amino-3-{[5-chloro-4-(5-{3-cyano-4-[(1-methylethyl)oxy]phenyl}-1,3,4-thiadiazol-2-yl)-2-fluorophenyl]oxy}-1-methylpropyl dihydrogen phosphate hydrochloride. ¹H NMR (400 MHz, DMSO-d₆) δ 8.35 (s, 1H), 8.27 (d, 1H), 8.10 (d, 1H), 7.65 (m, 1H), 7.45 (d, 1H), 4.93 (m, 2H), 4.40 (m, 2H), 3.57 (m, 1H), 1.4 (m, 9H); MS (EI) for C₂₂H₂₃ClFN₄O₆PS, found 557.30 (MH+).

(1S,2S)-2-Amino-3-{[5-chloro-2-fluoro-4-(5-{6-[(1-methylethyl)amino]pyridin-3-yl}-1,3,4-thiadiazol-2-yl)phenyl]oxy}-1-methylpropyl dihydrogen phosphate hydrochloride. ¹H NMR (400 MHz, DMSO-d₆) δ 8.60 (s, 1H), 8.50 (br s, 2H), 8.22 (m, 1H), 8.15 (d, 1H), 7.65 (d, 1H), 6.95 (m, 1H), 4.60 (m, 1H), 4.40 (m, 2H), 3.70 (m, (1H) 1.42 (d, 3H), 1.22 (d, 6H); MS (EI) for C₂₀H₂₄ClFN₅O₅PS, found 532.05 (MH+).

(1S,2R)-2-Amino-3-{[5-chloro-4-(5-{3-chloro-4-[(1-methylethyl)oxy]phenyl}-1,3,4-thiadiazol-2-yl)-2-fluorophenyl]oxy}-1-methylpropyl dihydrogen phosphate hydrochloride. ¹H NMR (400 MHz, CF₃COOD) δ 8.2 (d, 1H), 8.1 (s, 1H), 8.0 (d, 1H), 7.4 (d, 1H), 7.2 (d, 1H), 5.2 (m, 1H), 4.8 (m, 1H), 4.6 (m, 2H), 4.2 (m, 1H), 1.6 (d, 3H), 1.4 (d, 6H); MS (EI) for C₂₁H₂₃Cl₂FN₃O₆PS, found 566.1 (MH+).

(1R,2S)-2-Amino-3-{[5-chloro-2-fluoro-4-(5-{6-[(1-methylethyl)amino]pyridin-3-yl}-1,3,4-thiadiazol-2-yl)phenyl]oxy}-1-methylpropyl dihydrogen phosphate hydrochloride. ¹H NMR (400 MHz, DMSO-d₆) δ 8.60 (m, 3H), 8.28 (d, 1H), 8.15 (d, 1H), 7.68 (d, 1H), 7.00 (m, 1H), 4.65 (m, 1H), 4.52 (m, 1H), 4.40 (m, 1H), 4.20 (m, (1H), 3.70 (m, 1H), 1.40 (d, 3H), 1.25 (d, 6H); MS (EI) for C₂₀H₂₄ClFN₅O₅PS, found 532.1 (MH+).

(1S,2R)-2-Amino-3-{[5-chloro-2-fluoro-4-(5-{6-[(1-methylethyl)amino]pyridin-3-yl}-1,3,4-thiadiazol-2-yl)phenyl]oxy}-1-methylpropyl dihydrogen phosphate hydrochloride. ¹H NMR (400 MHz, DMSO-d₆) δ 8.55 (m, 3H), 8.20 (d, 1H), 8.10 (d, 1H), 7.70 (d, 1H), 6.90 (m, 1H), 4.65 (m, 1H), 4.50 (m, 1H), 4.40 (m, 1H), 4.15 (m, (1H), 3.70 (m, 1H), 1.40 (d, 3H), 1.25 (d, 6H); MS (EI) for C₂₀H₂₄ClFN₅O₅PS, found 531.95 (MH+).

(1R,2S)-2-Amino-3-{[5-chloro-2-fluoro-4-(5-{2-methyl-6-[(1-methylethyl)amino]pyridin-4-yl}-1,3,4-thiadiazol-2-yl)phenyl]oxy}-1-methylpropyl dihydrogen phosphate hydrochloride. ¹H NMR (400 MHz, CD₃OD) δ 8.25 (d, 1H), 7.60 (d, 1H), 7.50 (s, 1H), 7.30 (s, 1H), 4.75 (m, 1H), 4.60 (m, 1H), 4.45 (m, 1H), 4.15 (m, 1H), 3.85 (m, (1H), 2.60 (s, 3H), 1.55 (d, 3H), 1.35 (d, 6H); MS (EI) for C₂₁H₂₆ClFN₅O₅PS, found 546.1 (MH+).

(1S,2S)-2-Amino-3-{[5-chloro-2-fluoro-4-(5-{2-methyl-6-[(1-methylethyl)amino]pyridin-4-yl}-1,3,4-thiadiazol-2-yl)phenyl]oxy}-1-methylpropyl dihydrogen phosphate hydrochloride. ¹H NMR (400 MHz, CD₃OD) δ 8.25 (d, 1H), 7.55 (d, 1H), 7.50 (s, 1H), 7.30 (s, 1H), 4.75 (m, 1H), 4.45 (m, 2H), 4.15 (m, 1H), 3.80 (m, (1H), 2.60 (s, 3H), 1.55 (d, 3H), 1.35 (d, 6H); MS (EI) for C₂₁H₂₆ClFN₅O₅PS, found 546.1 (MH+).

### Example 11

### (2R)-2-Amino-3-{[5-chloro-2-fluoro-4-(5-{6-[(1-methylethyl)oxy]pyridin-3-yl}-1,3,4-thiadiazol-2-yl)phenyl]oxy}propan-1-ol hydrochloride

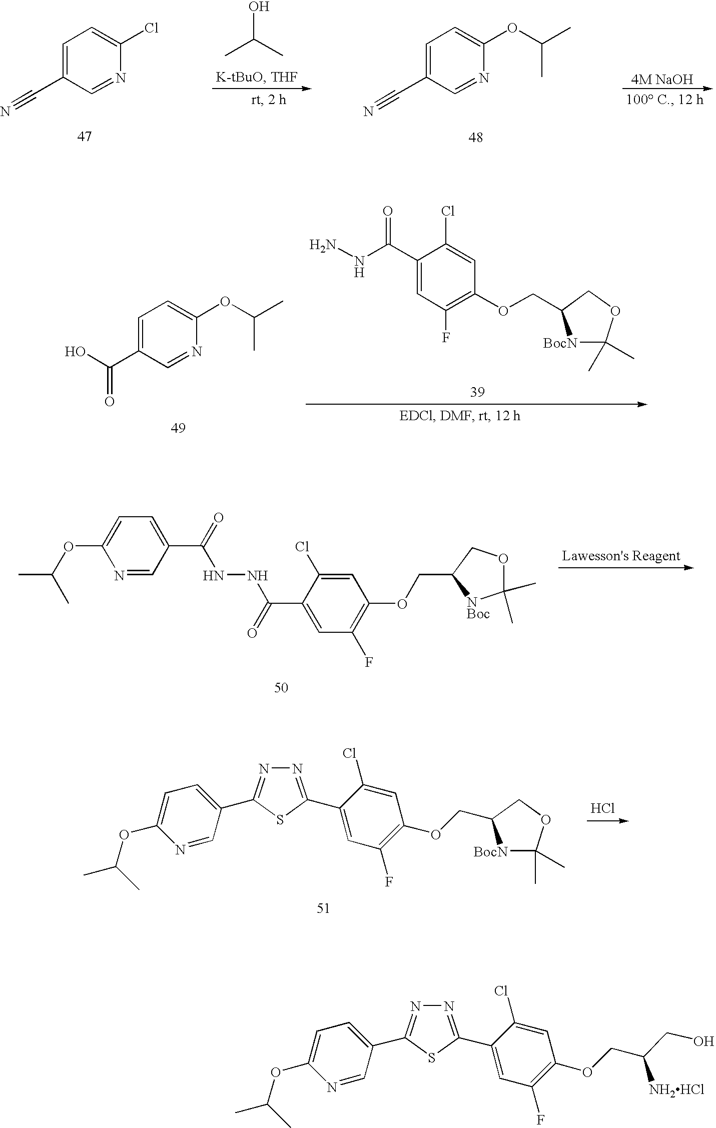

Step 1: 6-Isopropoxynicotinonitrile (48). To a stirring suspension of Potassium tert-butoxide (6.0 g, 54.12 mmol) in THF (200 mL) at 0° C. was added isopropanol (4.17 mL, 54.13 mmol) and the reaction mixture was stirred for 5 min. Compound 47 (5.0 g, 36.08 mmol) was added at 0° C. and the reaction mixture was stirred for 2 h at room temperature. The reaction mixture was concentrated in vacuo, the resulting residue was dissolved/suspended in water and extracted with EtOAc. The combined organic layers were washed with sat. NaCl, dried and concentrated to afford 48 (5.6 g) which was used as such for the next step.

Step 2: 6-Isopropoxynicotinic acid (49). A suspension of 48 (4.0 g, 24.6 mmol) in 4 M aq NaOH (120 mL) was heated at 100° C. for 12 h. The reaction mixture was concentrated to dryness, reconstituted with water (25 mL) and acidified with 1N HCl to pH 5. The resulting mixture was extracted with ethyl acetate, dried and concentrated to obtain 49 (4.5 g) which was used as such for the next step.

Step 3: (S)-tert-Butyl 4-((5-chloro-2-fluoro-4-(2-(6-isopropoxynicotinoyl)hydrazinecarbonyl)phenoxy)methyl)-2,2-dimethyloxazolidine-3-carboxylate (50). Intermediate 50 was synthesized from compounds 39 and 49 using the same or an analogous synthetic procedure to that of Example 2.

Step 4: (S)-tert-Butyl 4-((5-chloro-2-fluoro-4-(5-(6-isopropoxypyridin-3-yl)-1,3,4-thiadiazol-2-yl)phenoxy)methyl)-2,2-dimethyloxazolidine-3-carboxylate (51). Intermediate 51 was synthesized from compound 50 using the same or an analogous synthetic procedure to that of Example 3.

Step 5: (2R)-2-Amino-3-{[5-chloro-2-fluoro-4-(5-{6-[(1-methylethyl)oxy]pyridin-3-yl}-1,3,4-thiadiazol-2-yl)phenyl]oxy}propan-1-ol hydrochloride. The title compound was synthesized from compound 51 using the same or an analogous synthetic procedure to that of Example 4. ¹H NMR (400 MHz, DMSO-d₆) δ 8.8 (s, 1H), 8.3 (m, 3H), 8.1 (d, 1H), 7.7 (d, 1H), 7.0 (d, 1H), 5.5 (m, 1H), 5.4 (m, 1H), 4.4 (m, 2H), 3.7 (m, 2H), 3.6 (m, 1H), 1.4 (s, 6H); MS (EI) for C₁₉H₂₀ClFN₄O₃S, found 438.95 (MH+).

Using the same or analogous synthetic techniques in Example 11 and substituting with appropriate reagents, the following compounds were prepared.

2-Amino-3-{[5-chloro-2-fluoro-4-(5-{6-[(1-methylethyl)oxy]pyridin-3-yl}-1,3,4-thiadiazol-2-yl)phenyl]oxy}propan-1-ol hydrochloride. ¹H NMR (400 MHz, DMSO-d₆) δ 8.82 (s, 1H), 8.43 (br s, 3H, NH₃⁺), 8.31 (dd, 1H), 8.11 (d, 1H), 7.68 (d, 1H), 6.95 (d, 1H), 5.51 (br s, 1H), 5.33 (m, 1H), 4.42 (m, 2H), 3.71 (m, 2H), 3.55 (m, 1H), 1.35 (d, 6H); MS (EI) for C₁₉H₂₀ClFN₄O₃S, found 438.90 (MH+).

(2S)-2-Amino-3-{[5-chloro-2-fluoro-4-(5-{6-[(1-methylethyl)oxy]pyridin-3-yl}-1,3,4-thiadiazol-2-yl)phenyl]oxy}propan-1-ol hydrochloride. ¹H NMR (400 MHz, DMSO-d₆) δ 8.82 (s, 1H), 8.35 (d, 1H), 8.15 (m, 4H), 7.65 (d, 1H), 6.97 (d, 1H), 5.42 (br s, 1H), 5.35 (m, 1H), 4.40 (m, 2H), 3.65 (m, 3H), 1.35 (d, 6H); MS (EI) for C₁₉H₂₀ClFN₄O₃S, found 439.05 (MH+).

### Example 12

### 2-Amino-3-{[5-chloro-2-fluoro-4-(5-{6-[methyl(1-methylethyl)amino]pyridin-3-yl}-1,3,4-thiadiazol-2-yl)phenyl]oxy}propan-1-ol hydrochloride

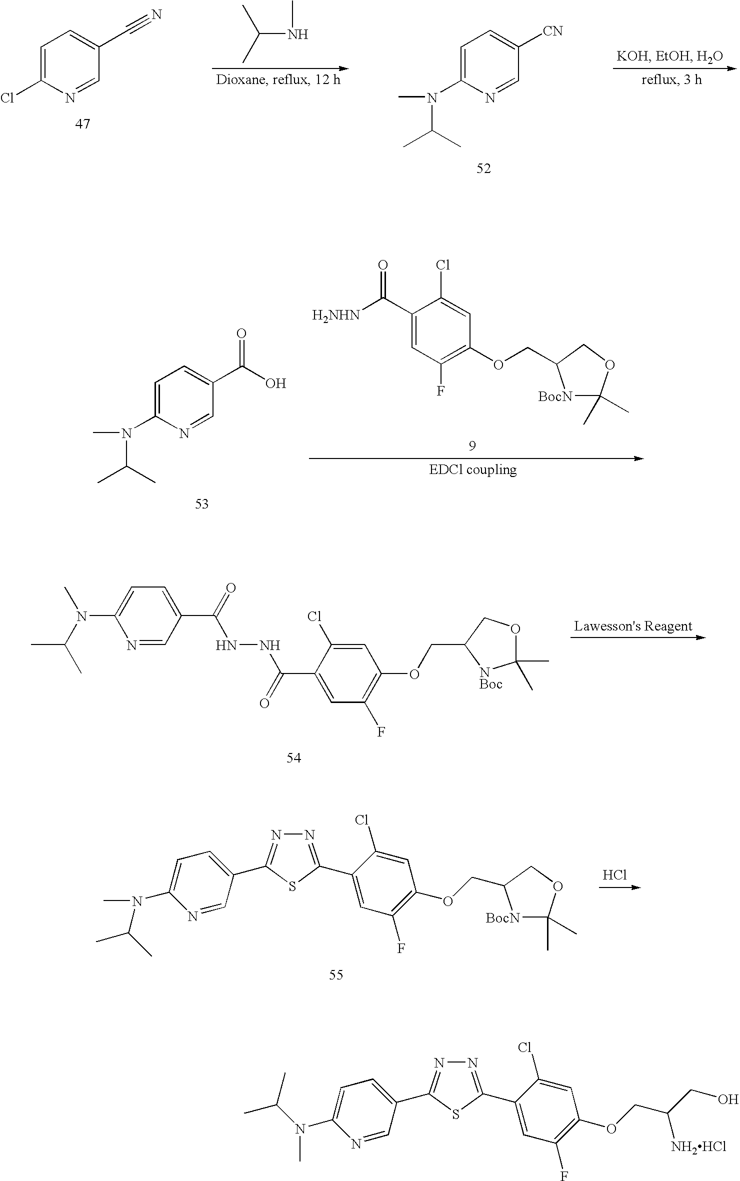

Step 1: 6-(Isopropyl(methyl)amino)nicotinonitrile (52). A solution of 47 (2.2 g, 15.88 mmol) and N-isopropylmethylamine (6.5 mL, 63.51 mmol) in dioxane (20 mL) was heated to 90° C. for 12 h. The reaction mixture was concentrated in vacuo and water added to the residue which was then basified with NaOH. The aqueous layer was extracted with EtOAc, dried and concentrated to afford 52 (2.7 g, 97%).

Step 2: 6-(Isopropyl(methyl)amino)nicotinic acid (53). To a stirring solution of 52 (2.7 g, 15 mmol) in ethanol (13.5 mL) was added 12% KOH solution (13.5 mL) and the reaction mixture was heated to 100° C. for 3 h. The reaction mixture was concentrated in vacuo. The resulting residue was cooled, then acidified to pH 4-5 with sat. citric acid. The resulting aqueous mixture was extracted with ethyl acetate, dried and concentrated to afford 53 (1.5 g) which was used as such for the next step.

Step 3: tert-Butyl 4-((5-chloro-2-fluoro-4-(2-(6-(isopropyl(methyl)amino)nicotinoyl)hydrazinecarbonyl)phenoxy)methyl)-2,2-dimethyloxazolidine-3-carboxylate (54). Intermediate 54 was synthesized from compounds 9 and 53 using the same or an analogous synthetic procedure to that of Example 2.

Step 4: tert-Butyl 4-((5-chloro-2-fluoro-4-(5-(6-(isopropyl(methyl)amino)pyridin-3-yl)-1,3,4-thiadiazol-2-yl)phenoxy)methyl)-2,2-dimethyloxazolidine-3-carboxylate (55). Intermediate 55 was synthesized from compound 54 using the same or an analogous synthetic procedure to that of Example 3.

Step 5: 2-Amino-3-{[5-chloro-2-fluoro-4-(5-{6-[methyl(1-methylethyl)amino]pyridin-3-yl}-1,3,4-thiadiazol-2-yl)phenyl]oxy}propan-1-ol hydrochloride. The title compound was synthesized from compound 55 using the same or an analogous synthetic procedure to that of Example 4. ¹H NMR (400 MHz, DMSO-d₆) δ 8.7 (s, 1H), 8.1 (m, 4H), 7.6 (d, 1H), 6.8 (d, 1H), 5.0 (m, 1H), 4.4 (m, 2H), 3.6 (m, 3H), 2.9 (s, 3H), 1.2 (d, 6H); MS (EI) for C₂₀H₂₃ClFN₅O₂S, found 452.10 (MH+).

### Example 13

### 2-Amino-3-{[5-chloro-2-fluoro-4-(5-{2-[methyl(1-methylethyl)amino]pyridin-4-yl}-1,3,4-thiadiazol-2-yl)phenyl]oxy}propan-1-ol hydrochloride

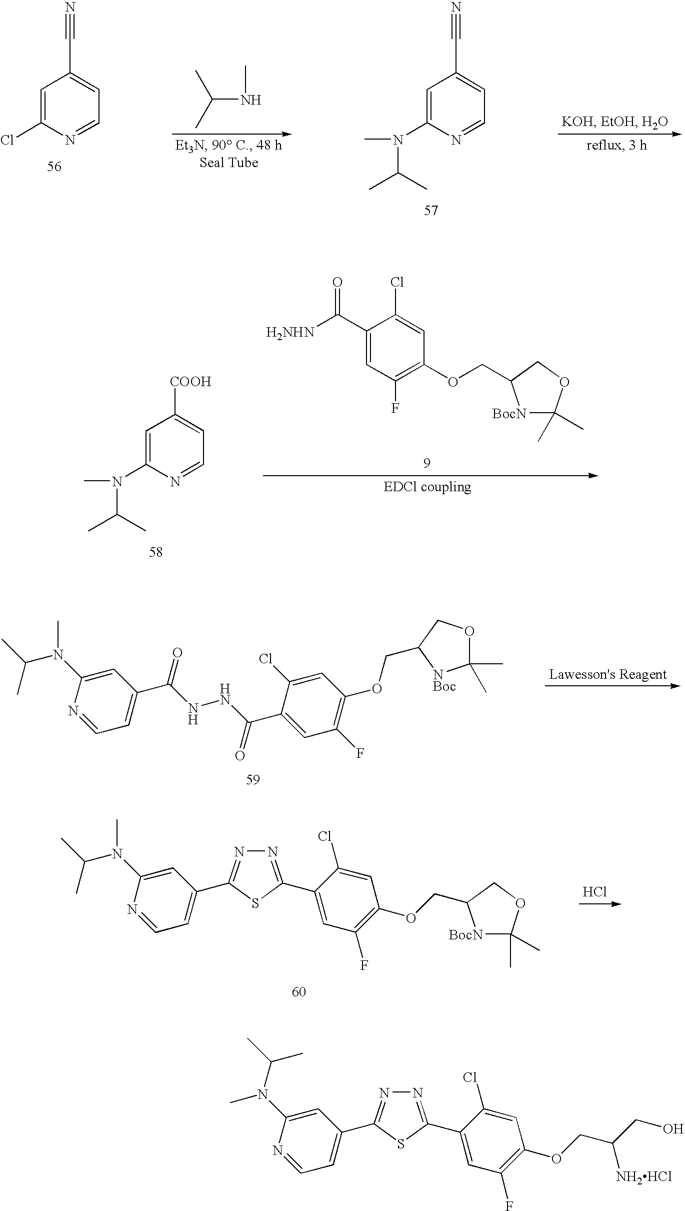

Step 1: 2-(Isopropyl(methyl)amino)isonicotinonitrile (57). A mixture of 56 (3.0 g, 22 mmol), N-isopropylmethylamine (13.9 mL, 129 mmol) and Et₃N (3.6 mL, 26 mmol) was heated at 90° C. for 48 h in a seal tube. The reaction mixture was concentrated in vacuo and water was added to the resulting residue. The aqueous mixture was extracted with EtOAc, dried over Na₂SO₄, concentrated and purified by column chromatography to afford 57 (2.6 g, 70%).

Step 2: 2-(Isopropyl(methyl)amino)isonicotinic acid (58). To a stirring solution of 57 (2.6 g, 14.86 mmol) in ethanol (13.5 mL) was added 12% KOH solution (13.5 mL) and the reaction mixture was heated to 100° C. for 3 h. The reaction mixture was concentrated in vacuo, cooled and acidified to pH 4-5 with sat. citric acid solution. The acidic aqueous mixture was extracted with ethyl acetate, dried and concentrated to afford 58 (1.4 g, 48.3%).

Step 3: tert-Butyl 4-((5-chloro-2-fluoro-4-(2-(2-(isopropyhmethyl)amino)isonicotinoyl)hydrazinecarbonyl)phenoxy)methyl)-2,2-dimethyloxazolidine-3-carboxylate (59). Intermediate 59 was synthesized from compounds 9 and 58 using the same or an analogous synthetic procedure to that of Example 2.

Step 4: (S)-tert-Butyl 4-((5-chloro-2-fluoro-4-(5-(2-(isopropyhmethyl)amino)pyridin-4-yl)-1,3,4-thiadiazol-2-yl)phenoxy)methyl)-2,2-dimethyloxazolidine-3-carboxylate (60). Intermediate 60 was synthesized from compound 59 using the same or an analogous synthetic procedure to that of Example 3.

Step 5: 2-Amino-3-{[5-chloro-2-fluoro-4-(5-{2-[methyl(1-methylethyl)amino]pyridin-4-yl}-1,3,4-thiadiazol-2-yl)phenyl]oxy}propan-1-ol hydrochloride. The title compound was synthesized from compound 60 using the same or an analogous synthetic procedure to that of Example 4. ¹H NMR (400 MHz, DMSO-d₆) δ 8.3-8.1 (m, 5H), 7.7 (d, 1H), 7.2 (m, 1H), 4.9 (m, 1H), 4.5-4.3 (m, 2H), 3.8-3.6 (m, 3H), 2.9 (s, 3H), 1.2 (d, 6H); MS (EI) for C₂₀H₂₃ClFN₅O₂S, found 452.20 (MH+).

### Example 14

### (2R)-2-Amino-3-{[5-chloro-4-(5-{3-chloro-4-[(1-methylethyl)amino]phenyl}-1,3,4-thiadiazol-2-yl)-2-fluorophenyl]oxy}propan-1-ol hydrochloride

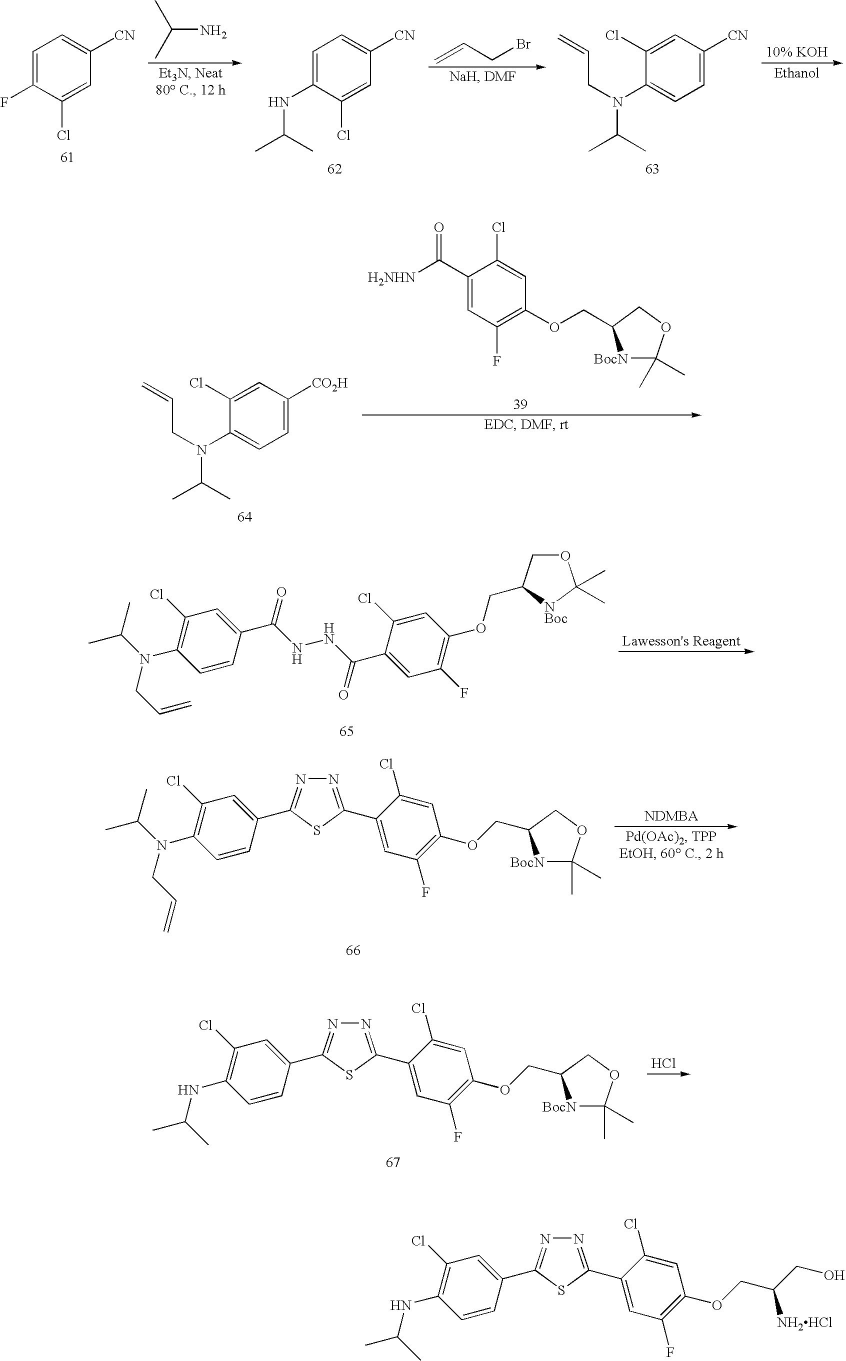

Step 1: 3-Chloro-4-(isopropylamino)benzonitrile (62). A reaction mixture of isopropylamine (50 mL), 61 (5.0 g, 32.15 mmol) and Et₃N (5.4 mL, 38.57 mmol) was heated in a sealed tube at 80° C. The reaction mixture was then cooled and diluted with water. The aqueous layer was extracted with ethyl acetate. The organic layer was dried and concentrated to afford 62 (6.0 g) as a colorless oil which was used as such for the next step.

Step 2: 4-(Allyl(isopropyl)amino)-3-chlorobenzonitrile (63). To an ice cooled solution of 62 (5.5 g, 28.25 mmol) in DMF (110 mL) was added NaH (1.24 g, 31.07 mmol) and the reaction mixture was stirred at the same temperature for 30 min. Allyl bromide (4.1 g, 33.90 mmol) was added and reaction was further stirred at the same temperature for 15 min and was then quenched with ice, stirred for 15 min at 0° C. and the resulting aqueous mixture was extracted with EtOAc. The organic layer was dried, concentrated and purified by column chromatography to obtain 63 as a colorless oil (6.53 g, 98%).

Step 3: 4-(Allyl(isopropyl)amino)-3-chlorobenzoic acid (64). To a stirred solution of 63 (6.3 g, 26.84 mmol) in EtOH (32 mL) was added 10% aqueous KOH (63 mL) solution and the reaction mixture was refluxed for 4 h. The reaction mixture was concentrated in vacuo and the resulting residue was neutralized to pH 5 with citric acid solution. The aqueous layer was extracted with ethyl acetate. The organic layer was dried and concentrated to afford a semi solid compound which was washed with ether (50 mL×2). The combined ether layers were concentrated to afford 64 (3.7 g, 54%) as a pale yellow oil, which was used as such for the next step.

Step 4: (S)-tert-Butyl 4-((4-(2-(4-(allyl(isopropyl)amino)-3-chlorobenzoyl)hydrazinecarbonyl)-5-chloro-2-fluorophenoxy)methyl)-2,2-dimethyloxazolidine-3-carboxylate (65). Intermediate 65 was synthesized from compounds 39 and 64 using the same or an analogous synthetic procedure to that of Example 2.

Step 5: (S)-tert-Butyl 4-((4-(5-(4-(allyl(isopropyl)amino)-3-chlorophenyl)-1,3,4-thiadiazol-2-yl)-5-chloro-2-fluorophenoxy)methyl)-2,2-dimethyloxazolidine-3-carboxylate (66). Intermediate 66 was synthesized from compound 65 using the same or an analogous synthetic procedure to that of Example 3.

Step 6: (S)-tert-Butyl 4-((5-chloro-4-(5-(3-chloro-4-(isopropylamino)phenyl)-1,3,4-thiadiazol-2-yl)-2-fluorophenoxy)methyl)-2,2-dimethyloxazolidine-3-carboxylate (67). A stirred solution of 66 (1.7 g, 2.78 mmol), 1,3-dimethyl barbituric acid (1.3 g, 8.33 mmol), Pd(OAc)₂ (0.624 g, 2.78 mmol) and TPP (0.73 g, 2.78 mmol) in ethanol (17 mL) was purged with Argon for 20 min followed by heating to 60° C. for 2 h. After cooling, the reaction mixture was filtered through Celite and the filtrate concentrated. Water was added to the resulting residue and the aqueous mxiture extracted with ethyl acetate. The organic layer was dried, concentrated and purified by column chromatography to obtain 67 (1.1 g, 68.7%).

Step 7: (2R)-2-Amino-3-{[5-chloro-4-(5-{3-chloro-4-[(1-methylethyl)amino]phenyl}-1,3,4-thiadiazol-2-yl)-2-fluorophenyl]oxy}propan-1-ol hydrochloride. The title compound was synthesized from compound 67 using the same or an analogous synthetic procedure to that of Example 4. ¹H NMR (400 MHz, DMSO-d₆) δ 8.2 (m, 3H), 7.9 (s, 1H), 7.8 (d, 1H), 7.6 (d, 1H), 6.9 (d, 1H), 5.5 (d, 1H), 5.4 (m, 1H), 4.4 (m, 2H), 3.7 (m, 4H), 1.2 (d, 6H); MS (EI) for C₂₀H₂₁Cl₂FN₄O₂S, found 471.15 (MH+).

Using the same or analogous synthetic techniques in Example 14 and substituting with appropriate reagents, the following compound was prepared.

(2S)-2-Amino-3-{[5-chloro-4-(5-{3-chloro-4-[(1-methylethyl)amino]phenyl}-1,3,4-thiadiazol-2-yl)-2-fluorophenyl]oxy}propan-1-ol hydrochloride. ¹H NMR (400 MHz, DMSO-d₆) δ 8.12 (m, 4H), 7.91 (s, 1H), 7.79 (d, 1H), 7.60 (d, 1H), 6.88 (d, 1H), 5.51 (d, 1H), 5.4 (m, 1H), 4.35 (m, 2H), 3.7 (m, 4H), 1.2 (d, 6H); MS (EI) for C₂₀H₂₁Cl₂FN₄O₂S, found 471.15 (MH+).

### Example 15

### 2-Amino-3-{[5-chloro-2-fluoro-4-(5-{6-[(1-methylethyl)amino]pyridin-3-yl}-1,3,4-thiadiazol-2-yl)phenyl]oxy}propan-1-ol hydrochloride

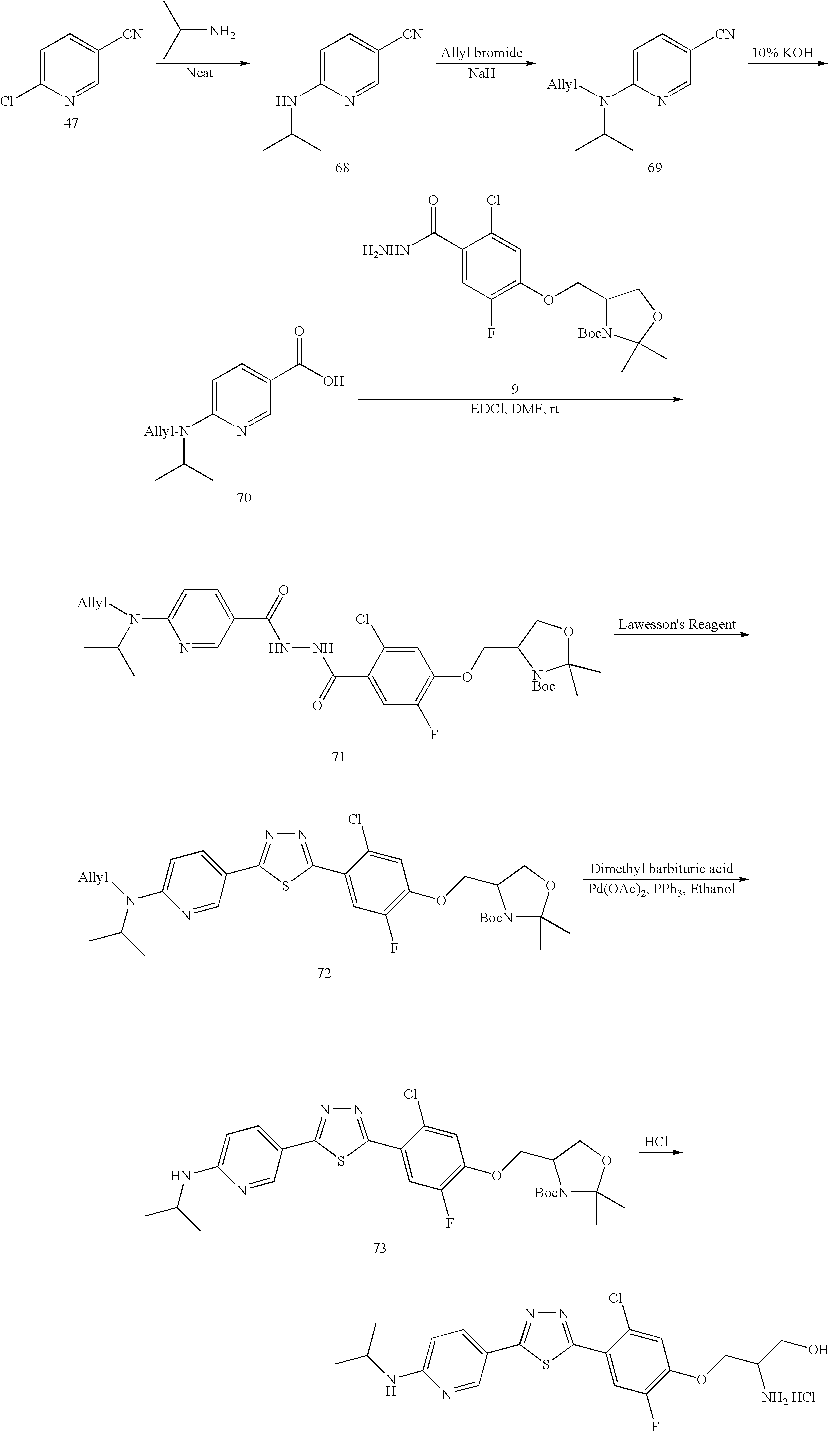

Step 1: 6-(Isopropylamino)nicotinonitrile (68). A mixture of isopropylamine (15.4 mL, 180.4 mmol) and 47 (5.0 g, 36.2 mmol) was heated in a sealed tube at 80° C. for 12 h. The reaction mixture was then cooled and concentrated. Water was added to the residue and the resulting aqueous mixture was extracted with ethyl acetate. The organic layer was washed with sat. NaCl, dried and concentrated to afford 68 (4.74 g, 81%) as a colorless oil which was used as such for the next step.

Step 2: 6-(Allyl(isopropyl)amino)nicotinonitrile (69). To an ice cooled solution of 68 (4.55 g, 28.26 mmol) in DMF (110 mL) was added NaH (1.24 g, 31.07 mmol) and the reaction mixture was stirred at the same temperature for 30 min. Allyl bromide (4.1 g, 33.90 mmol) was then added and reaction was further stirred at the same temperature for 2 h. The reaction mixture was then quenched with ice with stirring for 15 min at 0° C. and then further neutralized with citric acid solution. The resulting aqueous mixture was then extracted with EtOAc, dried and concentrated to obtain 69 (5.0 g, 88%) which was used as such in the next step.

Step 3: 6-(Allyl(isopropyl)amino)nicotinic acid (70). To a stirred solution of 69 (5.38 g, 26.77 mmol) in EtOH (32 mL) was added aqueous 10% KOH solution (63 mL) and the reaction mixture was refluxed for 4 h. The reaction mixture was then concentrated in vacuo and the resulting residue was neutralized to pH 5 with citric acid solution. The aqueous mixture was extracted with ethyl acetate. The organic layer was dried, concentrated and washed with pentane to afford 70 (4.2 g, 71.3%) as a white solid.

Step 4: tert-butyl 4-((4-(2-(6-(allyl(isopropyl)amino)nicotinoyl)hydrazine-carbonyl)-5-chloro-2-fluorophenoxy)methyl)-2,2-dimethyloxazolidine-3-carboxylate (71). Intermediate 71 was synthesized from compounds 9 and 70 using the same or an analogous synthetic procedure to that of Example 2.

Step 5: (S)-tert-Butyl 4-((5-chloro-2-fluoro-4-(5-(6-(isopropylamino)pyridin-3-yl)-1,3,4-thiadiazol-2-yl)phenoxy)methyl)-2,2-dimethyloxazolidine-3-carboxylate (72). Intermediate 72 was synthesized from compound 71 using the same or an analogous synthetic procedure to that of Example 3.

Step 6: (S)-tert-Butyl 4-((5-chloro-2-fluoro-4-(5-(6-(isopropylamino)pyridin-3-yl)-1,3,4-thiadiazol-2-yl)phenoxy)methyl)-2,2-dimethyloxazolidine-3-carboxylate (73). A stirred solution of 72 (1.1 g, 1.78 mmol), 1,3-dimethyl barbituric acid (1.1 g, 5.3 mmol), Pd(OAc)₂ (0.39 g, 1.78 mmol), and TPP (0.47 g, 1.78 mmol) in ethanol (15 mL) was purged with Argon for 20 min. followed by heating to 85° C. for 12 h. After cooling, the reaction mixture was filtered through Celite and the filtrate was concentrated. The resulting residue was diluted with water and extracted with ethyl acetate. The organic layer was dried, concentrated and purified by column chromatography to obtain 73 (0.8 g).

Step 7: 2-Amino-3-{[5-chloro-2-fluoro-4-(5-{6-[(1-methylethyl)amino]pyridin-3-yl}-1,3,4-thiadiazol-2-yl)phenyl]oxy}propan-1-ol hydrochloride. The title compound was synthesized from compound 73 using the same or an analogous synthetic procedure to that of Example 4. ¹H NMR (400 MHz, DMSO-d₆) δ 8.6 (s, 1H), 8.2 (m, 3H), 8.0 (d, 1H), 7.6 (d, 1H), 7.4 (s, 1H), 6.6 (d, 1H), 4.4 (m, 4H), 3.6 (m, 3H), 1.2 (d, 6H); MS (EI) for C₁₉H₂₁ClFN₅O₂S, found 438.15 (MH+).

Using the same or analogous synthetic techniques in Example 15 and substituting with appropriate reagents, the following compounds were prepared.

(2R)-2-Amino-3-{[5-chloro-2-fluoro-4-(5-{6-[(1-methylethyl)amino]pyridin-3-yl}-1,3,4-thiadiazol-2-yl)phenyl]oxy}propan-1-ol hydrochloride. ¹H NMR (400 MHz, DMSO-d₆) δ 8.6 (s, 1H), 8.3 (bs, 3H), 8.2 (m, 2H), 7.6 (d, 1H), 6.9 (m, 1H), 4.4 (m, 2H), 4.2 (m, 1H), 3.8 (m, 3H), 3.6 (m, 1H), 1.2 (d, 6H); MS (EI) for C₁₉H₂₁ClFN₅O₂S, found 438.25 (MH+).

(2S)-2-Amino-3-{[5-chloro-2-fluoro-4-(5-{6-[(1-methylethyl)amino]pyridin-3-yl}-1,3,4-thiadiazol-2-yl)phenyl]oxy}propan-1-ol hydrochloride. ¹H NMR (400 MHz, DMSO-d₆) δ 8.6 (s, 1H), 8.4 (bs, —NH₂, 2H), 8.2 (m, 1H), 8.15 (d, 1H), 7.6 (d, 1H), 6.9 (m, 1H), 4.4 (m, 2H), 4.2 (m, 1H), 4.0 (m, 1H), 3.7 (m, 2H), 3.6 (m, 1H), 1.2 (m, 6H); MS (EI) for C₁₉H₂₁ClFN₅O₂S, found 438.00 (MH+).

(2R,3S)-3-Amino-4-{[5-chloro-2-fluoro-4-(5-{6-[(1-methylethyl)amino]pyridin-3-yl}-1,3,4-thiadiazol-2-yl)phenyl]oxy}butan-2-ol hydrochloride. ¹H NMR (400 MHz, DMSO-d₆) δ 8.6 (s, 1H), 8.1 (m, 2H), 8.0 (d, 1H), 7.7 (d, 1H), 7.5 (m, 1H), 6.7 (d, 1H), 4.5-3.8 (m, 4H), 3.5 (m, 1H), 1.2 (m, 9H); MS (EI) for C₂₀H₂₃ClFN₅O₂S, found 452.05 (MH+).

(2S,3S)-3-Amino-4-{[5-chloro-2-fluoro-4-(5-{6-[(1-methylethyl)amino]pyridin-3-yl}-1,3,4-thiadiazol-2-yl)phenyl]oxy}butan-2-ol hydrochloride. ¹H NMR (400 MHz, DMSO-d₆) δ 8.6 (s, 1H), 8.2-8.0 (m, 4H), 7.6 (d, 1H), 6.6 (d, 1H), 4.4 (m, 2H), 4.1 (m, 1H), 3.9 (m, 1H), 3.4 (m, 2H), 1.2 (m, 9H); MS (EI) for C₂₀H₂₃ClFN₅O₂S, found 451.95 (MH+).

(2S,3R)-3-Amino-4-{[5-chloro-2-fluoro-4-(5-{6-[(1-methylethyl)amino]pyridin-3-yl}-1,3,4-thiadiazol-2-yl)phenyl]oxy}butan-2-ol hydrochloride. ¹H NMR (400 MHz, DMSO-d₆) δ 8.6 (s, 1H), 8.3 (m, 3H), 8.2 (m, 1H), 8.1 (d, 1H), 7.7 (d, 1H), 6.9 (m, 1H), 4.5 (m, 1H), 4.3 (m, 1H), 4.2 (m, 1H), 4.0 (m, 1H), 3.7 (m, 1H), 3.5 (m, 1H), 1.2 (m, 9H); MS (EI) for C₂₀H₂₃ClFN₅O₂S, found 452.05 (MH+).

(2R,3R)-3-Amino-4-{[5-chloro-2-fluoro-4-(5-{6-[(1-methylethyl)amino]pyridin-3-yl}-1,3,4-thiadiazol-2-yl)phenyl]oxy}butan-2-ol hydrochloride. ¹H NMR (400 MHz, D₂O) δ 8.2 (s, 1H), 8.0 (m, 1H), 7.7 (d, 1H), 7.3 (d, 1H), 6.8 (d, 1H), 4.3 (m, 2H), 4.0 (m, 1H), 3.7 (m, 1H), 3.5 (m, 1H), 1.3 (d, 3H), 1.1 (d, 6H); MS (EI) for C₂₀H₂₃ClFN₅O₂S, found 452.10 (MH+).

### Example 16

### (2R,3S)-3-Amino-4-{[5-chloro-4-(5-{5-chloro-6-[(1-methylethyl)amino]pyridin-3yl}-1,3,4-thiadiazol-2-yl)-2-fluorophenyl]oxy}butan-2-ol hydrochloride

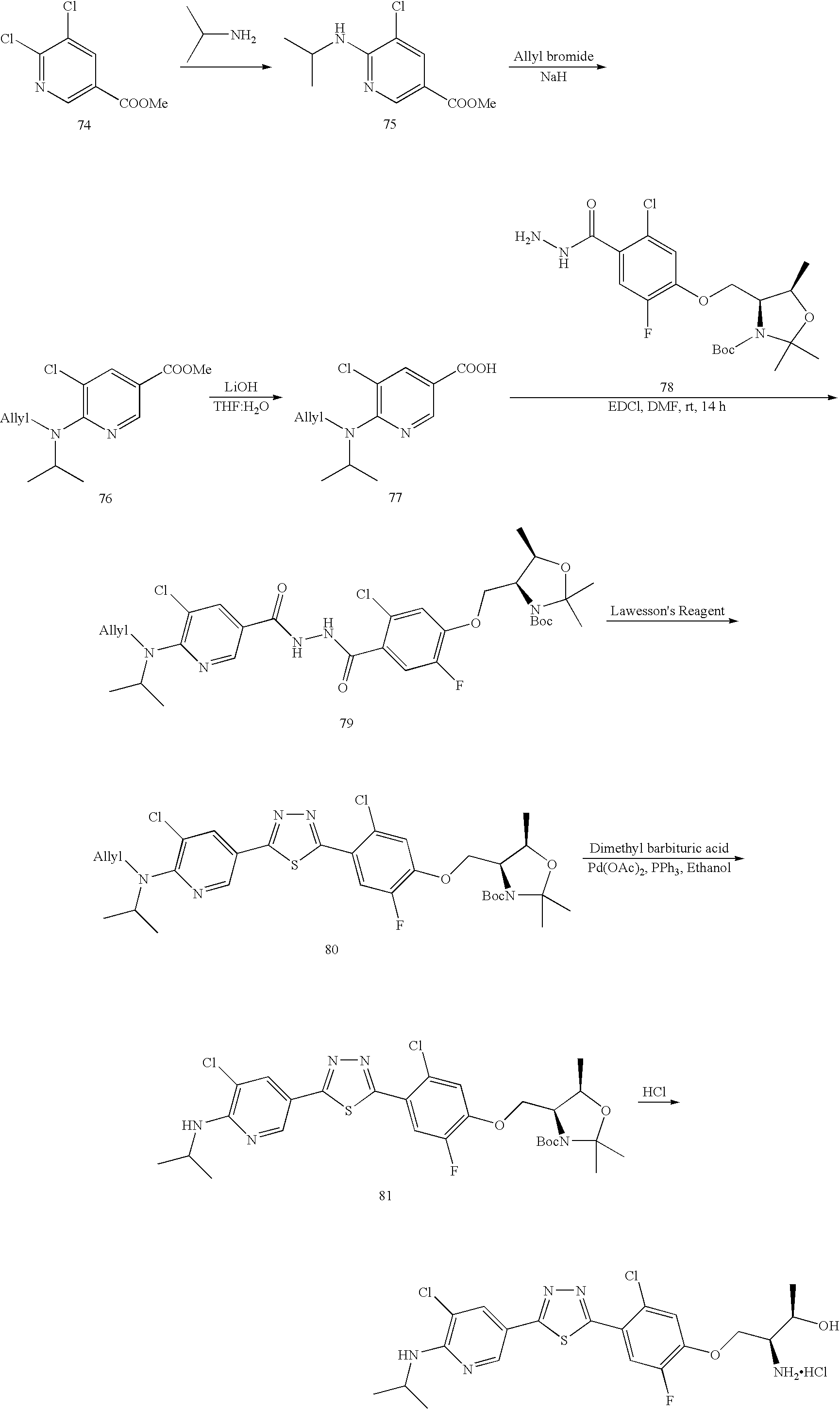

Step 1: Methyl 5-chloro-6-(isopropylamino)nicotinate (75). A mixture of 5,6-dichloronicotinic acid methyl ester 74 (6.0 g, 15.2 mmol) and isopropylamine (30 mL, 60.6 mmol) was stirred in a sealed vessel at 100° C. for 14 h. The reaction mixture was cooled to room temperature and concentrated. The residue was dissolved in ethyl acetate and washed with water, sat. NaCl, dried over Na₂SO₄, concentrated and purified by column chromatography to give 75 (4.4 g, 65. 9%).

Step 2: Methyl 6-(allyl(isopropyl)amino)-5-chloronicotinate (76). To an ice cooled solution of 75 (4.4 g, 19.29 mmol) in DMF (40 mL) was added NaH (0.84 g, 21.12 mmol) and the reaction mixture was stirred at the same temperature for 30 min. Allyl bromide (2 mL, 23.04 mmol) was added and the reaction was further stirred at the same temperature for 15 min and at room temperature for 2 h. The reaction mixture was then quenched with ice, stirred for 15 min at 0° C. and then further neutralized with citric acid solution. The resulting aqueous mixture was extracted with EtOAc, dried and concentrated to obtain 76 (5 g) which was used as such for the next step.

Step 3: 6-(Allyl(isopropyl)amino)-5-chloronicotinic acid (77). To a stirred solution of 76 (5.0 g, 18.6 mmol) in THF (15 mL) and water (15 ml) was added LiOH (3.1 g, 74 mmol) and the reaction mixture stirred for 12 h at room temperature. The reaction mixture was concentrated and the resulting residue acidified to pH 5 with citric acid solution. The aqueous mixture was extracted with EtOAc, dried and concentrated to afford 77 (4.2 g, 88.7%) which was used as such for the next step.

Step 4: (4S,5R)-tert-Butyl 4-((4-(2-(6-(allyl(isopropyl)amino)-5-chloronicotinoyl)hydrazinecarbonyl)-5-chloro-2-fluorophenoxy)methyl)-2,2,5-trimethyloxazolidine-3-carboxylate (79). Intermediate 78 was made in an analogous manner to intermediate 9 using the appropriate enantiomerically pure starting material in place of a racemic mixture. Intermediate 79 was synthesized from intermediates 77 and 78 using the same or an analogous synthetic procedure to that of Example 2.

Step 5: (4S,5R)-tert-Butyl 4-((4-(5-(6-(allyl(isopropyl)amino)-5-chloropyridin-3-yl)-1,3,4-thiadiazol-2-yl)-5-chloro-2-fluorophenoxy)methyl)-2,2,5-trimethyloxazolidine-3-carboxylate (80). Intermediate 80 was synthesized from intermediate 79 using the same or an analogous synthetic procedure to that of Example 3.

Step 6: (4S,5R)-tert-Butyl 4-((5-chloro-4-(5-(5-chloro-6-(isopropylamino)pyridin-3-yl)-1,3,4-thiadiazol-2-yl)-2-fluorophenoxy)methyl)-2,2,5-trimethyloxazolidine-3-carboxylate (81). A stirred solution of 80 (1.2 g, 1.8 mmol), 1,3-dimethyl barbituric acid (1.1 g, 7.2 mmol) and TPP (0.47 g, 1.8 mmol) in ethanol (10 mL) was purged with Argon for 20 min then Pd(OAc)₂ (0.4 g, 1.8 mmol) was added and again purged with argon for 15 min. The reaction mixture was then heated to 85° C. for 12 h. After cooling to room temperature, the reaction mixture was concentrated and purified by column chromatography to obtain 81 (0.4 g, 35.4%).

Step 7: (2R,3S)-3-Amino-4-{[5-chloro-4-(5-{5-chloro-6-[(1-methylethyl)amino]pyridin-3-yl}-1,3,4-thiadiazol-2-yl)-2-fluorophenyl]oxy}butan-2-ol hydrochloride. The title compound was synthesized from intermediate 81 using the same or an analogous synthetic procedure to that of Example 4. ¹H NMR (400 MHz, DMSO-d₆) δ 8.7 (s, 1H), 8.2 (m, 3H), 7.7 (m, 1H), 6.8 (m, 1H), 5.5 (m, 1H), 4.5 (m, 1H), 4.3 (m, 2H), 4.0 (m, 1H), 1.2 (m, 9H); MS (EI) for C₂₀H₂₂Cl₂FN₅O₂S, found 485.95 (MH+).

Using the same or analogous synthetic techniques in Example 16 and substituting with appropriate reagents, the following compound was prepared.

(2R)-2-Amino-3-{[5-chloro-4-(5-{5-chloro-6-[(1-methylethyl)amino]pyridin-3-yl}-1,3,4-thiadiazol-2-yl)-2-fluorophenyl]oxy}propan-1-ol hydrochloride. ¹H NMR (400 MHz, DMSO-d₆) δ 8.7 (s, 1H), 8.2 (m, 5H), 7.6 (d, 1H), 6.8 (d, 1H), 5.4 (br s, 1H), 4.4 (m, 3H), 3.7 (m, 3H), 1.2 (m, 6H); MS (EI) for C₁₉H₂₀Cl₂FN₅O₂S, found 471.95 (MH+).

### Example 17

### 5-[5-(4-{[(2R,3S)-2-Amino-3-hydroxybutyl]oxy}-2-chloro-5-fluorophenyl)-1,3,4-thiadiazol-2-yl]-2-[(1-methylethyl)oxy]benzonitrile hydrochloride

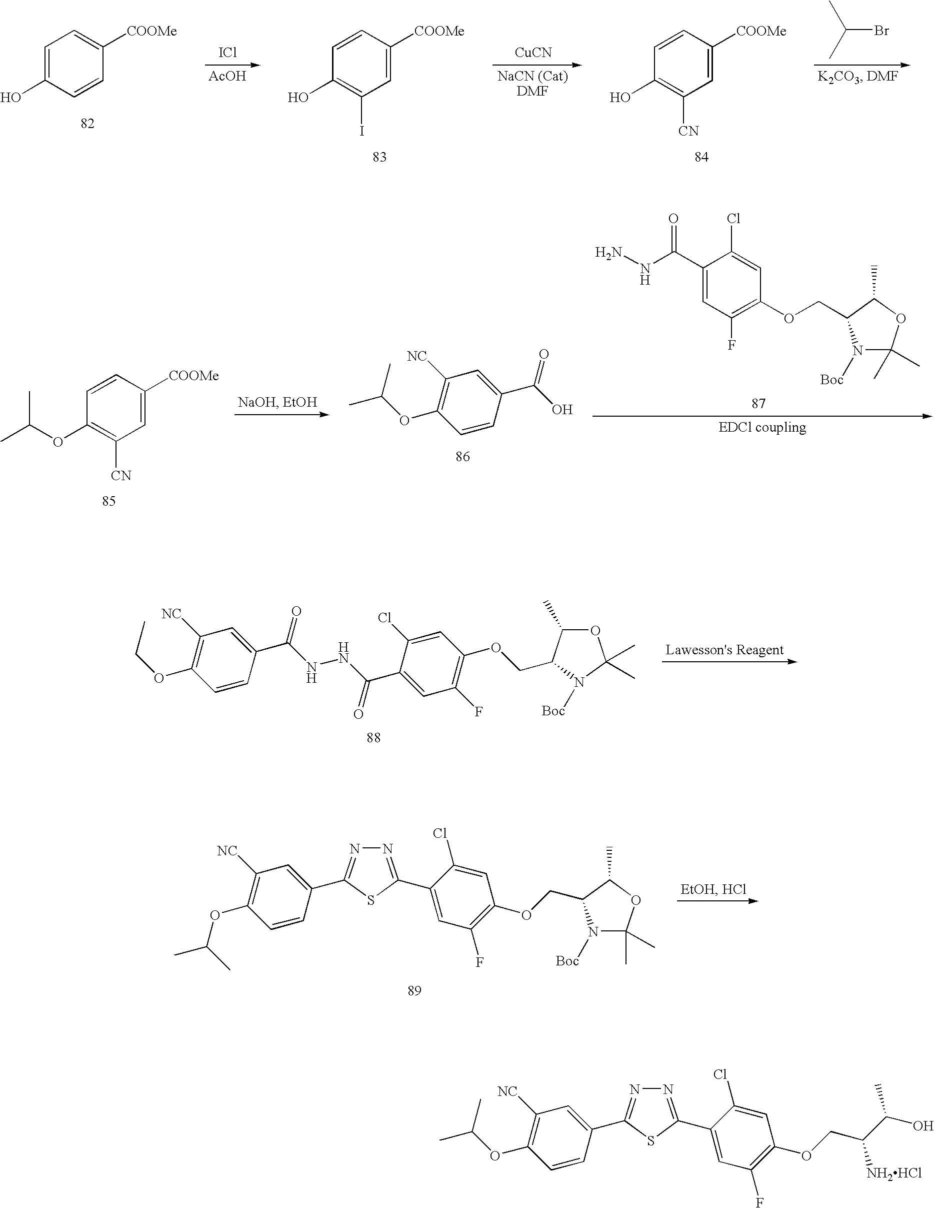

Step 1: Methyl 4-hydroxy-3-iodobenzoate (83). To a stirred solution of methyl-4-hydroxybenzoate 82 (20 g, 132 mmol) in AcOH (100 mL) heated at 65° C. was added ICl (21.3 g, 7.0 mL, 132 mmol) in AcOH (25 mL) dropwise over 40 min. The reaction mixture was further stirred at 65° C. for 8 h and then stirred an additional 16 h at room temperature. The precipitated product was isolated via filtration, washed with water and dried under vacuum to give 83 (20 g, 55%) as a white solid.

Step 2: Methyl 3-cyano-4-hydroxybenzoate (84). To a stirred solution of 83 (12 g, 44 mmol) in DMF (60 mL) was added CuCN (4.4 g, 48 mmol) and NaCN (0.24 g, 4.8 mmol) and the resulting mixture was heated to 105° C. for 18 h. The reaction mixture was allowed to cool to room temperature and any precipitates were removed via filteration and washed with ethyl acetate. The combined organics were diluted with water (200 mL), extracted with ethyl acetate, dried over Na₂SO₄ and concentrated to afford 84 (6.2 g, 80%) as a light yellow solid.

Step 3: Methyl 3-cyano-4-isopropoxybenzoate (85). To as stirred solution of 84 (6.2 g, 34 mmol) in DMF (25 mL) was added 2-bromopropane (6.34 g, 52 mmol) and K₂CO₃ (14 g, 103 mmol). The resulting reaction mixture was heated to 90° C. for 14 h. After cooling to room temperature, the reaction mixture was diluted with water (200 mL) and extracted with DCM. The combined organic layers were dried over Na₂SO₄, concentrated and purified by column chromatography to give 85 (7.0 g, 91%) as a thick oil.

Step 4: 3-Cyano-4-isopropoxybenzoic acid (86). To a stirred solution of 85 (7.0 g, 315 mmol) in a mixture of ethanol (30 mL) and THF (30 mL) was added 2M sodium hydroxide (20 mL, 41 mmol) and the resulting mixture was stirred at room temperature for 4 h. The reaction mixture was concentrated in vacuo, the residue was diluted with water (100 mL) and acidified with 2N HCl. The resulting aqueous mixture was extracted with ethyl acetate. The organic layer was washed with water, sat. NaCl, dried over Na₂SO₄. The crude compound was then stirred in 10% ether in hexane, filtered and dried to afford 86 (6 g, 92%) as an off-white solid.

Step 5: (4R,5S)-tert-Butyl 4-((5-chloro-4-(2-(3-cyano-4-isopropoxybenzoyl)hydrazinecarbonyl)-2-fluorophenoxy)methyl)-2,2,5-trimethyloxazolidine-3-carboxylate (88). Intermediate 87 was made in an analogous manner to intermediate 9 using the appropriate enantiomerically pure starting material in place of a racemic mixture. Intermediate 88 was synthesized from intermediates 86 and 87 using the same or an analogous synthetic procedure to that of Example 2.

Step 6: (4R,5S)-tert-Butyl 4-((5-chloro-4-(5-(3-cyano-4-isopropoxyphenyl)-1,3,4-thiadiazol-2-yl)-2-fluorophenoxy)methyl)-2,2,5-trimethyloxazolidine-3-carboxylate (89). Intermediate 89 was synthesized from intermediate 88 using the same or an analogous synthetic procedure to that of Example 3.

Step 7: 5-[5-(4-{[(2R,3S)-2-Amino-3-hydroxybutyl]oxy}-2-chloro-5-fluorophenyl)-1,3,4-thiadiazol-2-yl]-2-[(1-methylethyl)oxy]benzonitrile hydrochloride. The title compound was synthesized from intermediate 89 using the same or an analogous synthetic procedure to that of Example 4. ¹H NMR (400 MHz, DMSO-d₆) δ 8.45 (m, 1H), 8.4-8.3 (m, 3H), 8.2 (d, 1H), 7.7 (d, 1H), 7.5 (d, 1H), 5.5 (m, 1H), 4.9 (m, 1H), 4.5 (m, 1H), 4.4 (m, 1H), 4.0 (m, 1H), 3.5 (m, 1H), 1.4 (m, 6H), 1.2 (m, 3H); MS (EI) for C₂₂H₂₂ClFN₄O₃S, found 477.25 (MH+).

Using the same or analogous synthetic techniques in Example 17 and substituting with appropriate reagents, the following compounds were prepared.

(2S,3R)-3-Amino-4-{[5-chloro-4-(5-{3-chloro-4-[(1-methylethyl)oxy]phenyl}-1,3,4-thiadiazol-2-yl)-2-fluorophenyl]oxy}butan-2-ol hydrochloride. ¹H NMR (400 MHz, DMSO-d₆) δ 8.30 (br s, 3H), 8.20 (m, 2H), 8.00 (d, 1H), 7.75 (d, 1H), 7.40 (d, 1H), 5.50 (br, 1H), 4.90 (m, 1H), 4.55 (m, 1H), 4.40 (m, 1H), 4.10 (m, 1H), 3.50 (m, 1H), 1.40 (d, 6H), 1.25 (d, 3H); MS (EI) for C₂₁H₂₂Cl₂FN₃O₃S, found 486.15 (MH+).

(2S,3S)-3-Amino-4-{[5-chloro-4-(5-{3-chloro-4-[(1-methylethyl)oxy]phenyl}-1,3,4-thiadiazol-2-yl)-2-fluorophenyl]oxy}butan-2-ol hydrochloride. ¹H NMR (400 MHz, DMSO-d₆) δ 8.35 (br s, 3H), 8.10 (m, 2H), 8.00 (d, 1H), 7.65 (d, 1H), 7.40 (d, 1H), 5.60 (br, 1H), 4.85 (m, 1H), 4.40 (m, 2H), 3.95 (m, 1H), 3.45 (m, 1H), 1.40 (d, 6H), 1.25 (d, 3H); MS (EI) for C₂₁H₂₂Cl₂FN₃O₃S, found 486.15 (MH+).

(2R,3S)-3-Amino-4-{[5-chloro-4-(5-{3-chloro-4-[(1-methylethyl)oxy]phenyl}-1,3,4-thiadiazol-2-yl)-2-fluorophenyl]oxy}butan-2-ol hydrochloride. ¹H NMR (400 MHz, DMSO-d₆) 8.30 (br s, 3H), 8.15 (m, 2H), 8.00 (d, 1H), 7.70 (d, 1H), 7.40 (d, 1H), 5.45 (br, 1H), 4.85 (m, 1H), 4.50 (m, 1H), 4.35 (m, 1H), 4.05 (m, 1H), 3.50 (m, 1H), 1.40 (d, 6H), 1.25 (d, 3H); MS (EI) for C₂₁H₂₂Cl₂FN₃O₃S, found 486.15 (MH+).

(2R,3R)-3-Amino-4-{[5-chloro-4-(5-{3-chloro-4-[(1-methylethyl)oxy]phenyl}-1,3,4-thiadiazol-2-yl)-2-fluorophenyl]oxy}butan-2-ol hydrochloride. ¹H NMR (400 MHz, DMSO-d₆) δ 8.25 (br s, 3H), 8.15 (m, 2H), 8.00 (d, 1H), 7.65 (d, 1H), 7.40 (d, 1H), 5.60 (br, 1H), 4.85 (m, 1H), 4.40 (m, 2H), 3.95 (m, 1H), 3.45 (m, 1H), 1.40 (d, 6H), 1.25 (d, 3H); MS (EI) for C₂₁H₂₂Cl₂FN₃O₃S, found 486.15 (MH+).

5-[5-(4-{[(2S,3R)-2-Amino-3-hydroxybutyl]oxy}-2-chloro-5-fluorophenyl)-1,3,4-thiadiazol-2-yl]-2-[(1-methylethyl)oxy]benzonitrile hydrochloride. ¹H NMR (400 MHz, DMSO-d₆) δ 8.4 (s, 1H), 8.3 (d, 1H), 8.2 (m, 3H), 7.7 (d, 1H), 7.5 (d, 1H), 5.5 (m, 1H), 4.9 (m, 1H), 4.5 (m, 1H), 4.3 (m, 1H), 4.0 (m, 1H), 3.5 (m, 1H), 1.4 (d, 6H), 1.2 (d, 3H); MS (EI) for C₂₂H₂₂ClFN₄O₃S, found 476.95 (MH+).

5-[5-(4-{[(2S,3S)-2-Amino-3-hydroxybutyl]oxy}-2-chloro-5-fluorophenyl)-1,3,4-thiadiazol-2-yl]-2-[(1-methylethyl)oxy]benzonitrile hydrochloride. ¹H NMR (400 MHz, DMSO-d₆) δ 8.4 (s, 1H), 8.3 (d, 1H), 8.1 (m, 3H), 7.7 (d, 1H), 7.5 (d, 1H), 5.6 (m, 1H), 4.9 (m, 1H), 4.4 (m, 2H), 4.0 (m, 1H), 1.4 (d, 6H), 1.2 (d, 3H); MS (EI) for C₂₂H₂₂ClFN₄O₃S, found 476.95 (MH+).

(2R,3S)-3-Amino-4-{[5-chloro-2-fluoro-4-(5-{3-methyl-4-[(1-methylethyl)oxy]phenyl}-1,3,4-thiadiazol-2-yl)phenyl]oxy}butan-2-ol hydrochloride. ¹H NMR (400 MHz, DMSO-d₆) δ 8.35 (br s, 3H), 8.15 (d, 1H), 7.85 (m, 2H), 7.70 (d, 1H), 7.15 (d, 1H), 4.75 (m, 1H), 4.50 (m, 1H), 4.35 (m, 1H), 4.05 (m, 2H), 3.5 (m, 1H), 2.20 (s, 3H), 1.35 (m, 6H), 1.25 (m, 3H); MS (EI) for C₂₂H₂₅ClFN₃O₃S, found 466.10 (MH+).

(2S,3S)-3-Amino-4-{[5-chloro-2-fluoro-4-(5-{3-methyl-4-[(1-methylethyl)oxy]phenyl}-1,3,4-thiadiazol-2-yl)phenyl]oxy}butan-2-ol hydrochloride. ¹H NMR (400 MHz, DMSO-d₆) δ 8.30 (br s, 3H), 8.15 (d, 1H), 7.85 (m, 2H), 7.65 (d, 1H), 7.15 (d, 1H), 4.75 (m, 1H), 4.45 (m, 1H), 4.35 (m, 2H), 3.95 (m, 1H), 3.40 (m, 1H), 2.20 (s, 3H), 1.35 (m, 6H), 1.25 (m, 3H); MS (EI) for C₂₂H₂₅ClFN₃O₃S, found 466.10 (MH+).

### Example 18

### (2R,3S)-3-amino-4-{[5-chloro-2-fluoro-4-(5-{2-[(1-methylethyl)amino]pyridin-4-yl}-1,3,4-thiadiazol-2-yl)phenyl]oxy}butan-2-ol hydrochloride

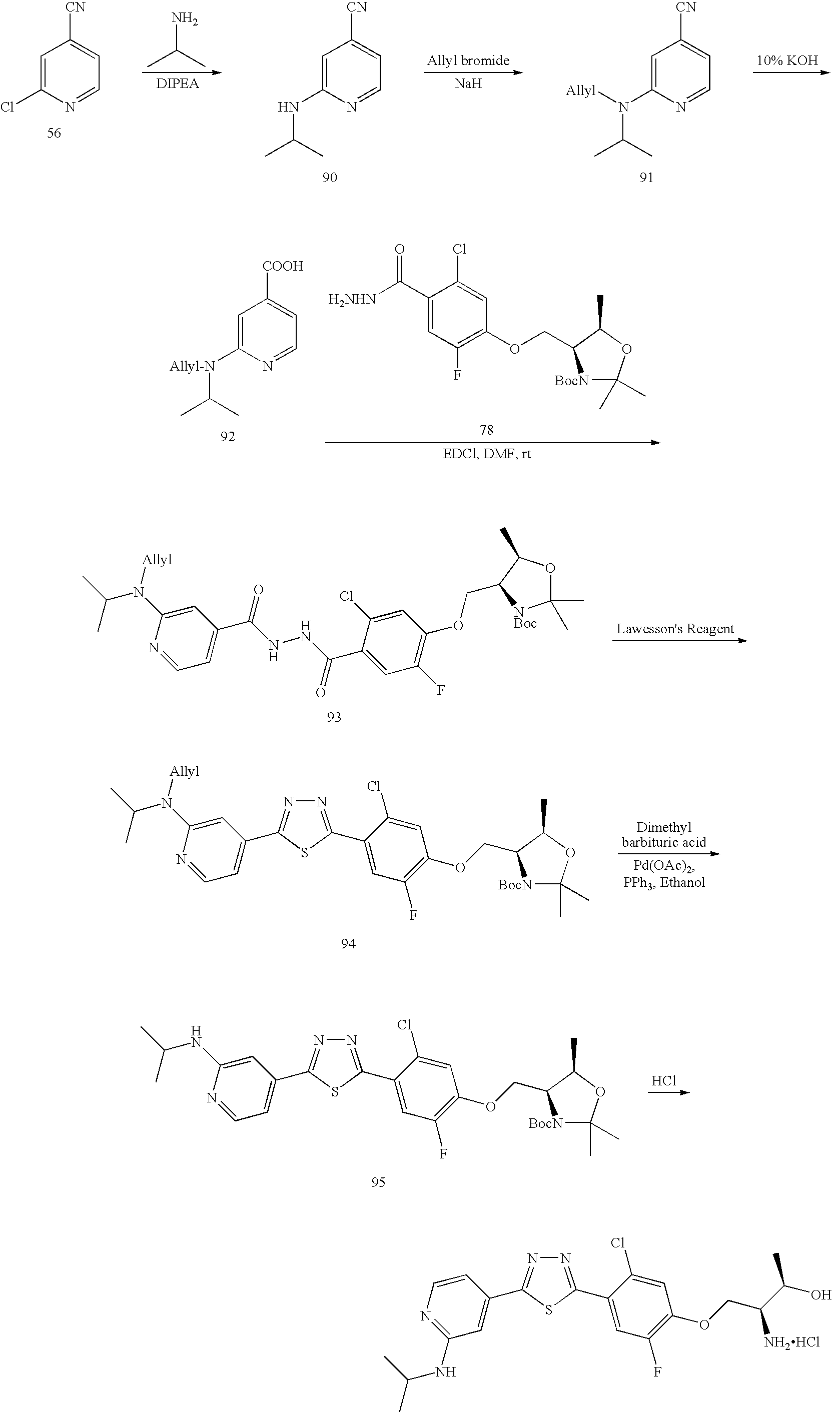

Step 1: 2-(Isopropylamino)isonicotinonitrile (90). A mixture of 56 (4.5 g, 32.4 mmol) and isopropylamine (20 mL, 327 mmol) was heated at 90° C. for 48 h in a sealed tube. The reaction mixture was concentrated in vacuo, water was added to the residue and resulting aqueous mixture was extracted with EtOAc, dried over Na₂SO₄, concentrated and purified by column chromatography to afford 90 (1.6 g, 30.8%).

Step 2: 2-(Allyl(isopropyl)amino)isonicotinonitrile (91). To an ice cooled solution of 90 (1.6 g, 9.93 mmol) in DMF (110 mL) was added NaH (0.262 g, 10.93 mmol) and the reaction mixture was stirred at the same temperature for 30 min. Allyl bromide (1.5 g, 12.4 mmol) was added and reaction was further stirred at the same temperature for 15 min and then quenched with ice with stirring for 15 min at 0° C. The resulting aqueous mixture was neutralized with citric acid solution and extracted with EtOAc, dried, concentrated and purified by column chromatography to obtain 91 as colorless oil (1.6 g, 80%).

Step 3: 2-(Allyl(isopropyl)amino)isonicotinic acid (92). To a stirred solution of 91 (1.6 g, 7.96 mmol) in EtOH (10 mL) was added aqueous 10% KOH (16 mL) and the reaction mixture was refluxed for 12 h. The reaction mixture was concentrated in vacuo and the resulting residue neutralized to pH 5 with citric acid solution. The aqueous layer was extracted with ethyl acetate, dried and concentrated to afford a semi solid compound which was washed with ether. The ether layer was concentrated to afford 92 (1.2 g, 68%) as a pale yellow oil, which was used as such for the next step.

Step 4: (4S,5R)-tert-Butyl (4-(2-(2-(allyl(isopropyl)amino)isonicotinoyl)hydrazinecarbonyl)-5-chloro-2-fluorophenoxy)methyl)-2,2,5-trimethyloxazolidine-3-carboxylate (93). Intermediate 93 was synthesized from intermediates 78 and 92 using the same or an analogous synthetic procedure to that of Example 2.

Step 4: (4S,5R)-tert-Butyl 4-((4-(5-(2-(allyl(isopropyl)amino)pyridin-4-yl)-1,3,4-thiadiazol-2-yl)-5-chloro-2-fluorophenoxy)methyl)-2,2,5-trimethyloxazolidine-3-carboxylate (94). Intermediate 94 was synthesized from intermediate 93 using the same or an analogous synthetic procedure to that of Example 3.

Step 5: (4S,5R)-tert-Butyl 4-((5-chloro-2-fluoro-4-(5-(2-(isopropylamino)pyridin-4-yl)-1,3,4-thiadiazol-2-yl)phenoxy)methyl)-2,2,5-trimethyloxazolidine-3-carboxylate (95). A stirred solution of 94 (0.6 g, 0.95 mmol), 1,3-dimethyl barbituric acid (0.591 g, 3.79 mmol), Pd(OAc)₂ (0.213 g, 0.95 mmol) and TPP (0.249 g, 0.95 mmol) in ethanol (5 mL) was purged with Argon for 20 min followed by heating at 85° C. for 12 h. The reaction mixture was then cooled, filtered through Celite and concentrated. Water was added to the resulting residue and the aqueous mixture extracted with ethyl acetate, dried, concentrated and purified by column chromatography to obtain 95 (0.5 g, 88.9%).

Step 6: (2R,3S)-3-Amino-4-{[5-chloro-2-fluoro-4-(5-{2-[(1-methylethyl)amino]pyridin-4-yl}-1,3,4-thiadiazol-2-yl)phenyl]oxy}butan-2-ol hydrochloride. The title compound was synthesized from intermediate 95 using the same or an analogous synthetic procedure to that of Example 4. ¹H NMR (400 MHz, DMSO-d₆) δ 8.3-8.1 (m, 4H), 7.7 (d, 1H), 7.3 (m, 1H), 7.2 (m, 1H), 4.5 (m, 1H), 4.3 (m, 2H), 4.1 (m, 2H), 3.5 (m, 1H), 1.2 (m, 9H); MS (EI) for C₂₀H₂₃ClFN₅O₂S, found 452.00 (MH+).

Using the same or analogous synthetic techniques in Example 18 and substituting with appropriate reagents, the following compound was prepared.

(2S,3S)-3-Amino-4-{[5-chloro-2-fluoro-4-(5-{2-[(1-methylethyl)amino]pyridin-4-yl}-1,3,4-thiadiazol-2-yl)phenyl]oxy}butan-2-ol hydrochloride. MS (EI) for C₂₀H₂₃ClFN₅O₂S, found 452.05 (MH+).

### Example 19

### (2R,3S)-3-Amino-4-{[5-chloro-2-fluoro-4-(5-{2-methyl-6-[(1-methylethyl)amino]pyridin-4-yl}-1,3,4-thiadiazol-2-yl)phenyl]oxy}butan-2-ol hydrochloride

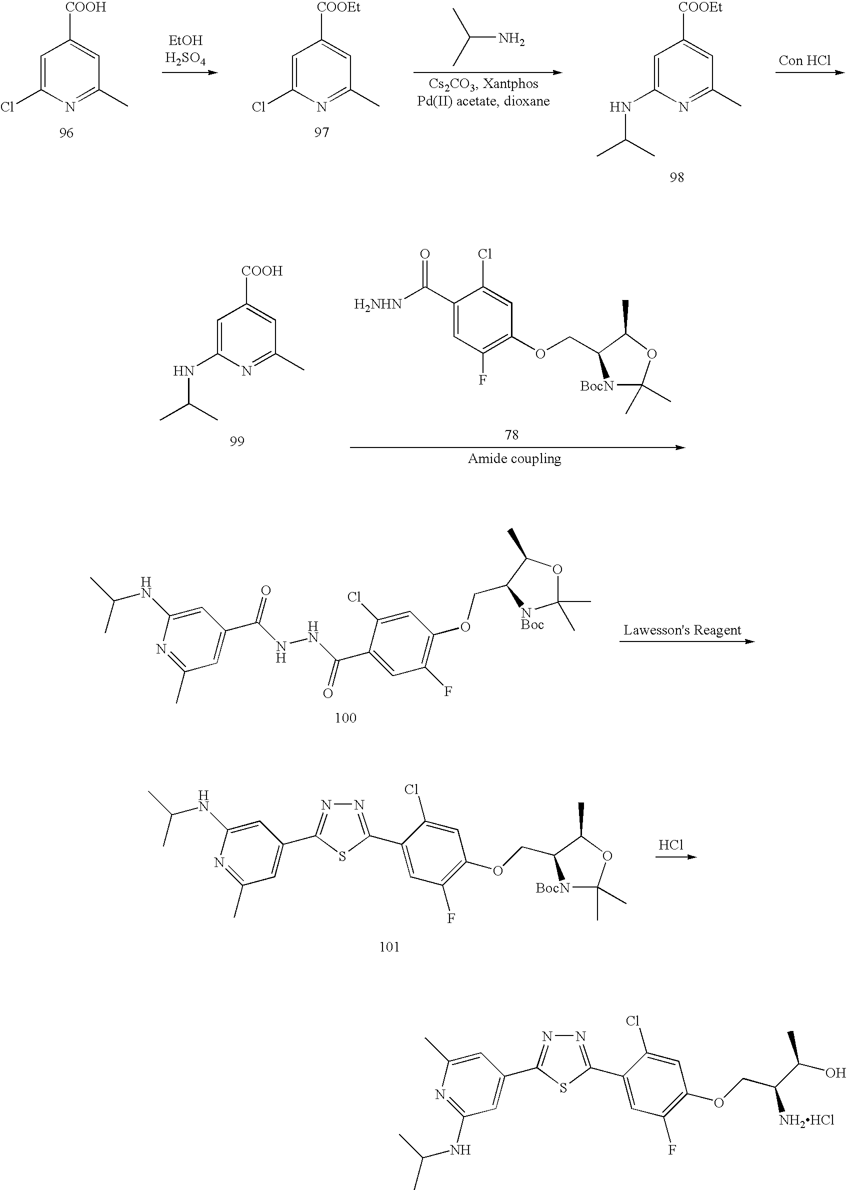

Step 1: Ethyl 2-chloro-6-methylisonicotinate (97). To a stirred solution of 96 (7.0 g, 40.79 mmol) in ethanol (70 mL) was added conc H₂SO₄ (2 mL) at 0° C. dropwise followed by heating at 80° C. for 12 h. The reaction mixture was cooled to room temperature and concentrated in vacuo. The resulting residue was diluted with ethyl acetate and washed with water, sodium bicarbonate solution and sat. NaCl. The organic layer was dried over anhydrous Na₂SO₄ and concentrated to afford 97 (7 g, 86%) as a white solid.

Step 2: Ethyl 2-(isopropylamino)-6-methylisonicotinate (98). To a stirred solution of ester 97 (5.0 g, 25.1 mmol) in dry dioxane (100 mL), Cs₂CO₃ (24.5 g, 75.3 mmol) and isopropylamine (12.6 mL, 8.8 g, 150.01 mmol) was added. The mixture was degassed and kept under nitrogen atmosphere and Xantphos (4.36 g, 7.53 mmol) and Pd(II) acetate (1.12 g, 5.02 mmol) were added. The reaction mixture was stirred in a sealed vessel at 85° C. for 14 h. The reaction mixture was cooled to room temperature, filtered and concentrated. The crude compound was purified by column chromatography to give ester 98 (1.2 g, 21%).

Step 3: 2-(Isopropylamino)-6-methylisonicotinic acid (99). A stirred solution of 98 (0.5 g, 2.2 mmol) in conc HCl (20 mL) was heated at 75° C. for 8 h before it was cooled to room temperature and concentrated. The residue was dried under high vacuum and washed with pentane and diethyl ether and then azeotroped with toluene to afford 99 (0.45 g, 77%) as a hydrochloride salt.

Step 4: (4S,5R)-tert-Butyl 4-((5-chloro-2-fluoro-4-(2-(2-(isopropylamino)-6-methylisonicotinoyl)hydrazinecarbonyl)phenoxy)methyl)-2,2,5-trimethyloxazolidine-3-carboxylate (100). Intermediate 100 was synthesized from intermediates 78 and 99 using the same or an analogous synthetic procedure to that of Example 2.

Step 5: (4S,5R)-tert-Butyl 4-((5-chloro-2-fluoro-4-(5-(2-(isopropylamino)-6-methylpyridin-4-yl)-1,3,4-thiadiazol-2-yl)phenoxy)methyl)-2,2,5-trimethyloxazolidine-3-carboxylate (101). Intermediate 101 was synthesized from intermediate 100 using the same or an analogous synthetic procedure to that of Example 3.

Step 6: (2R,3S)-3-Amino-4-{[5-chloro-2-fluoro-4-(5-{2-methyl-6-[(1-methylethyl)amino]pyridin-4-yl}-1,3,4-thiadiazol-2-yl)phenyl]oxy}butan-2-ol hydrochloride. The title compound was synthesized from intermediate 101 using the same or an analogous synthetic procedure to that of Example 4. ¹H NMR (400 MHz, DMSO-d₆) δ 8.3 (d, 1H), 7.6 (d, 1H), 7.5 (s, 1H), 7.3 (s, 1H), 4.6 (m, 1H), 4.4 (m, 1H), 4.2 (m, 2H), 3.7 (m, 1H), 2.6 (s, 3H), 1.4 (m, 9H); MS (EI) for C₂₁H₂₅ClFN₅O₂S, found 466.05 (MH+).

Using the same or analogous synthetic techniques in Example 19 and substituting with appropriate reagents, the following compound was prepared.

(2S,3S)-3-Amino-4-{[5-chloro-2-fluoro-4-(5-{2-methyl-6-[(1-methylethyl)amino]pyridin-4-yl}-1,3,4-thiadiazol-2-yl)phenyl]oxy}butan-2-ol hydrochloride. ¹H NMR (400 MHz, CD₃OD) δ 8.25 (d, 1H), 7.55 (d, 1H), 7.50 (s, 1H), 7.30 (s, 1H), 4.40 (m, 2H), 4.10 (m, 2H), 3.50 (m, 1H), 2.60 (s, 3H), 1.35 (m, 9H); MS (EI) for C₂₁H₂₅ClFN₅O₂S, found 466.10 (MH+).

### Example 20

### (2R,3S)-3-Amino-4-{[5-chloro-2-fluoro-4-(5-{5-methyl-6-[(1-methylethyl)amino]pyridin-3-yl}-1,3,4-thiadiazol-2-yl)phenyl]oxy}butan-2-ol hydrochloride

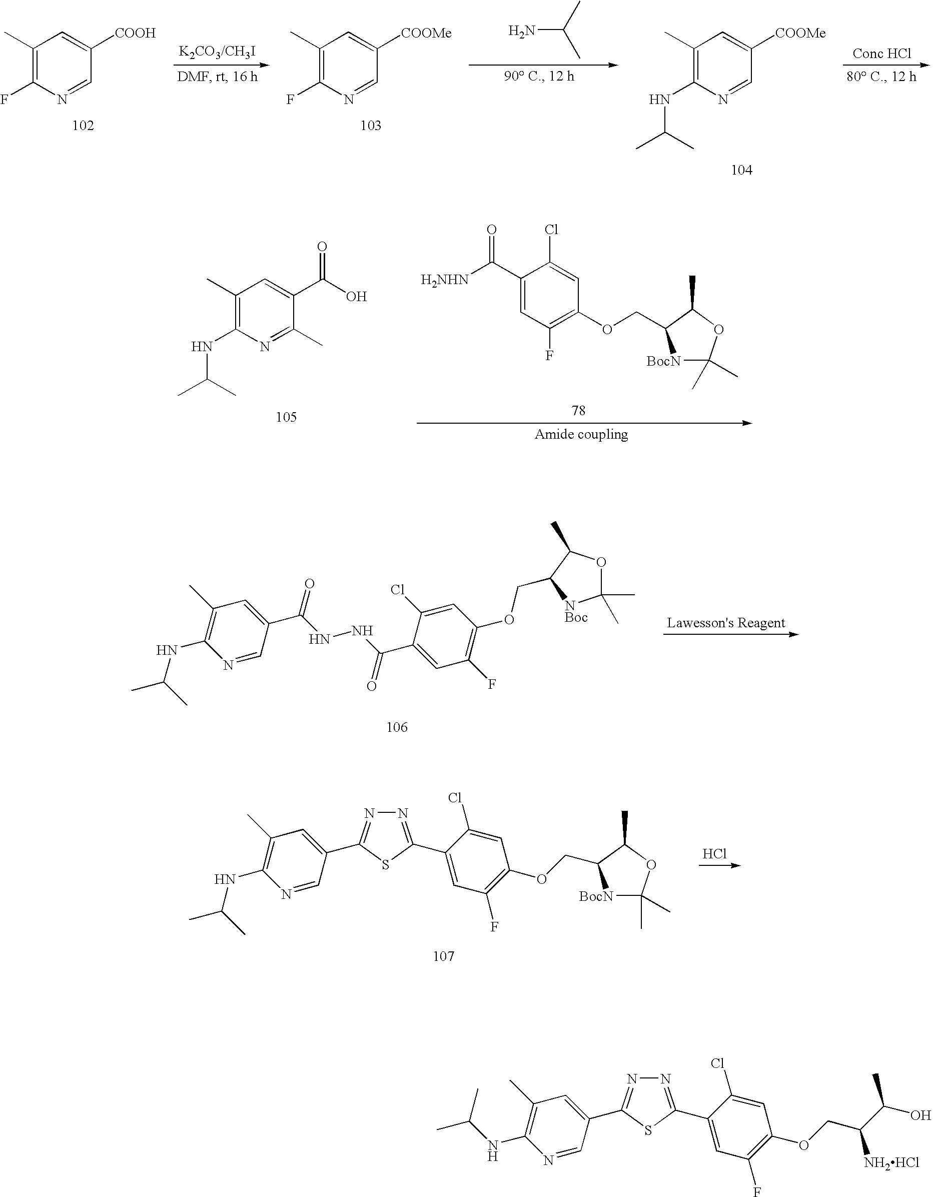

Step 1: Methyl 6-fluoro-5-methylnicotinate (103). A mixture of 5-methyl-6-fluoro-nicotinic acid 102 (7.0 g, 45 mmol), K₂CO₃ (13.7 g, 99 mmol) and methyl iodide (9.58 g, 67 mmol) in DMF (200 mL) was stirred for 16 h at room temperature. After dilution with water (50 mL), the reaction mixture was extracted with EtOAc (50 mL). The combined extracts were washed successively with sat. aqueous NaHCO₃ solution (20 mL), sat. NaCl (2×20 mL) and dried (Na₂SO₄). Filtration and evaporation of the solvent gave the product 103 (6.5 g, 85%).

Step 2: Methyl 6-(isopropylamino)-5-methylnicotinate (104). In a sealed tube, a reaction mixture of isopropylamine (50 mL) and 103 (6.5 g, 38 mmol) was heated at 90° C. for 12 h. After it was cooled to room temperature, volatiles were removed in vacuo. The obtained residue was diluted with water and extracted with EtOAc. The combined organic layers were dried over Na₂SO₄ and concentrated to afford 104 (7.9 g, 98.4%).

Step 3: 6-(Isopropylamino)-5-methylnicotinic acid (105). A stirred mixture of 104 (4.5 g, 21 mmol) in conc HCl (20 mL) was heated at 80° C. for 12 h. After being cooled to room temperature, it was concentrated. The resulting residue was dried under high vacuum and washed with pentane and diethyl ether and then azeotroped with toluene to afford 105 (4.0 g, 95%) as the hydrochloride salt.

Step 4: (4S,5R)-tert-Butyl 4-((5-chloro-2-fluoro-4-(2-(6-(isopropylamino)-5-methylnicotinoyl)-hydrazinecarbonyl)phenoxy)methyl)-2,2,5-trimethyloxazolidine-3-carboxylate (106). Intermediate 106 was synthesized from intermediates 78 and 105 using the same or an analogous synthetic procedure to that of Example 2.

Step 5: (4S,5R)-tert-Butyl 4-((5-chloro-2-fluoro-4-(5-(6-(isopropylamino)-5-methylpyridin-3-yl)-1,3,4-thiadiazol-2-yl)phenoxy)methyl)-2,2,5-trimethyloxazolidine-3-carboxylate (107). Intermediate 107 was synthesized from intermediate 106 using the same or an analogous synthetic procedure to that of Example 3.

Step 6: (2R,3S)-3-Amino-4-{[5-chloro-2-fluoro-4-(5-{5-methyl-6-[(1-methylethyl)amino]pyridin-3-yl}-1,3,4-thiadiazol-2-yl)phenyl]oxy}butan-2-ol hydrochloride. The title compound was synthesized from intermediate 107 using the same or an analogous synthetic procedure to that of Example 4. ¹H NMR (400 MHz, DMSO-d₆) δ 8.5 (s, 1H), 8.3 (bs, 2H), 8.2-8.1 (m, 2H), 7.7 (d, 1H), 4.5 (m, 1H), 4.4 (m, 2H), 4.0 (m, 2H), 3.5 (m, 1H), 2.2 (s, 3H), 1.3-1.2 (m, 9H); MS (EI) for C₂₁H₂₅ClFN₅O₂S, found 466.10 (MH+).

Using the same or analogous synthetic techniques in Example 20 and substituting with appropriate reagents, the following compounds were prepared.

(2S,3S)-3-Amino-4-{[5-chloro-2-fluoro-4-(5-{5-methyl-6-[(1-methylethyl)amino]pyridin-3-yl}-1,3,4-thiadiazol-2-yl)phenyl]oxy}butan-2-ol hydrochloride. ¹H NMR (400 MHz, CD₃OD) δ 8.40 (d, 2H), 8.20 (d, 1H), 7.55 (d, 1H), 4.40 (m, 2H), 4.10 (m, 2H), 3.45 (m, 1H), 2.40 (s, 3H), 1.45 (d, 6H), 1.35 (d, 3H); MS (EI) for C₂₁H₂₅ClFN₅O₂S, found 466.10 (MH+).

(2R,3R)-3-Amino-4-{[5-chloro-2-fluoro-4-(5-{5-methyl-6-[(1-methylethyl)amino]pyridin-3-yl}-1,3,4-thiadiazol-2-yl)phenyl]oxy}butan-2-ol hydrochloride. ¹H NMR (400 MHz, DMSO-d₆) δ 8.45 (s, 1H), 8.30 (bs, 3H), 8.10 (m, 2H), 7.65 (d, 1H), 4.40 (m, 2H), 4.00 (m, 2H), 3.70 (m, 1H), 3.35 (m, 1H), 2.25 (s, 3H), 1.25 (m, 9H); MS (EI) for C₂₁H₂₅ClFN₅O₂S, found 466.10 (MH+).

### Example 21

### (R)-2-Amino-3-(5-chloro-4-(5-(2-chloro-6-(oxetan-3-yloxy)pyridin-4-yl)-1,3,4-thiadiazol-2-yl)-2-fluorophenoxy)propan-1-ol trifluoroacetic acid salt

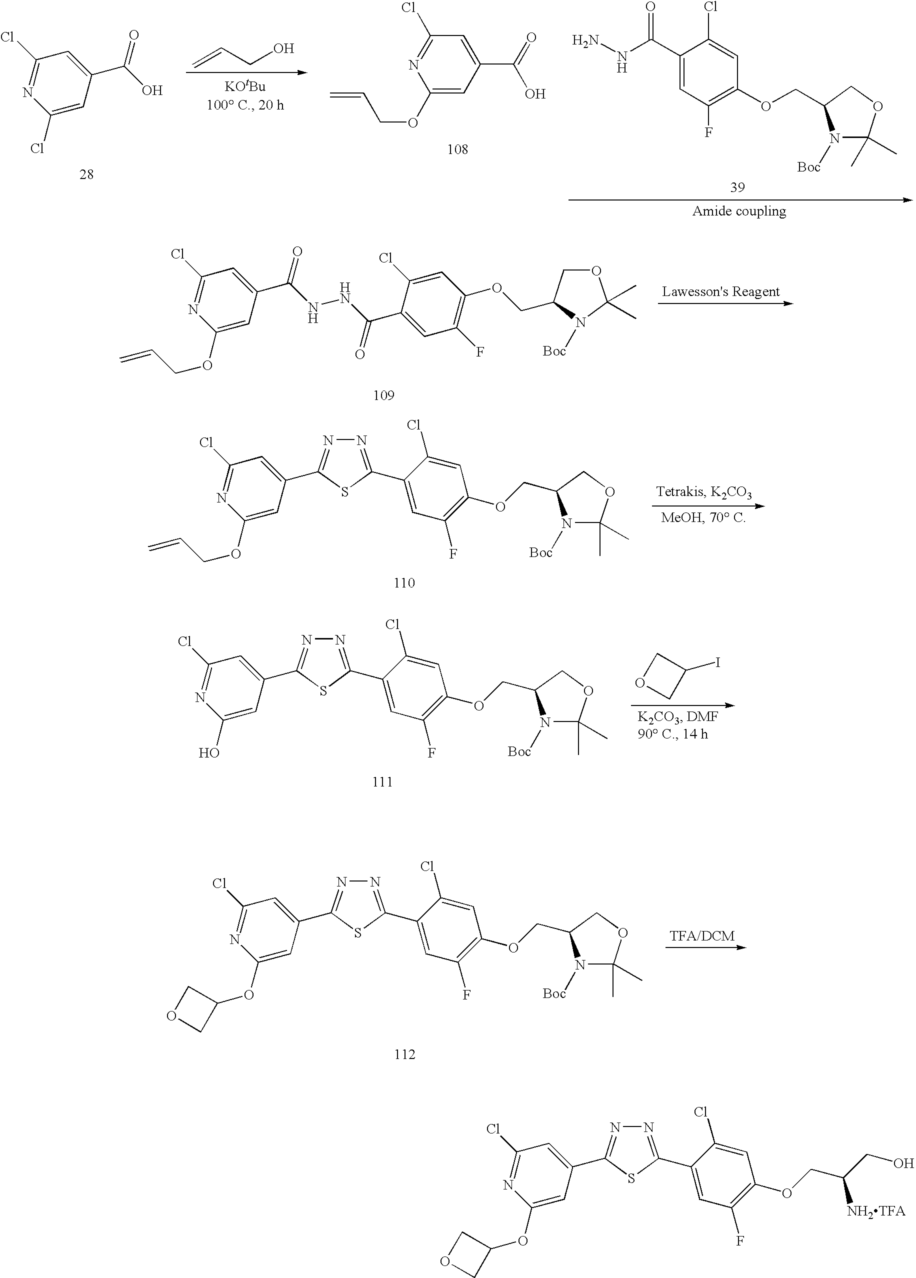

Step 1: 2-(Allyloxy)-6-chloroisonicotinic acid (108). Potassium tert-butoxide (5.85 g, 52 mmol) was added to allyl alcohol (25 mL) and the resulting mixture was stirred for 5 min at room temperature. Compound 28 (5.0 g, 26 mmol) was added to the reaction mixture and it was heated for 20 h at 100° C. After cooling to room temperature, the reaction mixture was concentrated in vacuo. Water was added to the resulting residue and the mixture acidified with citric acid and extracted with ether. The organic layer was dried and concentrated to afford 108 (3.0 g, 54.5%).

Step 2: (S)-tert-Butyl 4-((4-(2-(2-(allyloxy)-6-chloroisonicotinoyl)hydrazinecarbonyl)-5-chloro-2-fluorophenoxy)methyl)-2,2-dimethyloxazolidine-3-carboxylate (109). Intermediate 109 was synthesized from intermediates 39 and 108 using the same or an analogous synthetic procedure to that of Example 2.

Step 3: (S)-tert-Butyl 4-((4-(5-(2-(allyloxy)-6-chloropyridin-4-yl)-1,3,4-thiadiazol-2-yl)-5-chloro-2-fluorophenoxy)methyl)-2,2-dimethyloxazolidine-3-carboxylate (110). Intermediate 110 was synthesized from intermediate 109 using the same or an analogous synthetic procedure to that of Example 3.

Step 4: (S)-tert-Butyl 4-((5-chloro-4-(5-(2-chloro-6-hydroxypyridin-4-yl)-1,3,4-thiadiazol-2-yl)-2-fluorophenoxy)methyl)-2,2-dimethyloxazolidine-3-carboxylate (111). To a solution of 110 (3.5 g, 5.7 mmol) in MeOH (35 mL) at room temperature was added tetrakis (0.099 g, 0.08 mmol) and the reaction mixture purged with nitrogen. Potassium carbonate (2.35 g, 17.1 mmol) was added and again the reaction mixture was purged with nitrogen followed by heating at 70° C. for 12 h. After cooling to room temperature, the reaction mixture was concentrated in vacuo and neutralized with citric acid and diluted with water. The aqueous phase was extracted with ethyl acetate. The organic layer was dried, concentrated and purified by column chromatography to obtain 111 (2.0 g, 61%).

Step 5: (S)-tert-Butyl 4-((5-chloro-4-(5-(2-chloro-6-(oxetan-3-yloxy)pyridin-4-yl)-1,3,4-thiadiazol-2-yl)-2-fluorophenoxy)methyl)-2,2-dimethyloxazolidine-3-carboxylate (112). To as stirred solution of 111 (0.6 g, 1.05 mmol) in DMF (10 mL) was added 3-iodooxetane (0.289 g, 1.57 mmol) and K₂CO₃ (0.29 g, 2.1 mmol) and it was heated at 90° C. for 14 h. After cooling to room temperature, the reaction mixture was diluted with water (200 mL) and extracted with ethyl acetate. The combined organic layers were dried over Na₂SO₄ and concentrated to give 112 (0.6 g, 92%).

Step 6: (R)-2-Amino-3-(5-chloro-4-(5-(2-chloro-6-(oxetan-3-yloxy)pyridin-4-yl)-1,3,4-thiadiazol-2-yl)-2-fluorophenoxy)propan-1-ol trifluoroacetic acid salt. Compound 112 (0.7 g, 1.11 mmol) was dissolved in DCM (4 mL) and cooled to 0° C. TFA in DCM (1:1, 5 mL) was added and the resulting reaction mixture was stirred at 0° C. for 10 min followed by stirring at room temperature for 1 h. The reaction mixture was concentrated in vacuo and purified by preperative HPLC to give the title compound (0.15 g, 22.3%). ¹H NMR (400 MHz, DMSO-d₆) δ 8.5 (s, 1H), 8.4 (s, 1H), 8.2 (m, 3H), 7.7 (d, 1H), 5.7 (m, 1H), 5.0 (m, 2H), 4.8 (m, 2H), 4.4 (m, 2H), 4.0-3.6 (m, 4H); MS (EI) for C₁₉H₁₇Cl₂FN₄O₄S, found 487.05 (MH+).

### Example 22

### (2R)-2-Amino-3-{[5-chloro-2-fluoro-4-(5-{2-[(1-methylethyl)amino]pyrimidin-5-yl}-1,3,4-thiadiazol-2-yl)phenyl]oxy}propan-1-ol hydrochloride

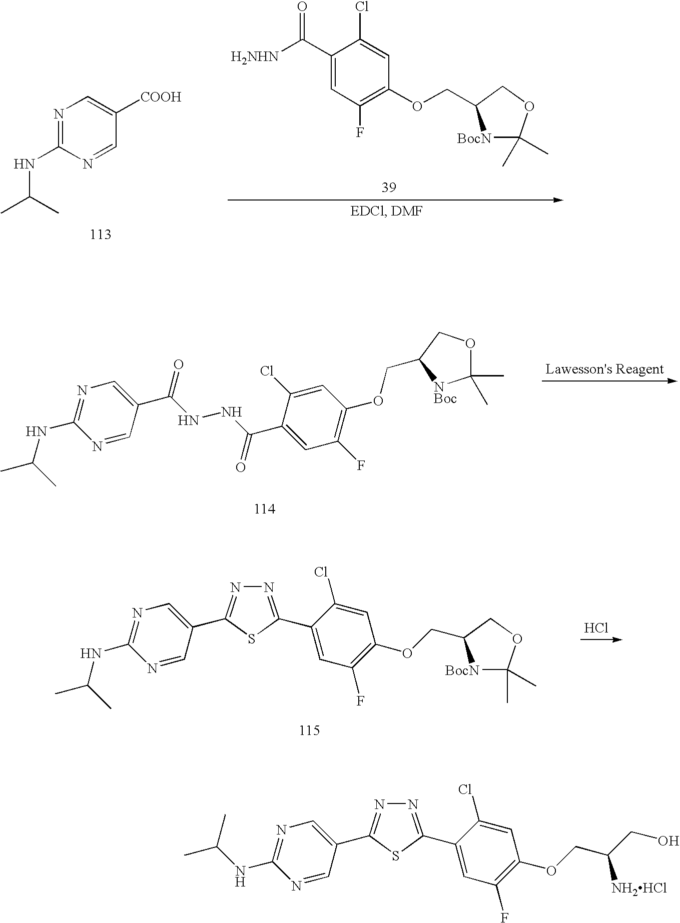

Step 1: (S)-tert-Butyl 4-((5-chloro-2-fluoro-4-(2-(2-(isopropylamino)pyrimidine-5-carbonyl)hydrazinecarbonyl)phenoxy)methyl)-2,2-dimethyloxazolidine-3-carboxylate (114). Intermediate 114 was synthesized from intermediates 39 and 113 using the same or an analogous synthetic procedure to that of Example 2.

Step 2: (S)-tert-Butyl 4-((5-chloro-2-fluoro-4-(5-(2-(isopropylamino)pyrimidin-5-yl)-1,3,4-thiadiazol-2-yl)phenoxy)methyl)-2,2-dimethyloxazolidine-3-carboxylate (115). Intermediate 115 was synthesized from intermediate 114 using the same or an analogous synthetic procedure to that of Example 3.

Step 3: (2R)-2-Amino-3-{[5-chloro-2-fluoro-4-(5-{2-[(1-methylethyl)amino]pyrimidin-5-yl}-1,3,4-thiadiazol-2-yl)phenyl]oxy}propan-1-ol hydrochloride. The title compound was synthesized from intermediate 115 using the same or an analogous synthetic procedure to that of Example 4. ¹H NMR (400 MHz, DMSO-d₆) δ 8.9 (bs, 1H), 8.3 (bs, 2H), 8.1 (d, 1H), 8.0 (m, 1H), 7.6 (d, 1H), 4.4 (m, 2H), 4.2 (m, 1H), 3.7 (m, 3H), 1.2 (m, 6H); MS (EI) for C₁₈H₂₀ClFN₆O₂S, found 439.00 (MH+).

Using the same or analogous synthetic techniques in Example 22 and substituting with appropriate reagents, the following compound was prepared.

(2S)-2-Amino-3-{[5-chloro-2-fluoro-4-(5-{2-[(1-methylethyl)amino]pyrimidin-5-yl}-1,3,4-thiadiazol-2-yl)phenyl]oxy}propan-1-ol hydrochloride. ¹H NMR (400 MHz, DMSO-d₆) δ 8.9 (bs, 1H), 8.3 (bs, 2H), 8.1 (d, 1H), 8.0 (m, 1H), 7.6 (d, 1H), 4.4 (m, 2H), 4.2 (m, 1H), 3.7 (m, 2H), 3.6 (m, 1H), 1.2 (m, 6H); MS (EI) for C₁₈H₂₀ClFN₆O₂S, found 439.10 (MH+).

### Biological Examples

Suitable in vitro assays for measuring S1P1 and S1P5 agonist activity are known in the art. All Compounds in Table 1 were tested in one or more of the following biological assays and were found to be agonists of S1P1 and/or S1P5. As such compounds of Formula I are useful for treating diseases, particularly autoimmune disease in which S1P1 and/or S1P5 activity contributes to the pathology and/or symptomatology of the disease, for example, multiple sclerosis and graft-versus host disease. Suitable in vivo models for autoimmune diseases are known to those of ordinary skill in the art and are also described below, e.g. models for autoimmune-mediated inflammation, multiple sclerosis, graft-versus host disease, and osteoporosis. Following the examples disclosed herein, as well as that disclosed in the art, a person of ordinary skill in the art can determine the S1P1 and S1P5 agonist activity of a compound of this invention and its usefulness for treating a disease.

### Biological Example 1

### CNG cAMP Assay

Frozen HEK293 cells expressing the CNG channel and S1P₁ (BD Biosciences, San Jose, Calif.) are thawed and plated into the wells of a black, clear bottom, 384-well CellBind plate (Corning, Corning, N.Y.) at 14,000 cells per well. HEK293 cells expressing the CNG channel and CB1 (BD Biosciences) are cultured and plated under the same conditions. The cells are incubated for 16 h at 37° C. in complete DMEM medium (Invitrogen Carlsbad, Calif.) containing 10% FBS (HyClone Logan, Utah), 250 μg/mL geneticin (Invitrogen), and 1 μg/mL puromycin (Sigma-Aldrich, St. Louis, Mo.). A membrane potential dye (BD Biosciences) is added and the plates are incubated for 2-2.5 h at room temperature.

Test compounds are tested at maximum concentrations of 10 μM. Compounds are diluted in DMSO (10 concentration points, 3-fold each) and added to the assay plate at final DMSO concentrations of 1.8%. For each compound, there are duplicate assay plates and each assay plate have duplicate wells per concentration point. Test compounds are added to the cells in a DPBS solution containing 25 μM Ro 20-1724 (Sigma-Aldrich), 500 nM of the A2b receptor agonist NECA (Sigma-Aldrich) and 10 nM (EC₉₅) of S1P (Avanti Alabaster, Ala.) and incubated for 90 min. The assay plate is read before compound addition (T₀) and after the 90 min incubation (T₉₀) using an EnVision plate reader (PerkinElmer, Waltham, Mass.) at an excitation wavelength of 350 nm and an emission wavelength of 590 nm. The T₉₀/T₀ ratio is determined for each concentration of the test compounds. The percent agonist activity is determined as [(test compound−DMSO alone control)/(NECA alone control−DMSO alone control)*100]. The percent activities are plotted against compound concentration to determine EC₅₀ using XLFit (IDBS, Alameda, Calif.). The control used for calculating rEC50 in the S1P₁ CNG agonist assay is DMSO.

### Biological Example 2

### S1P1 β-Arrestin Recruitment Assay

For the Tango™ β-arrestin recruitment assay, the cytoplasmic C-terminus of S1P₁ was tethered to the tTA transcriptional activator with a linker that contains a cleavage site for the N1a protease from tobacco etch virus (TEV protease). The C-terminus of the human β-arrestin2 protein was fused to TEV protease. Binding of an agonist recruits the βl -arrestin-TEV fusion protein to the receptor resulting in cleavage of the linker and released of tTA to enter the nucleus and subsequently activated a tTA-dependent luciferase reporter gene.

Assay 2a: Frozen HEK293 cells transiently transfected with receptor cDNAs for S1P₁ (Invitrogen) are thawed and suspended in 10 mL of Pro293a-CDM culture medium (Invitrogen) supplemented with 4 mM L-Glutamine (Invitrogen), 1× Pen/Strep (100 units/mL penicillin and 100 μg/mL streptomycin, Invitrogen) and 0.1% fatty acid free BSA (Sigma-Aldrich). Cells are added to the wells of a 384-well white opaque bottom assay plate (PerkinElmer) at 3,000-6,000 cells per well and the plate is incubated for approximately 4 h in a 37° C. incubator. Test compounds are tested at maximum concentrations of 10 μM for the agonist assays. Compounds are diluted in DMSO (10 concentration points, 3-fold each) and added to the assay plate at a 1% final DMSO concentration. For each compound, there are duplicate assay plates and each assay plate has duplicate wells per concentration point. The plate is incubated at 37° C. for 30 min. The efficacy control is 5 μM S1P (Avanti). Following agonist addition, the assay plates are incubated in a 37° C. incubator for 16-18 h. Luciferase assay reagent is added and luminescence measured in an EnVision plate reader (PerkinElmer). To determine agonist activity, percent activity is calculated as [(test compound−background)/(positive control−background)*100], where background is the luminescence of the DMSO alone control and the positive control is the luminescence from cells incubated with the efficacy control 5 μM S1P. The percent activities are plotted against compound concentration to determine EC₅₀ using XLFit (IDBS).

Assay 2b: Alternatively, U2OS cells expressing the reporter gene and S1P₁ (Invitrogen) were added to the wells of a 384-well white opaque bottom assay plate (PerkinElmer) at 0.3125×10⁶ cells per well. The cells were serum starved for 48 h in Freestyle medium (Invitrogen). Test compounds were tested at maximum concentrations of 1 μM for the agonist assay. Compounds were diluted in DMSO (10 concentration points, 3-fold each) and added to the assay plate at a 1% final DMSO concentration. The efficacy control was 1 μM S1P (Avanti). For each compound, there were duplicate assay plates and each assay plate had duplicate wells per concentration point. The plate was incubated overnight at 37° C. The GeneBLAzer β-lactamase assay reagent (Invitrogen) was added and the plates were incubated for an additional 2 h at room temperature. Fluorescence was measured using an EnVision plate reader (PerkinElmer, Waltham, Mass.) at an excitation wavelength of 409 nm and emission wavelengths of 460 nm and 530 nm. The emission intensity at each wavelength was background subtracted against wells containing medium only and the F_{460 nm}/F_{530 nm} ratio determined for each concentration of the test compounds. Percent activity was calculated as [(test compound ratio−DMSO ratio)/(positive control ratio−DMSO ratio)*100], where the positive control and DMSO ratios are from cells incubated with the efficacy control 1 μM S1P and 1% DMSO, respectively. The percent activities were plotted against compound concentration to determine EC₅₀ using XLFit (IDBS).

### Biological Example 3

### hS1P1R GTPγS and GTP-Eu Binding Assays

Assay 3a: The hS1P1R GTPγS binding assay was carried out at room temperature in 96 well non-binding surface assay plates. The reaction in each well contained 4 μg hS1P1R (hEdg1) membrane protein (Lonza), 30 μM GDP, 0.1 nM [³⁵S]GTPγS, 0.25% fatty acid free BSA, and serially diluted hS1P1R agonist compound in 200 μL assay buffer (25 mM Tris-HCl PH 7.9, 100 mM NaCl, 3 mM MgCl2, and 0.2 mM EGTA). After one hour of incubation, 0.9 mg of WGA (Wheat Germ Agglutinin) SPA beads in 50 μL of assay buffer was added to each well. The SPA beads were spun down after an additional one hour incubation. The radioactivity of the bound GTPγS was counted by reading the assay plate using a MicroBeta.

Assay 3b: The DELFIA GTP-Eu binding assay (PerkinElmer) is a time-resolved fluorometric assay based on GDP-GTP exchange. CHO cell membranes (Lonza) expressing human S1P₁ are incubated in 96-well filter plates (Pall, East Hills, N.Y.) in a final volume of 100 μL/well buffer containing 40 μg/mL membrane, 50 mM HEPES, 2 μM GDP, 10 mM MgCl₂, 100 mM NaCl, 500 μg/mL Saponin and test compound. Test compounds are tested at maximum concentrations of 10 μM. Compounds are diluted (10 concentration points, 3-fold each) and added to the assay plate at a 1% final DMSO concentration. For each compound, there are duplicate assay plates and each assay plate has duplicate wells per concentration point. The plates are incubated for 30 min at room temperature on a plate shaker at low speed. GTP-Eu is added to each well (10 μL, 10 nM final concentration) and the plate is incubated for an additional 30 min with slow shaking The wells are washed with ice cold GTP washing buffer (3×150 μL) using a vacuum manifold and the assay plates read in an EnVision plate reader (PerkinElmer) at an excitation wavelength of 340 nm and an emission wavelength of 615 nm. To determine agonist activity, percent activity is calculated as [(test compound−background)/(positive control−background)*100], where background is the fluorescence in absence of compound and the positive control is the fluorescence from membranes incubated with 1 μM S1P (Avanti). The percent activities are plotted against compound concentration to determine IC₅₀ or EC₅₀ using XLFit (IDBS).

### Biological Example 4

### hS1P5R GTPγS Binding Assay

The hS1PR5 GTPγS binding assay was carried out at room temperature in 96-well non-binding surface assay plates. The reaction in each well contained 5 μg hS1PR5 (hEdg8) membrane protein from CHO cells expressing hS1PR5, 30 μM GDP, 0.1 nM [³⁵S]GTPγS, 0.25% fatty acid free BSA, and serially-diluted Compound of the Invention in 200 μL assay buffer (25 mM Tris-HCl PH 7.9, 100 mM NaCl, 3 mM MgCl2, and 0.2 mM EGTA). After one hour of incubation, 0.9 mg of WGA (Wheat Germ Agglutinin) SPA beads in 50 μL of assay buffer was added to each well. The SPA beads were spun down after an additional one hour incubation. The radioactivity of the bound GTPγS was counted by reading the assay plate using a MicroBeta.

### Example 5

### S1P3 β-Arrestin Recruitment Assay

This assay was conducted using the procedures described in Assay 2a, replacing S1P1 with S1P3.

### Example 6

### S1P2 β-Arrestin Recruitment Assay

This assay was conducted using the procedures described in Assay 2a, replacing S1P1 with S1P2.

### Table 2: Results

Table 2 gives EC₅₀ data (unless otherwise indicated) for the compounds in Table 1 and are in nM units. Assay 2b is the Tango™ β-arrestin Recruitment Assay in U2OS cells, as described in Biological Example 2. Assay 3a is the hS1P1R GTPγS Binding Assay as described in Biological Example 3. Assay 4 is the hS1P5R GTPγS Binding Assay as described in Biological Example 4. Assay 5 is the Tango™ β-arrestin Recruitment Assay in HEK293 cells, as described in Biological Example 5.

EC₅₀'s were measured unless otherwise noted. An “*” indicates that rEC₅₀ (relative EC₅₀) was measured rather than EC₅₀.

For assay 5, F means the compound has an EC₅₀ or relative EC₅₀ of less than or equal to 250 nM and G means the compound has an EC₅₀ or relative EC₅₀ of greater than 250 nM.

For assays 3a, 2b and 4, “A” means the compound has an EC₅₀ or relative EC₅₀ of less than or equal to 10 nM. “B” means the compound has an EC₅₀ or relative EC₅₀ greater than 10 nM but less than or equal to 50 nM. “C” means the compound has an EC₅₀ or relative EC₅₀ greater than 50 nM but less than or equal to 250 nM. “D” means the compound has an EC₅₀ or relative EC₅₀ greater than 250 nM but less than or equal to 2800 nM. “E” means the compound has an EC₅₀ or relative EC₅₀ greater than 2800 nM but less than 10000 nM. In the table, “nt” means the Compound was not tested and “na” means the compound was tested but had no measurable activity under the assay conditions employed.

TABLE 2

| Entry | | Assay | Assay | | |
|---|---|---|---|---|---|
| No. | ACD-generated Name | 3a | 2b | Assay 4 | Assay 5 |
| 1 | (2R)-2-amino-3-{[5-chloro-4-(5-{3-chloro-4-[(1- | B | B | B | na |
| | methylethyl)oxy]phenyl}-1,3,4-thiadiazol-2-yl)-2- | | | | |
| | fluorophenyl]oxy}propan-1-ol | | | | |
| 2 | (2R)-2-amino-3-{[4-(5-{3-bromo-4-[(1- | A | B | B | na |
| | methylethyl)oxy]phenyl}-1,3,4-thiadiazol-2-yl)-5-chloro-2- | | | | |
| | fluorophenyl]oxy}propan-1-ol | | | | |
| 3 | (2R)-2-amino-3-[(5-chloro-2-fluoro-4-{5-[4-(2- | D | C | D | na |
| | methylpropyl)phenyl]-1,3,4-thiadiazol-2-yl}phenyl)oxy]propan- | | | | |
| | 1-ol | | | | |
| 4 | 2-amino-3-{[5-chloro-2-fluoro-4-(5-imidazo[2,1-b][1,3]thiazol- | na | C | nt | nt |
| | 6-yl-1,3,4-thiadiazol-2-yl)phenyl]oxy}propan-1-ol | | | | |
| 5 | 2-amino-3-{[5-chloro-4-(5-{2-chloro-6-[methyl(1- | B | A | D | F |
| | methylethyl)amino]pyridin-4-yl}-1,3,4-thiadiazol-2-yl)-2- | | | | |
| | fluorophenyl]oxy}propan-1-ol | | | | |
| 6 | 2-amino-3-{[5-chloro-4-(5-{5-chloro-6-[(1- | B | A | D | F |
| | methylethyl)oxy]pyridin-3-yl}-1,3,4-thiadiazol-2-yl)-2- | | | | |
| | fluorophenyl]oxy}propan-1-ol | | | | |
| 7 | (2R)-2-amino-3-{[5-chloro-2-fluoro-4-(5-{4- | C | D | nt | nt |
| | [(trifluoromethyl)oxy]phenyl}-1,3,4-thiadiazol-2- | | | | |
| | yl)phenyl]oxy}propan-1-ol | | | | |
| 8 | (2R)-2-amino-3-{[5-chloro-4-(5-{3-chloro-4- | C | D | nt | nt |
| | [(trifluoromethyl)oxy]phenyl}-1,3,4-thiadiazol-2-yl)-2- | | | | |
| | fluorophenyl]oxy}propan-1-ol | | | | |
| 9 | (2R)-2-amino-3-{[5-chloro-2-fluoro-4-(5-{4-[(1- | C | C | D | nt |
| | methylethyl)oxy]phenyl}-1,3,4-thiadiazol-2- | | | | |
| | yl)phenyl]oxy}propan-1-ol | | | | |
| 10 | (2S)-2-amino-3-{[5-chloro-4-(5-{3-chloro-4-[(1- | A | A | B | F |
| | methylethyl)oxy]phenyl}-1,3,4-thiadiazol-2-yl)-2- | | | | |
| | fluorophenyl]oxy}propan-1-ol | | | | |
| 11 | (2S)-2-amino-3-{[4-(5-{3-bromo-4-[(1- | A | A | B | F |
| | methylethyl)oxy]phenyl}-1,3,4-thiadiazol-2-yl)-5-chloro-2- | | | | |
| | fluorophenyl]oxy}propan-1-ol | | | | |
| 12 | (2R)-2-amino-3-{[4-(5-{3-bromo-4-[(1- | A | A | A | F* |
| | methylethyl)oxy]phenyl}-1,3,4-thiadiazol-2-yl)-5-chloro-2- | | | | |
| | fluorophenyl]oxy}propyl dihydrogen phosphate | | | | |
| 13 | (2S)-2-amino-3-{[5-chloro-4-(5-{3-chloro-4-[(1- | A | A | A | F |
| | methylethyl)oxy]phenyl}-1,3,4-thiadiazol-2-yl)-2- | | | | |
| | fluorophenyl]oxy}propyl dihydrogen phosphate | | | | |
| 14 | 2-amino-3-{[5-chloro-4-(5-{6-chloro-5-[(1- | B | A | nt | F |
| | methylethyl)oxy]pyridin-2-yl}-1,3,4-thiadiazol-2-yl)-2- | | | | |
| | fluorophenyl]oxy}propan-1-ol | | | | |
| 15 | (2R)-2-amino-3-({5-chloro-4-[5-(3-chlorophenyl)-1,3,4- | D | B | D | na |
| | thiadiazol-2-yl]-2-fluorophenyl}oxy)propan-1-ol | | | | |
| 16 | (2R)-2-amino-3-{[5-chloro-4-(5-{3-chloro-4-[(1- | A | A | B | F* |
| | methylethyl)oxy]phenyl}-1,3,4-thiadiazol-2-yl)-2- | | | | |
| | fluorophenyl]oxy}propyl dihydrogen phosphate | | | | |
| 17 | (2R)-2-amino-3-[(5-chloro-4-{5-[3-chloro-4- | B | C | C | na |
| | (propyloxy)phenyl]-1,3,4-thiadiazol-2-yl}-2- | | | | |
| | fluorophenyl)oxy]propan-1-ol | | | | |
| 18 | (2R)-2-amino-3-{[5-chloro-2-fluoro-4-(5-{4-[(1- | A | B | B | na |
| | methylethyl)oxy]-3-(trifluoromethyl)phenyl}-1,3,4-thiadiazol- | | | | |
| | 2-yl)phenyl]oxy}propan-1-ol | | | | |
| 19 | 2-amino-3-{[5-chloro-2-fluoro-4-(5-{6-[(1- | C | A | D | G |
| | methylethyl)oxy]pyridin-3-yl}-1,3,4-thiadiazol-2- | | | | |
| | yl)phenyl]oxy}propan-1-ol | | | | |
| 20 | (2R)-2-amino-3-{[5-chloro-4-(5-{5-chloro-6-[(1- | C | D | D | na |
| | methylethyl)oxy]pyridin-3-yl}-1,3,4-thiadiazol-2-yl)-2- | | | | |
| | fluorophenyl]oxy}propan-1-ol | | | | |
| 21 | (2R)-2-amino-3-{[5-chloro-4-(5-{2-chloro-6-[methyl(1- | C | B | C | na |
| | methylethyl)amino]pyridin-4-yl}-1,3,4-thiadiazol-2-yl)-2- | | | | |
| | fluorophenyl]oxy}propan-1-ol | | | | |
| 22 | (2R)-2-amino-3-{[5-chloro-4-(5-{6-chloro-5-[(1- | B | B | C | na |
| | methylethyl)oxy]pyridin-2-yl}-1,3,4-thiadiazol-2-yl)-2- | | | | |
| | fluorophenyl]oxy}propan-1-ol | | | | |
| 23 | (2R)-2-amino-3-({4-[5-(3-bromo-5-chlorophenyl)-1,3,4- | D | D | D | nt |
| | thiadiazol-2-yl]-5-chloro-2-fluorophenyl}oxy)propan-1-ol | | | | |
| 24 | (2R)-2-amino-3-({4-[5-(3-bromo-5-fluorophenyl)-1,3,4- | D | C | D | na |
| | thiadiazol-2-yl]-5-chloro-2-fluorophenyl}oxy)propan-1-ol | | | | |
| 25 | (2R)-2-amino-3-[(5-chloro-4-{5-[3-chloro-4-(ethyloxy)phenyl]- | B | B | C | na |
| | 1,3,4-thiadiazol-2-yl}-2-fluorophenyl)oxy]propan-1-ol | | | | |
| 26 | (2R)-2-amino-3-{[5-chloro-4-(5-{3,5-dichloro-4-[(1- | C | D | D | nt |
| | methylethyl)oxy]phenyl}-1,3,4-thiadiazol-2-yl)-2- | | | | |
| | fluorophenyl]oxy}propan-1-ol | | | | |
| 27 | (2R)-2-amino-3-{[4-(5-{3-bromo-5-[(1- | C | D | nt | nt |
| | methylethyl)oxy]phenyl}-1,3,4-thiadiazol-2-yl)-5-chloro-2- | | | | |
| | fluorophenyl]oxy}propan-1-ol | | | | |
| 28 | (2R)-2-amino-3-{[4-(5-{3-bromo-5- | C | C | nt | na |
| | [(trifluoromethyl)oxy]phenyl}-1,3,4-thiadiazol-2-yl)-5-chloro- | | | | |
| | 2-fluorophenyl]oxy}propan-1-ol | | | | |
| 29 | (2S)-2-amino-3-{[4-(5-{3-bromo-4-[(1- | A | A | A | F |
| | methylethyl)oxy]phenyl}-1,3,4-thiadiazol-2-yl)-5-chloro-2- | | | | |
| | fluorophenyl]oxy}propyl dihydrogen phosphate | | | | |
| 30 | (2R)-2-amino-3-({5-chloro-4-[5-(3,5-dibromophenyl)-1,3,4- | D | D | E | nt |
| | thiathazol-2-yl]-2-fluorophenyl}oxy)propan-1-ol | | | | |
| 31 | (2S)-2-amino-3-{[5-chloro-4-(5-{2-chloro-6-[methyl(1- | C | A | nt | F |
| | methylethyl)amino]pyridin-4-yl}-1,3,4-thiadiazol-2-yl)-2- | | | | |
| | fluorophenyl]oxy}propan-1-ol | | | | |
| 32 | (2S)-2-amino-3-{[5-chloro-4-(5-{5-chloro-6-[(1- | B | A | D | F |
| | methylethyl)oxy]pyridin-3-yl}-1,3,4-thiadiazol-2-yl)-2- | | | | |
| | fluorophenyl]oxy}propan-1-ol | | | | |
| 33 | (2S)-2-amino-3-{[5-chloro-4-(5-{6-chloro-5-[(1- | B | A | nt | F |
| | methylethyl)oxy]pyridin-2-yl}-1,3,4-thiadiazol-2-yl)-2- | | | | |
| | fluorophenyl]oxy}propan-1-ol | | | | |
| 34 | (2R)-2-amino-3-{[3-chloro-4-(5-{3-chloro-4-[(1- | B | C | nt | na |
| | methylethyl)oxy]phenyl}-1,3,4-thiadiazol-2- | | | | |
| | yl)phenyl]oxy}propan-1-ol | | | | |
| 35 | (2R)-2-amino-3-{[5-chloro-2-fluoro-4-(5-{6-[(1- | D | C | D | na |
| | methylethyl)oxy]pyridin-3-yl}-1,3,4-thiadiazol-2- | | | | |
| | yl)phenyl]oxy}propan-1-ol | | | | |
| 36 | 2-amino-3-{[5-chloro-2-fluoro-4-(5-{6-[methyl(1- | D | A | nt | F |
| | methylethyl)amino]pyridin-3-yl}-1,3,4-thiadiazol-2- | | | | |
| | yl)phenyl]oxy}propan-1-ol | | | | |
| 37 | 5-[5-(4-{[(2R)-2-amino-3-hydroxypropyl]oxy}-2-chloro-5- | A | A | B | na |
| | fluorophenyl)-1,3,4-thiadiazol-2-yl]-2-[(1- | | | | |
| | methylethyl)oxy]benzonitrile | | | | |
| 38 | (2S)-2-amino-3-{[5-chloro-2-fluoro-4-(5-{6-[(1- | B | A | D | F |
| | methylethyl)oxy]pyridin-3-yl}-1,3,4-thiadiazol-2- | | | | |
| | yl)phenyl]oxy}propan-1-ol | | | | |
| 39 | (2R)-2-amino-3-{[2,6-dichloro-4-(5-{3-chloro-4-[(1- | A | C | B | na |
| | methylethyl)oxy]phenyl}-1,3,4-thiadiazol-2- | | | | |
| | yl)phenyl]oxy}propan-1-ol | | | | |
| 40 | (2R)-2-amino-3-{[2-chloro-4-(5-{3-chloro-4-[(1- | A | B | B | na |
| | methylethyl)oxy]phenyl}-1,3,4-thiadiazol-2-yl)-6- | | | | |
| | fluorophenyl]oxy}propan-1-ol | | | | |
| 41 | 2-amino-3-{[5-chloro-2-fluoro-4-(5-{2-[methyl(1- | D | A | na | na |
| | methylethyl)amino]pyridin-4-yl}-1,3,4-thiadiazol-2- | | | | |
| | yl)phenyl]oxy}propan-1-ol | | | | |
| 42 | (2R)-2-amino-3-{[5-chloro-2-fluoro-4-(5-{3-fluoro-4-[(1- | B | C | C | nt |
| | methylethyl)oxy]phenyl}-1,3,4-thiadiazol-2- | | | | |
| | yl)phenyl]oxy}propan-1-ol | | | | |
| 43 | (2R)-2-amino-3-{[5-chloro-4-(5-{3-chloro-4-[(1- | A | C | B | na |
| | methylethyl)amino]phenyl}-1,3,4-thiadiazol-2-yl)-2- | | | | |
| | fluorophenyl]oxy}propan-1-ol | | | | |
| 44 | 2-amino-3-{[5-chloro-2-fluoro-4-(5-{6-[(1- | D | A | D | na |
| | methylethyl)amino]pyridin-3-yl}-1,3,4-thiadiazol-2- | | | | |
| | yl)phenyl]oxy}propan-1-ol | | | | |
| 45 | (2S)-2-amino-3-{[5-chloro-4-(5-{3-chloro-4-[(1- | A | A | B | G* |
| | methylethyl)amino]phenyl}-1,3,4-thiadiazol-2-yl)-2- | | | | |
| | fluorophenyl]oxy}propan-1-ol | | | | |
| 46 | (2R,3S)-3-amino-4-{[5-chloro-2-fluoro-4-(5-{6-[(1- | D | B | D | na |
| | methylethyl)amino]pyridin-3-yl}-1,3,4-thiadiazol-2- | | | | |
| | yl)phenyl]oxy}butan-2-ol | | | | |
| 47 | (2R)-2-amino-3-{[5-chloro-2-fluoro-4-(5-{6-[(1- | A | A | A | F |
| | methylethyl)amino]pyridin-3-yl}-1,3,4-thiadiazol-2- | | | | |
| | yl)phenyl]oxy}propyl dihydrogen phosphate | | | | |
| 48 | (2R,3S)-3-amino-4-{[5-chloro-4-(5-{5-chloro-6-[(1- | B | A | D | na |
| | methylethyl)amino]pyridin-3-yl}-1,3,4-thiadiazol-2-yl)-2- | | | | |
| | fluorophenyl]oxy}butan-2-ol | | | | |
| 49 | 5-[5-(4-{[(2R)-2-amino-3-hydroxypropyl]oxy}-2-chloro-5- | A | A | C | na |
| | fluorophenyl)-1,3,4-thiadiazol-2-yl]-2-(oxetan-3- | | | | |
| | yloxy)benzonitrile | | | | |
| 50 | (1R,2S)-2-amino-3-{[5-chloro-4-(5-{3-cyano-4-[(1- | A | A | A | F |
| | methylethyl)oxy]phenyl}-1,3,4-thiadiazol-2-yl)-2- | | | | |
| | fluorophenyl]oxy}-1-methylpropyl dihydrogen phosphate | | | | |
| 51 | (1R,2S)-2-amino-3-{[5-chloro-4-(5-{3-chloro-4-[(1- | A | A | A | F |
| | methylethyl)oxy]phenyl}-1,3,4-thiadiazol-2-yl)-2- | | | | |
| | fluorophenyl]oxy}-1-methylpropyl dihydrogen phosphate | | | | |
| 52 | (2S)-2-amino-3-{[5-chloro-2-fluoro-4-(5-{6-[(1- | A | A | A | G* |
| | methylethyl)amino]pyridin-3-yl}-1,3,4-thiadiazol-2- | | | | |
| | yl)phenyl]oxy}propyl dihydrogen phosphate | | | | |
| 53 | (1S,2S)-2-amino-3-{[5-chloro-4-(5-{3-cyano-4-[(1- | A | A | A | na |
| | methylethyl)oxy]phenyl}-1,3,4-thiadiazol-2-yl)-2- | | | | |
| | fluorophenyl]oxy}-1-methylpropyl dihydrogen phosphate | | | | |
| 54 | (1S,2S)-2-amino-3-{[5-chloro-2-fluoro-4-(5-{6-[(1- | A | A | B | G |
| | methylethyl)amino]pyridin-3-yl}-1,3,4-thiadiazol-2- | | | | |
| | yl)phenyl]oxy}-1-methylpropyl dihydrogen phosphate | | | | |
| 55 | 5-[5-(4-{[(2R,3S)-2-amino-3-hydroxybutyl]oxy}-2-chloro-5- | A | A | A | F |
| | fluorophenyl)-1,3,4-thiadiazol-2-yl]-2-[(1- | | | | |
| | methylethyl)oxy]benzonitrile | | | | |
| 56 | (2S,3S)-3-amino-4-{[5-chloro-2-fluoro-4-(5-{6-[(1- | D | D | na | na |
| | methylethyl)amino]pyridin-3-yl}-1,3,4-thiadiazol-2- | | | | |
| | yl)phenyl]oxy}butan-2-ol | | | | |
| 57 | (2R,3S)-3-amino-4-{[5-chloro-2-fluoro-4-(5-{2-[(1- | C | C | D | na |
| | methylethyl)amino]pyridin-4-yl}-1,3,4-thiadiazol-2- | | | | |
| | yl)phenyl]oxy}butan-2-ol | | | | |
| 58 | (2S,3S)-3-amino-4-{[5-chloro-2-fluoro-4-(5-{2-[(1- | C | D | D | nt |
| | methylethyl)amino]pyridin-4-yl}-1,3,4-thiadiazol-2- | | | | |
| | yl)phenyl]oxy}butan-2-ol | | | | |
| 59 | (2S,3R)-3-amino-4-{[5-chloro-2-fluoro-4-(5-{6-[(1- | D | A | D | na |
| | methylethyl)amino]pyridin-3-yl}-1,3,4-thiadiazol-2- | | | | |
| | yl)phenyl]oxy}butan-2-ol | | | | |
| 60 | (2R,3S)-3-amino-4-{[5-chloro-2-fluoro-4-(5-{3-methyl-4-[(1- | A | B | B | na |
| | methylethyl)oxy]phenyl}-1,3,4-thiadiazol-2- | | | | |
| | yl)phenyl]oxy}butan-2-ol | | | | |
| 61 | (2S,3S)-3-amino-4-{[5-chloro-2-fluoro-4-(5-{3-methyl-4-[(1- | A | C | B | na |
| | methylethyl)oxy]phenyl}-1,3,4-thiadiazol-2- | | | | |
| | yl)phenyl]oxy}butan-2-ol | | | | |
| 62 | (2R,3S)-3-amino-4-{[5-chloro-2-fluoro-4-(5-{2-methyl-6-[(1- | A | A | B | na |
| | methylethyl)amino]pyridin-4-yl}-1,3,4-thiadiazol-2- | | | | |
| | yl)phenyl]oxy}butan-2-ol | | | | |
| 63 | (2S,3S)-3-amino-4-{[5-chloro-2-fluoro-4-(5-{2-methyl-6-[(1- | A | B | B | G |
| | methylethyl)amino]pyridin-4-yl}-1,3,4-thiadiazol-2- | | | | |
| | yl)phenyl]oxy}butan-2-ol | | | | |
| 64 | (1S,2R)-2-amino-3-{[5-chloro-4-(5-{3-chloro-4-[(1- | A | A | A | F* |
| | methylethyl)oxy]phenyl}-1,3,4-thiadiazol-2-yl)-2- | | | | |
| | fluorophenyl]oxy}-1-methylpropyl dihydrogen phosphate | | | | |
| 65 | (2R,3S)-3-amino-4-{[5-chloro-2-fluoro-4-(5-{5-methyl-6-[(1- | C | B | E | na |
| | methylethyl)amino]pyridin-3-yl}-1,3,4-thiadiazol-2- | | | | |
| | yl)phenyl]oxy}butan-2-ol | | | | |
| 66 | (2S,3S)-3-amino-4-{[5-chloro-2-fluoro-4-(5-{5-methyl-6-[(1- | nt | D* | nt | nt |
| | methylethyl)amino]pyridin-3-yl}-1,3,4-thiadiazol-2- | | | | |
| | yl)phenyl]oxy}butan-2-ol | | | | |
| 67 | (2R,3R)-3-amino-4-{[5-chloro-2-fluoro-4-(5-{5-methyl-6-[(1- | B | A | D | na |
| | methylethyl)amino]pyridin-3-yl}-1,3,4-thiadiazol-2- | | | | |
| | yl)phenyl]oxy}butan-2-ol | | | | |
| 68 | (2R,3R)-3-amino-4-{[5-chloro-2-fluoro-4-(5-{6-[(1- | C | A | D | na |
| | methylethyl)amino]pyridin-3-yl}-1,3,4-thiadiazol-2- | | | | |
| | yl)phenyl]oxy}butan-2-ol | | | | |
| 69 | (1S,2S)-2-amino-3-{[5-chloro-4-(5-{3-chloro-4-[(1- | A | A | B | G* |
| | methylethyl)oxy]phenyl}-1,3,4-thiadiazol-2-yl)-2- | | | | |
| | fluorophenyl]oxy}-1-methylpropyl dihydrogen phosphate | | | | |
| 70 | (1R,2R)-2-amino-3-{[5-chloro-4-(5-{3-chloro-4-[(1- | A | A | A | F |
| | methylethyl)oxy]phenyl}-1,3,4-thiadiazol-2-yl)-2- | | | | |
| | fluorophenyl]oxy}-1-methylpropyl dihydrogen phosphate | | | | |
| 71 | (2S,3R)-3-amino-4-{[5-chloro-4-(5-{3-chloro-4-[(1- | A | A* | B | F |
| | methylethyl)oxy]phenyl}-1,3,4-thiadiazol-2-yl)-2- | | | | |
| | fluorophenyl]oxy}butan-2-ol | | | | |
| 72 | (2S,3S)-3-amino-4-{[5-chloro-4-(5-{3-chloro-4-[(1- | B | A | C | na |
| | methylethyl)oxy]phenyl}-1,3,4-thiadiazol-2-yl)-2- | | | | |
| | fluorophenyl]oxy}butan-2-ol | | | | |
| 73 | (2R,3S)-3-amino-4-{[5-chloro-4-(5-{3-chloro-4-[(1- | A | A | B | na |
| | methylethyl)oxy]phenyl}-1,3,4-thiadiazol-2-yl)-2- | | | | |
| | fluorophenyl]oxy}butan-2-ol | | | | |
| 74 | (2R,3R)-3-amino-4-{[5-chloro-4-(5-{3-chloro-4-[(1- | A | A | B | G |
| | methylethyl)oxy]phenyl}-1,3,4-thiadiazol-2-yl)-2- | | | | |
| | fluorophenyl]oxy}butan-2-ol | | | | |
| 75 | (2R)-2-amino-3-{[5-chloro-2-fluoro-4-(5-{6-[(1- | D | B | D | na |
| | methylethyl)amino]pyridin-3-yl}-1,3,4-thiadiazol-2- | | | | |
| | yl)phenyl]oxy}propan-1-ol | | | | |
| 76 | 5-[5-(4-{[(2R)-2-amino-3-hydroxypropyl]oxy}-2-chloro-5- | A | B | C | na |
| | fluorophenyl)-1,3,4-thiadiazol-2-yl]-2-(ethyloxy)benzonitrile | | | | |
| 77 | (2S)-2-amino-3-{[5-chloro-2-fluoro-4-(5-{6-[(1- | D | A | D | na |
| | methylethyl)amino]pyridin-3-yl}-1,3,4-thiadiazol-2- | | | | |
| | yl)phenyl]oxy}propan-1-ol | | | | |
| 78 | 5-[5-(4-{[(2R)-2-amino-3-hydroxypropyl]oxy}-2-chloro-5- | C | B | D | na |
| | fluorophenyl)-1,3,4-thiadiazol-2-yl]-2-{[2- | | | | |
| | (methyloxy)ethyl]oxy}benzonitrile | | | | |
| 79 | (2S)-2-amino-3-{[5-chloro-4-(5-{3-cyano-4-[(1- | A | A | A | F |
| | methylethyl)oxy]phenyl}-1,3,4-thiadiazol-2-yl)-2- | | | | |
| | fluorophenyl]oxy}propyl dihydrogen phosphate | | | | |
| 80 | 5-[5-(4-{[(2R)-2-amino-3-hydroxypropyl]oxy}-2-chloro-5- | D | D* | D | na |
| | fluorophenyl)-1,3,4-thiadiazol-2-yl]-2-[(1- | | | | |
| | methylethyl)oxy]benzamide | | | | |
| 81 | (2R)-2-amino-3-{[5-chloro-4-(5-{5-chloro-6-[(1- | B | B | D | na |
| | methylethyl)amino]pyridin-3-yl}-1,3,4-thiadiazol-2-yl)-2- | | | | |
| | fluorophenyl]oxy}propan-1-ol | | | | |
| 82 | 5-[5-(4-{[(2S,3R)-2-amino-3-hydroxybutyl]oxy}-2-chloro-5- | A | A | B | na |
| | fluorophenyl)-1,3,4-thiadiazol-2-yl]-2-[(1- | | | | |
| | methylethyl)oxy]benzonitrile | | | | |
| 83 | 5-[5-(4-{[(2S,3S)-2-amino-3-hydroxybutyl]oxy}-2-chloro-5- | A | A | B | na |
| | fluorophenyl)-1,3,4-thiadiazol-2-yl]-2-[(1- | | | | |
| | methylethyl)oxy]benzonitrile | | | | |
| 84 | (2R)-2-amino-3-[(5-chloro-2-fluoro-4-{5-[4-phenyl-5- | nt | D | nt | nt |
| | (trifluoromethyl)-2-thienyl]-1,3,4-thiadiazol-2- | | | | |
| | yl}phenyl)oxy]propan-1-ol | | | | |
| 85 | (1R,2S)-2-amino-3-{[5-chloro-2-fluoro-4-(5-{6-[(1- | A | A | B | na |
| | methylethyl)amino]pyridin-3-yl}-1,3,4-thiadiazol-2- | | | | |
| | yl)phenyl]oxy}-1-methylpropyl dihydrogen phosphate | | | | |
| 86 | 5-[5-(4-{[(2R,3R)-2-amino-3-hydroxybutyl]oxy}-2-chloro-5- | A | A | B | G* |
| | fluorophenyl)-1,3,4-thiadiazol-2-yl]-2-[(1- | | | | |
| | methylethyl)oxy]benzonitrile | | | | |
| 87 | (2R)-2-amino-3-(5-chloro-4-{5-[2-chloro-6-(oxetan-3- | nt | nt | nt | nt |
| | yloxy)pyridin-4-yl]-1,3,4-thiadiazol-2-yl}-2- | | | | |
| | fluorophenoxy)propan-1-ol | | | | |
| 88 | (2R)-2-amino-3-(5-chloro-4-{5-[3-chloro-4-(oxetan-3- | nt | nt | nt | nt |
| | yloxy)phenyl]-1,3,4-thiadiazol-2-yl}-2-fluorophenoxy)propan- | | | | |
| | 1-ol | | | | |
| 89 | (2R)-2-amino-3-(5-chloro-2-fluoro-4-{5-[2-(propan-2- | C | B | D | na |
| | ylamino)pyrimidin-5-yl]-1,3,4-thiadiazol-2-yl}phenoxy)propan- | | | | |
| | 1-ol | | | | |
| 90 | (2S)-2-amino-3-(5-chloro-2-fluoro-4-{5-[2-(propan-2- | C | A* | D | F |
| | ylamino)pyrimidin-5-yl]-1,3,4-thiadiazol-2-yl}phenoxy)propan- | | | | |
| | 1-ol | | | | |
| 91 | (2R)-2-amino-3-[(5-chloro-2-fluoro-4-{5-[4- | C | D | D | nt |
| | (propyloxy)phenyl]-1,3,4-thiadiazol-2-yl}phenyl)oxy]propan-1- | | | | |
| | ol | | | | |
| 92 | (2R)-2-amino-3-({5-chloro-4-[5-(3,5-dichlorophenyl)-1,3,4- | D | D | nt | na |
| | thiadiazol-2-yl]-2-fluorophenyl}oxy)propan-1-ol | | | | |
| 93 | (1S,2R)-2-amino-3-{[5-chloro-2-fluoro-4-(5-{6-[(1- | A | A* | B | G |
| | methylethyl)amino]pyridin-3-yl}-1,3,4-thiadiazol-2- | | | | |
| | yl)phenyl]oxy}-1-methylpropyl dihydrogen phosphate | | | | |
| 94 | (1R,2S)-2-amino-3-{[5-chloro-2-fluoro-4-(5-{2-methyl-6-[(1- | A | A* | B | F |
| | methylethyl)amino]pyridin-4-yl}-1,3,4-thiadiazol-2- | | | | |
| | yl)phenyl]oxy}-1-methylpropyl dihydrogen phosphate | | | | |
| 95 | (1S,2S)-2-amino-3-{[5-chloro-2-fluoro-4-(5-{2-methyl-6-[(1- | A | A | C | G |
| | methylethyl)amino]pyridin-4-yl}-1,3,4-thiadiazol-2- | | | | |
| | yl)phenyl]oxy}-1-methylpropyl dihydrogen phosphate | | | | |
| 96 | (2S,3R)-3-amino-4-{[5-chloro-2-fluoro-4-(5-{5-methyl-6-[(1- | D | A | E | na |
| | methylethyl)amino]pyridin-3-yl}-1,3,4-thiadiazol-2- | | | | |
| | yl)phenyl]oxy}butan-2-ol | | | | |
| 97 | (2R,3R)-3-amino-4-{[5-chloro-2-fluoro-4-(5-{2-methyl-6-[(1- | A | A | C | G |
| | methylethyl)amino]pyridin-4-yl}-1,3,4-thiadiazol-2- | | | | |
| | yl)phenyl]oxy}butan-2-ol | | | | |
| 98 | (2S,3R)-3-amino-4-{[5-chloro-2-fluoro-4-(5-{2-methyl-6-[(1- | A | A | C | F |
| | methylethyl)amino]pyridin-4-yl}-1,3,4-thiadiazol-2- | | | | |
| | yl)phenyl]oxy}butan-2-ol | | | | |
| 99 | (1R,2R)-2-amino-3-{[5-chloro-2-fluoro-4-(5-{2-methyl-6-[(1- | A | A | B | F |
| | methylethyl)amino]pyridin-4-yl}-1,3,4-thiadiazol-2- | | | | |
| | yl)phenyl]oxy}-1-methylpropyl dihydrogen phosphate | | | | |
| 100 | (1S,2R)-2-amino-3-{[5-chloro-2-fluoro-4-(5-{2-methyl-6-[(1- | A | A | B | nt |
| | methylethyl)amino]pyridin-4-yl}-1,3,4-thiadiazol-2- | | | | |
| | yl)phenyl]oxy}-1-methylpropyl dihydrogen phosphate | | | | |
| 101 | (1R,2R)-2-amino-3-{[5-chloro-2-fluoro-4-(5-{6-[(1- | A | A | A | G |
| | methylethyl)amino]pyridin-3-yl}-1,3,4-thiadiazol-2- | | | | |
| | yl)phenyl]oxy}-1-methylpropyl dihydrogen phosphate | | | | |
| 102 | (1R,2S)-2-amino-3-{[5-chloro-2-fluoro-4-(5-{3-methyl-4-[(1- | A | A* | A | F |
| | methylethyl)oxy]phenyl}-1,3,4-thiadiazol-2-yl)phenyl]oxy}-1- | | | | |
| | methylpropyl dihydrogen phosphate | | | | |
| 103 | (2R,3S)-3-amino-4-{[5-chloro-2-fluoro-4-(5-{6-methyl-5-[(1- | B | B | B | G |
| | methylethyl)amino]pyridin-2-yl}-1,3,4-thiadiazol-2- | | | | |
| | yl)phenyl]oxy}butan-2-ol | | | | |
| 104 | (1S,2S)-2-amino-3-{[5-chloro-2-fluoro-4-(5-{3-methyl-4-[(1- | nt | A* | A | G |
| | methylethyl)oxy]phenyl}-1,3,4-thiadiazol-2-yl)phenyl]oxy}-1- | | | | |
| | methylpropyl dihydrogen phosphate | | | | |
| 105 | (2S,3S)-3-amino-4-(5-chloro-2-fluoro-4-{5-[6-methyl-5- | nt | C | C | G |
| | (propan-2-ylamino)pyridin-2-yl]-1,3,4-thiadiazol-2- | | | | |
| | yl}phenoxy)butan-2-ol | | | | |
| 106 | (2S,3S)-3-amino-4-(5-chloro-2-fluoro-4-{5-[6-methyl-4- | nt | C | C | G |
| | (propan-2-ylamino)pyridin-2-yl]-1,3,4-thiadiazol-2- | | | | |
| | yl}phenoxy)butan-2-ol | | | | |
| 107 | 4-[5-(4-{[(2R)-2-amino-3-hydroxypropyl]oxy}-2-chloro-5- | nt | nt | nt | nt |
| | fluorophenyl)-1,3,4-thiadiazol-2-yl]-6-(oxetan-3-yloxy)pyridin- | | | | |
| | 2-ol | | | | |

### Example 7

### Lymphocyte PD Assay in Mice

Suppression of circulating lymphocytes was assessed as a pharmacodynamic (PD) endpoint in 6-10 week old C57B1/6 male mice (Taconic Farms, Germantown, N.Y.). Upon arrival, mice were acclimated to the vivarium (12 h light cycle, 12 h dark cycle) for a minimum of 3 days prior to the initiation of a study. During the study, animals were provided food and water ad libitum and housed in a room conditioned at 70-75° F. All animals were examined daily for health assessment.

Compounds of the Invention were suspended or dissolved in vehicle for administration of 0.1 mL/animal based on mean body weight of group. Compounds were administered using a disposable gavage needle (20G, Braintree Scientific, Braintree, Mass.). Blood was collected into EDTA-coated tubes (Microvette 100 with EDTA, Sarstedt, Newton, N.C.) from the retro-orbital sinus of isoflurane-anesthetized animals 24 or 32 h post-dose. Mice were then euthanized by cervical dislocation. For some experiments, samples of tissues were collected for measurement of compound levels. Samples were frozen immediately on dry ice and stored at −18° C. until assayed. After collection, blood samples were placed on a rock 'n roller mixer (Drew Scientific, Inc., Waterbury, Conn.) for at least 10 min and then a complete blood count (CBC) analysis was performed using the Hemavet 1700 Flexible Veterinary Multi-Species Hematology System (Drew Scientific). Samples were then placed on ice and, within 4 h, centrifuged to obtain plasma which was then stored frozen at 20° C. until analysis for compound levels. CBC readouts included white blood cells; total and % of total for the following: neutrophils, lymphocytes, monocytes, eosinophils, basophils, and nucleated red blood cells (RBC); RBC; hemoglobin; hematocrit; mean corpuscular volume, hemoglobin (HGB) total and concentration; RBC distribution width; platelets; and mean platelet volume. All mouse PD data consists of group sizes of 8 and are expressed as means±SEM. Statistical analysis of each experimental endpoint was conducted with GraphPad Prism. All compound exposure data are based on n=4/group and are expressed as means±SD.

### Example 8

### Lymphocyte PD Assay in Rats

Suppression of circulating lymphocytes was assessed as a PD endpoint in jugular vein canulated Sprague Dawely (SD) rats 6-8 weeks of age and weighing approximately 200 g (Taconic Farms, Germantown, N.Y.). Prior initiating a study, rats were acclimated to the vivarium facility (12 h light cycle, 12 h dark cycle) for a minimum of 2 days. During a study, animals were provided food and water ad libitum and housed in a room conditioned at 70-75° F. and 60% relative humidity. All animals were examined daily for health assessment.

The Compound of the Invention were formulated and animals was dosed at a volume of 4 mL/kg. At indicated timepoints, whole blood was collected via jugular vein canulas into EDTA-coated tubes and hematology analysis was performed on an Abbott Cell-Dyn 3700 hematology analyzer. Readouts included white blood cells (total, differential, and % of total), neutrophils, lymphocytes, monocytes, eosinophils, basophils, RBC, HGB, hematocrit, mean corpuscule volume, mean corpuscule HGB concentration, RBC distribution width, platelets, and mean platelet volume.

As an example, Table 3 shows the effect on lymphocyte counts 24 hours after oral administration of a single dose at 3 mg/kg of Compounds of the Invention to Female CD rats (Sprague Dawley) as compared to a group of animals treated with vechicle only. This demonstrates that the compounds have a surprisingly long duration of action.

TABLE 3

| Table 1 Cmpd No. | Lymphocyte Counts (p < 0.05) |
|---|---|
| 1 | −80% |
| 2 | −93% |
| 37 | −93% |
| 44 | −94% |
| 62 | −79% |
| 72 | −86% |
| 73 | −89% |
| 77 | −94% |

### Biological Examples 9-12

### In Vivo Models

### Example 9

### Delayed-Type Hypersensitivity (DTH) Model

Blood lymphocyte numbers, essential for the development of efficient immune responses, are maintained by recirculation through secondary lymphoid organs. Signaling of S1P through S1P1 has been shown to exclusively modulate egress of lymphocyte including 70% of activated T cells from lymph nodes. Delayed-type hypersensitivity (DTH) is an immune response mediated by a variety of inflammatory cells, including neutrophils, macrophages and T cells (Kobayashi et al. 2001, Black 1999). DTH develops in two phases, a sensitization phase, in which T cells are sensitized and memory T cells are formed, and an elicitation phase, in which T cell recall responses are induced upon secondary challenge with antigen. This second phase results in recruitment of inflammatory cells such as neutrophils and macrophages to the injection site of an intradermally applied antigen in a previously sensitized host, which causes swelling 24 h to 48 h post antigen challenge. The DTH assay (primarily done in mice) is an in vivo manifestation of a cell-mediated immunity reaction, and the response to antigen representation modulated by immunosuppressive treatment can be measured.

C57B1/6 male mice (10 mice per group) are immunized on day zero by subcutaneous injection at the base of the tail with 100 μL of 2 mg/mL methylated BSA emulsified with Complete Freunds Adjuvans (CFA, Sigma). Once-daily for twice-daily administration of a Compound of the Invention occurrs for 10 days. On day 10 after immunization, mice receive a second booster injection at the base of tail of an emulsified mixture of 2 mg/mL methylated BSA in Incomplete Freund's Adjuvans. On day 13 animals are challenged subcutaneously in the left hind footpad with 20 μL of 10 mg/mL methylated BSA in sterile water (water for injection). Animals are injected with an equal volume of sterile water into the right hind footpad as a control. Twenty four hours later (dose day 14) the right and left hind foot paws are transected at the medial and lateral malleolus, weighed, and the weight difference induced by injected antigen determined and compared to weight differences of vehicle treated non-sensitized and sensitized control groups. The increase in paw weights comparing left and right hind paw for each treatment group are analyzed for differences of treatment with a Compound of the Invention compared to vehicle control group using the Mann-Whitney non-parametric test statistic with minimal significance level set at p<0.05.

### Example 10

### Allograft Model

The rodent allograft model is an in vivo assay for assessing tissue rejection (ie, from transplantation) in response to chronic and/or sub-chronic immunosuppressive treatment (Chiba et al, 2005). Rejection is caused by T lymphocytes of the recipient responding to the foreign major histocompatibility complex of the donor graft. The transplanted organ (eg, skin) represents a continuous source of HLA alloantigens capable of inducing a rejection response at any time post transplantation. Because it cannot be eliminated, the allograft continuously activates the immune system, resulting in lifelong overproduction of cytokines, constant cytotoxic activity, and sustained alteration in the graft vasculature. Therefore, lifelong immunosuppression is required to ensure allograft survival. In this model skin from donor rats (male Lewis; histocompatibility RT-1¹) is surgically engrafted onto a dorsal region of recipient rats (male F344; histocompatibility RT-1^{1v1}). Administration of compound occurs immediately after surgery for a predetermined duration. Skin allografts are monitored daily for rejection.

On the day of surgery male Lewis donor rats are anesthetized with Isoflurane and skin aseptically harvested from the tail. Male F344 acceptor rats (8 per group) previously shaved (1-2 days prior to surgery) in the designated engraftment area are anesthetized with Isoflurane and a full thickness skin graft bed on the medial flank removed and discarded. The skin graft bed removed is equivalent in size to the donor skin to be engrafted. The prepared donor skin is then secured on the prepared graft bed with spot tissue glue or by 4 to 8 nonsilk sutures, and covered with sterile Vaseline gauze and wrapped with a bandage. All surgery takes place on heated pads with sterile surgical equipment. Animals are monitored and turned every 20 minutes until ambulatory before returning to cages, water and food. Initiation of administration of a Compound of the Invention (once-daily or twice-daily) occurrs when the animals fully recovered from anesthesia for a period of 14 days. On day 5 post-surgery, surgical bandaging is removed and the grafts assessed daily for rejection (necrosis of the graft tissue following by scabbing and sloughing from the graft bed site). An allograft is scored as “rejected” when it sloughed from the graft bed site. A positive effect in this model is delayed rejection of the allograft in response to treatment with a Compound of the Invention when compared to vehicle-treated control animals.

### Example 11

### Experimental Autoimmune Encephalomyelitis (EAE) Model

Multiple sclerosis is a demyelinating disease of the CNS. The main features of the disease are focal areas of demyelination and inflammation mediated by macrophages and t-lymphocytes. These cells develop in the peripheral lymphoid organs and travel to the CNS causing an autoimmune response. The development of T cells is controlled largely by the expression of various cytokines as well as cellular adhesion molecules. The EAE model today is the most thoroughly studied animal model for human autoimmune diseases. Mice are immunized with myelin-derived peptide PLP and clinical parameters of disease (bodyweight loss and paralysis) are monitored daily. The endpoint is the analysis of the extent of inflammation in brain and spinal cord.

C57B1/6 mice develop chronic paralysis after immunization with MOG₃₅₋₅₅ peptide. Mice develop EAE 8-14 days after immunization and stay chronically paralyzed for 30-40 days after onset of disease. Female C57B1/6 mice are subcutaneously injected with MOG₃₅₋₅₅ peptide emulsified in Complete Freund's Adjuvant at two sites on the back, injecting 0.1 mL at each site. Within 2 h of injection, pertussis toxin (aids in brain penetration of the MOG peptide) is administered intraperitoneally. A second injection of pertussis toxin is administered 22-26 h after the MOG₃₅₋₅₅ peptide injection. Onset of EAE is typically 7 days after immunization. EAE is scored on a scale of 0-5 with 0 being no obvious changes in motor functions, while 5 indicates complete paralysis. Mice are administered a Compound of the Invention (once-daily or twice-daily) on the day of MOG₃₅₋₅₅ peptide injection and monitored for paralysis and compared to vehicle-treated control animals. A positive effect in this model is delayed onset/severity of EAE.

### Example 12

### Osteoporosis Model

Methods described in Nature 2009, 458(7237), 524-528, which is herein incorporated by reference, can be used to determine whether a Compound of the Invention is able to prevent bone density loss.

The foregoing invention has been described in some detail by way of illustration and example, for purposes of clarity and understanding. The invention has been described with reference to various specific embodiments and techniques. However, it should be understood that many variations and modifications may be made while remaining within the spirit and scope of the invention. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled. All patents, patent applications and publications cited in this application are hereby incorporated by reference in their entirety for all purposes to the same extent as if each individual patent, patent application or publication were so individually denoted.

1. A compound of Formula I: 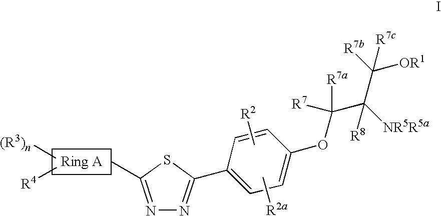
or a stereoisomer or mixture of isomers thereof and optionally as a pharmaceutically acceptable salt thereof, where
R¹ is hydrogen or —P(O)(OR⁶)₂;
R² and R^{2a} are independently hydrogen, cyano, halo, alkyl, haloalkyl, alkoxy, or haloalkoxy;
Ring A is phenyl, 5-membered heteroaryl, 6-membered heteroaryl, or imidazo[2,1-b]thiazolyl;
each R³ is independently cyano, halo, alkyl, haloalkyl, hydroxy, alkoxy, haloalkoxy, alkoxyalkyloxy, cycloalkyloxy, heterocycloalkyloxy, nitro, phenyl, amino, alkylamino, dialkylamino, aminocarbonyl, alkylcarbonylamino, or alkoxycarbonylamino;
n is 0, 1, or 2;
R⁴ is hydrogen, alkyl, cyano, halo, or haloalkyl;
R⁵ is hydrogen or alkyl;
R^{5a} is hydrogen, alkyl, alkylcarbonyl, or alkoxycarbonyl;
each R⁶ is independently hydrogen or alkyl;
R⁷, R^{7a}, R^{7b}, and R^{7c} are independently hydrogen or alkyl; and
R⁸ is hydrogen, alkyl, or hydroxyalkyl.

2. The Compound of claim 1 where R⁷, R^{7a}, R^{7b}, and R⁸ are hydrogen, or a stereoisomer or mixture of isomers thereof and optionally as a pharmaceutically acceptable salt thereof.

3. The Compound of claim 2 where Ring A is phenyl, thienyl, pyridyl, pyrimidinyl, or imidazo[2,1-b]thiazolyl, each of which is substituted with (R³)ₙ and R⁴, and n is 1 or 2; or a stereoisomer or mixture of isomers thereof and optionally as a pharmaceutically acceptable salt thereof.

4. The Compound of claim 2 where Ring A is phenyl or pyridyl, each of which is substituted with (R³)ₙ and R⁴, and n is 1 or 2; or a stereoisomer or mixture of isomers thereof and optionally as a pharmaceutically acceptable salt thereof.

5. The Compound of claim 4 where R² and R^{2a} are halo, or a stereoisomer or mixture of isomers thereof and optionally as a pharmaceutically acceptable salt thereof.

6. The Compound of claim 5 where R⁵ is hydrogen and R^{5a} is hydrogen or alkoxycarbonyl, or a stereoisomer or mixture of isomers thereof and optionally as a pharmaceutically acceptable salt thereof.

7. The Compound of claim 6 where R^{7c} is hydrogen, or a stereoisomer or mixture of isomers thereof and optionally as a pharmaceutically acceptable salt thereof.

8. The Compound of claim 6 where R^{7c} is methyl, or a stereoisomer or mixture of isomers thereof and optionally as a pharmaceutically acceptable salt thereof.

9. The Compound of claim 7 where R¹ is hydrogen, or a stereoisomer or mixture of isomers thereof and optionally as a pharmaceutically acceptable salt thereof.

10. The Compound of claim 7 where R¹ is —P(O)(OR⁶)₂ and both R⁶ are hydrogen or both R⁶ are tert-butyl, or a stereoisomer or mixture of isomers thereof and optionally as a pharmaceutically acceptable salt thereof.

11. The Compound of claim 9 where n is 1 or 2, or a stereoisomer or mixture of isomers thereof and optionally as a pharmaceutically acceptable salt thereof.

12. (canceled)

13. The Compound of claim 2 where Ring A is pyridinyl; R⁴ is hydrogen; and n is 1 and R³ is alkoxy or alkylamino; or n is 2 and one R³ is alkyl and the second R³ is alkylamino; or n is 2 and one R³ is alkoxy or dialkylamino and the second R³ is halo; or a stereoisomer or mixture of isomers thereof and optionally as a pharmaceutically acceptable salt thereof.

14. The Compound of claim 2 where Ring A is phenyl, R⁴ is hydrogen, n is 2, one R³ is halo, cyano, alkyl, or haloalkyl and the second R³ is alkoxy or alkylamino, or a stereoisomer or mixture of isomers thereof and optionally as a pharmaceutically acceptable salt thereof.

15. The Compound of claim 2 where Ring A is phenyl; R⁴ is hydrogen; n is 2; and one R³ is halo and the second R³ is alkoxy; or one R³ is cyano and the second R³ is alkoxy; or one R³ is alkyl and the second R³ is alkoxy; or one R³ is haloalkyl and the second R³ is alkoxy; or R³ is one halo and the second R³ is alkylamino; or a stereoisomer or mixture of isomers thereof and optionally as a pharmaceutically acceptable salt thereof.

16. The Compound of claim 1 optionally as a pharmaceutically acceptable salt thereof, selected from compounds with the following numbers, as numbered in Table 1,
```
  1
  2
  3
  4
  5
  6
  7
  8
  9
10
11
12
13
14
15
16
17
18
19
20
21
22
24
25
26
27
28
29
30
31
32
33
34
35
36
37
38
39
40
41
42
43
44
45
46
47
48
49
50
51
52
53
54
55
56
57
58
59
60
61
62
63
64
65
66
67
68
69
70
71
72
73
74
75
76
77
78
79
80
81
82
83
84
85
86
87
88
89
90
91
92
93
94
95
96
97
98
99
100
101
102
103
104
105
and
106.
```

17. A pharmaceutical composition which comprises a compound of claim 1 or a single stereoisomer or a mixture of isomers thereof, additionally optionally as a pharmaceutically acceptable salt thereof, and a pharmaceutically acceptable carrier, excipient, or diluent.

18. A method for treating a disease, disorder, or syndrome which method comprises administering to a patient a therapeutically effective amount of a compound, or a single stereoisomer or a mixture of isomers thereof, of claim 1 optionally as a pharmaceutically acceptable salt thereof, or a pharmaceutical composition according to claim 17.

19. The method of claim 18 where the disease is an autoimmune disease.

20. The method of claim 19 where the autoimmune disease is mutiple schlerosis, psoriasis, inflammatory bowel disease, graft-versus-host disease, autoimmune-induced inflammation, or rheumatoid arthritis.

21. The method of claim 18 where the disease is osteoporosis.

22. The Compound of claim 8 where R¹ is hydrogen, or a stereoisomer or mixture of isomers thereof and optionally as a pharmaceutically acceptable salt thereof.

23. The Compound of claim 8 where R¹ is —P(O)(OR⁶)₂ and both R⁶ are hydrogen or both R⁶ are tert-butyl, or a stereoisomer or mixture of isomers thereof and optionally as a pharmaceutically acceptable salt thereof.

24. The Compound of claim 22 where n is 1 or 2, or a stereoisomer or mixture of isomers thereof and optionally as a pharmaceutically acceptable salt thereof.

25. The Compound of claim 10 where n is 1 or 2, or a stereoisomer or mixture of isomers thereof and optionally as a pharmaceutically acceptable salt thereof.

26. The Compound of claim 23 where n is 1 or 2, or a stereoisomer or mixture of isomers thereof and optionally as a pharmaceutically acceptable salt thereof.
